SEQUENCE LISTING (38 sequences)

SEQ ID NO: 1 - DNA, 11916 bp, Artificial Sequence
  Description of Artificial Sequence Synthetic plasmid
taccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccaaatagtaggaattacccacctgtacaagtgctgaaaacttggatgaataagcccgtttgccattttctactgctattttaaatcttttctgtatttgtctgcatttgtctttacccttgaataaatgttttagttgttttttttcaattatgactgtgtttaacagacattattacaataatatgtaatagtagtacacactattattatgtaatagtacttgttgactgtatttgagaactggaccgagtgagtgttacgtcacccattcaaaatgacttacttctggctccaacaaaatgaagttaattcagttgccatttttcactgtatggacatcgccgtgttggagctagacgtcgccaataagcaagaaagagccgagatgcgtcgagtctgagtcaccgttcctatggcaacccctctaaccaatcagaagtaagcttgttggaagtccacagcctaccacttgaaagcgggctgcacaaaatctgtcaaacgttttgaacgttggatgtgagagcacatacttttattaaggcatctggttggtcagtttataatatcaacaacttgggctacagaaaagaaaagttattacagaaattatgattaacaagtacatgttaaataaagattttaatatgaatgccaccactggagcattcatgccatttggagcttcttcctgtttggatcactagaaggaggaggtcactcattacagttctcatatacagtcgttggttggttggttggttggttggttggtagattgattgattgattgattgattgattgattgattgattgattgattgattgattgattgattgattgattgattgattgattgattgattgattgattgattggtagtcaaaataagaaataatttccacagattcattacagaaatgattaaatgcatacataaaaaactggggggggggggatacaacaacacacttaagtcacatttgcctacgtaaagaaaagtaaagaaaatcaatagctatattttacatctcctttttttgctgtcttttaattagccttgttttgctgttatcttgattgaaactgtaacttcttcacctgcttcttttctttgtaggttttgccagcccagaaacaggcatggctgtgcaaggccagagcaccatgcacaatgcgctgcatgtcttcatgaacggctcaatgtcctcagtccagggctcagccaacgaccccatcttcctccttcaccatgctttcattgacaggtaacaaacacgtcatgacattagactgcacagtttttgacaaagttcatacaatctgttgtttatagctgctacaattagtgaagtttgtgaatgtacttggatgagcagcgaaagatcaattgagatcaattgttagagtttggttgccctgcagagcaaagaacaaaaaataatctggtggctttactgcgtgaggttattattggtggaatagaaacacaaaacataattgcatttatttgtttaattttttatcttatcttaactttcatcttgcatatttgtttcttacatcatttctagcatctttgagcgctggctaagaactcatcagcctccccggtccatctacccacgtaccaatgcaccaattggccacaatgacggctactacatggtgccattccttcctctttataggaatggagactacctcctgtccaacaaggctcttggatacgagtacgcctacctgttggacccaggtcattgcacaacaccagaaatgccctctgatctgcaaaagacgtgaatatctgttcagacacccatatccactctgttccacacaggtcagaggtttgtccaggagttcttgacagaggtgtaaaaagtactcaaaaattttactcaagtgaaagtacaagtacttagggaaaattttactcaattaaaagtaaaagtatctggctagaatcttacttgagtaaaagtaaaaaagtactccattaaaattgtacttgagtattaaggaagtaaaagtaaaagcaagaaagaaaactagagattcttgtttaagcttttaatctcaaaaaacattaaatgaaatgcatacaaggttttatcctgctttagaactgtttgtatttaattatcaaactataagacagacaatctaatgccagtacacgctactcaaagttgtaaaacctcagatttaacttcagtagaagctgattctcaaaattgttagtgtcaagcctagctcttttggggctgaaaagcaatcctgcagtgctgaaaagcctctcacaggcagccgatgcgggaagaggtgtattagtcttgatagagaggctgcaaatagcaggaaacgtgagcagagactccctggtgtctgaaacacaggccagatgggccctcgagcaggaaacagctatgaccatgattacgccaagctatcaactttgtatagaaaagttgcttcaggggtgtcgcttggttatttccaaaaatcaaattaattttattaaactattagaacgagcatgttttgtctatatgctacagaagataaaaaataataggagttaacagttataaaacaacacactttgtttctattgattgttgaccacactggggtctcattaagttagattaaagacacactaactgggtcaaaagcagcagattgatttcatagcaccagggtaaactttctaacacttttacggcaatcatatacattaaaattaaatacagaccacgactgaacaaggaggatgatctccaatattaaacaaagagacttgtgcctatttctctgagggtaaacatgacctctcaagttagcaagttggttttaacactacaaaaatagttaagactgcaatcccagaataaagtattggttttaaccaatcaatatagtacagtaaacatccatttgttttgttgaaacgttaaacaaatctgaccaaagctattagcttatataaaacaggtttgccttctatgtagctgaaaacaccacaggcccgattttgctactgtgtaaaacatttcagcaagatttttttattgcatttttttttactgaatcgttcaaacattttatcattttagtttgttcattcattgcaactggaaaaacaacacatcacacaaccgcacatatttcagcaataagtacaataaaacactcaaataaaaaaaacattttaaatctctttgtatttttgaccgctgtttcgcgtaatttcacggtaaaactctggaaatctccactacattcctctcagcggctcctctcaatgacagctgaagaagtgacgcggctgcctgctgtgttttgattggtcgaattcactggaggcttccagaacagtgtagagtctgaacgggtgcgcgctctgctgtatttaaagggcgaaagagagaccgcagagaaactcaaccgaagagaagcgacttgacaaagaagaaaagagcagcctgacaggactttttccccgacgaggtgtttattcgctctatttaagaatctactgtaaggtaagtctcaatatattgtactctattggctaatcagaattatatagagattatatgtacttaatgtcaaaaaatcaactttgtatatgtaatctttttacatgtggactgcctatgttcatcttattttaggtctactagaaaattatatttcccgttttcacaataaggatttttaaaaaaagcaatgaacagacgggcatttactttatgttgctgacattattttatatgagcataataaccataaatactagcaaatgtcctaaatgaatttgtgttaatgttgtctacaaaagaaaattagcgttttacttgtacaactaataataacttggttattaagagaatttcacttgttgactagaaaaatcctttcataatgaaacaattgcaccataaattgtataaatataaaattaattctaattgtttttttttcctgccaagtttgtacaaaaaagcaggctgcatgcaggagcaaatgaatggaatgactctggtgaaggaagagggcgataaagaggagggcaagcaggagccagaggttgtgtatgagactaattgtcactgggatggttgctgcagagagtttgacacccaagagcagcttgtgcatcacataaataatgaccatattcatggagaaaaaaaggaatttgtgtgtcgctgggaggaatgttcaagagaacagaaaccctttaaggcccagtatatgctggtggtgcacatgcgcagacacacaggagagaagcctcataaatgcacgtttgaaggctgttcaaaggcatattccagattagagaacctgaaaacacatttgcgttcacacacaggagagaagccatatgtgtgtgaacatgaaggctgcaacaaagccttttcgaatgcatcagaccgagccaaacaccagaatcggacacactctaatgagaaaccctatgtgtgtaaaattcctggttgcactaaacggtacactgacccaagctccttaaggaaacatgttaaaactgtacatggtcctgaagcccatgtcaccaaaaaacaacgtggggagacatacccacgacccccacctcagccacgagaacctggagcaaccactgaccaaaaggagtatacccatgctacctcaaaacaggatgaatgtctgcaagtcaagtcaatcaaaacagaaaagcccatgacgtctcagtcaagccctggcagtcagtcttcatgcagcagtgaccagtccatcaacggcccccatgtcagccagggagtccagcttactgtgaacagtattgggcttgcgggggaggtggcaatgcttgaggacatggaaaatgaagaggaggatgaggagaacagagaaggagagaaggaggatgcccccattatggactctacagtatccacagcaacatcctctgcggcaacaatagttttgcagactcatcgaagtgttggacggcccctacgctggatggagcatgttaagatggagaggttaaggcaggtgaatggagctgtgcctaggctaggattcctgcctccaacaccgccccgtaaaggccaggccatgcctgccctcacaggaaagggtcaatgccttgggaggatgacttctgtgagggtgcctccatgccctgagccgtccaacacagagctgacaattctgagtttgctgcatgaacgacgggacagtagtggcagcaacactagctcagcttacctgagcagcagccgtcgctcatctggcatctccccctgcttttccagtcgctgttctagccaggcttcactgtcagagtacagcccccatcgacatatccataatctcagtgcaactgattcttatgaccctatttccactgatgcctctcgacgctccagtgaagccagccagtatgatggaggaagttcaagtgcagtggtgtttggtggaagtggagttttcaagggcagtggtatgggatcaagaggggatctgaacctcactccagctcaacactatagcctaaaggccaagtatgcagcagcaacaggtggacctcctcccacacccttgcccagtatggggcatataaacctaaagactcactttgctttaaaggaagacagaaaggacactggccagcttacactaccaccactggtcaggtcacgtcgctgtagtgatggtgcacacaactcgcatgacagtcaagccgtgtatcaacgaaagggaacatactttggtggtggtccagggaaaaatgatcgaagagctagtgatcctacacatcacaaacaaaatcttagttcttatggcttgccacaagtacgacatttcagtagtttaaacaatatgacttcatcacccaatcctgctagttatggttactcaaacagagaagatcacagtctgaacctacagaattcaacctgtcatgtagatagctttcagagtggtctgcactccccttgtcctcctagcatcatggagcaatcagcacttgagaacattttcatgaaccaggatggtcttttgttacgtgtcaatgaaaacatccagtctgatttcatgcagtatctccattcccaagatcaggaatcaataattatccaagacaaccataggcctattttcaatcctgcagatgaccaaaacttcctcagtccacagcagcagcaagagggggtagagcaagaagacactcgtatagatctgatgcctatccagtcgaatgaagtgagctctgctagtgctgatgttactattccaaggcagccgtttcagaggtgtggcagatggtcagaccagagtggctcagtaagcaacgcctttggtcgatttggaaacatggttgtacaacagcagcctatagattttattgatcagcactcagctcttcaacacatgggaggtataagctatgggtgcagaaattcagagcagcatctacaagtagcaaaaccagcccaagagaagtccaggatgaaaacaggcattaagactgagtcttcctcaaccttccaactggaatccaaacacagcaactttggtcgaccagacttctctcatatcttttcggcccccattcatcaaaaatgttcccagcatacagttgtatcccaatcttatccccaacaaaatcaaattacaaatcaagatagcagcaacctctttttgcaaagaccaggtaactgcagcttactcttggcttaccggcctcagccagctgtcaacacttcttactccactaggcaggatattaacagccataatgtacaatcatgccatccacaaagcttgaacaatagactaggcacaagttgttccctttcaaatcaggtttccgttgtgaagcttgatcaagttaatcaccttcaaatcaactcttgctcaacaattcagcaacaaaagcctgtgtttattgataagtgttttgacccagaagagtccaagctctctcacgccatagtacagtcatgtctaatggaatcattgatggcagagaaagaaagaacttccaaagttcttctgtcatctggggttggtcaggtgactagcaaagtagaaagaagcccagctgaaaaagttggtttaggaatttgttccattggtggtgatagttatgatcagggcagtgtagtgtcttcctctcgactcagtaattctcatgattcctttgcatttcacagtgtggatccaggcactaacaacatggctattagtgacatgaactccctcctcaccaccctcgcagaagagagtaaatttctggcaatcatccaaaagtttgtgaactcccccacacccagctttcttgtacaaagtggacatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctcgagctcaagcttcgaattctgcagtcgacggtaccgcgggcccgggatccaccggatcttgaccaactttattatacatagttgataattcactggccgtcgttttacatcgatgatgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagatcagatctgcgaagatacggccacgggtgctcttgatcctgtggctgattttggactgtgctgctcgcagctgctgatgaatcacatacttcctccattttcttccactgattgactgttataatttccctaatttccaggtcaaggtgctgtgcattgtggtaatagatgtgacatgacgtcacttccaaaggaccaatgaacatgtctgaccaatttcatataatgtgaaaacgattttcataggcagaataaataacatttaaattaaactgggcatcagcgcaattcaattggtttggtaatagcaagggaaaatagaatgaagtgatctccaaaaaataagtactttttgactgtaaataaaattgtaaggagtaaaaagtacttttttttctaaaaaaatgtaattaagtaaaagtaaaagtattgatttttaattgtactcaagtaaagtaaaaatccccaaaaataatacttaagtacagtaatcaagtaaaattactcaagtactttacacctctggttcttgaccccctaccttcagcaagcccagcagatccactagttctagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtg

SEQ ID NO: 2 - DNA, 12727 bp, Artificial Sequence
  Description of Artificial Sequence Synthetic plasmid
taccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccaaatagtaggaattacccacctgtacaagtgctgaaaacttggatgaataagcccgtttgccattttctactgctattttaaatcttttctgtatttgtctgcatttgtctttacccttgaataaatgttttagttgttttttttcaattatgactgtgtttaacagacattattacaataatatgtaatagtagtacacactattattatgtaatagtacttgttgactgtatttgagaactggaccgagtgagtgttacgtcacccattcaaaatgacttacttctggctccaacaaaatgaagttaattcagttgccatttttcactgtatggacatcgccgtgttggagctagacgtcgccaataagcaagaaagagccgagatgcgtcgagtctgagtcaccgttcctatggcaacccctctaaccaatcagaagtaagcttgttggaagtccacagcctaccacttgaaagcgggctgcacaaaatctgtcaaacgttttgaacgttggatgtgagagcacatacttttattaaggcatctggttggtcagtttataatatcaacaacttgggctacagaaaagaaaagttattacagaaattatgattaacaagtacatgttaaataaagattttaatatgaatgccaccactggagcattcatgccatttggagcttcttcctgtttggatcactagaaggaggaggtcactcattacagttctcatatacagtcgttggttggttggttggttggttggttggtagattgattgattgattgattgattgattgattgattgattgattgattgattgattgattgattgattgattgattgattgattgattgattgattgattgattggtagtcaaaataagaaataatttccacagattcattacagaaatgattaaatgcatacataaaaaactggggggggggggatacaacaacacacttaagtcacatttgcctacgtaaagaaaagtaaagaaaatcaatagctatattttacatctcctttttttgctgtcttttaattagccttgttttgctgttatcttgattgaaactgtaacttcttcacctgcttcttttctttgtaggttttgccagcccagaaacaggcatggctgtgcaaggccagagcaccatgcacaatgcgctgcatgtcttcatgaacggctcaatgtcctcagtccagggctcagccaacgaccccatcttcctccttcaccatgctttcattgacaggtaacaaacacgtcatgacattagactgcacagtttttgacaaagttcatacaatctgttgtttatagctgctacaattagtgaagtttgtgaatgtacttggatgagcagcgaaagatcaattgagatcaattgttagagtttggttgccctgcagagcaaagaacaaaaaataatctggtggctttactgcgtgaggttattattggtggaatagaaacacaaaacataattgcatttatttgtttaattttttatcttatcttaactttcatcttgcatatttgtttcttacatcatttctagcatctttgagcgctggctaagaactcatcagcctccccggtccatctacccacgtaccaatgcaccaattggccacaatgacggctactacatggtgccattccttcctctttataggaatggagactacctcctgtccaacaaggctcttggatacgagtacgcctacctgttggacccaggtcattgcacaacaccagaaatgccctctgatctgcaaaagacgtgaatatctgttcagacacccatatccactctgttccacacaggtcagaggtttgtccaggagttcttgacagaggtgtaaaaagtactcaaaaattttactcaagtgaaagtacaagtacttagggaaaattttactcaattaaaagtaaaagtatctggctagaatcttacttgagtaaaagtaaaaaagtactccattaaaattgtacttgagtattaaggaagtaaaagtaaaagcaagaaagaaaactagagattcttgtttaagcttttaatctcaaaaaacattaaatgaaatgcatacaaggttttatcctgctttagaactgtttgtatttaattatcaaactataagacagacaatctaatgccagtacacgctactcaaagttgtaaaacctcagatttaacttcagtagaagctgattctcaaaattgttagtgtcaagcctagctcttttggggctgaaaagcaatcctgcagtgctgaaaagcctctcacaggcagccgatgcgggaagaggtgtattagtcttgatagagaggctgcaaatagcaggaaacgtgagcagagactccctggtgtctgaaacacaggccagatgggccctcgagcaggaaacagctatgaccatgattacgccaagctatcaactttgtatagaaaagttgcttcaggggtgtcgcttggttatttccaaaaatcaaattaattttattaaactattagaacgagcatgttttgtctatatgctacagaagataaaaaataataggagttaacagttataaaacaacacactttgtttctattgattgttgaccacactggggtctcattaagttagattaaagacacactaactgggtcaaaagcagcagattgatttcatagcaccagggtaaactttctaacacttttacggcaatcatatacattaaaattaaatacagaccacgactgaacaaggaggatgatctccaatattaaacaaagagacttgtgcctatttctctgagggtaaacatgacctctcaagttagcaagttggttttaacactacaaaaatagttaagactgcaatcccagaataaagtattggttttaaccaatcaatatagtacagtaaacatccatttgttttgttgaaacgttaaacaaatctgaccaaagctattagcttatataaaacaggtttgccttctatgtagctgaaaacaccacaggcccgattttgctactgtgtaaaacatttcagcaagatttttttattgcatttttttttactgaatcgttcaaacattttatcattttagtttgttcattcattgcaactggaaaaacaacacatcacacaaccgcacatatttcagcaataagtacaataaaacactcaaataaaaaaaacattttaaatctctttgtatttttgaccgctgtttcgcgtaatttcacggtaaaactctggaaatctccactacattcctctcagcggctcctctcaatgacagctgaagaagtgacgcggctgcctgctgtgttttgattggtcgaattcactggaggcttccagaacagtgtagagtctgaacgggtgcgcgctctgctgtatttaaagggcgaaagagagaccgcagagaaactcaaccgaagagaagcgacttgacaaagaagaaaagagcagcctgacaggactttttccccgacgaggtgtttattcgctctatttaagaatctactgtaaggtaagtctcaatatattgtactctattggctaatcagaattatatagagattatatgtacttaatgtcaaaaaatcaactttgtatatgtaatctttttacatgtggactgcctatgttcatcttattttaggtctactagaaaattatatttcccgttttcacaataaggatttttaaaaaaagcaatgaacagacgggcatttactttatgttgctgacattattttatatgagcataataaccataaatactagcaaatgtcctaaatgaatttgtgttaatgttgtctacaaaagaaaattagcgttttacttgtacaactaataataacttggttattaagagaatttcacttgttgactagaaaaatcctttcataatgaaacaattgcaccataaattgtataaatataaaattaattctaattgtttttttttcctgccaagtttgtacaaaaaagcaggctgcatgcaggagcaaatgaatggaatgactctggtgaaggaagagggcgataaagaggagggcaagcaggagccagaggttgtgtatgagactaattgtcactgggatggttgctgcagagagtttgacacccaagagcagcttgtgcatcacataaataatgaccatattcatggagaaaaaaaggaatttgtgtgtcgctgggaggaatgttcaagagaacagaaaccctttaaggcccagtatatgctggtggtgcacatgcgcagacacacaggagagaagcctcataaatgcacgtttgaaggctgttcaaaggcatattccagattagagaacctgaaaacacatttgcgttcacacacaggagagaagccatatgtgtgtgaacatgaaggctgcaacaaagccttttcgaatgcatcagaccgagccaaacaccagaatcggacacactctaatgagaaaccctatgtgtgtaaaattcctggttgcactaaacggtacactgacccaagctccttaaggaaacatgttaaaactgtacatggtcctgaagcccatgtcaccaaaaaacaacgtggggagacatacccacgacccccacctcagccacgagaacctggagcaaccactgaccaaaaggagtatacccatgctacctcaaaacaggatgaatgtctgcaagtcaagtcaatcaaaacagaaaagcccatgacgtctcagtcaagccctggcagtcagtcttcatgcagcagtgaccagtccatcaacggcccccatgtcagccagggagtccagcttactgtgaacagtattgggcttgcgggggaggtggcaatgcttgaggacatggaaaatgaagaggaggatgaggagaacagagaaggagagaaggaggatgcccccattatggactctacagtatccacagcaacatcctctgcggcaacaatagttttgcagactcatcgaagtgttggacggcccctacgctggatggagcatgttaagatggagaggttaaggcaggtgaatggagctgtgcctaggctaggattcctgcctccaacaccgccccgtaaaggccaggccatgcctgccctcacaggaaagggtcaatgccttgggaggatgacttctgtgagggtgcctccatgccctgagccgtccaacacagagctgacaattctgagtttgctgcatgaacgacgggacagtagtggcagcaacactagctcagcttacctgagcagcagccgtcgctcatctggcatctccccctgcttttccagtcgctgttctagccaggcttcactgtcagagtacagcccccatcgacatatccataatctcagtgcaactgattcttatgaccctatttccactgatgcctctcgacgctccagtgaagccagccagtatgatggaggaagttcaagtgcagtggtgtttggtggaagtggagttttcaagggcagtggtatgggatcaagaggggatctgaacctcactccagctcaacactatagcctaaaggccaagtatgcagcagcaacaggtggacctcctcccacacccttgcccagtatggggcatataaacctaaagactcactttgctttaaaggaagacagaaaggacactggccagcttacactaccaccactggtcaggtcacgtcgctgtagtgatggtgcacacaactcgcatgacagtcaagccgtgtatcaacgaaagggaacatactttggtggtggtccagggaaaaatgatcgaagagctagtgatcctacacatcacaaacaaaatcttagttcttatggcttgccacaagtacgacatttcagtagtttaaacaatatgacttcatcacccaatcctgctagttatggttactcaaacagagaagatcacagtctgaacctacagaattcaacctgtcatgtagatagctttcagagtggtctgcactccccttgtcctcctagcatcatggagcaatcagcacttgagaacattttcatgaaccaggatggtcttttgttacgtgtcaatgaaaacatccagtctgatttcatgcagtatctccattcccaagatcaggaatcaataattatccaagacaaccataggcctattttcaatcctgcagatgaccaaaacttcctcagtccacagcagcagcaagagggggtagagcaagaagacactcgtatagatctgatgcctatccagtcgaatgaagtgagctctgctagtgctgatgttactattccaaggcagccgtttcagaggtgtggcagatggtcagaccagagtggctcagtaagcaacgcctttggtcgatttggaaacatggttgtacaacagcagcctatagattttattgatcagcactcagctcttcaacacatgggaggtataagctatgggtgcagaaattcagagcagcatctacaagtagcaaaaccagcccaagagaagtccaggatgaaaacaggcattaagactgagtcttcctcaaccttccaactggaatccaaacacagcaactttggtcgaccagacttctctcatatcttttcggcccccattcatcaaaaatgttcccagcatacagttgtatcccaatcttatccccaacaaaatcaaattacaaatcaagatagcagcaacctctttttgcaaagaccaggtaactgcagcttactcttggcttaccggcctcagccagctgtcaacacttcttactccactaggcaggatattaacagccataatgtacaatcatgccatccacaaagcttgaacaatagactaggcacaagttgttccctttcaaatcaggtttccgttgtgaagcttgatcaagttaatcaccttcaaatcaactcttgctcaacaattcagcaacaaaagcctgtgtttattgataagtgttttgacccagaagagtccaagctctctcacgccatagtacagtcatgtctaatggaatcattgatggcagagaaagaaagaacttccaaagttcttctgtcatctggggttggtcaggtgactagcaaagtagaaagaagcccagctgaaaaagttggtttaggaatttgttccattggtggtgatagttatgatcagggcagtgtagtgtcttcctctcgactcagtaattctcatgattcctttgcatttcacagtgtggatccaggcactaacaacatggctattagtgacatgaactccctcctcaccaccctcgcagaagagagtaaatttctggcaatcatccaaaagtttgtgaactcccccacacccagctttcttgtacaaagtggccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaaccatggctccaaagaagaagcgtaaggtaaatcgatcgaattcaaggcctctcgagcctctagaaggtggcggaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaactgcagccctatagtgagtcgtattacgtagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttccaactttattatacatagttgataattcactggccgtcgttttacatcgatgatgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagatcagatctgcgaagatacggccacgggtgctcttgatcctgtggctgattttggactgtgctgctcgcagctgctgatgaatcacatacttcctccattttcttccactgattgactgttataatttccctaatttccaggtcaaggtgctgtgcattgtggtaatagatgtgacatgacgtcacttccaaaggaccaatgaacatgtctgaccaatttcatataatgtgaaaacgattttcataggcagaataaataacatttaaattaaactgggcatcagcgcaattcaattggtttggtaatagcaagggaaaatagaatgaagtgatctccaaaaaataagtactttttgactgtaaataaaattgtaaggagtaaaaagtacttttttttctaaaaaaatgtaattaagtaaaagtaaaagtattgatttttaattgtactcaagtaaagtaaaaatccccaaaaataatacttaagtacagtaatcaagtaaaattactcaagtactttacacctctggttcttgaccccctaccttcagcaagcccagcagatccactagttctagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtg

SEQ ID NO: 3 - DNA, 12065 bp, Artificial Sequence
  Description of Artificial Sequence Synthetic plasmid
taccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccaaatagtaggaattacccacctgtacaagtgctgaaaacttggatgaataagcccgtttgccattttctactgctattttaaatcttttctgtatttgtctgcatttgtctttacccttgaataaatgttttagttgttttttttcaattatgactgtgtttaacagacattattacaataatatgtaatagtagtacacactattattatgtaatagtacttgttgactgtatttgagaactggaccgagtgagtgttacgtcacccattcaaaatgacttacttctggctccaacaaaatgaagttaattcagttgccatttttcactgtatggacatcgccgtgttggagctagacgtcgccaataagcaagaaagagccgagatgcgtcgagtctgagtcaccgttcctatggcaacccctctaaccaatcagaagtaagcttgttggaagtccacagcctaccacttgaaagcgggctgcacaaaatctgtcaaacgttttgaacgttggatgtgagagcacatacttttattaaggcatctggttggtcagtttataatatcaacaacttgggctacagaaaagaaaagttattacagaaattatgattaacaagtacatgttaaataaagattttaatatgaatgccaccactggagcattcatgccatttggagcttcttcctgtttggatcactagaaggaggaggtcactcattacagttctcatatacagtcgttggttggttggttggttggttggttggtagattgattgattgattgattgattgattgattgattgattgattgattgattgattgattgattgattgattgattgattgattgattgattgattgattgattggtagtcaaaataagaaataatttccacagattcattacagaaatgattaaatgcatacataaaaaactggggggggggggatacaacaacacacttaagtcacatttgcctacgtaaagaaaagtaaagaaaatcaatagctatattttacatctcctttttttgctgtcttttaattagccttgttttgctgttatcttgattgaaactgtaacttcttcacctgcttcttttctttgtaggttttgccagcccagaaacaggcatggctgtgcaaggccagagcaccatgcacaatgcgctgcatgtcttcatgaacggctcaatgtcctcagtccagggctcagccaacgaccccatcttcctccttcaccatgctttcattgacaggtaacaaacacgtcatgacattagactgcacagtttttgacaaagttcatacaatctgttgtttatagctgctacaattagtgaagtttgtgaatgtacttggatgagcagcgaaagatcaattgagatcaattgttagagtttggttgccctgcagagcaaagaacaaaaaataatctggtggctttactgcgtgaggttattattggtggaatagaaacacaaaacataattgcatttatttgtttaattttttatcttatcttaactttcatcttgcatatttgtttcttacatcatttctagcatctttgagcgctggctaagaactcatcagcctccccggtccatctacccacgtaccaatgcaccaattggccacaatgacggctactacatggtgccattccttcctctttataggaatggagactacctcctgtccaacaaggctcttggatacgagtacgcctacctgttggacccaggtcattgcacaacaccagaaatgccctctgatctgcaaaagacgtgaatatctgttcagacacccatatccactctgttccacacaggtcagaggtttgtccaggagttcttgacagaggtgtaaaaagtactcaaaaattttactcaagtgaaagtacaagtacttagggaaaattttactcaattaaaagtaaaagtatctggctagaatcttacttgagtaaaagtaaaaaagtactccattaaaattgtacttgagtattaaggaagtaaaagtaaaagcaagaaagaaaactagagattcttgtttaagcttttaatctcaaaaaacattaaatgaaatgcatacaaggttttatcctgctttagaactgtttgtatttaattatcaaactataagacagacaatctaatgccagtacacgctactcaaagttgtaaaacctcagatttaacttcagtagaagctgattctcaaaattgttagtgtcaagcctagctcttttggggctgaaaagcaatcctgcagtgctgaaaagcctctcacaggcagccgatgcgggaagaggtgtattagtcttgatagagaggctgcaaatagcaggaaacgtgagcagagactccctggtgtctgaaacacaggccagatgggccctcgagcaggaaacagctatgaccatgattacgccaagctatcaactttgtatagaaaagttgcttcaggggtgtcgcttggttatttccaaaaatcaaattaattttattaaactattagaacgagcatgttttgtctatatgctacagaagataaaaaataataggagttaacagttataaaacaacacactttgtttctattgattgttgaccacactggggtctcattaagttagattaaagacacactaactgggtcaaaagcagcagattgatttcatagcaccagggtaaactttctaacacttttacggcaatcatatacattaaaattaaatacagaccacgactgaacaaggaggatgatctccaatattaaacaaagagacttgtgcctatttctctgagggtaaacatgacctctcaagttagcaagttggttttaacactacaaaaatagttaagactgcaatcccagaataaagtattggttttaaccaatcaatatagtacagtaaacatccatttgttttgttgaaacgttaaacaaatctgaccaaagctattagcttatataaaacaggtttgccttctatgtagctgaaaacaccacaggcccgattttgctactgtgtaaaacatttcagcaagatttttttattgcatttttttttactgaatcgttcaaacattttatcattttagtttgttcattcattgcaactggaaaaacaacacatcacacaaccgcacatatttcagcaataagtacaataaaacactcaaataaaaaaaacattttaaatctctttgtatttttgaccgctgtttcgcgtaatttcacggtaaaactctggaaatctccactacattcctctcagcggctcctctcaatgacagctgaagaagtgacgcggctgcctgctgtgttttgattggtcgaattcactggaggcttccagaacagtgtagagtctgaacgggtgcgcgctctgctgtatttaaagggcgaaagagagaccgcagagaaactcaaccgaagagaagcgacttgacaaagaagaaaagagcagcctgacaggactttttccccgacgaggtgtttattcgctctatttaagaatctactgtaaggtaagtctcaatatattgtactctattggctaatcagaattatatagagattatatgtacttaatgtcaaaaaatcaactttgtatatgtaatctttttacatgtggactgcctatgttcatcttattttaggtctactagaaaattatatttcccgttttcacaataaggatttttaaaaaaagcaatgaacagacgggcatttactttatgttgctgacattattttatatgagcataataaccataaatactagcaaatgtcctaaatgaatttgtgttaatgttgtctacaaaagaaaattagcgttttacttgtacaactaataataacttggttattaagagaatttcacttgttgactagaaaaatcctttcataatgaaacaattgcaccataaattgtataaatataaaattaattctaattgtttttttttcctgccaagtttgtacaaaaaagcaggctgcatgcaggagcaaatgaatggaatgactctggtgaaggaagagggcgataaagaggagggcaagcaggagccagaggttgtgtatgagactaattgtcactgggatggttgctgcagagagtttgacacccaagagcagcttgtgcatcacataaataatgaccatattcatggagaaaaaaaggaatttgtgtgtcgctgggaggaatgttcaagagaacagaaaccctttaaggcccagtatatgctggtggtgcacatgcgcagacacacaggagagaagcctcataaatgcacgtttgaaggctgttcaaaggcatattccagattagagaacctgaaaacacatttgcgttcacacacaggagagaagccatatgtgtgtgaacatgaaggctgcaacaaagccttttcgaatgcatcagaccgagccaaacaccagaatcggacacactctaatgagaaaccctatgtgtgtaaaattcctggttgcactaaacggtacactgacccaagctccttaaggaaacatgttaaaactgtacatggtcctgaagcccatgtcaccaaaaaacaacgtggggagacatacccacgacccccacctcagccacgagaacctggagcaaccactgaccaaaaggagtatacccatgctacctcaaaacaggatgaatgtctgcaagtcaagtcaatcaaaacagaaaagcccatgacgtctcagtcaagccctggcagtcagtcttcatgcagcagtgaccagtccatcaacggcccccatgtcagccagggagtccagcttactgtgaacagtattgggcttgcgggggaggtggcaatgcttgaggacatggaaaatgaagaggaggatgaggagaacagagaaggagagaaggaggatgcccccattatggactctacagtatccacagcaacatcctctgcggcaacaatagttttgcagactcatcgaagtgttggacggcccctacgctggatggagcatgttaagatggagaggttaaggcaggtgaatggagctgtgcctaggctaggattcctgcctccaacaccgccccgtaaaggccaggccatgcctgccctcacaggaaagggtcaatgccttgggaggatgacttctgtgagggtgcctccatgccctgagccgtccaacacagagctgacaattctgagtttgctgcatgaacgacgggacagtagtggcagcaacactagctcagcttacctgagcagcagccgtcgctcatctggcatctccccctgcttttccagtcgctgttctagccaggcttcactgtcagagtacagcccccatcgacatatccataatctcagtgcaactgattcttatgaccctatttccactgatgcctctcgacgctccagtgaagccagccagtatgatggaggaagttcaagtgcagtggtgtttggtggaagtggagttttcaagggcagtggtatgggatcaagaggggatctgaacctcactccagctcaacactatagcctaaaggccaagtatgcagcagcaacaggtggacctcctcccacacccttgcccagtatggggcatataaacctaaagactcactttgctttaaaggaagacagaaaggacactggccagcttacactaccaccactggtcaggtcacgtcgctgtagtgatggtgcacacaactcgcatgacagtcaagccgtgtatcaacgaaagggaacatactttggtggtggtccagggaaaaatgatcgaagagctagtgatcctacacatcacaaacaaaatcttagttcttatggcttgccacaagtacgacatttcagtagtttaaacaatatgacttcatcacccaatcctgctagttatggttactcaaacagagaagatcacagtctgaacctacagaattcaacctgtcatgtagatagctttcagagtggtctgcactccccttgtcctcctagcatcatggagcaatcagcacttgagaacattttcatgaaccaggatggtcttttgttacgtgtcaatgaaaacatccagtctgatttcatgcagtatctccattcccaagatcaggaatcaataattatccaagacaaccataggcctattttcaatcctgcagatgaccaaaacttcctcagtccacagcagcagcaagagggggtagagcaagaagacactcgtatagatctgatgcctatccagtcgaatgaagtgagctctgctagtgctgatgttactattccaaggcagccgtttcagaggtgtggcagatggtcagaccagagtggctcagtaagcaacgcctttggtcgatttggaaacatggttgtacaacagcagcctatagattttattgatcagcactcagctcttcaacacatgggaggtataagctatgggtgcagaaattcagagcagcatctacaagtagcaaaaccagcccaagagaagtccaggatgaaaacaggcattaagactgagtcttcctcaaccttccaactggaatccaaacacagcaactttggtcgaccagacttctctcatatcttttcggcccccattcatcaaaaatgttcccagcatacagttgtatcccaatcttatccccaacaaaatcaaattacaaatcaagatagcagcaacctctttttgcaaagaccaggtaactgcagcttactcttggcttaccggcctcagccagctgtcaacacttcttactccactaggcaggatattaacagccataatgtacaatcatgccatccacaaagcttgaacaatagactaggcacaagttgttccctttcaaatcaggtttccgttgtgaagcttgatcaagttaatcaccttcaaatcaactcttgctcaacaattcagcaacaaaagcctgtgtttattgataagtgttttgacccagaagagtccaagctctctcacgccatagtacagtcatgtctaatggaatcattgatggcagagaaagaaagaacttccaaagttcttctgtcatctggggttggtcaggtgactagcaaagtagaaagaagcccagctgaaaaagttggtttaggaatttgttccattggtggtgatagttatgatcagggcagtgtagtgtcttcctctcgactcagtaattctcatgattcctttgcatttcacagtgtggatccaggcactaacaacatggctattagtgacatgaactccctcctcaccaccctcgcagaagagagtaaatttctggcaatcatccaaaagtttgtgaactcccccacacccagctttcttgtacaaagtggttatggtgagcaagggcgaggacgacaacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgccgaggtcaagaccacctacaaggccaagaagcccgtgcagctgcccggcgcctacaacgtcaacatcaagctggacatcacctcccacaacgaggactacaccatcgtggaacagtacgagcgcgccgagggccgccactccaccggcggcatggacgagctgtacaagtaaggcgcgcctctagaactatagtgagtcgtattacgtagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttccaactttattatacatagttgataattcactggccgtcgttttacatcgatgatgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagatcagatctgcgaagatacggccacgggtgctcttgatcctgtggctgattttggactgtgctgctcgcagctgctgatgaatcacatacttcctccattttcttccactgattgactgttataatttccctaatttccaggtcaaggtgctgtgcattgtggtaatagatgtgacatgacgtcacttccaaaggaccaatgaacatgtctgaccaatttcatataatgtgaaaacgattttcataggcagaataaataacatttaaattaaactgggcatcagcgcaattcaattggtttggtaatagcaagggaaaatagaatgaagtgatctccaaaaaataagtactttttgactgtaaataaaattgtaaggagtaaaaagtacttttttttctaaaaaaatgtaattaagtaaaagtaaaagtattgatttttaattgtactcaagtaaagtaaaaatccccaaaaataatacttaagtacagtaatcaagtaaaattactcaagtactttacacctctggttcttgaccccctaccttcagcaagcccagcagatccactagttctagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtg

SEQ ID NO: 4 - DNA, 11883 bp, Artificial Sequence
  Description of Artificial Sequence Synthetic plasmid
taccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccaaatagtaggaattacccacctgtacaagtgctgaaaacttggatgaataagcccgtttgccattttctactgctattttaaatcttttctgtatttgtctgcatttgtctttacccttgaataaatgttttagttgttttttttcaattatgactgtgtttaacagacattattacaataatatgtaatagtagtacacactattattatgtaatagtacttgttgactgtatttgagaactggaccgagtgagtgttacgtcacccattcaaaatgacttacttctggctccaacaaaatgaagttaattcagttgccatttttcactgtatggacatcgccgtgttggagctagacgtcgccaataagcaagaaagagccgagatgcgtcgagtctgagtcaccgttcctatggcaacccctctaaccaatcagaagtaagcttgttggaagtccacagcctaccacttgaaagcgggctgcacaaaatctgtcaaacgttttgaacgttggatgtgagagcacatacttttattaaggcatctggttggtcagtttataatatcaacaacttgggctacagaaaagaaaagttattacagaaattatgattaacaagtacatgttaaataaagattttaatatgaatgccaccactggagcattcatgccatttggagcttcttcctgtttggatcactagaaggaggaggtcactcattacagttctcatatacagtcgttggttggttggttggttggttggttggtagattgattgattgattgattgattgattgattgattgattgattgattgattgattgattgattgattgattgattgattgattgattgattgattgattgattggtagtcaaaataagaaataatttccacagattcattacagaaatgattaaatgcatacataaaaaactggggggggggggatacaacaacacacttaagtcacatttgcctacgtaaagaaaagtaaagaaaatcaatagctatattttacatctcctttttttgctgtcttttaattagccttgttttgctgttatcttgattgaaactgtaacttcttcacctgcttcttttctttgtaggttttgccagcccagaaacaggcatggctgtgcaaggccagagcaccatgcacaatgcgctgcatgtcttcatgaacggctcaatgtcctcagtccagggctcagccaacgaccccatcttcctccttcaccatgctttcattgacaggtaacaaacacgtcatgacattagactgcacagtttttgacaaagttcatacaatctgttgtttatagctgctacaattagtgaagtttgtgaatgtacttggatgagcagcgaaagatcaattgagatcaattgttagagtttggttgccctgcagagcaaagaacaaaaaataatctggtggctttactgcgtgaggttattattggtggaatagaaacacaaaacataattgcatttatttgtttaattttttatcttatcttaactttcatcttgcatatttgtttcttacatcatttctagcatctttgagcgctggctaagaactcatcagcctccccggtccatctacccacgtaccaatgcaccaattggccacaatgacggctactacatggtgccattccttcctctttataggaatggagactacctcctgtccaacaaggctcttggatacgagtacgcctacctgttggacccaggtcattgcacaacaccagaaatgccctctgatctgcaaaagacgtgaatatctgttcagacacccatatccactctgttccacacaggtcagaggtttgtccaggagttcttgacagaggtgtaaaaagtactcaaaaattttactcaagtgaaagtacaagtacttagggaaaattttactcaattaaaagtaaaagtatctggctagaatcttacttgagtaaaagtaaaaaagtactccattaaaattgtacttgagtattaaggaagtaaaagtaaaagcaagaaagaaaactagagattcttgtttaagcttttaatctcaaaaaacattaaatgaaatgcatacaaggttttatcctgctttagaactgtttgtatttaattatcaaactataagacagacaatctaatgccagtacacgctactcaaagttgtaaaacctcagatttaacttcagtagaagctgattctcaaaattgttagtgtcaagcctagctcttttggggctgaaaagcaatcctgcagtgctgaaaagcctctcacaggcagccgatgcgggaagaggtgtattagtcttgatagagaggctgcaaatagcaggaaacgtgagcagagactccctggtgtctgaaacacaggccagatgggccctcgagcaggaaacagctatgaccatgattacgccaagctatcaactttgtatagaaaagttgcttcaggggtgtcgcttggttatttccaaaaatcaaattaattttattaaactattagaacgagcatgttttgtctatatgctacagaagataaaaaataataggagttaacagttataaaacaacacactttgtttctattgattgttgaccacactggggtctcattaagttagattaaagacacactaactgggtcaaaagcagcagattgatttcatagcaccagggtaaactttctaacacttttacggcaatcatatacattaaaattaaatacagaccacgactgaacaaggaggatgatctccaatattaaacaaagagacttgtgcctatttctctgagggtaaacatgacctctcaagttagcaagttggttttaacactacaaaaatagttaagactgcaatcccagaataaagtattggttttaaccaatcaatatagtacagtaaacatccatttgttttgttgaaacgttaaacaaatctgaccaaagctattagcttatataaaacaggtttgccttctatgtagctgaaaacaccacaggcccgattttgctactgtgtaaaacatttcagcaagatttttttattgcatttttttttactgaatcgttcaaacattttatcattttagtttgttcattcattgcaactggaaaaacaacacatcacacaaccgcacatatttcagcaataagtacaataaaacactcaaataaaaaaaacattttaaatctctttgtatttttgaccgctgtttcgcgtaatttcacggtaaaactctggaaatctccactacattcctctcagcggctcctctcaatgacagctgaagaagtgacgcggctgcctgctgtgttttgattggtcgaattcactggaggcttccagaacagtgtagagtctgaacgggtgcgcgctctgctgtatttaaagggcgaaagagagaccgcagagaaactcaaccgaagagaagcgacttgacaaagaagaaaagagcagcctgacaggactttttccccgacgaggtgtttattcgctctatttaagaatctactgtaaggtaagtctcaatatattgtactctattggctaatcagaattatatagagattatatgtacttaatgtcaaaaaatcaactttgtatatgtaatctttttacatgtggactgcctatgttcatcttattttaggtctactagaaaattatatttcccgttttcacaataaggatttttaaaaaaagcaatgaacagacgggcatttactttatgttgctgacattattttatatgagcataataaccataaatactagcaaatgtcctaaatgaatttgtgttaatgttgtctacaaaagaaaattagcgttttacttgtacaactaataataacttggttattaagagaatttcacttgttgactagaaaaatcctttcataatgaaacaattgcaccataaattgtataaatataaaattaattctaattgtttttttttcctgccaagtttgtacaaaaaagcaggcttcgaaggagatagaaccatggaggagggcaagcaggagccagaggttgtgtatgagactaattgtcactgggatggttgctgcagagagtttgacacccaagagcagcttgtgcatcacataaataatgaccatattcatggagaaaaaaaggaatttgtgtgtcgctgggaggaatgttcaagagaacagaaaccctttaaggcccagtatatgctggtggtgcacatgcgcagacacacaggagagaagcctcataaatgcacgtttgaaggctgttcaaaggcatattccagattagagaacctgaaaacacatttgcgttcacacacaggagagaagccatatgtgtgtgaacatgaaggctgcaacaaagccttttcgaatgcatcagaccgagccaaacaccagaatcggacacactctaatgagaaaccctatgtgtgtaaaattcctggttgcactaaacggtacactgacccaagctccttaaggaaacatgttaaaactgtacatggtcctgaagcccatgtcaccaaaaaacaacgtggggagacatacccacgacccccacctcagccacgagaacctggagcaaccactgaccaaaaggagtatacccatgctacctcaaaacaggatgaatgtctgcaagtcaagtcaatcaaaacagaaaagcccatgacgtctcagtcaagccctggcagtcagtcttcatgcagcagtgaccagtccatcaacggcccccatgtcagccagggagtccagcttactgtgaacagtattgggcttgcgggggaggtggcaatgcttgaggacatggaaaatgaagaggaggatgaggagaacagagaaggagagaaggaggatgcccccattatggactctacagtatccacagcaacatcctctgcggcaacaatagttttgcagactcatcgaagtgttggacggcccctacgctggatggagcatgttaagatggagaggttaaggcaggtgaatggagctgtgcctaggctaggattcctgcctccaacaccgccccgtaaaggccaggccatgcctgccctcacaggaaagggtcaatgccttgggaggatgacttctgtgagggtgcctccatgccctgagccgtccaacacagagctgacaattctgagtttgctgcatgaacgacgggacagtagtggcagcaacactagctcagcttacctgagcagcagccgtcgctcatctggcatctccccctgcttttccagtcgctgttctagccaggcttcactgtcagagtacagcccccatcgacatatccataatctcagtgcaactgattcttatgaccctatttccactgatgcctctcgacgctccagtgaagccagccagtatgatggaggaagttcaagtgcagtggtgtttggtggaagtggagttttcaagggcagtggtatgggatcaagaggggatctgaacctcactccagctcaacactatagcctaaaggccaagtatgcagcagcaacaggtggacctcctcccacacccttgcccagtatggggcatataaacctaaagactcactttgctttaaaggaagacagaaaggacactggccagcttacactaccaccactggtcaggtcacgtcgctgtagtgatggtgcacacaactcgcatgacagtcaagccgtgtatcaacgaaagggaacatactttggtggtggtccagggaaaaatgatcgaagagctagtgatcctacacatcacaaacaaaatcttagttcttatggcttgccacaagtacgacatttcagtagtttaaacaatatgacttcatcacccaatcctgctagttatggttactcaaacagagaagatcacagtctgaacctacagaattcaacctgtcatgtagatagctttcagagtggtctgcactccccttgtcctcctagcatcatggagcaatcagcacttgagaacattttcatgaaccaggatggtcttttgttacgtgtcaatgaaaacatccagtctgatttcatgcagtatctccattcccaagatcaggaatcaataattatccaagacaaccataggcctattttcaatcctgcagatgaccaaaacttcctcagtccacagcagcagcaagagggggtagagcaagaagacactcgtatagatctgatgcctatccagtcgaatgaagtgagctctgctagtgctgatgttactattccaaggcagccgtttcagaggtgtggcagatggtcagaccagagtggctcagtaagcaacgcctttggtcgatttggaaacatggttgtacaacagcagcctatagattttattgatcagcactcagctcttcaacacatgggaggtataagctatgggtgcagaaattcagagcagcatctacaagtagcaaaaccagcccaagagaagtccaggatgaaaacaggcattaagactgagtcttcctcaaccttccaactggaatccaaacacagcaactttggtcgaccagacttctctcatatcttttcggcccccattcatcaaaaatgttcccagcatacagttgtatcccaatcttatccccaacaaaatcaaattacaaatcaagatagcagcaacctctttttgcaaagaccaggtaactgcagcttactcttggcttaccggcctcagccagctgtcaacacttcttactccactaggcaggatattaacagccataatgtacaatcatgccatccacaaagcttgaacaatagactaggcacaagttgttccctttcaaatcaggtttccgttgtgaagcttgatcaagttaatcaccttcaaatcaactcttgctcaacaattcagcaacaaaagcctgtgtttattgataagtgttttgacccagaagagtccaagctctctcacgccatagtacagtcatgtctaatggaatcattgatggcagagaaagaaagaacttccaaagttcttctgtcatctggggttggtcaggtgactagcaaagtagaaagaagcccagctgaaaaagttggtttaggaatttgttccattggtggtgatagttatgatcagggcagtgtagtgtcttcctctcgactcagtaattctcatgattcctttgcatttcacagtgtggatccaggcactaacaacatggctattagtgacatgaactccctcctcaccaccctcgcagaagagagtaaatttctggcaatcatccaaaagtttgtgaactcccccacacccagctttcttgtacaaagtggacatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctcgagctcaagcttcgaattctgcagtcgacggtaccgcgggcccgggatccaccggatcttgaccaactttattatacatagttgataattcactggccgtcgttttacatcgatgatgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagatcagatctgcgaagatacggccacgggtgctcttgatcctgtggctgattttggactgtgctgctcgcagctgctgatgaatcacatacttcctccattttcttccactgattgactgttataatttccctaatttccaggtcaaggtgctgtgcattgtggtaatagatgtgacatgacgtcacttccaaaggaccaatgaacatgtctgaccaatttcatataatgtgaaaacgattttcataggcagaataaataacatttaaattaaactgggcatcagcgcaattcaattggtttggtaatagcaagggaaaatagaatgaagtgatctccaaaaaataagtactttttgactgtaaataaaattgtaaggagtaaaaagtacttttttttctaaaaaaatgtaattaagtaaaagtaaaagtattgatttttaattgtactcaagtaaagtaaaaatccccaaaaataatacttaagtacagtaatcaagtaaaattactcaagtactttacacctctggttcttgaccccctaccttcagcaagcccagcagatccactagttctagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtg

SEQ ID NO: 5 - DNA, 12694 bp, Artificial Sequence
  Description of Artificial Sequence Synthetic plasmid
taccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccaaatagtaggaattacccacctgtacaagtgctgaaaacttggatgaataagcccgtttgccattttctactgctattttaaatcttttctgtatttgtctgcatttgtctttacccttgaataaatgttttagttgttttttttcaattatgactgtgtttaacagacattattacaataatatgtaatagtagtacacactattattatgtaatagtacttgttgactgtatttgagaactggaccgagtgagtgttacgtcacccattcaaaatgacttacttctggctccaacaaaatgaagttaattcagttgccatttttcactgtatggacatcgccgtgttggagctagacgtcgccaataagcaagaaagagccgagatgcgtcgagtctgagtcaccgttcctatggcaacccctctaaccaatcagaagtaagcttgttggaagtccacagcctaccacttgaaagcgggctgcacaaaatctgtcaaacgttttgaacgttggatgtgagagcacatacttttattaaggcatctggttggtcagtttataatatcaacaacttgggctacagaaaagaaaagttattacagaaattatgattaacaagtacatgttaaataaagattttaatatgaatgccaccactggagcattcatgccatttggagcttcttcctgtttggatcactagaaggaggaggtcactcattacagttctcatatacagtcgttggttggttggttggttggttggttggtagattgattgattgattgattgattgattgattgattgattgattgattgattgattgattgattgattgattgattgattgattgattgattgattgattgattggtagtcaaaataagaaataatttccacagattcattacagaaatgattaaatgcatacataaaaaactggggggggggggatacaacaacacacttaagtcacatttgcctacgtaaagaaaagtaaagaaaatcaatagctatattttacatctcctttttttgctgtcttttaattagccttgttttgctgttatcttgattgaaactgtaacttcttcacctgcttcttttctttgtaggttttgccagcccagaaacaggcatggctgtgcaaggccagagcaccatgcacaatgcgctgcatgtcttcatgaacggctcaatgtcctcagtccagggctcagccaacgaccccatcttcctccttcaccatgctttcattgacaggtaacaaacacgtcatgacattagactgcacagtttttgacaaagttcatacaatctgttgtttatagctgctacaattagtgaagtttgtgaatgtacttggatgagcagcgaaagatcaattgagatcaattgttagagtttggttgccctgcagagcaaagaacaaaaaataatctggtggctttactgcgtgaggttattattggtggaatagaaacacaaaacataattgcatttatttgtttaattttttatcttatcttaactttcatcttgcatatttgtttcttacatcatttctagcatctttgagcgctggctaagaactcatcagcctccccggtccatctacccacgtaccaatgcaccaattggccacaatgacggctactacatggtgccattccttcctctttataggaatggagactacctcctgtccaacaaggctcttggatacgagtacgcctacctgttggacccaggtcattgcacaacaccagaaatgccctctgatctgcaaaagacgtgaatatctgttcagacacccatatccactctgttccacacaggtcagaggtttgtccaggagttcttgacagaggtgtaaaaagtactcaaaaattttactcaagtgaaagtacaagtacttagggaaaattttactcaattaaaagtaaaagtatctggctagaatcttacttgagtaaaagtaaaaaagtactccattaaaattgtacttgagtattaaggaagtaaaagtaaaagcaagaaagaaaactagagattcttgtttaagcttttaatctcaaaaaacattaaatgaaatgcatacaaggttttatcctgctttagaactgtttgtatttaattatcaaactataagacagacaatctaatgccagtacacgctactcaaagttgtaaaacctcagatttaacttcagtagaagctgattctcaaaattgttagtgtcaagcctagctcttttggggctgaaaagcaatcctgcagtgctgaaaagcctctcacaggcagccgatgcgggaagaggtgtattagtcttgatagagaggctgcaaatagcaggaaacgtgagcagagactccctggtgtctgaaacacaggccagatgggccctcgagcaggaaacagctatgaccatgattacgccaagctatcaactttgtatagaaaagttgcttcaggggtgtcgcttggttatttccaaaaatcaaattaattttattaaactattagaacgagcatgttttgtctatatgctacagaagataaaaaataataggagttaacagttataaaacaacacactttgtttctattgattgttgaccacactggggtctcattaagttagattaaagacacactaactgggtcaaaagcagcagattgatttcatagcaccagggtaaactttctaacacttttacggcaatcatatacattaaaattaaatacagaccacgactgaacaaggaggatgatctccaatattaaacaaagagacttgtgcctatttctctgagggtaaacatgacctctcaagttagcaagttggttttaacactacaaaaatagttaagactgcaatcccagaataaagtattggttttaaccaatcaatatagtacagtaaacatccatttgttttgttgaaacgttaaacaaatctgaccaaagctattagcttatataaaacaggtttgccttctatgtagctgaaaacaccacaggcccgattttgctactgtgtaaaacatttcagcaagatttttttattgcatttttttttactgaatcgttcaaacattttatcattttagtttgttcattcattgcaactggaaaaacaacacatcacacaaccgcacatatttcagcaataagtacaataaaacactcaaataaaaaaaacattttaaatctctttgtatttttgaccgctgtttcgcgtaatttcacggtaaaactctggaaatctccactacattcctctcagcggctcctctcaatgacagctgaagaagtgacgcggctgcctgctgtgttttgattggtcgaattcactggaggcttccagaacagtgtagagtctgaacgggtgcgcgctctgctgtatttaaagggcgaaagagagaccgcagagaaactcaaccgaagagaagcgacttgacaaagaagaaaagagcagcctgacaggactttttccccgacgaggtgtttattcgctctatttaagaatctactgtaaggtaagtctcaatatattgtactctattggctaatcagaattatatagagattatatgtacttaatgtcaaaaaatcaactttgtatatgtaatctttttacatgtggactgcctatgttcatcttattttaggtctactagaaaattatatttcccgttttcacaataaggatttttaaaaaaagcaatgaacagacgggcatttactttatgttgctgacattattttatatgagcataataaccataaatactagcaaatgtcctaaatgaatttgtgttaatgttgtctacaaaagaaaattagcgttttacttgtacaactaataataacttggttattaagagaatttcacttgttgactagaaaaatcctttcataatgaaacaattgcaccataaattgtataaatataaaattaattctaattgtttttttttcctgccaagtttgtacaaaaaagcaggcttcgaaggagatagaaccatggaggagggcaagcaggagccagaggttgtgtatgagactaattgtcactgggatggttgctgcagagagtttgacacccaagagcagcttgtgcatcacataaataatgaccatattcatggagaaaaaaaggaatttgtgtgtcgctgggaggaatgttcaagagaacagaaaccctttaaggcccagtatatgctggtggtgcacatgcgcagacacacaggagagaagcctcataaatgcacgtttgaaggctgttcaaaggcatattccagattagagaacctgaaaacacatttgcgttcacacacaggagagaagccatatgtgtgtgaacatgaaggctgcaacaaagccttttcgaatgcatcagaccgagccaaacaccagaatcggacacactctaatgagaaaccctatgtgtgtaaaattcctggttgcactaaacggtacactgacccaagctccttaaggaaacatgttaaaactgtacatggtcctgaagcccatgtcaccaaaaaacaacgtggggagacatacccacgacccccacctcagccacgagaacctggagcaaccactgaccaaaaggagtatacccatgctacctcaaaacaggatgaatgtctgcaagtcaagtcaatcaaaacagaaaagcccatgacgtctcagtcaagccctggcagtcagtcttcatgcagcagtgaccagtccatcaacggcccccatgtcagccagggagtccagcttactgtgaacagtattgggcttgcgggggaggtggcaatgcttgaggacatggaaaatgaagaggaggatgaggagaacagagaaggagagaaggaggatgcccccattatggactctacagtatccacagcaacatcctctgcggcaacaatagttttgcagactcatcgaagtgttggacggcccctacgctggatggagcatgttaagatggagaggttaaggcaggtgaatggagctgtgcctaggctaggattcctgcctccaacaccgccccgtaaaggccaggccatgcctgccctcacaggaaagggtcaatgccttgggaggatgacttctgtgagggtgcctccatgccctgagccgtccaacacagagctgacaattctgagtttgctgcatgaacgacgggacagtagtggcagcaacactagctcagcttacctgagcagcagccgtcgctcatctggcatctccccctgcttttccagtcgctgttctagccaggcttcactgtcagagtacagcccccatcgacatatccataatctcagtgcaactgattcttatgaccctatttccactgatgcctctcgacgctccagtgaagccagccagtatgatggaggaagttcaagtgcagtggtgtttggtggaagtggagttttcaagggcagtggtatgggatcaagaggggatctgaacctcactccagctcaacactatagcctaaaggccaagtatgcagcagcaacaggtggacctcctcccacacccttgcccagtatggggcatataaacctaaagactcactttgctttaaaggaagacagaaaggacactggccagcttacactaccaccactggtcaggtcacgtcgctgtagtgatggtgcacacaactcgcatgacagtcaagccgtgtatcaacgaaagggaacatactttggtggtggtccagggaaaaatgatcgaagagctagtgatcctacacatcacaaacaaaatcttagttcttatggcttgccacaagtacgacatttcagtagtttaaacaatatgacttcatcacccaatcctgctagttatggttactcaaacagagaagatcacagtctgaacctacagaattcaacctgtcatgtagatagctttcagagtggtctgcactccccttgtcctcctagcatcatggagcaatcagcacttgagaacattttcatgaaccaggatggtcttttgttacgtgtcaatgaaaacatccagtctgatttcatgcagtatctccattcccaagatcaggaatcaataattatccaagacaaccataggcctattttcaatcctgcagatgaccaaaacttcctcagtccacagcagcagcaagagggggtagagcaagaagacactcgtatagatctgatgcctatccagtcgaatgaagtgagctctgctagtgctgatgttactattccaaggcagccgtttcagaggtgtggcagatggtcagaccagagtggctcagtaagcaacgcctttggtcgatttggaaacatggttgtacaacagcagcctatagattttattgatcagcactcagctcttcaacacatgggaggtataagctatgggtgcagaaattcagagcagcatctacaagtagcaaaaccagcccaagagaagtccaggatgaaaacaggcattaagactgagtcttcctcaaccttccaactggaatccaaacacagcaactttggtcgaccagacttctctcatatcttttcggcccccattcatcaaaaatgttcccagcatacagttgtatcccaatcttatccccaacaaaatcaaattacaaatcaagatagcagcaacctctttttgcaaagaccaggtaactgcagcttactcttggcttaccggcctcagccagctgtcaacacttcttactccactaggcaggatattaacagccataatgtacaatcatgccatccacaaagcttgaacaatagactaggcacaagttgttccctttcaaatcaggtttccgttgtgaagcttgatcaagttaatcaccttcaaatcaactcttgctcaacaattcagcaacaaaagcctgtgtttattgataagtgttttgacccagaagagtccaagctctctcacgccatagtacagtcatgtctaatggaatcattgatggcagagaaagaaagaacttccaaagttcttctgtcatctggggttggtcaggtgactagcaaagtagaaagaagcccagctgaaaaagttggtttaggaatttgttccattggtggtgatagttatgatcagggcagtgtagtgtcttcctctcgactcagtaattctcatgattcctttgcatttcacagtgtggatccaggcactaacaacatggctattagtgacatgaactccctcctcaccaccctcgcagaagagagtaaatttctggcaatcatccaaaagtttgtgaactcccccacacccagctttcttgtacaaagtggccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaaccatggctccaaagaagaagcgtaaggtaaatcgatcgaattcaaggcctctcgagcctctagaaggtggcggaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaactgcagccctatagtgagtcgtattacgtagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttccaactttattatacatagttgataattcactggccgtcgttttacatcgatgatgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagatcagatctgcgaagatacggccacgggtgctcttgatcctgtggctgattttggactgtgctgctcgcagctgctgatgaatcacatacttcctccattttcttccactgattgactgttataatttccctaatttccaggtcaaggtgctgtgcattgtggtaatagatgtgacatgacgtcacttccaaaggaccaatgaacatgtctgaccaatttcatataatgtgaaaacgattttcataggcagaataaataacatttaaattaaactgggcatcagcgcaattcaattggtttggtaatagcaagggaaaatagaatgaagtgatctccaaaaaataagtactttttgactgtaaataaaattgtaaggagtaaaaagtacttttttttctaaaaaaatgtaattaagtaaaagtaaaagtattgatttttaattgtactcaagtaaagtaaaaatccccaaaaataatacttaagtacagtaatcaagtaaaattactcaagtactttacacctctggttcttgaccccctaccttcagcaagcccagcagatccactagttctagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtg

SEQ ID NO: 6 - DNA, 12032 bp, Artificial Sequence
  Description of Artificial Sequence Synthetic plasmid
taccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccaaatagtaggaattacccacctgtacaagtgctgaaaacttggatgaataagcccgtttgccattttctactgctattttaaatcttttctgtatttgtctgcatttgtctttacccttgaataaatgttttagttgttttttttcaattatgactgtgtttaacagacattattacaataatatgtaatagtagtacacactattattatgtaatagtacttgttgactgtatttgagaactggaccgagtgagtgttacgtcacccattcaaaatgacttacttctggctccaacaaaatgaagttaattcagttgccatttttcactgtatggacatcgccgtgttggagctagacgtcgccaataagcaagaaagagccgagatgcgtcgagtctgagtcaccgttcctatggcaacccctctaaccaatcagaagtaagcttgttggaagtccacagcctaccacttgaaagcgggctgcacaaaatctgtcaaacgttttgaacgttggatgtgagagcacatacttttattaaggcatctggttggtcagtttataatatcaacaacttgggctacagaaaagaaaagttattacagaaattatgattaacaagtacatgttaaataaagattttaatatgaatgccaccactggagcattcatgccatttggagcttcttcctgtttggatcactagaaggaggaggtcactcattacagttctcatatacagtcgttggttggttggttggttggttggttggtagattgattgattgattgattgattgattgattgattgattgattgattgattgattgattgattgattgattgattgattgattgattgattgattgattgattggtagtcaaaataagaaataatttccacagattcattacagaaatgattaaatgcatacataaaaaactggggggggggggatacaacaacacacttaagtcacatttgcctacgtaaagaaaagtaaagaaaatcaatagctatattttacatctcctttttttgctgtcttttaattagccttgttttgctgttatcttgattgaaactgtaacttcttcacctgcttcttttctttgtaggttttgccagcccagaaacaggcatggctgtgcaaggccagagcaccatgcacaatgcgctgcatgtcttcatgaacggctcaatgtcctcagtccagggctcagccaacgaccccatcttcctccttcaccatgctttcattgacaggtaacaaacacgtcatgacattagactgcacagtttttgacaaagttcatacaatctgttgtttatagctgctacaattagtgaagtttgtgaatgtacttggatgagcagcgaaagatcaattgagatcaattgttagagtttggttgccctgcagagcaaagaacaaaaaataatctggtggctttactgcgtgaggttattattggtggaatagaaacacaaaacataattgcatttatttgtttaattttttatcttatcttaactttcatcttgcatatttgtttcttacatcatttctagcatctttgagcgctggctaagaactcatcagcctccccggtccatctacccacgtaccaatgcaccaattggccacaatgacggctactacatggtgccattccttcctctttataggaatggagactacctcctgtccaacaaggctcttggatacgagtacgcctacctgttggacccaggtcattgcacaacaccagaaatgccctctgatctgcaaaagacgtgaatatctgttcagacacccatatccactctgttccacacaggtcagaggtttgtccaggagttcttgacagaggtgtaaaaagtactcaaaaattttactcaagtgaaagtacaagtacttagggaaaattttactcaattaaaagtaaaagtatctggctagaatcttacttgagtaaaagtaaaaaagtactccattaaaattgtacttgagtattaaggaagtaaaagtaaaagcaagaaagaaaactagagattcttgtttaagcttttaatctcaaaaaacattaaatgaaatgcatacaaggttttatcctgctttagaactgtttgtatttaattatcaaactataagacagacaatctaatgccagtacacgctactcaaagttgtaaaacctcagatttaacttcagtagaagctgattctcaaaattgttagtgtcaagcctagctcttttggggctgaaaagcaatcctgcagtgctgaaaagcctctcacaggcagccgatgcgggaagaggtgtattagtcttgatagagaggctgcaaatagcaggaaacgtgagcagagactccctggtgtctgaaacacaggccagatgggccctcgagcaggaaacagctatgaccatgattacgccaagctatcaactttgtatagaaaagttgcttcaggggtgtcgcttggttatttccaaaaatcaaattaattttattaaactattagaacgagcatgttttgtctatatgctacagaagataaaaaataataggagttaacagttataaaacaacacactttgtttctattgattgttgaccacactggggtctcattaagttagattaaagacacactaactgggtcaaaagcagcagattgatttcatagcaccagggtaaactttctaacacttttacggcaatcatatacattaaaattaaatacagaccacgactgaacaaggaggatgatctccaatattaaacaaagagacttgtgcctatttctctgagggtaaacatgacctctcaagttagcaagttggttttaacactacaaaaatagttaagactgcaatcccagaataaagtattggttttaaccaatcaatatagtacagtaaacatccatttgttttgttgaaacgttaaacaaatctgaccaaagctattagcttatataaaacaggtttgccttctatgtagctgaaaacaccacaggcccgattttgctactgtgtaaaacatttcagcaagatttttttattgcatttttttttactgaatcgttcaaacattttatcattttagtttgttcattcattgcaactggaaaaacaacacatcacacaaccgcacatatttcagcaataagtacaataaaacactcaaataaaaaaaacattttaaatctctttgtatttttgaccgctgtttcgcgtaatttcacggtaaaactctggaaatctccactacattcctctcagcggctcctctcaatgacagctgaagaagtgacgcggctgcctgctgtgttttgattggtcgaattcactggaggcttccagaacagtgtagagtctgaacgggtgcgcgctctgctgtatttaaagggcgaaagagagaccgcagagaaactcaaccgaagagaagcgacttgacaaagaagaaaagagcagcctgacaggactttttccccgacgaggtgtttattcgctctatttaagaatctactgtaaggtaagtctcaatatattgtactctattggctaatcagaattatatagagattatatgtacttaatgtcaaaaaatcaactttgtatatgtaatctttttacatgtggactgcctatgttcatcttattttaggtctactagaaaattatatttcccgttttcacaataaggatttttaaaaaaagcaatgaacagacgggcatttactttatgttgctgacattattttatatgagcataataaccataaatactagcaaatgtcctaaatgaatttgtgttaatgttgtctacaaaagaaaattagcgttttacttgtacaactaataataacttggttattaagagaatttcacttgttgactagaaaaatcctttcataatgaaacaattgcaccataaattgtataaatataaaattaattctaattgtttttttttcctgccaagtttgtacaaaaaagcaggcttcgaaggagatagaaccatggaggagggcaagcaggagccagaggttgtgtatgagactaattgtcactgggatggttgctgcagagagtttgacacccaagagcagcttgtgcatcacataaataatgaccatattcatggagaaaaaaaggaatttgtgtgtcgctgggaggaatgttcaagagaacagaaaccctttaaggcccagtatatgctggtggtgcacatgcgcagacacacaggagagaagcctcataaatgcacgtttgaaggctgttcaaaggcatattccagattagagaacctgaaaacacatttgcgttcacacacaggagagaagccatatgtgtgtgaacatgaaggctgcaacaaagccttttcgaatgcatcagaccgagccaaacaccagaatcggacacactctaatgagaaaccctatgtgtgtaaaattcctggttgcactaaacggtacactgacccaagctccttaaggaaacatgttaaaactgtacatggtcctgaagcccatgtcaccaaaaaacaacgtggggagacatacccacgacccccacctcagccacgagaacctggagcaaccactgaccaaaaggagtatacccatgctacctcaaaacaggatgaatgtctgcaagtcaagtcaatcaaaacagaaaagcccatgacgtctcagtcaagccctggcagtcagtcttcatgcagcagtgaccagtccatcaacggcccccatgtcagccagggagtccagcttactgtgaacagtattgggcttgcgggggaggtggcaatgcttgaggacatggaaaatgaagaggaggatgaggagaacagagaaggagagaaggaggatgcccccattatggactctacagtatccacagcaacatcctctgcggcaacaatagttttgcagactcatcgaagtgttggacggcccctacgctggatggagcatgttaagatggagaggttaaggcaggtgaatggagctgtgcctaggctaggattcctgcctccaacaccgccccgtaaaggccaggccatgcctgccctcacaggaaagggtcaatgccttgggaggatgacttctgtgagggtgcctccatgccctgagccgtccaacacagagctgacaattctgagtttgctgcatgaacgacgggacagtagtggcagcaacactagctcagcttacctgagcagcagccgtcgctcatctggcatctccccctgcttttccagtcgctgttctagccaggcttcactgtcagagtacagcccccatcgacatatccataatctcagtgcaactgattcttatgaccctatttccactgatgcctctcgacgctccagtgaagccagccagtatgatggaggaagttcaagtgcagtggtgtttggtggaagtggagttttcaagggcagtggtatgggatcaagaggggatctgaacctcactccagctcaacactatagcctaaaggccaagtatgcagcagcaacaggtggacctcctcccacacccttgcccagtatggggcatataaacctaaagactcactttgctttaaaggaagacagaaaggacactggccagcttacactaccaccactggtcaggtcacgtcgctgtagtgatggtgcacacaactcgcatgacagtcaagccgtgtatcaacgaaagggaacatactttggtggtggtccagggaaaaatgatcgaagagctagtgatcctacacatcacaaacaaaatcttagttcttatggcttgccacaagtacgacatttcagtagtttaaacaatatgacttcatcacccaatcctgctagttatggttactcaaacagagaagatcacagtctgaacctacagaattcaacctgtcatgtagatagctttcagagtggtctgcactccccttgtcctcctagcatcatggagcaatcagcacttgagaacattttcatgaaccaggatggtcttttgttacgtgtcaatgaaaacatccagtctgatttcatgcagtatctccattcccaagatcaggaatcaataattatccaagacaaccataggcctattttcaatcctgcagatgaccaaaacttcctcagtccacagcagcagcaagagggggtagagcaagaagacactcgtatagatctgatgcctatccagtcgaatgaagtgagctctgctagtgctgatgttactattccaaggcagccgtttcagaggtgtggcagatggtcagaccagagtggctcagtaagcaacgcctttggtcgatttggaaacatggttgtacaacagcagcctatagattttattgatcagcactcagctcttcaacacatgggaggtataagctatgggtgcagaaattcagagcagcatctacaagtagcaaaaccagcccaagagaagtccaggatgaaaacaggcattaagactgagtcttcctcaaccttccaactggaatccaaacacagcaactttggtcgaccagacttctctcatatcttttcggcccccattcatcaaaaatgttcccagcatacagttgtatcccaatcttatccccaacaaaatcaaattacaaatcaagatagcagcaacctctttttgcaaagaccaggtaactgcagcttactcttggcttaccggcctcagccagctgtcaacacttcttactccactaggcaggatattaacagccataatgtacaatcatgccatccacaaagcttgaacaatagactaggcacaagttgttccctttcaaatcaggtttccgttgtgaagcttgatcaagttaatcaccttcaaatcaactcttgctcaacaattcagcaacaaaagcctgtgtttattgataagtgttttgacccagaagagtccaagctctctcacgccatagtacagtcatgtctaatggaatcattgatggcagagaaagaaagaacttccaaagttcttctgtcatctggggttggtcaggtgactagcaaagtagaaagaagcccagctgaaaaagttggtttaggaatttgttccattggtggtgatagttatgatcagggcagtgtagtgtcttcctctcgactcagtaattctcatgattcctttgcatttcacagtgtggatccaggcactaacaacatggctattagtgacatgaactccctcctcaccaccctcgcagaagagagtaaatttctggcaatcatccaaaagtttgtgaactcccccacacccagctttcttgtacaaagtggttatggtgagcaagggcgaggacgacaacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgccgaggtcaagaccacctacaaggccaagaagcccgtgcagctgcccggcgcctacaacgtcaacatcaagctggacatcacctcccacaacgaggactacaccatcgtggaacagtacgagcgcgccgagggccgccactccaccggcggcatggacgagctgtacaagtaaggcgcgcctctagaactatagtgagtcgtattacgtagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttccaactttattatacatagttgataattcactggccgtcgttttacatcgatgatgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagatcagatctgcgaagatacggccacgggtgctcttgatcctgtggctgattttggactgtgctgctcgcagctgctgatgaatcacatacttcctccattttcttccactgattgactgttataatttccctaatttccaggtcaaggtgctgtgcattgtggtaatagatgtgacatgacgtcacttccaaaggaccaatgaacatgtctgaccaatttcatataatgtgaaaacgattttcataggcagaataaataacatttaaattaaactgggcatcagcgcaattcaattggtttggtaatagcaagggaaaatagaatgaagtgatctccaaaaaataagtactttttgactgtaaataaaattgtaaggagtaaaaagtacttttttttctaaaaaaatgtaattaagtaaaagtaaaagtattgatttttaattgtactcaagtaaagtaaaaatccccaaaaataatacttaagtacagtaatcaagtaaaattactcaagtactttacacctctggttcttgaccccctaccttcagcaagcccagcagatccactagttctagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtg

SEQ ID NO: 7 - DNA, 11966 bp, Artificial Sequence
  Description of Artificial Sequence Synthetic plasmid
taccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccaaatagtaggaattacccacctgtacaagtgctgaaaacttggatgaataagcccgtttgccattttctactgctattttaaatcttttctgtatttgtctgcatttgtctttacccttgaataaatgttttagttgttttttttcaattatgactgtgtttaacagacattattacaataatatgtaatagtagtacacactattattatgtaatagtacttgttgactgtatttgagaactggaccgagtgagtgttacgtcacccattcaaaatgacttacttctggctccaacaaaatgaagttaattcagttgccatttttcactgtatggacatcgccgtgttggagctagacgtcgccaataagcaagaaagagccgagatgcgtcgagtctgagtcaccgttcctatggcaacccctctaaccaatcagaagtaagcttgttggaagtccacagcctaccacttgaaagcgggctgcacaaaatctgtcaaacgttttgaacgttggatgtgagagcacatacttttattaaggcatctggttggtcagtttataatatcaacaacttgggctacagaaaagaaaagttattacagaaattatgattaacaagtacatgttaaataaagattttaatatgaatgccaccactggagcattcatgccatttggagcttcttcctgtttggatcactagaaggaggaggtcactcattacagttctcatatacagtcgttggttggttggttggttggttggttggtagattgattgattgattgattgattgattgattgattgattgattgattgattgattgattgattgattgattgattgattgattgattgattgattgattgattggtagtcaaaataagaaataatttccacagattcattacagaaatgattaaatgcatacataaaaaactggggggggggggatacaacaacacacttaagtcacatttgcctacgtaaagaaaagtaaagaaaatcaatagctatattttacatctcctttttttgctgtcttttaattagccttgttttgctgttatcttgattgaaactgtaacttcttcacctgcttcttttctttgtaggttttgccagcccagaaacaggcatggctgtgcaaggccagagcaccatgcacaatgcgctgcatgtcttcatgaacggctcaatgtcctcagtccagggctcagccaacgaccccatcttcctccttcaccatgctttcattgacaggtaacaaacacgtcatgacattagactgcacagtttttgacaaagttcatacaatctgttgtttatagctgctacaattagtgaagtttgtgaatgtacttggatgagcagcgaaagatcaattgagatcaattgttagagtttggttgccctgcagagcaaagaacaaaaaataatctggtggctttactgcgtgaggttattattggtggaatagaaacacaaaacataattgcatttatttgtttaattttttatcttatcttaactttcatcttgcatatttgtttcttacatcatttctagcatctttgagcgctggctaagaactcatcagcctccccggtccatctacccacgtaccaatgcaccaattggccacaatgacggctactacatggtgccattccttcctctttataggaatggagactacctcctgtccaacaaggctcttggatacgagtacgcctacctgttggacccaggtcattgcacaacaccagaaatgccctctgatctgcaaaagacgtgaatatctgttcagacacccatatccactctgttccacacaggtcagaggtttgtccaggagttcttgacagaggtgtaaaaagtactcaaaaattttactcaagtgaaagtacaagtacttagggaaaattttactcaattaaaagtaaaagtatctggctagaatcttacttgagtaaaagtaaaaaagtactccattaaaattgtacttgagtattaaggaagtaaaagtaaaagcaagaaagaaaactagagattcttgtttaagcttttaatctcaaaaaacattaaatgaaatgcatacaaggttttatcctgctttagaactgtttgtatttaattatcaaactataagacagacaatctaatgccagtacacgctactcaaagttgtaaaacctcagatttaacttcagtagaagctgattctcaaaattgttagtgtcaagcctagctcttttggggctgaaaagcaatcctgcagtgctgaaaagcctctcacaggcagccgatgcgggaagaggtgtattagtcttgatagagaggctgcaaatagcaggaaacgtgagcagagactccctggtgtctgaaacacaggccagatgggccctcgagcaggaaacagctatgaccatgattacgccaagctatcaactttgtatagaaaagttgcttcaggggtgtcgcttggttatttccaaaaatcaaattaattttattaaactattagaacgagcatgttttgtctatatgctacagaagataaaaaataataggagttaacagttataaaacaacacactttgtttctattgattgttgaccacactggggtctcattaagttagattaaagacacactaactgggtcaaaagcagcagattgatttcatagcaccagggtaaactttctaacacttttacggcaatcatatacattaaaattaaatacagaccacgactgaacaaggaggatgatctccaatattaaacaaagagacttgtgcctatttctctgagggtaaacatgacctctcaagttagcaagttggttttaacactacaaaaatagttaagactgcaatcccagaataaagtattggttttaaccaatcaatatagtacagtaaacatccatttgttttgttgaaacgttaaacaaatctgaccaaagctattagcttatataaaacaggtttgccttctatgtagctgaaaacaccacaggcccgattttgctactgtgtaaaacatttcagcaagatttttttattgcatttttttttactgaatcgttcaaacattttatcattttagtttgttcattcattgcaactggaaaaacaacacatcacacaaccgcacatatttcagcaataagtacaataaaacactcaaataaaaaaaacattttaaatctctttgtatttttgaccgctgtttcgcgtaatttcacggtaaaactctggaaatctccactacattcctctcagcggctcctctcaatgacagctgaagaagtgacgcggctgcctgctgtgttttgattggtcgaattcactggaggcttccagaacagtgtagagtctgaacgggtgcgcgctctgctgtatttaaagggcgaaagagagaccgcagagaaactcaaccgaagagaagcgacttgacaaagaagaaaagagcagcctgacaggactttttccccgacgaggtgtttattcgctctatttaagaatctactgtaaggtaagtctcaatatattgtactctattggctaatcagaattatatagagattatatgtacttaatgtcaaaaaatcaactttgtatatgtaatctttttacatgtggactgcctatgttcatcttattttaggtctactagaaaattatatttcccgttttcacaataaggatttttaaaaaaagcaatgaacagacgggcatttactttatgttgctgacattattttatatgagcataataaccataaatactagcaaatgtcctaaatgaatttgtgttaatgttgtctacaaaagaaaattagcgttttacttgtacaactaataataacttggttattaagagaatttcacttgttgactagaaaaatcctttcataatgaaacaattgcaccataaattgtataaatataaaattaattctaattgtttttttttcctgccaagtttgtacaaaaaagcaggcttcgaaggagatagaaccatggaggagaaacccgaacccgaggccatttatgagacgaactgccactgggagagctgcagcaaagagttcgacacgcaggagcagctggtgcatcacatcaataatgagcacatccacggagagaagaaggagttcgtttgccactggaaggactgttcccgagagcagcgtccgtttaaagcgcagtacatgctggtggtccacatgcgcagacacaccggagaaaaaccacacaagtgcacttttgaaggctgtaataaagcatactcgcggctggagaacctgaagacgcatctgcgctcacacaccggagagaaaccctacgtttgtgaacacgagggctgtaacaaggctttctctaatgcttcagatcgagcaaagcaccagaacagaacacactccaatgagaaaccgtacatttgtaagattcccggctgtacgaagcgctacacagacccgagctctctgaggaagcatgttaagacggtacacggaccggaggcgcatataacgaaaaaacaccgtggcgacaccggccctcgtcctcctggtctgaccactgcaggacagagctctgagctgctgatagagaaggaggagagaaaccgagaagattgtaaactactcgctccagacaacacactgaaatctcagccgagccctggtggccagtcttcctgcagtagcgaacgttctccactagggagcgccaataataatgacagcggtgtggagatgaatcttaatgcggcgggaagtctggaagatctgaccacacaggaggacagcgggaatgctggtgtgtccgaatccagcgctaccatcagctcggggggaatgtgcatgtctgtacaggccttgaagaggttggaaaacctgaagatcgacaaactaaagcaaattcgtcggccgacgccacctgggcgcaacgcaggaaacaaactaccagctctctcagcaacaggagagatgatgagcatgtgtgctccctcgcccctgctctccaaccggcgtgtgatggagctttcagctcccgatatgggtggagtcactggaatgtcctgccctccgaacgaccggcgcggcagcggcaccagcagcctcagctctgcttacaccgtgagccgccggtcatcaatggtctcgccatacctgtccagcagacgctccagtgacgtgtctcactgccagtctgtgatggggggtgaggttcctggtgacccgctgtctccgcagaacagccaaagagccggactgtgccagaattctggaggtttaccaggtcttccgagtcttactcctgcacagcagtatagtctaaaggctaaatatgcagcagcaacaggaggtccaccccctaccccgcttcccaacatggaccaggctggcactcctgcaagacatgtgggattcttgagggaatgccaaggccagcctcttcctccatttctgcagcaaggtggcacaagaagacacagtgctaatgccgaatatggcacaggagtgatctatccacatcaggcacctggcaacaacacaagacgtgccagcgatccggtgcgatccgcggccgacccgcagggtttacccaaagtgcagcgatttaacagcttgagcaacgtgtctcttatgagtcgccggaatgcactgcagcagtgcggatctgatgctgctctgagcaggcacatgtattcgcctcgaccacctagtatcactgaaaatgtcatgatggaggccatgggtatggatgggaacaccgaaggcagacagcagggcaacatgatcccaggtggagaccgaagctacatgggctaccagcacaatcctcatcaagcaagtcagctgtcccctgggcaagaaagtctaggatgcattgaccaagtttatcagtctcaaatgcaaggtcagtatcagcgtgaggagagttgctccaccggcgtaatgggtcaggctgacattgcaaataatctactacagcaagctgagtatgggatgagcacttgtcagctcagtccctcaggtccacactatcctagccaaggggatggatcaggaccttggggacaaaccaaccagctccacagccctgggatgcaataccagggtgcaggaatgcaaggacagcactacactcaacagggcatatatgaccctacatctaaccctaaccttcaaagagttactgtaaaaccagagcaattccatccatcaatgggaggatctagttcttgccaaaacactaaagctctgcaccagaataggcataatgcaaatatgcaaacatatccactacaaggccaaggtattatgaataggtcctccagtgcaagttgtgacttccatcattcccaaatgggaacacaaccaaaccaggggggatcattccagagtggtactggtataaaccttgccctggcagaaagccgcagatcacaaacaccaatgcatcaaatgaaggaaatgatggtcaggaactatgtgcaatcccagcaagctctcctatgggaacagcagcaagagcagagtgtgtccgaaaaacctgatggcatggacatgggacaaacacagatgatgcagcacagtccgcagcatcaacaagccaaccagaatctttatcctggtaacacttaccaaggctacccaaatcagaacttgatgagcccacagcaaaatcgagttcctggatctgtcaaagagcaaatgcaaagctcctgctatgggccagacatgattccaaggccaccccaagtgcgaaaatctttaagtcgccaaaacagcctttcccaacaagcaggtggagcttacctgggaagcccacctcacctcagcccggtccactctacagctagtcccagaagaggagttagactcccgccagttcaacaacaacaacaacagcagcagcagcattcagaaaacttcaacaacaacaacaacccaatgtactactcaggccagatgcacatgcaccacgacctagagaagactccagagggtccatgccttgctcagcagcacctaacaagctcagaccccactacaaaaccaacatcaatctcttatccagaccctgcccccatgtccaatgccttagaacacctagacttggagaacgctcagattgactttacctccatcattgatgaccaagaaccttcatcttacagtccaatcaatgcacctataggacacaatcaatgctcctctcagacgtcctctcgccttacaacaccccaaaactccatcacacttcccagtggcctttctaatatggcaatcggagacatgagctcaatgttgacatcccttgctggggaaaacaagtatctaaacacgttatcttacccagctttcttgtacaaagtggacatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctcgagctcaagcttcgaattctgcagtcgacggtaccgcgggcccgggatccaccggatcttgaccaactttattatacatagttgataattcactggccgtcgttttacatcgatgatgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagatcagatctgcgaagatacggccacgggtgctcttgatcctgtggctgattttggactgtgctgctcgcagctgctgatgaatcacatacttcctccattttcttccactgattgactgttataatttccctaatttccaggtcaaggtgctgtgcattgtggtaatagatgtgacatgacgtcacttccaaaggaccaatgaacatgtctgaccaatttcatataatgtgaaaacgattttcataggcagaataaataacatttaaattaaactgggcatcagcgcaattcaattggtttggtaatagcaagggaaaatagaatgaagtgatctccaaaaaataagtactttttgactgtaaataaaattgtaaggagtaaaaagtacttttttttctaaaaaaatgtaattaagtaaaagtaaaagtattgatttttaattgtactcaagtaaagtaaaaatccccaaaaataatacttaagtacagtaatcaagtaaaattactcaagtactttacacctctggttcttgaccccctaccttcagcaagcccagcagatccactagttctagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtg

SEQ ID NO: 8 - DNA, 12777 bp, Artificial Sequence
  Description of Artificial Sequence Synthetic plasmid
taccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccaaatagtaggaattacccacctgtacaagtgctgaaaacttggatgaataagcccgtttgccattttctactgctattttaaatcttttctgtatttgtctgcatttgtctttacccttgaataaatgttttagttgttttttttcaattatgactgtgtttaacagacattattacaataatatgtaatagtagtacacactattattatgtaatagtacttgttgactgtatttgagaactggaccgagtgagtgttacgtcacccattcaaaatgacttacttctggctccaacaaaatgaagttaattcagttgccatttttcactgtatggacatcgccgtgttggagctagacgtcgccaataagcaagaaagagccgagatgcgtcgagtctgagtcaccgttcctatggcaacccctctaaccaatcagaagtaagcttgttggaagtccacagcctaccacttgaaagcgggctgcacaaaatctgtcaaacgttttgaacgttggatgtgagagcacatacttttattaaggcatctggttggtcagtttataatatcaacaacttgggctacagaaaagaaaagttattacagaaattatgattaacaagtacatgttaaataaagattttaatatgaatgccaccactggagcattcatgccatttggagcttcttcctgtttggatcactagaaggaggaggtcactcattacagttctcatatacagtcgttggttggttggttggttggttggttggtagattgattgattgattgattgattgattgattgattgattgattgattgattgattgattgattgattgattgattgattgattgattgattgattgattgattggtagtcaaaataagaaataatttccacagattcattacagaaatgattaaatgcatacataaaaaactggggggggggggatacaacaacacacttaagtcacatttgcctacgtaaagaaaagtaaagaaaatcaatagctatattttacatctcctttttttgctgtcttttaattagccttgttttgctgttatcttgattgaaactgtaacttcttcacctgcttcttttctttgtaggttttgccagcccagaaacaggcatggctgtgcaaggccagagcaccatgcacaatgcgctgcatgtcttcatgaacggctcaatgtcctcagtccagggctcagccaacgaccccatcttcctccttcaccatgctttcattgacaggtaacaaacacgtcatgacattagactgcacagtttttgacaaagttcatacaatctgttgtttatagctgctacaattagtgaagtttgtgaatgtacttggatgagcagcgaaagatcaattgagatcaattgttagagtttggttgccctgcagagcaaagaacaaaaaataatctggtggctttactgcgtgaggttattattggtggaatagaaacacaaaacataattgcatttatttgtttaattttttatcttatcttaactttcatcttgcatatttgtttcttacatcatttctagcatctttgagcgctggctaagaactcatcagcctccccggtccatctacccacgtaccaatgcaccaattggccacaatgacggctactacatggtgccattccttcctctttataggaatggagactacctcctgtccaacaaggctcttggatacgagtacgcctacctgttggacccaggtcattgcacaacaccagaaatgccctctgatctgcaaaagacgtgaatatctgttcagacacccatatccactctgttccacacaggtcagaggtttgtccaggagttcttgacagaggtgtaaaaagtactcaaaaattttactcaagtgaaagtacaagtacttagggaaaattttactcaattaaaagtaaaagtatctggctagaatcttacttgagtaaaagtaaaaaagtactccattaaaattgtacttgagtattaaggaagtaaaagtaaaagcaagaaagaaaactagagattcttgtttaagcttttaatctcaaaaaacattaaatgaaatgcatacaaggttttatcctgctttagaactgtttgtatttaattatcaaactataagacagacaatctaatgccagtacacgctactcaaagttgtaaaacctcagatttaacttcagtagaagctgattctcaaaattgttagtgtcaagcctagctcttttggggctgaaaagcaatcctgcagtgctgaaaagcctctcacaggcagccgatgcgggaagaggtgtattagtcttgatagagaggctgcaaatagcaggaaacgtgagcagagactccctggtgtctgaaacacaggccagatgggccctcgagcaggaaacagctatgaccatgattacgccaagctatcaactttgtatagaaaagttgcttcaggggtgtcgcttggttatttccaaaaatcaaattaattttattaaactattagaacgagcatgttttgtctatatgctacagaagataaaaaataataggagttaacagttataaaacaacacactttgtttctattgattgttgaccacactggggtctcattaagttagattaaagacacactaactgggtcaaaagcagcagattgatttcatagcaccagggtaaactttctaacacttttacggcaatcatatacattaaaattaaatacagaccacgactgaacaaggaggatgatctccaatattaaacaaagagacttgtgcctatttctctgagggtaaacatgacctctcaagttagcaagttggttttaacactacaaaaatagttaagactgcaatcccagaataaagtattggttttaaccaatcaatatagtacagtaaacatccatttgttttgttgaaacgttaaacaaatctgaccaaagctattagcttatataaaacaggtttgccttctatgtagctgaaaacaccacaggcccgattttgctactgtgtaaaacatttcagcaagatttttttattgcatttttttttactgaatcgttcaaacattttatcattttagtttgttcattcattgcaactggaaaaacaacacatcacacaaccgcacatatttcagcaataagtacaataaaacactcaaataaaaaaaacattttaaatctctttgtatttttgaccgctgtttcgcgtaatttcacggtaaaactctggaaatctccactacattcctctcagcggctcctctcaatgacagctgaagaagtgacgcggctgcctgctgtgttttgattggtcgaattcactggaggcttccagaacagtgtagagtctgaacgggtgcgcgctctgctgtatttaaagggcgaaagagagaccgcagagaaactcaaccgaagagaagcgacttgacaaagaagaaaagagcagcctgacaggactttttccccgacgaggtgtttattcgctctatttaagaatctactgtaaggtaagtctcaatatattgtactctattggctaatcagaattatatagagattatatgtacttaatgtcaaaaaatcaactttgtatatgtaatctttttacatgtggactgcctatgttcatcttattttaggtctactagaaaattatatttcccgttttcacaataaggatttttaaaaaaagcaatgaacagacgggcatttactttatgttgctgacattattttatatgagcataataaccataaatactagcaaatgtcctaaatgaatttgtgttaatgttgtctacaaaagaaaattagcgttttacttgtacaactaataataacttggttattaagagaatttcacttgttgactagaaaaatcctttcataatgaaacaattgcaccataaattgtataaatataaaattaattctaattgtttttttttcctgccaagtttgtacaaaaaagcaggcttcgaaggagatagaaccatggaggagaaacccgaacccgaggccatttatgagacgaactgccactgggagagctgcagcaaagagttcgacacgcaggagcagctggtgcatcacatcaataatgagcacatccacggagagaagaaggagttcgtttgccactggaaggactgttcccgagagcagcgtccgtttaaagcgcagtacatgctggtggtccacatgcgcagacacaccggagaaaaaccacacaagtgcacttttgaaggctgtaataaagcatactcgcggctggagaacctgaagacgcatctgcgctcacacaccggagagaaaccctacgtttgtgaacacgagggctgtaacaaggctttctctaatgcttcagatcgagcaaagcaccagaacagaacacactccaatgagaaaccgtacatttgtaagattcccggctgtacgaagcgctacacagacccgagctctctgaggaagcatgttaagacggtacacggaccggaggcgcatataacgaaaaaacaccgtggcgacaccggccctcgtcctcctggtctgaccactgcaggacagagctctgagctgctgatagagaaggaggagagaaaccgagaagattgtaaactactcgctccagacaacacactgaaatctcagccgagccctggtggccagtcttcctgcagtagcgaacgttctccactagggagcgccaataataatgacagcggtgtggagatgaatcttaatgcggcgggaagtctggaagatctgaccacacaggaggacagcgggaatgctggtgtgtccgaatccagcgctaccatcagctcggggggaatgtgcatgtctgtacaggccttgaagaggttggaaaacctgaagatcgacaaactaaagcaaattcgtcggccgacgccacctgggcgcaacgcaggaaacaaactaccagctctctcagcaacaggagagatgatgagcatgtgtgctccctcgcccctgctctccaaccggcgtgtgatggagctttcagctcccgatatgggtggagtcactggaatgtcctgccctccgaacgaccggcgcggcagcggcaccagcagcctcagctctgcttacaccgtgagccgccggtcatcaatggtctcgccatacctgtccagcagacgctccagtgacgtgtctcactgccagtctgtgatggggggtgaggttcctggtgacccgctgtctccgcagaacagccaaagagccggactgtgccagaattctggaggtttaccaggtcttccgagtcttactcctgcacagcagtatagtctaaaggctaaatatgcagcagcaacaggaggtccaccccctaccccgcttcccaacatggaccaggctggcactcctgcaagacatgtgggattcttgagggaatgccaaggccagcctcttcctccatttctgcagcaaggtggcacaagaagacacagtgctaatgccgaatatggcacaggagtgatctatccacatcaggcacctggcaacaacacaagacgtgccagcgatccggtgcgatccgcggccgacccgcagggtttacccaaagtgcagcgatttaacagcttgagcaacgtgtctcttatgagtcgccggaatgcactgcagcagtgcggatctgatgctgctctgagcaggcacatgtattcgcctcgaccacctagtatcactgaaaatgtcatgatggaggccatgggtatggatgggaacaccgaaggcagacagcagggcaacatgatcccaggtggagaccgaagctacatgggctaccagcacaatcctcatcaagcaagtcagctgtcccctgggcaagaaagtctaggatgcattgaccaagtttatcagtctcaaatgcaaggtcagtatcagcgtgaggagagttgctccaccggcgtaatgggtcaggctgacattgcaaataatctactacagcaagctgagtatgggatgagcacttgtcagctcagtccctcaggtccacactatcctagccaaggggatggatcaggaccttggggacaaaccaaccagctccacagccctgggatgcaataccagggtgcaggaatgcaaggacagcactacactcaacagggcatatatgaccctacatctaaccctaaccttcaaagagttactgtaaaaccagagcaattccatccatcaatgggaggatctagttcttgccaaaacactaaagctctgcaccagaataggcataatgcaaatatgcaaacatatccactacaaggccaaggtattatgaataggtcctccagtgcaagttgtgacttccatcattcccaaatgggaacacaaccaaaccaggggggatcattccagagtggtactggtataaaccttgccctggcagaaagccgcagatcacaaacaccaatgcatcaaatgaaggaaatgatggtcaggaactatgtgcaatcccagcaagctctcctatgggaacagcagcaagagcagagtgtgtccgaaaaacctgatggcatggacatgggacaaacacagatgatgcagcacagtccgcagcatcaacaagccaaccagaatctttatcctggtaacacttaccaaggctacccaaatcagaacttgatgagcccacagcaaaatcgagttcctggatctgtcaaagagcaaatgcaaagctcctgctatgggccagacatgattccaaggccaccccaagtgcgaaaatctttaagtcgccaaaacagcctttcccaacaagcaggtggagcttacctgggaagcccacctcacctcagcccggtccactctacagctagtcccagaagaggagttagactcccgccagttcaacaacaacaacaacagcagcagcagcattcagaaaacttcaacaacaacaacaacccaatgtactactcaggccagatgcacatgcaccacgacctagagaagactccagagggtccatgccttgctcagcagcacctaacaagctcagaccccactacaaaaccaacatcaatctcttatccagaccctgcccccatgtccaatgccttagaacacctagacttggagaacgctcagattgactttacctccatcattgatgaccaagaaccttcatcttacagtccaatcaatgcacctataggacacaatcaatgctcctctcagacgtcctctcgccttacaacaccccaaaactccatcacacttcccagtggcctttctaatatggcaatcggagacatgagctcaatgttgacatcccttgctggggaaaacaagtatctaaacacgttatcttacccagctttcttgtacaaagtggccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaaccatggctccaaagaagaagcgtaaggtaaatcgatcgaattcaaggcctctcgagcctctagaaggtggcggaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaactgcagccctatagtgagtcgtattacgtagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttccaactttattatacatagttgataattcactggccgtcgttttacatcgatgatgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagatcagatctgcgaagatacggccacgggtgctcttgatcctgtggctgattttggactgtgctgctcgcagctgctgatgaatcacatacttcctccattttcttccactgattgactgttataatttccctaatttccaggtcaaggtgctgtgcattgtggtaatagatgtgacatgacgtcacttccaaaggaccaatgaacatgtctgaccaatttcatataatgtgaaaacgattttcataggcagaataaataacatttaaattaaactgggcatcagcgcaattcaattggtttggtaatagcaagggaaaatagaatgaagtgatctccaaaaaataagtactttttgactgtaaataaaattgtaaggagtaaaaagtacttttttttctaaaaaaatgtaattaagtaaaagtaaaagtattgatttttaattgtactcaagtaaagtaaaaatccccaaaaataatacttaagtacagtaatcaagtaaaattactcaagtactttacacctctggttcttgaccccctaccttcagcaagcccagcagatccactagttctagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtg

SEQ ID NO: 9 - DNA, 12095 bp, Artificial Sequence
  Description of Artificial Sequence Synthetic plasmid
taccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccaaatagtaggaattacccacctgtacaagtgctgaaaacttggatgaataagcccgtttgccattttctactgctattttaaatcttttctgtatttgtctgcatttgtctttacccttgaataaatgttttagttgttttttttcaattatgactgtgtttaacagacattattacaataatatgtaatagtagtacacactattattatgtaatagtacttgttgactgtatttgagaactggaccgagtgagtgttacgtcacccattcaaaatgacttacttctggctccaacaaaatgaagttaattcagttgccatttttcactgtatggacatcgccgtgttggagctagacgtcgccaataagcaagaaagagccgagatgcgtcgagtctgagtcaccgttcctatggcaacccctctaaccaatcagaagtaagcttgttggaagtccacagcctaccacttgaaagcgggctgcacaaaatctgtcaaacgttttgaacgttggatgtgagagcacatacttttattaaggcatctggttggtcagtttataatatcaacaacttgggctacagaaaagaaaagttattacagaaattatgattaacaagtacatgttaaataaagattttaatatgaatgccaccactggagcattcatgccatttggagcttcttcctgtttggatcactagaaggaggaggtcactcattacagttctcatatacagtcgttggttggttggttggttggttggttggtagattgattgattgattgattgattgattgattgattgattgattgattgattgattgattgattgattgattgattgattgattgattgattgattgattgattggtagtcaaaataagaaataatttccacagattcattacagaaatgattaaatgcatacataaaaaactggggggggggggatacaacaacacacttaagtcacatttgcctacgtaaagaaaagtaaagaaaatcaatagctatattttacatctcctttttttgctgtcttttaattagccttgttttgctgttatcttgattgaaactgtaacttcttcacctgcttcttttctttgtaggttttgccagcccagaaacaggcatggctgtgcaaggccagagcaccatgcacaatgcgctgcatgtcttcatgaacggctcaatgtcctcagtccagggctcagccaacgaccccatcttcctccttcaccatgctttcattgacaggtaacaaacacgtcatgacattagactgcacagtttttgacaaagttcatacaatctgttgtttatagctgctacaattagtgaagtttgtgaatgtacttggatgagcagcgaaagatcaattgagatcaattgttagagtttggttgccctgcagagcaaagaacaaaaaataatctggtggctttactgcgtgaggttattattggtggaatagaaacacaaaacataattgcatttatttgtttaattttttatcttatcttaactttcatcttgcatatttgtttcttacatcatttctagcatctttgagcgctggctaagaactcatcagcctccccggtccatctacccacgtaccaatgcaccaattggccacaatgacggctactacatggtgccattccttcctctttataggaatggagactacctcctgtccaacaaggctcttggatacgagtacgcctacctgttggacccaggtcattgcacaacaccagaaatgccctctgatctgcaaaagacgtgaatatctgttcagacacccatatccactctgttccacacaggtcagaggtttgtccaggagttcttgacagaggtgtaaaaagtactcaaaaattttactcaagtgaaagtacaagtacttagggaaaattttactcaattaaaagtaaaagtatctggctagaatcttacttgagtaaaagtaaaaaagtactccattaaaattgtacttgagtattaaggaagtaaaagtaaaagcaagaaagaaaactagagattcttgtttaagcttttaatctcaaaaaacattaaatgaaatgcatacaaggttttatcctgctttagaactgtttgtatttaattatcaaactataagacagacaatctaatgccagtacacgctactcaaagttgtaaaacctcagatttaacttcagtagaagctgattctcaaaattgttagtgtcaagcctagctcttttggggctgaaaagcaatcctgcagtgctgaaaagcctctcacaggcagccgatgcgggaagaggtgtattagtcttgatagagaggctgcaaatagcaggaaacgtgagcagagactccctggtgtctgaaacacaggccagatgggccctcgagcaggaaacagctatgaccatgattacgccaagctatcaactttgtatagaaaagttgcttcaggggtgtcgcttggttatttccaaaaatcaaattaattttattaaactattagaacgagcatgttttgtctatatgctacagaagataaaaaataataggagttaacagttataaaacaacacactttgtttctattgattgttgaccacactggggtctcattaagttagattaaagacacactaactgggtcaaaagcagcagattgatttcatagcaccagggtaaactttctaacacttttacggcaatcatatacattaaaattaaatacagaccacgactgaacaaggaggatgatctccaatattaaacaaagagacttgtgcctatttctctgagggtaaacatgacctctcaagttagcaagttggttttaacactacaaaaatagttaagactgcaatcccagaataaagtattggttttaaccaatcaatatagtacagtaaacatccatttgttttgttgaaacgttaaacaaatctgaccaaagctattagcttatataaaacaggtttgccttctatgtagctgaaaacaccacaggcccgattttgctactgtgtaaaacatttcagcaagatttttttattgcatttttttttactgaatcgttcaaacattttatcattttagtttgttcattcattgcaactggaaaaacaacacatcacacaaccgcacatatttcagcaataagtacaataaaacactcaaataaaaaaaacattttaaatctctttgtatttttgaccgctgtttcgcgtaatttcacggtaaaactctggaaatctccactacattcctctcagcggctcctctcaatgacagctgaagaagtgacgcggctgcctgctgtgttttgattggtcgaattcactggaggcttccagaacagtgtagagtctgaacgggtgcgcgctctgctgtatttaaagggcgaaagagagaccgcagagaaactcaaccgaagagaagcgacttgacaaagaagaaaagagcagcctgacaggactttttccccgacgaggtgtttattcgctctatttaagaatctactgtaaggtaagtctcaatatattgtactctattggctaatcagaattatatagagattatatgtacttaatgtcaaaaaatcaactttgtatatgtaatctttttacatgtggactgcctatgttcatcttattttaggtctactagaaaattatatttcccgttttcacaataaggatttttaaaaaaagcaatgaacagacgggcatttactttatgttgctgacattattttatatgagcataataaccataaatactagcaaatgtcctaaatgaatttgtgttaatgttgtctacaaaagaaaattagcgttttacttgtacaactaataataacttggttattaagagaatttcacttgttgactagaaaaatcctttcataatgaaacaattgcaccataaattgtataaatataaaattaattctaattgtttttttttcctgccaagtttgtacaaaaaagcaggctgaggagaaacccgaacccgaggccatttatgagacgaactgccactgggagagctgcagcaaagagttcgacacgcaggagcagctggtgcatcacatcaataatgagcacatccacggagagaagaaggagttcgtttgccactggaaggactgttcccgagagcagcgtccgtttaaagcgcagtacatgctggtggtccacatgcgcagacacaccggagaaaaaccacacaagtgcacttttgaaggctgtaataaagcatactcgcggctggagaacctgaagacgcatctgcgctcacacaccggagagaaaccctacgtttgtgaacacgagggctgtaacaaggctttctctaatgcttcagatcgagcaaagcaccagaacagaacacactccaatgagaaaccgtacatttgtaagattcccggctgtacgaagcgctacacagacccgagctctctgaggaagcatgttaagacggtacacggaccggaggcgcatataacgaaaaaacaccgtggcgacaccggccctcgtcctcctggtctgaccactgcaggacagagctctgagctgctgatagagaaggaggagagaaaccgagaagattgtaaactactcgctccagacaacacactgaaatctcagccgagccctggtggccagtcttcctgcagtagcgaacgttctccactagggagcgccaataataatgacagcggtgtggagatgaatcttaatgcggcgggaagtctggaagatctgaccacacaggaggacagcgggaatgctggtgtgtccgaatccagcgctaccatcagctcggggggaatgtgcatgtctgtacaggccttgaagaggttggaaaacctgaagatcgacaaactaaagcaaattcgtcggccgacgccacctgggcgcaacgcaggaaacaaactaccagctctctcagcaacaggagagatgatgagcatgtgtgctccctcgcccctgctctccaaccggcgtgtgatggagctttcagctcccgatatgggtggagtcactggaatgtcctgccctccgaacgaccggcgcggcagcggcaccagcagcctcagctctgcttacaccgtgagccgccggtcatcaatggtctcgccatacctgtccagcagacgctccagtgacgtgtctcactgccagtctgtgatggggggtgaggttcctggtgacccgctgtctccgcagaacagccaaagagccggactgtgccagaattctggaggtttaccaggtcttccgagtcttactcctgcacagcagtatagtctaaaggctaaatatgcagcagcaacaggaggtccaccccctaccccgcttcccaacatggaccaggctggcactcctgcaagacatgtgggattcttgagggaatgccaaggccagcctcttcctccatttctgcagcaaggtggcacaagaagacacagtgctaatgccgaatatggcacaggagtgatctatccacatcaggcacctggcaacaacacaagacgtgccagcgatccggtgcgatccgcggccgacccgcagggtttacccaaagtgcagcgatttaacagcttgagcaacgtgtctcttatgagtcgccggaatgcactgcagcagtgcggatctgatgctgctctgagcaggcacatgtattcgcctcgaccacctagtatcactgaaaatgtcatgatggaggccatgggtatggatgggaacaccgaaggcagacagcagggcaacatgatcccaggtggagaccgaagctacatgggctaccagcacaatcctcatcaagcaagtcagctgtcccctgggcaagaaagtctaggatgcattgaccaagtttatcagtctcaaatgcaaggtcagtatcagcgtgaggagagttgctccaccggcgtaatgggtcaggctgacattgcaaataatctactacagcaagctgagtatgggatgagcacttgtcagctcagtccctcaggtccacactatcctagccaaggggatggatcaggaccttggggacaaaccaaccagctccacagccctgggatgcaataccagggtgcaggaatgcaaggacagcactacactcaacagggcatatatgaccctacatctaaccctaaccttcaaagagttactgtaaaaccagagcaattccatccatcaatgggaggatctagttcttgccaaaacactaaagctctgcaccagaataggcataatgcaaatatgcaaacatatccactacaaggccaaggtattatgaataggtcctccagtgcaagttgtgacttccatcattcccaaatgggaacacaaccaaaccaggggggatcattccagagtggtactggtataaaccttgccctggcagaaagccgcagatcacaaacaccaatgcatcaaatgaaggaaatgatggtcaggaactatgtgcaatcccagcaagctctcctatgggaacagcagcaagagcagagtgtgtccgaaaaacctgatggcatggacatgggacaaacacagatgatgcagcacagtccgcagcatcaacaagccaaccagaatctttatcctggtaacacttaccaaggctacccaaatcagaacttgatgagcccacagcaaaatcgagttcctggatctgtcaaagagcaaatgcaaagctcctgctatgggccagacatgattccaaggccaccccaagtgcgaaaatctttaagtcgccaaaacagcctttcccaacaagcaggtggagcttacctgggaagcccacctcacctcagcccggtccactctacagctagtcccagaagaggagttagactcccgccagttcaacaacaacaacaacagcagcagcagcattcagaaaacttcaacaacaacaacaacccaatgtactactcaggccagatgcacatgcaccacgacctagagaagactccagagggtccatgccttgctcagcagcacctaacaagctcagaccccactacaaaaccaacatcaatctcttatccagaccctgcccccatgtccaatgccttagaacacctagacttggagaacgctcagattgactttacctccatcattgatgaccaagaaccttcatcttacagtccaatcaatgcacctataggacacaatcaatgctcctctcagacgtcctctcgccttacaacaccccaaaactccatcacacttcccagtggcctttctaatatggcaatcggagacatgagctcaatgttgacatcccttgctggggaaaacaagtatctaaacacgttatcttacccagctttcttgtacaaagtggttatggtgagcaagggcgaggacgacaacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgccgaggtcaagaccacctacaaggccaagaagcccgtgcagctgcccggcgcctacaacgtcaacatcaagctggacatcacctcccacaacgaggactacaccatcgtggaacagtacgagcgcgccgagggccgccactccaccggcggcatggacgagctgtacaagtaaggcgcgcctctagaactatagtgagtcgtattacgtagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttccaactttattatacatagttgataattcactggccgtcgttttacatcgatgatgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagatcagatctgcgaagatacggccacgggtgctcttgatcctgtggctgattttggactgtgctgctcgcagctgctgatgaatcacatacttcctccattttcttccactgattgactgttataatttccctaatttccaggtcaaggtgctgtgcattgtggtaatagatgtgacatgacgtcacttccaaaggaccaatgaacatgtctgaccaatttcatataatgtgaaaacgattttcataggcagaataaataacatttaaattaaactgggcatcagcgcaattcaattggtttggtaatagcaagggaaaatagaatgaagtgatctccaaaaaataagtactttttgactgtaaataaaattgtaaggagtaaaaagtacttttttttctaaaaaaatgtaattaagtaaaagtaaaagtattgatttttaattgtactcaagtaaagtaaaaatccccaaaaataatacttaagtacagtaatcaagtaaaattactcaagtactttacacctctggttcttgaccccctaccttcagcaagcccagcagatccactagttctagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtg

SEQ ID NO: 10 - DNA, 11774 bp, Artificial Sequence
  Description of Artificial Sequence Synthetic plasmid
taccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccaaatagtaggaattacccacctgtacaagtgctgaaaacttggatgaataagcccgtttgccattttctactgctattttaaatcttttctgtatttgtctgcatttgtctttacccttgaataaatgttttagttgttttttttcaattatgactgtgtttaacagacattattacaataatatgtaatagtagtacacactattattatgtaatagtacttgttgactgtatttgagaactggaccgagtgagtgttacgtcacccattcaaaatgacttacttctggctccaacaaaatgaagttaattcagttgccatttttcactgtatggacatcgccgtgttggagctagacgtcgccaataagcaagaaagagccgagatgcgtcgagtctgagtcaccgttcctatggcaacccctctaaccaatcagaagtaagcttgttggaagtccacagcctaccacttgaaagcgggctgcacaaaatctgtcaaacgttttgaacgttggatgtgagagcacatacttttattaaggcatctggttggtcagtttataatatcaacaacttgggctacagaaaagaaaagttattacagaaattatgattaacaagtacatgttaaataaagattttaatatgaatgccaccactggagcattcatgccatttggagcttcttcctgtttggatcactagaaggaggaggtcactcattacagttctcatatacagtcgttggttggttggttggttggttggttggtagattgattgattgattgattgattgattgattgattgattgattgattgattgattgattgattgattgattgattgattgattgattgattgattgattgattggtagtcaaaataagaaataatttccacagattcattacagaaatgattaaatgcatacataaaaaactggggggggggggatacaacaacacacttaagtcacatttgcctacgtaaagaaaagtaaagaaaatcaatagctatattttacatctcctttttttgctgtcttttaattagccttgttttgctgttatcttgattgaaactgtaacttcttcacctgcttcttttctttgtaggttttgccagcccagaaacaggcatggctgtgcaaggccagagcaccatgcacaatgcgctgcatgtcttcatgaacggctcaatgtcctcagtccagggctcagccaacgaccccatcttcctccttcaccatgctttcattgacaggtaacaaacacgtcatgacattagactgcacagtttttgacaaagttcatacaatctgttgtttatagctgctacaattagtgaagtttgtgaatgtacttggatgagcagcgaaagatcaattgagatcaattgttagagtttggttgccctgcagagcaaagaacaaaaaataatctggtggctttactgcgtgaggttattattggtggaatagaaacacaaaacataattgcatttatttgtttaattttttatcttatcttaactttcatcttgcatatttgtttcttacatcatttctagcatctttgagcgctggctaagaactcatcagcctccccggtccatctacccacgtaccaatgcaccaattggccacaatgacggctactacatggtgccattccttcctctttataggaatggagactacctcctgtccaacaaggctcttggatacgagtacgcctacctgttggacccaggtcattgcacaacaccagaaatgccctctgatctgcaaaagacgtgaatatctgttcagacacccatatccactctgttccacacaggtcagaggtttgtccaggagttcttgacagaggtgtaaaaagtactcaaaaattttactcaagtgaaagtacaagtacttagggaaaattttactcaattaaaagtaaaagtatctggctagaatcttacttgagtaaaagtaaaaaagtactccattaaaattgtacttgagtattaaggaagtaaaagtaaaagcaagaaagaaaactagagattcttgtttaagcttttaatctcaaaaaacattaaatgaaatgcatacaaggttttatcctgctttagaactgtttgtatttaattatcaaactataagacagacaatctaatgccagtacacgctactcaaagttgtaaaacctcagatttaacttcagtagaagctgattctcaaaattgttagtgtcaagcctagctcttttggggctgaaaagcaatcctgcagtgctgaaaagcctctcacaggcagccgatgcgggaagaggtgtattagtcttgatagagaggctgcaaatagcaggaaacgtgagcagagactccctggtgtctgaaacacaggccagatgggccctcgagcaggaaacagctatgaccatgattacgccaagctatcaactttgtatagaaaagttgcttcaggggtgtcgcttggttatttccaaaaatcaaattaattttattaaactattagaacgagcatgttttgtctatatgctacagaagataaaaaataataggagttaacagttataaaacaacacactttgtttctattgattgttgaccacactggggtctcattaagttagattaaagacacactaactgggtcaaaagcagcagattgatttcatagcaccagggtaaactttctaacacttttacggcaatcatatacattaaaattaaatacagaccacgactgaacaaggaggatgatctccaatattaaacaaagagacttgtgcctatttctctgagggtaaacatgacctctcaagttagcaagttggttttaacactacaaaaatagttaagactgcaatcccagaataaagtattggttttaaccaatcaatatagtacagtaaacatccatttgttttgttgaaacgttaaacaaatctgaccaaagctattagcttatataaaacaggtttgccttctatgtagctgaaaacaccacaggcccgattttgctactgtgtaaaacatttcagcaagatttttttattgcatttttttttactgaatcgttcaaacattttatcattttagtttgttcattcattgcaactggaaaaacaacacatcacacaaccgcacatatttcagcaataagtacaataaaacactcaaataaaaaaaacattttaaatctctttgtatttttgaccgctgtttcgcgtaatttcacggtaaaactctggaaatctccactacattcctctcagcggctcctctcaatgacagctgaagaagtgacgcggctgcctgctgtgttttgattggtcgaattcactggaggcttccagaacagtgtagagtctgaacgggtgcgcgctctgctgtatttaaagggcgaaagagagaccgcagagaaactcaaccgaagagaagcgacttgacaaagaagaaaagagcagcctgacaggactttttccccgacgaggtgtttattcgctctatttaagaatctactgtaaggtaagtctcaatatattgtactctattggctaatcagaattatatagagattatatgtacttaatgtcaaaaaatcaactttgtatatgtaatctttttacatgtggactgcctatgttcatcttattttaggtctactagaaaattatatttcccgttttcacaataaggatttttaaaaaaagcaatgaacagacgggcatttactttatgttgctgacattattttatatgagcataataaccataaatactagcaaatgtcctaaatgaatttgtgttaatgttgtctacaaaagaaaattagcgttttacttgtacaactaataataacttggttattaagagaatttcacttgttgactagaaaaatcctttcataatgaaacaattgcaccataaattgtataaatataaaattaattctaattgtttttttttcctgccaagtttgtacaaaaaagcaggcttcgaaggagatagaaccatggatgaatgcaagcaggagcccgaggccgtgtatgaaaccaactgccactgggaaggatgtgccaaagagtacgacacgcaggaccagctcgttcatcacatcagtaatgaccacatccatggagagaagaaggagtttgtgtgtcgatgggtggagtgctctcgagaacagaagcctttcaaggcccagtacatgcttgtggtccacatgcgtcgacacactggggagaagccccataagtgcacatttgaaggttgctcgaaggcttattctcgactggaaaatctcaaaactcacttgaggtcccacacaggggagaaaccttacgtatgcgagcacgagggctgcaacaaggctttttccaacgcttcagaccgggccaagcaccagaaccgcacgcactccaatgagaaaccatatgtgtgtaagatcccaggttgcacaaaacgttatacagaccccagttcactcaggaagcatgtgaagactgttcacggtccagaagcacatgtcactaagaagcagcgcggcgatgcaccgccgaaacctcacccccctaaagggaatggagaaaatgaggcccacacaaagcatgtgagggggagaacagatgggtcaggagaggccaacagcaccaccagaggcgtggaggactgccaacatgtcaagacgatcaagactgaaaacacagtgatgtatcagtccagccctggtggtcagtcgtcatgtagcagtgagccatcgccacttggcagtgccaccaacaatgacagtggagtagagatggccatgcacagtggaggcagtttgggggacctgagtgctttagatgacacacctgtggtcgattctaccggttctccaggtacttctgctggagttggtctccagctgcgtaaaaacagggcgggactactacagcttgagcacatcaagaaggaaaaactaaaaactgtacgagactcctgctcctgggcaaatgcaccacctcaagtccgaaataccaagctgcctccaataccctctattgactcactgctggacgctccaaatatggggactcaaatgtctgtacctccaacccagcatttaggagacttgtcttcatatgagatgacaatgcttaatcagctacatgagcgcagagatagctccaccagcacaatgagctcagcatatataagccgtcggtcctctggcatttcaccttgctactccagtcgtcgctcaagtgaggcttcccaatttggagtccgcaacaacaatgtcagctcagcagactcctatgacccgatctccacagatctgtcacgtcgttccagtgaagcaagccaatgtggtggtacaggtgggctcccaagtttactaaatctcacaccagctcaacactacagtcttaaggcgaaatatgctgcagccactgggggtgcaccaccaacacctctgcccaacatggatcgcatgagccttaaaactagaatggccatgtacaatgactcgcaggacagttcagcacatttgcaccatgcccaaggtgtagtcaactcaagacgctgcagcgacactgggtacggtgcccctggcatgatgcctcatgaagttccagccaatcttccacgacgagcaagcgatcctgttcgtcgtacaactcttgacccactctccttgccaagagttcagcggttcaacagcatcagcaacatgaacctatcacgtctgcctgcatatgatcgacgtgctttcaacatgcagaacaacacttggtctgatggaagcttgcaccgtcatcccttcagccaaaggccacctagcatatcagagaacattttaatggaaaatatggccactgatggaggagatcaacaaggggatgatttggtgcttcccgatgacatggtacagtacctcaggtcccagaatggtaactcaaatcacgattctgggatttctgtcaatggcggtcatgctctagagttccatggtaatatgacctcccaacaacaacagttctatgggcaacgcagaatgggcatggcaggcatcaatggaagtcacgtagagcctgttccagaacaaatagctgatccgcagggcatgaacaaaaacaacatgcctgtgcagtggaatgaggtcagttctggttctgcagatgcagtttcaagggttccaaagcaacagcaacaacaactcagagggaacttgacagttgttcagcagaagcagaactttggctcttaccaaggatttggatcaaaccagcagattgtaccaatgagccagaatctggctagtctacagcaggcttatccacagagaaacatccagaggatgaacactgtccagcagtttcgccagtccatttccaatccatgtcagaatatcggtgagcaagtaaaccggcaggacctcagttacaattccaatcaacgtctaatatgcaatagcatgggcccgcccaatgggagaatggtgcaaaaccaggagctccagaattatagatcaaacttgatgcatatgaataacatcaatcagcaaaattacgttcctctaactcagacagcttttcagaatgcaggcagaggactgatacagcccagacctccgtccgaacccaaaccattgaataggcaacactcaggttcaggtatggttcagccaaacggttgctccctgtccaatgtcaactcctcagaagctagcccaaagagaccaggtgaagtcggtcaacacagcaatgggaatggcactatgtactattcaggagagatccatatgttggacaacggcattgattatggttctcccatgtctccgtgcacaaaccaagcccctgttgcttctgtgtctacaatggcatctcctggtgtcaaccaggtcaccagcacagtggactccactcagtcccttgaccacactcaaatagactttgatgccatgttggatgacggagaccattcgagtctaatgtctggcactttaagtccaggccttctgcagagtatttcgcagaattcctcaaggctcaccaccccgcgtaactctgttaccctggcctcagtaccggcaggtattggcaacatggctattggtgacatgagctcaatgttaacggcccttgcggaagaaagcaaattcttaaacatgatggcttacccagctttcttgtacaaagtggacatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctcgagctcaagcttcgaattctgcagtcgacggtaccgcgggcccgggatccaccggatcttgaccaactttattatacatagttgataattcactggccgtcgttttacatcgatgatgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagatcagatctgcgaagatacggccacgggtgctcttgatcctgtggctgattttggactgtgctgctcgcagctgctgatgaatcacatacttcctccattttcttccactgattgactgttataatttccctaatttccaggtcaaggtgctgtgcattgtggtaatagatgtgacatgacgtcacttccaaaggaccaatgaacatgtctgaccaatttcatataatgtgaaaacgattttcataggcagaataaataacatttaaattaaactgggcatcagcgcaattcaattggtttggtaatagcaagggaaaatagaatgaagtgatctccaaaaaataagtactttttgactgtaaataaaattgtaaggagtaaaaagtacttttttttctaaaaaaatgtaattaagtaaaagtaaaagtattgatttttaattgtactcaagtaaagtaaaaatccccaaaaataatacttaagtacagtaatcaagtaaaattactcaagtactttacacctctggttcttgaccccctaccttcagcaagcccagcagatccactagttctagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtg

SEQ ID NO: 11 - DNA, 12585 bp, Artificial Sequence
  Description of Artificial Sequence Synthetic plasmid
taccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccaaatagtaggaattacccacctgtacaagtgctgaaaacttggatgaataagcccgtttgccattttctactgctattttaaatcttttctgtatttgtctgcatttgtctttacccttgaataaatgttttagttgttttttttcaattatgactgtgtttaacagacattattacaataatatgtaatagtagtacacactattattatgtaatagtacttgttgactgtatttgagaactggaccgagtgagtgttacgtcacccattcaaaatgacttacttctggctccaacaaaatgaagttaattcagttgccatttttcactgtatggacatcgccgtgttggagctagacgtcgccaataagcaagaaagagccgagatgcgtcgagtctgagtcaccgttcctatggcaacccctctaaccaatcagaagtaagcttgttggaagtccacagcctaccacttgaaagcgggctgcacaaaatctgtcaaacgttttgaacgttggatgtgagagcacatacttttattaaggcatctggttggtcagtttataatatcaacaacttgggctacagaaaagaaaagttattacagaaattatgattaacaagtacatgttaaataaagattttaatatgaatgccaccactggagcattcatgccatttggagcttcttcctgtttggatcactagaaggaggaggtcactcattacagttctcatatacagtcgttggttggttggttggttggttggttggtagattgattgattgattgattgattgattgattgattgattgattgattgattgattgattgattgattgattgattgattgattgattgattgattgattgattggtagtcaaaataagaaataatttccacagattcattacagaaatgattaaatgcatacataaaaaactggggggggggggatacaacaacacacttaagtcacatttgcctacgtaaagaaaagtaaagaaaatcaatagctatattttacatctcctttttttgctgtcttttaattagccttgttttgctgttatcttgattgaaactgtaacttcttcacctgcttcttttctttgtaggttttgccagcccagaaacaggcatggctgtgcaaggccagagcaccatgcacaatgcgctgcatgtcttcatgaacggctcaatgtcctcagtccagggctcagccaacgaccccatcttcctccttcaccatgctttcattgacaggtaacaaacacgtcatgacattagactgcacagtttttgacaaagttcatacaatctgttgtttatagctgctacaattagtgaagtttgtgaatgtacttggatgagcagcgaaagatcaattgagatcaattgttagagtttggttgccctgcagagcaaagaacaaaaaataatctggtggctttactgcgtgaggttattattggtggaatagaaacacaaaacataattgcatttatttgtttaattttttatcttatcttaactttcatcttgcatatttgtttcttacatcatttctagcatctttgagcgctggctaagaactcatcagcctccccggtccatctacccacgtaccaatgcaccaattggccacaatgacggctactacatggtgccattccttcctctttataggaatggagactacctcctgtccaacaaggctcttggatacgagtacgcctacctgttggacccaggtcattgcacaacaccagaaatgccctctgatctgcaaaagacgtgaatatctgttcagacacccatatccactctgttccacacaggtcagaggtttgtccaggagttcttgacagaggtgtaaaaagtactcaaaaattttactcaagtgaaagtacaagtacttagggaaaattttactcaattaaaagtaaaagtatctggctagaatcttacttgagtaaaagtaaaaaagtactccattaaaattgtacttgagtattaaggaagtaaaagtaaaagcaagaaagaaaactagagattcttgtttaagcttttaatctcaaaaaacattaaatgaaatgcatacaaggttttatcctgctttagaactgtttgtatttaattatcaaactataagacagacaatctaatgccagtacacgctactcaaagttgtaaaacctcagatttaacttcagtagaagctgattctcaaaattgttagtgtcaagcctagctcttttggggctgaaaagcaatcctgcagtgctgaaaagcctctcacaggcagccgatgcgggaagaggtgtattagtcttgatagagaggctgcaaatagcaggaaacgtgagcagagactccctggtgtctgaaacacaggccagatgggccctcgagcaggaaacagctatgaccatgattacgccaagctatcaactttgtatagaaaagttgcttcaggggtgtcgcttggttatttccaaaaatcaaattaattttattaaactattagaacgagcatgttttgtctatatgctacagaagataaaaaataataggagttaacagttataaaacaacacactttgtttctattgattgttgaccacactggggtctcattaagttagattaaagacacactaactgggtcaaaagcagcagattgatttcatagcaccagggtaaactttctaacacttttacggcaatcatatacattaaaattaaatacagaccacgactgaacaaggaggatgatctccaatattaaacaaagagacttgtgcctatttctctgagggtaaacatgacctctcaagttagcaagttggttttaacactacaaaaatagttaagactgcaatcccagaataaagtattggttttaaccaatcaatatagtacagtaaacatccatttgttttgttgaaacgttaaacaaatctgaccaaagctattagcttatataaaacaggtttgccttctatgtagctgaaaacaccacaggcccgattttgctactgtgtaaaacatttcagcaagatttttttattgcatttttttttactgaatcgttcaaacattttatcattttagtttgttcattcattgcaactggaaaaacaacacatcacacaaccgcacatatttcagcaataagtacaataaaacactcaaataaaaaaaacattttaaatctctttgtatttttgaccgctgtttcgcgtaatttcacggtaaaactctggaaatctccactacattcctctcagcggctcctctcaatgacagctgaagaagtgacgcggctgcctgctgtgttttgattggtcgaattcactggaggcttccagaacagtgtagagtctgaacgggtgcgcgctctgctgtatttaaagggcgaaagagagaccgcagagaaactcaaccgaagagaagcgacttgacaaagaagaaaagagcagcctgacaggactttttccccgacgaggtgtttattcgctctatttaagaatctactgtaaggtaagtctcaatatattgtactctattggctaatcagaattatatagagattatatgtacttaatgtcaaaaaatcaactttgtatatgtaatctttttacatgtggactgcctatgttcatcttattttaggtctactagaaaattatatttcccgttttcacaataaggatttttaaaaaaagcaatgaacagacgggcatttactttatgttgctgacattattttatatgagcataataaccataaatactagcaaatgtcctaaatgaatttgtgttaatgttgtctacaaaagaaaattagcgttttacttgtacaactaataataacttggttattaagagaatttcacttgttgactagaaaaatcctttcataatgaaacaattgcaccataaattgtataaatataaaattaattctaattgtttttttttcctgccaagtttgtacaaaaaagcaggcttcgaaggagatagaaccatggatgaatgcaagcaggagcccgaggccgtgtatgaaaccaactgccactgggaaggatgtgccaaagagtacgacacgcaggaccagctcgttcatcacatcagtaatgaccacatccatggagagaagaaggagtttgtgtgtcgatgggtggagtgctctcgagaacagaagcctttcaaggcccagtacatgcttgtggtccacatgcgtcgacacactggggagaagccccataagtgcacatttgaaggttgctcgaaggcttattctcgactggaaaatctcaaaactcacttgaggtcccacacaggggagaaaccttacgtatgcgagcacgagggctgcaacaaggctttttccaacgcttcagaccgggccaagcaccagaaccgcacgcactccaatgagaaaccatatgtgtgtaagatcccaggttgcacaaaacgttatacagaccccagttcactcaggaagcatgtgaagactgttcacggtccagaagcacatgtcactaagaagcagcgcggcgatgcaccgccgaaacctcacccccctaaagggaatggagaaaatgaggcccacacaaagcatgtgagggggagaacagatgggtcaggagaggccaacagcaccaccagaggcgtggaggactgccaacatgtcaagacgatcaagactgaaaacacagtgatgtatcagtccagccctggtggtcagtcgtcatgtagcagtgagccatcgccacttggcagtgccaccaacaatgacagtggagtagagatggccatgcacagtggaggcagtttgggggacctgagtgctttagatgacacacctgtggtcgattctaccggttctccaggtacttctgctggagttggtctccagctgcgtaaaaacagggcgggactactacagcttgagcacatcaagaaggaaaaactaaaaactgtacgagactcctgctcctgggcaaatgcaccacctcaagtccgaaataccaagctgcctccaataccctctattgactcactgctggacgctccaaatatggggactcaaatgtctgtacctccaacccagcatttaggagacttgtcttcatatgagatgacaatgcttaatcagctacatgagcgcagagatagctccaccagcacaatgagctcagcatatataagccgtcggtcctctggcatttcaccttgctactccagtcgtcgctcaagtgaggcttcccaatttggagtccgcaacaacaatgtcagctcagcagactcctatgacccgatctccacagatctgtcacgtcgttccagtgaagcaagccaatgtggtggtacaggtgggctcccaagtttactaaatctcacaccagctcaacactacagtcttaaggcgaaatatgctgcagccactgggggtgcaccaccaacacctctgcccaacatggatcgcatgagccttaaaactagaatggccatgtacaatgactcgcaggacagttcagcacatttgcaccatgcccaaggtgtagtcaactcaagacgctgcagcgacactgggtacggtgcccctggcatgatgcctcatgaagttccagccaatcttccacgacgagcaagcgatcctgttcgtcgtacaactcttgacccactctccttgccaagagttcagcggttcaacagcatcagcaacatgaacctatcacgtctgcctgcatatgatcgacgtgctttcaacatgcagaacaacacttggtctgatggaagcttgcaccgtcatcccttcagccaaaggccacctagcatatcagagaacattttaatggaaaatatggccactgatggaggagatcaacaaggggatgatttggtgcttcccgatgacatggtacagtacctcaggtcccagaatggtaactcaaatcacgattctgggatttctgtcaatggcggtcatgctctagagttccatggtaatatgacctcccaacaacaacagttctatgggcaacgcagaatgggcatggcaggcatcaatggaagtcacgtagagcctgttccagaacaaatagctgatccgcagggcatgaacaaaaacaacatgcctgtgcagtggaatgaggtcagttctggttctgcagatgcagtttcaagggttccaaagcaacagcaacaacaactcagagggaacttgacagttgttcagcagaagcagaactttggctcttaccaaggatttggatcaaaccagcagattgtaccaatgagccagaatctggctagtctacagcaggcttatccacagagaaacatccagaggatgaacactgtccagcagtttcgccagtccatttccaatccatgtcagaatatcggtgagcaagtaaaccggcaggacctcagttacaattccaatcaacgtctaatatgcaatagcatgggcccgcccaatgggagaatggtgcaaaaccaggagctccagaattatagatcaaacttgatgcatatgaataacatcaatcagcaaaattacgttcctctaactcagacagcttttcagaatgcaggcagaggactgatacagcccagacctccgtccgaacccaaaccattgaataggcaacactcaggttcaggtatggttcagccaaacggttgctccctgtccaatgtcaactcctcagaagctagcccaaagagaccaggtgaagtcggtcaacacagcaatgggaatggcactatgtactattcaggagagatccatatgttggacaacggcattgattatggttctcccatgtctccgtgcacaaaccaagcccctgttgcttctgtgtctacaatggcatctcctggtgtcaaccaggtcaccagcacagtggactccactcagtcccttgaccacactcaaatagactttgatgccatgttggatgacggagaccattcgagtctaatgtctggcactttaagtccaggccttctgcagagtatttcgcagaattcctcaaggctcaccaccccgcgtaactctgttaccctggcctcagtaccggcaggtattggcaacatggctattggtgacatgagctcaatgttaacggcccttgcggaagaaagcaaattcttaaacatgatggcttacccagctttcttgtacaaagtggccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaaccatggctccaaagaagaagcgtaaggtaaatcgatcgaattcaaggcctctcgagcctctagaaggtggcggaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaactgcagccctatagtgagtcgtattacgtagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttccaactttattatacatagttgataattcactggccgtcgttttacatcgatgatgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagatcagatctgcgaagatacggccacgggtgctcttgatcctgtggctgattttggactgtgctgctcgcagctgctgatgaatcacatacttcctccattttcttccactgattgactgttataatttccctaatttccaggtcaaggtgctgtgcattgtggtaatagatgtgacatgacgtcacttccaaaggaccaatgaacatgtctgaccaatttcatataatgtgaaaacgattttcataggcagaataaataacatttaaattaaactgggcatcagcgcaattcaattggtttggtaatagcaagggaaaatagaatgaagtgatctccaaaaaataagtactttttgactgtaaataaaattgtaaggagtaaaaagtacttttttttctaaaaaaatgtaattaagtaaaagtaaaagtattgatttttaattgtactcaagtaaagtaaaaatccccaaaaataatacttaagtacagtaatcaagtaaaattactcaagtactttacacctctggttcttgaccccctaccttcagcaagcccagcagatccactagttctagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtg

SEQ ID NO: 12 - DNA, 11923 bp, Artificial Sequence
  Description of Artificial Sequence Synthetic plasmid
taccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccaaatagtaggaattacccacctgtacaagtgctgaaaacttggatgaataagcccgtttgccattttctactgctattttaaatcttttctgtatttgtctgcatttgtctttacccttgaataaatgttttagttgttttttttcaattatgactgtgtttaacagacattattacaataatatgtaatagtagtacacactattattatgtaatagtacttgttgactgtatttgagaactggaccgagtgagtgttacgtcacccattcaaaatgacttacttctggctccaacaaaatgaagttaattcagttgccatttttcactgtatggacatcgccgtgttggagctagacgtcgccaataagcaagaaagagccgagatgcgtcgagtctgagtcaccgttcctatggcaacccctctaaccaatcagaagtaagcttgttggaagtccacagcctaccacttgaaagcgggctgcacaaaatctgtcaaacgttttgaacgttggatgtgagagcacatacttttattaaggcatctggttggtcagtttataatatcaacaacttgggctacagaaaagaaaagttattacagaaattatgattaacaagtacatgttaaataaagattttaatatgaatgccaccactggagcattcatgccatttggagcttcttcctgtttggatcactagaaggaggaggtcactcattacagttctcatatacagtcgttggttggttggttggttggttggttggtagattgattgattgattgattgattgattgattgattgattgattgattgattgattgattgattgattgattgattgattgattgattgattgattgattgattggtagtcaaaataagaaataatttccacagattcattacagaaatgattaaatgcatacataaaaaactggggggggggggatacaacaacacacttaagtcacatttgcctacgtaaagaaaagtaaagaaaatcaatagctatattttacatctcctttttttgctgtcttttaattagccttgttttgctgttatcttgattgaaactgtaacttcttcacctgcttcttttctttgtaggttttgccagcccagaaacaggcatggctgtgcaaggccagagcaccatgcacaatgcgctgcatgtcttcatgaacggctcaatgtcctcagtccagggctcagccaacgaccccatcttcctccttcaccatgctttcattgacaggtaacaaacacgtcatgacattagactgcacagtttttgacaaagttcatacaatctgttgtttatagctgctacaattagtgaagtttgtgaatgtacttggatgagcagcgaaagatcaattgagatcaattgttagagtttggttgccctgcagagcaaagaacaaaaaataatctggtggctttactgcgtgaggttattattggtggaatagaaacacaaaacataattgcatttatttgtttaattttttatcttatcttaactttcatcttgcatatttgtttcttacatcatttctagcatctttgagcgctggctaagaactcatcagcctccccggtccatctacccacgtaccaatgcaccaattggccacaatgacggctactacatggtgccattccttcctctttataggaatggagactacctcctgtccaacaaggctcttggatacgagtacgcctacctgttggacccaggtcattgcacaacaccagaaatgccctctgatctgcaaaagacgtgaatatctgttcagacacccatatccactctgttccacacaggtcagaggtttgtccaggagttcttgacagaggtgtaaaaagtactcaaaaattttactcaagtgaaagtacaagtacttagggaaaattttactcaattaaaagtaaaagtatctggctagaatcttacttgagtaaaagtaaaaaagtactccattaaaattgtacttgagtattaaggaagtaaaagtaaaagcaagaaagaaaactagagattcttgtttaagcttttaatctcaaaaaacattaaatgaaatgcatacaaggttttatcctgctttagaactgtttgtatttaattatcaaactataagacagacaatctaatgccagtacacgctactcaaagttgtaaaacctcagatttaacttcagtagaagctgattctcaaaattgttagtgtcaagcctagctcttttggggctgaaaagcaatcctgcagtgctgaaaagcctctcacaggcagccgatgcgggaagaggtgtattagtcttgatagagaggctgcaaatagcaggaaacgtgagcagagactccctggtgtctgaaacacaggccagatgggccctcgagcaggaaacagctatgaccatgattacgccaagctatcaactttgtatagaaaagttgcttcaggggtgtcgcttggttatttccaaaaatcaaattaattttattaaactattagaacgagcatgttttgtctatatgctacagaagataaaaaataataggagttaacagttataaaacaacacactttgtttctattgattgttgaccacactggggtctcattaagttagattaaagacacactaactgggtcaaaagcagcagattgatttcatagcaccagggtaaactttctaacacttttacggcaatcatatacattaaaattaaatacagaccacgactgaacaaggaggatgatctccaatattaaacaaagagacttgtgcctatttctctgagggtaaacatgacctctcaagttagcaagttggttttaacactacaaaaatagttaagactgcaatcccagaataaagtattggttttaaccaatcaatatagtacagtaaacatccatttgttttgttgaaacgttaaacaaatctgaccaaagctattagcttatataaaacaggtttgccttctatgtagctgaaaacaccacaggcccgattttgctactgtgtaaaacatttcagcaagatttttttattgcatttttttttactgaatcgttcaaacattttatcattttagtttgttcattcattgcaactggaaaaacaacacatcacacaaccgcacatatttcagcaataagtacaataaaacactcaaataaaaaaaacattttaaatctctttgtatttttgaccgctgtttcgcgtaatttcacggtaaaactctggaaatctccactacattcctctcagcggctcctctcaatgacagctgaagaagtgacgcggctgcctgctgtgttttgattggtcgaattcactggaggcttccagaacagtgtagagtctgaacgggtgcgcgctctgctgtatttaaagggcgaaagagagaccgcagagaaactcaaccgaagagaagcgacttgacaaagaagaaaagagcagcctgacaggactttttccccgacgaggtgtttattcgctctatttaagaatctactgtaaggtaagtctcaatatattgtactctattggctaatcagaattatatagagattatatgtacttaatgtcaaaaaatcaactttgtatatgtaatctttttacatgtggactgcctatgttcatcttattttaggtctactagaaaattatatttcccgttttcacaataaggatttttaaaaaaagcaatgaacagacgggcatttactttatgttgctgacattattttatatgagcataataaccataaatactagcaaatgtcctaaatgaatttgtgttaatgttgtctacaaaagaaaattagcgttttacttgtacaactaataataacttggttattaagagaatttcacttgttgactagaaaaatcctttcataatgaaacaattgcaccataaattgtataaatataaaattaattctaattgtttttttttcctgccaagtttgtacaaaaaagcaggcttcgaaggagatagaaccatggatgaatgcaagcaggagcccgaggccgtgtatgaaaccaactgccactgggaaggatgtgccaaagagtacgacacgcaggaccagctcgttcatcacatcagtaatgaccacatccatggagagaagaaggagtttgtgtgtcgatgggtggagtgctctcgagaacagaagcctttcaaggcccagtacatgcttgtggtccacatgcgtcgacacactggggagaagccccataagtgcacatttgaaggttgctcgaaggcttattctcgactggaaaatctcaaaactcacttgaggtcccacacaggggagaaaccttacgtatgcgagcacgagggctgcaacaaggctttttccaacgcttcagaccgggccaagcaccagaaccgcacgcactccaatgagaaaccatatgtgtgtaagatcccaggttgcacaaaacgttatacagaccccagttcactcaggaagcatgtgaagactgttcacggtccagaagcacatgtcactaagaagcagcgcggcgatgcaccgccgaaacctcacccccctaaagggaatggagaaaatgaggcccacacaaagcatgtgagggggagaacagatgggtcaggagaggccaacagcaccaccagaggcgtggaggactgccaacatgtcaagacgatcaagactgaaaacacagtgatgtatcagtccagccctggtggtcagtcgtcatgtagcagtgagccatcgccacttggcagtgccaccaacaatgacagtggagtagagatggccatgcacagtggaggcagtttgggggacctgagtgctttagatgacacacctgtggtcgattctaccggttctccaggtacttctgctggagttggtctccagctgcgtaaaaacagggcgggactactacagcttgagcacatcaagaaggaaaaactaaaaactgtacgagactcctgctcctgggcaaatgcaccacctcaagtccgaaataccaagctgcctccaataccctctattgactcactgctggacgctccaaatatggggactcaaatgtctgtacctccaacccagcatttaggagacttgtcttcatatgagatgacaatgcttaatcagctacatgagcgcagagatagctccaccagcacaatgagctcagcatatataagccgtcggtcctctggcatttcaccttgctactccagtcgtcgctcaagtgaggcttcccaatttggagtccgcaacaacaatgtcagctcagcagactcctatgacccgatctccacagatctgtcacgtcgttccagtgaagcaagccaatgtggtggtacaggtgggctcccaagtttactaaatctcacaccagctcaacactacagtcttaaggcgaaatatgctgcagccactgggggtgcaccaccaacacctctgcccaacatggatcgcatgagccttaaaactagaatggccatgtacaatgactcgcaggacagttcagcacatttgcaccatgcccaaggtgtagtcaactcaagacgctgcagcgacactgggtacggtgcccctggcatgatgcctcatgaagttccagccaatcttccacgacgagcaagcgatcctgttcgtcgtacaactcttgacccactctccttgccaagagttcagcggttcaacagcatcagcaacatgaacctatcacgtctgcctgcatatgatcgacgtgctttcaacatgcagaacaacacttggtctgatggaagcttgcaccgtcatcccttcagccaaaggccacctagcatatcagagaacattttaatggaaaatatggccactgatggaggagatcaacaaggggatgatttggtgcttcccgatgacatggtacagtacctcaggtcccagaatggtaactcaaatcacgattctgggatttctgtcaatggcggtcatgctctagagttccatggtaatatgacctcccaacaacaacagttctatgggcaacgcagaatgggcatggcaggcatcaatggaagtcacgtagagcctgttccagaacaaatagctgatccgcagggcatgaacaaaaacaacatgcctgtgcagtggaatgaggtcagttctggttctgcagatgcagtttcaagggttccaaagcaacagcaacaacaactcagagggaacttgacagttgttcagcagaagcagaactttggctcttaccaaggatttggatcaaaccagcagattgtaccaatgagccagaatctggctagtctacagcaggcttatccacagagaaacatccagaggatgaacactgtccagcagtttcgccagtccatttccaatccatgtcagaatatcggtgagcaagtaaaccggcaggacctcagttacaattccaatcaacgtctaatatgcaatagcatgggcccgcccaatgggagaatggtgcaaaaccaggagctccagaattatagatcaaacttgatgcatatgaataacatcaatcagcaaaattacgttcctctaactcagacagcttttcagaatgcaggcagaggactgatacagcccagacctccgtccgaacccaaaccattgaataggcaacactcaggttcaggtatggttcagccaaacggttgctccctgtccaatgtcaactcctcagaagctagcccaaagagaccaggtgaagtcggtcaacacagcaatgggaatggcactatgtactattcaggagagatccatatgttggacaacggcattgattatggttctcccatgtctccgtgcacaaaccaagcccctgttgcttctgtgtctacaatggcatctcctggtgtcaaccaggtcaccagcacagtggactccactcagtcccttgaccacactcaaatagactttgatgccatgttggatgacggagaccattcgagtctaatgtctggcactttaagtccaggccttctgcagagtatttcgcagaattcctcaaggctcaccaccccgcgtaactctgttaccctggcctcagtaccggcaggtattggcaacatggctattggtgacatgagctcaatgttaacggcccttgcggaagaaagcaaattcttaaacatgatggcttacccagctttcttgtacaaagtggttatggtgagcaagggcgaggacgacaacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgccgaggtcaagaccacctacaaggccaagaagcccgtgcagctgcccggcgcctacaacgtcaacatcaagctggacatcacctcccacaacgaggactacaccatcgtggaacagtacgagcgcgccgagggccgccactccaccggcggcatggacgagctgtacaagtaaggcgcgcctctagaactatagtgagtcgtattacgtagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttccaactttattatacatagttgataattcactggccgtcgttttacatcgatgatgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagatcagatctgcgaagatacggccacgggtgctcttgatcctgtggctgattttggactgtgctgctcgcagctgctgatgaatcacatacttcctccattttcttccactgattgactgttataatttccctaatttccaggtcaaggtgctgtgcattgtggtaatagatgtgacatgacgtcacttccaaaggaccaatgaacatgtctgaccaatttcatataatgtgaaaacgattttcataggcagaataaataacatttaaattaaactgggcatcagcgcaattcaattggtttggtaatagcaagggaaaatagaatgaagtgatctccaaaaaataagtactttttgactgtaaataaaattgtaaggagtaaaaagtacttttttttctaaaaaaatgtaattaagtaaaagtaaaagtattgatttttaattgtactcaagtaaagtaaaaatccccaaaaataatacttaagtacagtaatcaagtaaaattactcaagtactttacacctctggttcttgaccccctaccttcagcaagcccagcagatccactagttctagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtg

SEQ ID NO: 13 - DNA, 7457 bp, Artificial Sequence
  Description of Artificial Sequence Synthetic plasmid
ccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtacaggtcattgcacaacaccagaaatgccctctgatctgcaaaagacgtgaatatctgttcagacacccatatccactctgttccacacaggtcagaggtttgtccaggagttcttgacagaggtgtaaaaagtactcaaaaattttactcaagtgaaagtacaagtacttagggaaaattttactcaattaaaagtaaaagtatctggctagaatcttacttgagtaaaagtaaaaaagtactccattaaaattgtacttgagtattaaggaagtaaaagtaaaagcaagaaagaaaactagagattcttgtttaagcttttaatctcaaaaaacattaaatgaaatgcatacaaggttttatcctgctttagaactgtttgtatttaattatcaaactataagacagacaatctaatgccagtacacgctactcaaagttgtaaaacctcagatttaacttcagtagaagctgattctcaaaattgttagtgtcaagcctagctcttttggggctgaaaagcaatcctgcagtgctgaaaagcctctcacaggcagccgatgcgggaagaggtgtattagtcttgatagagaggctgcaaatagcaggaaacgtgagcagagactccctggtgtctgaaacacaggccagatgggccgatatcaactttgtatagaaaagttgagctgctccagcacttccccctcccggcgttgatgtgaggggctccgcctgggtggtctccctctctctccactttggcgagctgttgcttagcagctaataataggagatgtttgcaaagtaatttgcctcttcctcggccaatgagaaccagagcacttttccatcaaaccacccttttcagggtttgcaaaggggctacctctccaaatgttactgcaacaggatcagaggtagactggcggaatccgctcgccactttcttctgctttaaaacgagttacgcgctatagctggacgaatgaagaccgagggatctatacggattcgcgcacataatgttgggatggagttggaagactttggctcaaggaaagactagagaatatttcttgaagttgaacccggtggatgcgcgtgtcgcgaatcttggattattattattatcgcctccgtttaacgcgccgcgtttgcctttaactttcttcagcgcttcttctgcctcccgtttggactcaactttgtacaaaaaagcaggctggaccatggctccaaagaagaagcgtaaggtaaatcgatcgaattcaaggcctctcgagcctctagaaggtggcggaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaacacccagctttcttgtacaaagttgatgtagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaattcgcggccgcggcgccaatgcattgggccctcgagcatatgagatctgtcccgatctttctcttctgtgctgtcaatttagagtagtttttgaggtaatttgggtgcattctctgtaaaaacaacaaataaatatttatattgtatgcaataaatgtaacaattacacatacactctctgattacagcattaaagggttagttcacccaaaaatgaaaagatgtcattaatgactcaccctcatgtcgttccaagcccgtaacacctccgttcatcttcggaacacagtttaagatattttagatttagtccgagagctttctgtccctccactgaaaatgtatgtacggtatactgtccatgtccagaaaggtaataaaaacatcaaagtagtccatgtgacatcagtgggttaggtagaattatttgaagcatcgaaaatacattttggtccaaaaataacaaaacctacgactttattcagcattgtagcgtcactgcgaagtcgtgaacgcggattgacaacagacccggaagagaatacaatgccgaataaagtcgtaggttttgttatttttggaccaaaatgtattcgatgcttcaaaaaattctaactaacccactgatgtcacatggactactttgatgtttttattacctttctggacatggacagtataccgtacatacattctcaatggaggcacagaaagctctcggactaaatctaaaatatcttaaactgtgttctgaagatgaacggaggtcttacgggcttggaacgacatgagggcgagtcattaatgacattattttcatttttgggtgaactaaccctttaataaaactgacatttgcagccaattgcacactttataatgttaaatacttgttgattttagaaatacagtagcatttagtgacaatcaaacattaattttatttctgattatattgacaaaaacacaggactgtaaggctgacatgacagggtgaattgatttcattacattagttttgatggtaacacttttacaataaggttcgttagttaactacattagttaacatgaactaataatgaactgcacgtatacatgttaatttcaacatttactaatactttatatcttgttaacattagttaatgcactgtgaactaacatgaacaaacaatgaacatctgtatttctattaactaacgttaacaaagattaacagatacagtaacaaatgtattgctcatggttatttaatgttagttaatacatgaactaatcaacctcattgtaaagtgttaccgttttgacaatacaacatgtttggaaaaaaatttctcgcttggttttcttgtgttttgcatgaattcaaacacaatgagagcacggaatgataatgattattcaaagctgcaataggcgccaaatttcccgtggacctaacgttaccaattacaaatgcattttagcattttattacgtgagccttatattgtcaatcgcatgcattagtgaaaataggcctactacacatataaaaggcgcgccatcaacgtcaaaacatgcacacccaaaaaaaaaaaaaaaaaaaaaaaaaaaaactatcagtaaaacaaaatacttaatgtacttacctcaatatgcttccttaggtttgatggcgaacttttgaaggccgatatttctttattaagcgggagacagaggacacatttcatcttgaatgaatctttatttacaccacttaaagaaaagaattcgcgaagatacggccacgggtgctcttgatcctgtggctgattttggactgtgctgctcgcagctgctgatgaatcacatacttcctccattttcttccactgattgactgttataatttccctaatttccaggtcaaggtgctgtgcattgtggtaatagatgtgacatgacgtcacttccaaaggaccaatgaacatgtctgaccaatttcatataatgtgaaaacgattttcataggcagaataaataacatttaaattaaactgggcatcagcgcaattcaattggtttggtaatagcaagggaaaatagaatgaagtgatctccaaaaaataagtactttttgactgtaaataaaattgtaaggagtaaaaagtacttttttttctaaaaaaatgtaattaagtaaaagtaaaagtattgatttttaattgtactcaagtaaagtaaaaatccccaaaaataatacttaagtacagtaatcaagtaaaattactcaagtactttacacctctggttcttgaccccctaccttcagcaagcccagcagatccactagttctagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtg

SEQ ID NO: 14 - DNA, 8937 bp, Artificial Sequence
  Description of Artificial Sequence Synthetic plasmid
ccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtacaggtcattgcacaacaccagaaatgccctctgatctgcaaaagacgtgaatatctgttcagacacccatatccactctgttccacacaggtcagaggtttgtccaggagttcttgacagaggtgtaaaaagtactcaaaaattttactcaagtgaaagtacaagtacttagggaaaattttactcaattaaaagtaaaagtatctggctagaatcttacttgagtaaaagtaaaaaagtactccattaaaattgtacttgagtattaaggaagtaaaagtaaaagcaagaaagaaaactagagattcttgtttaagcttttaatctcaaaaaacattaaatgaaatgcatacaaggttttatcctgctttagaactgtttgtatttaattatcaaactataagacagacaatctaatgccagtacacgctactcaaagttgtaaaacctcagatttaacttcagtagaagctgattctcaaaattgttagtgtcaagcctagctcttttggggctgaaaagcaatcctgcagtgctgaaaagcctctcacaggcagccgatgcgggaagaggtgtattagtcttgatagagaggctgcaaatagcaggaaacgtgagcagagactccctggtgtctgaaacacaggccagatgggccgatatcaactttgtatagaaaagttgctgtcgtttgatatccatgcttcgttcttttggagcttacctttacacaagcagtgacgggctgtggccgtttatgtttaggggctgtgtgatggcagatttctggacagcgcatggtcatctccccagtttatttatccaaggcctctagacctcggtccggacctccctcagtttgttaggcttaaaggggccccctaccgcatggagcaattcctcagattccacattagctcccattcccagctgcccttaccctcatcaaattaaacaatttcgagaaatgtgctcaacgctgccacagtatacaagcagacgtacccttgtgctttcccttagtgtcaatggtgctatatgctaagacatttaaatttgaactaatattttgccttccactgaattgcagtgaaaaaataatcaaatgacaggtggaccagatgcatgacatgggcttcatatcatattttctgtagggtatttgttgatttggcatatattttgtcattataagcatgcttaggtttctcaaacctttgcattgttctacaagtcagataattgattgagtcacatttggccatttacaattgaaaaaatcatctggaatagtcttgataaggagtctaatctgaaaagccattactctgaaacatcataaaataaccttaagtgttaagtaaaatcaacgtgttgtccaattcaaaatgtgtattatttctgtaatattttgaacatttttacaaagaatgacaaataaaacatcttaagattacctgaaatttgaaatatttatagaaataaattgatcaaagttttaattttctcatataatctaacaaaaatgtgttgtcaagaatagttttttggacaagctcagcaaaggatttatatgggtaaaaggctttttttttctggccatgaatagtgactatagtatttttaccatgatttacacaagacgcccattaagaaataagtcagacattaatttactgatatattaaaacattattttaaggaccatagtgcagcctcaaaatgctaattcattgccatttaaagtctttatattgtactttagccatttttatttacaagattttgatttgattttgatttattttttttacacttattttaaaaaaaaacttgttcacccatattttgatcttttttcaagtaatttgaaacattaagaagactctagcatttaatatgctttatgaaaacaggacgtcatatgttttatcatcatttaacaatcagtacatgttattcatgtttgtaaaataaaaattagtcaagaaataatttgatgttttaatactctcatcaattattttatcggctctacgtttttgtaagtacgcatcagaccagcgaagattaatctgtaaagtctccctctgcatccgactgtcagcataaaagaaatgtgtggctgcagtatcaatagatggcctgtgtgatagcgtgttaaaatacgagctgagctattgggaatgtgcggagcggagcagcagtgcagtgaaagagctgctccagcacttccccctcccggcgttgatgtgaggggctccgcctgggtggtctccctctctctccactttggcgagctgttgcttagcagctaataataggagatgtttgcaaagtaatttgcctcttcctcggccaatgagaaccagagcacttttccatcaaaccacccttttcagggtttgcaaaggggctacctctccaaatgttactgcaacaggatcagaggtagactggcggaatccgctcgccactttcttctgctttaaaacgagttacgcgctatagctggacgaatgaagaccgagggatctatacggattcgcgcacataatgttgggatggagttggaagactttggctcaaggaaagactagagaatatttcttgaagttgaacccggtggatgcgcgtgtcgcgaatcttggattattattattatcgcctccgtttaacgcgccgcgtttgcctttaactttcttcagcgcttcttctgcctcccgtttggactcaactttgtacaaaaaagcaggctacaccatggctccaaagaagaagcgtaaggtaatggtgagcaagggcgaggaggacaacatggccatcatcaaggagttcatgcgcttcaaggtgcacatggagggctccgtgaacggccgcgagttcgagatcgagggcgagggcgagggccgcccctacgagggcacccagaccgccaagctgaaggtgaccaagggcggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgccgaggtcaagaccacctacaaggccaagaagcccgtgcagctgcccggcgcctacaacgtcaacatcaagctggacatcacctcccacaacgaggactacaccatcgtggaacagtacgagcgcgccgagggccgccactccaccggcggcatggacgagctgtacaagtaagacccagctttcttgtacaaagttgatgtagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaattcgcggccgcggcgccaatgcattgggccctcgagcatatgagatctgtcccgatctttctcttctgtgctgtcaatttagagtagtttttgaggtaatttgggtgcattctctgtaaaaacaacaaataaatatttatattgtatgcaataaatgtaacaattacacatacactctctgattacagcattaaagggttagttcacccaaaaatgaaaagatgtcattaatgactcaccctcatgtcgttccaagcccgtaacacctccgttcatcttcggaacacagtttaagatattttagatttagtccgagagctttctgtccctccactgaaaatgtatgtacggtatactgtccatgtccagaaaggtaataaaaacatcaaagtagtccatgtgacatcagtgggttaggtagaattatttgaagcatcgaaaatacattttggtccaaaaataacaaaacctacgactttattcagcattgtagcgtcactgcgaagtcgtgaacgcggattgacaacagacccggaagagaatacaatgccgaataaagtcgtaggttttgttatttttggaccaaaatgtattcgatgcttcaaaaaattctaactaacccactgatgtcacatggactactttgatgtttttattacctttctggacatggacagtataccgtacatacattctcaatggaggcacagaaagctctcggactaaatctaaaatatcttaaactgtgttctgaagatgaacggaggtcttacgggcttggaacgacatgagggcgagtcattaatgacattattttcatttttgggtgaactaaccctttaataaaactgacatttgcagccaattgcacactttataatgttaaatacttgttgattttagaaatacagtagcatttagtgacaatcaaacattaattttatttctgattatattgacaaaaacacaggactgtaaggctgacatgacagggtgaattgatttcattacattagttttgatggtaacacttttacaataaggttcgttagttaactacattagttaacatgaactaataatgaactgcacgtatacatgttaatttcaacatttactaatactttatatcttgttaacattagttaatgcactgtgaactaacatgaacaaacaatgaacatctgtatttctattaactaacgttaacaaagattaacagatacagtaacaaatgtattgctcatggttatttaatgttagttaatacatgaactaatcaacctcattgtaaagtgttaccgttttgacaatacaacatgtttggaaaaaaatttctcgcttggttttcttgtgttttgcatgaattcaaacacaatgagagcacggaatgataatgattattcaaagctgcaataggcgccaaatttcccgtggacctaacgttaccaattacaaatgcattttagcattttattacgtgagccttatattgtcaatcgcatgcattagtgaaaataggcctactacacatataaaaggcgcgccatcaacgtcaaaacatgcacacccaaaaaaaaaaaaaaaaaaaaaaaaaaaaactatcagtaaaacaaaatacttaatgtacttacctcaatatgcttccttaggtttgatggcgaacttttgaaggccgatatttctttattaagcgggagacagaggacacatttcatcttgaatgaatctttatttacaccacttaaagaaaagaattcgcgaagatacggccacgggtgctcttgatcctgtggctgattttggactgtgctgctcgcagctgctgatgaatcacatacttcctccattttcttccactgattgactgttataatttccctaatttccaggtcaaggtgctgtgcattgtggtaatagatgtgacatgacgtcacttccaaaggaccaatgaacatgtctgaccaatttcatataatgtgaaaacgattttcataggcagaataaataacatttaaattaaactgggcatcagcgcaattcaattggtttggtaatagcaagggaaaatagaatgaagtgatctccaaaaaataagtactttttgactgtaaataaaattgtaaggagtaaaaagtacttttttttctaaaaaaatgtaattaagtaaaagtaaaagtattgatttttaattgtactcaagtaaagtaaaaatccccaaaaataatacttaagtacagtaatcaagtaaaattactcaagtactttacacctctggttcttgaccccctaccttcagcaagcccagcagatccactagttctagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtg

SEQ ID NO: 15 - DNA, 7460 bp, Artificial Sequence
  Description of Artificial Sequence Synthetic plasmid
ccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtacaggtcattgcacaacaccagaaatgccctctgatctgcaaaagacgtgaatatctgttcagacacccatatccactctgttccacacaggtcagaggtttgtccaggagttcttgacagaggtgtaaaaagtactcaaaaattttactcaagtgaaagtacaagtacttagggaaaattttactcaattaaaagtaaaagtatctggctagaatcttacttgagtaaaagtaaaaaagtactccattaaaattgtacttgagtattaaggaagtaaaagtaaaagcaagaaagaaaactagagattcttgtttaagcttttaatctcaaaaaacattaaatgaaatgcatacaaggttttatcctgctttagaactgtttgtatttaattatcaaactataagacagacaatctaatgccagtacacgctactcaaagttgtaaaacctcagatttaacttcagtagaagctgattctcaaaattgttagtgtcaagcctagctcttttggggctgaaaagcaatcctgcagtgctgaaaagcctctcacaggcagccgatgcgggaagaggtgtattagtcttgatagagaggctgcaaatagcaggaaacgtgagcagagactccctggtgtctgaaacacaggccagatgggccgatatcaactttgtatagaaaagttgagctgctccagcacttccccctcccggcgttgatgtgaggggctccgcctgggtggtctccctctctctccactttggcgagctgttgcttagcagctaataataggagatgtttgcaaagtaatttgcctcttcctcggccaatgagaaccagagcacttttccatcaaaccacccttttcagggtttgcaaaggggctacctctccaaatgttactgcaacaggatcagaggtagactggcggaatccgctcgccactttcttctgctttaaaacgagttacgcgctatagctggacgaatgaagaccgagggatctatacggattcgcgcacataatgttgggatggagttggaagactttggctcaaggaaagactagagaatatttcttgaagttgaacccggtggatgcgcgtgtcgcgaatcttggattattattattatcgcctccgtttaacgcgccgcgtttgcctttaactttcttcagcgcttcttctgcctcccgtttggactcaactttgtacaaaaaagcaggctggaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggaggaggaagatctaagctgaaccctcctgatgagagtggccccggctgcatgagctgcaagtgtgtgctctcctgagacccagctttcttgtacaaagttgatgtagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaattcgcggccgcggcgccaatgcattgggccctcgagcatatgagatctgtcccgatctttctcttctgtgctgtcaatttagagtagtttttgaggtaatttgggtgcattctctgtaaaaacaacaaataaatatttatattgtatgcaataaatgtaacaattacacatacactctctgattacagcattaaagggttagttcacccaaaaatgaaaagatgtcattaatgactcaccctcatgtcgttccaagcccgtaacacctccgttcatcttcggaacacagtttaagatattttagatttagtccgagagctttctgtccctccactgaaaatgtatgtacggtatactgtccatgtccagaaaggtaataaaaacatcaaagtagtccatgtgacatcagtgggttaggtagaattatttgaagcatcgaaaatacattttggtccaaaaataacaaaacctacgactttattcagcattgtagcgtcactgcgaagtcgtgaacgcggattgacaacagacccggaagagaatacaatgccgaataaagtcgtaggttttgttatttttggaccaaaatgtattcgatgcttcaaaaaattctaactaacccactgatgtcacatggactactttgatgtttttattacctttctggacatggacagtataccgtacatacattctcaatggaggcacagaaagctctcggactaaatctaaaatatcttaaactgtgttctgaagatgaacggaggtcttacgggcttggaacgacatgagggcgagtcattaatgacattattttcatttttgggtgaactaaccctttaataaaactgacatttgcagccaattgcacactttataatgttaaatacttgttgattttagaaatacagtagcatttagtgacaatcaaacattaattttatttctgattatattgacaaaaacacaggactgtaaggctgacatgacagggtgaattgatttcattacattagttttgatggtaacacttttacaataaggttcgttagttaactacattagttaacatgaactaataatgaactgcacgtatacatgttaatttcaacatttactaatactttatatcttgttaacattagttaatgcactgtgaactaacatgaacaaacaatgaacatctgtatttctattaactaacgttaacaaagattaacagatacagtaacaaatgtattgctcatggttatttaatgttagttaatacatgaactaatcaacctcattgtaaagtgttaccgttttgacaatacaacatgtttggaaaaaaatttctcgcttggttttcttgtgttttgcatgaattcaaacacaatgagagcacggaatgataatgattattcaaagctgcaataggcgccaaatttcccgtggacctaacgttaccaattacaaatgcattttagcattttattacgtgagccttatattgtcaatcgcatgcattagtgaaaataggcctactacacatataaaaggcgcgccatcaacgtcaaaacatgcacacccaaaaaaaaaaaaaaaaaaaaaaaaaaaaactatcagtaaaacaaaatacttaatgtacttacctcaatatgcttccttaggtttgatggcgaacttttgaaggccgatatttctttattaagcgggagacagaggacacatttcatcttgaatgaatctttatttacaccacttaaagaaaagaattcgcgaagatacggccacgggtgctcttgatcctgtggctgattttggactgtgctgctcgcagctgctgatgaatcacatacttcctccattttcttccactgattgactgttataatttccctaatttccaggtcaaggtgctgtgcattgtggtaatagatgtgacatgacgtcacttccaaaggaccaatgaacatgtctgaccaatttcatataatgtgaaaacgattttcataggcagaataaataacatttaaattaaactgggcatcagcgcaattcaattggtttggtaatagcaagggaaaatagaatgaagtgatctccaaaaaataagtactttttgactgtaaataaaattgtaaggagtaaaaagtacttttttttctaaaaaaatgtaattaagtaaaagtaaaagtattgatttttaattgtactcaagtaaagtaaaaatccccaaaaataatacttaagtacagtaatcaagtaaaattactcaagtactttacacctctggttcttgaccccctaccttcagcaagcccagcagatccactagttctagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtg

SEQ ID NO: 16 - DNA, 7436 bp, Artificial Sequence
  Description of Artificial Sequence Synthetic plasmid
ccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtacaggtcattgcacaacaccagaaatgccctctgatctgcaaaagacgtgaatatctgttcagacacccatatccactctgttccacacaggtcagaggtttgtccaggagttcttgacagaggtgtaaaaagtactcaaaaattttactcaagtgaaagtacaagtacttagggaaaattttactcaattaaaagtaaaagtatctggctagaatcttacttgagtaaaagtaaaaaagtactccattaaaattgtacttgagtattaaggaagtaaaagtaaaagcaagaaagaaaactagagattcttgtttaagcttttaatctcaaaaaacattaaatgaaatgcatacaaggttttatcctgctttagaactgtttgtatttaattatcaaactataagacagacaatctaatgccagtacacgctactcaaagttgtaaaacctcagatttaacttcagtagaagctgattctcaaaattgttagtgtcaagcctagctcttttggggctgaaaagcaatcctgcagtgctgaaaagcctctcacaggcagccgatgcgggaagaggtgtattagtcttgatagagaggctgcaaatagcaggaaacgtgagcagagactccctggtgtctgaaacacaggccagatgggccgatatcaactttgtatagaaaagttgagctgctccagcacttccccctcccggcgttgatgtgaggggctccgcctgggtggtctccctctctctccactttggcgagctgttgcttagcagctaataataggagatgtttgcaaagtaatttgcctcttcctcggccaatgagaaccagagcacttttccatcaaaccacccttttcagggtttgcaaaggggctacctctccaaatgttactgcaacaggatcagaggtagactggcggaatccgctcgccactttcttctgctttaaaacgagttacgcgctatagctggacgaatgaagaccgagggatctatacggattcgcgcacataatgttgggatggagttggaagactttggctcaaggaaagactagagaatatttcttgaagttgaacccggtggatgcgcgtgtcgcgaatcttggattattattattatcgcctccgtttaacgcgccgcgtttgcctttaactttcttcagcgcttcttctgcctcccgtttggactcaactttgtacaaaaaagcaggctccaccatggtgagcaagggcgaggaggacaacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgccgaggtcaagaccacctacaaggccaagaagcccgtgcagctgcccggcgcctacaacgtcaacatcaagctggacatcacctcccacaacgaggactacaccatcgtggaacagtacgagcgcgccgagggccgccactccaccggcggcatggacgagctgtacaagaagctgaaccctcctgatgagagtggccccggctgcatgagctgcaagtgtgtgctctcctgagacccagctttcttgtacaaagttgatgtagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaattcgcggccgcggcgccaatgcattgggccctcgagcatatgagatctgtcccgatctttctcttctgtgctgtcaatttagagtagtttttgaggtaatttgggtgcattctctgtaaaaacaacaaataaatatttatattgtatgcaataaatgtaacaattacacatacactctctgattacagcattaaagggttagttcacccaaaaatgaaaagatgtcattaatgactcaccctcatgtcgttccaagcccgtaacacctccgttcatcttcggaacacagtttaagatattttagatttagtccgagagctttctgtccctccactgaaaatgtatgtacggtatactgtccatgtccagaaaggtaataaaaacatcaaagtagtccatgtgacatcagtgggttaggtagaattatttgaagcatcgaaaatacattttggtccaaaaataacaaaacctacgactttattcagcattgtagcgtcactgcgaagtcgtgaacgcggattgacaacagacccggaagagaatacaatgccgaataaagtcgtaggttttgttatttttggaccaaaatgtattcgatgcttcaaaaaattctaactaacccactgatgtcacatggactactttgatgtttttattacctttctggacatggacagtataccgtacatacattctcaatggaggcacagaaagctctcggactaaatctaaaatatcttaaactgtgttctgaagatgaacggaggtcttacgggcttggaacgacatgagggcgagtcattaatgacattattttcatttttgggtgaactaaccctttaataaaactgacatttgcagccaattgcacactttataatgttaaatacttgttgattttagaaatacagtagcatttagtgacaatcaaacattaattttatttctgattatattgacaaaaacacaggactgtaaggctgacatgacagggtgaattgatttcattacattagttttgatggtaacacttttacaataaggttcgttagttaactacattagttaacatgaactaataatgaactgcacgtatacatgttaatttcaacatttactaatactttatatcttgttaacattagttaatgcactgtgaactaacatgaacaaacaatgaacatctgtatttctattaactaacgttaacaaagattaacagatacagtaacaaatgtattgctcatggttatttaatgttagttaatacatgaactaatcaacctcattgtaaagtgttaccgttttgacaatacaacatgtttggaaaaaaatttctcgcttggttttcttgtgttttgcatgaattcaaacacaatgagagcacggaatgataatgattattcaaagctgcaataggcgccaaatttcccgtggacctaacgttaccaattacaaatgcattttagcattttattacgtgagccttatattgtcaatcgcatgcattagtgaaaataggcctactacacatataaaaggcgcgccatcaacgtcaaaacatgcacacccaaaaaaaaaaaaaaaaaaaaaaaaaaaaactatcagtaaaacaaaatacttaatgtacttacctcaatatgcttccttaggtttgatggcgaacttttgaaggccgatatttctttattaagcgggagacagaggacacatttcatcttgaatgaatctttatttacaccacttaaagaaaagaattcgcgaagatacggccacgggtgctcttgatcctgtggctgattttggactgtgctgctcgcagctgctgatgaatcacatacttcctccattttcttccactgattgactgttataatttccctaatttccaggtcaaggtgctgtgcattgtggtaatagatgtgacatgacgtcacttccaaaggaccaatgaacatgtctgaccaatttcatataatgtgaaaacgattttcataggcagaataaataacatttaaattaaactgggcatcagcgcaattcaattggtttggtaatagcaagggaaaatagaatgaagtgatctccaaaaaataagtactttttgactgtaaataaaattgtaaggagtaaaaagtacttttttttctaaaaaaatgtaattaagtaaaagtaaaagtattgatttttaattgtactcaagtaaagtaaaaatccccaaaaataatacttaagtacagtaatcaagtaaaattactcaagtactttacacctctggttcttgaccccctaccttcagcaagcccagcagatccactagttctagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtg

SEQ ID NO: 17 - DNA, 7849 bp, Artificial Sequence
  Description of Artificial Sequence Synthetic plasmid
ccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtacaggtcattgcacaacaccagaaatgccctctgatctgcaaaagacgtgaatatctgttcagacacccatatccactctgttccacacaggtcagaggtttgtccaggagttcttgacagaggtgtaaaaagtactcaaaaattttactcaagtgaaagtacaagtacttagggaaaattttactcaattaaaagtaaaagtatctggctagaatcttacttgagtaaaagtaaaaaagtactccattaaaattgtacttgagtattaaggaagtaaaagtaaaagcaagaaagaaaactagagattcttgtttaagcttttaatctcaaaaaacattaaatgaaatgcatacaaggttttatcctgctttagaactgtttgtatttaattatcaaactataagacagacaatctaatgccagtacacgctactcaaagttgtaaaacctcagatttaacttcagtagaagctgattctcaaaattgttagtgtcaagcctagctcttttggggctgaaaagcaatcctgcagtgctgaaaagcctctcacaggcagccgatgcgggaagaggtgtattagtcttgatagagaggctgcaaatagcaggaaacgtgagcagagactccctggtgtctgaaacacaggccagatgggccgatatcaactttgtatagaaaagttgaccacccatgttcacccatattttgatcttttttcaagtaatttgaaacattaagaagactctagcatttaatatgctttatgaaaacaggacgtcatatgttttatcatcatttaacaatcagtacatgttattcatgtttgtaaaataaaaattagtcaagaaataatttgatgttttaatactctcatcaattattttatcggctctacgtttttgtaagtacgcatcagaccagcgaagattaatctgtaaagtctccctctgcatccgactgtcagcataaaagaaatgtgtggctgcagtatcaatagatggcctgtgtgatagcgtgttaaaatacgagctgagctattgggaatgtgcggagcggagcagcagtgcagtgaaagagctgctccagcacttccccctcccggcgttgatgtgaggggctccgcctgggtggtctccctctctctccactttggcgagctgttgcttagcagctaataataggagatgtttgcaaagtaatttgcctcttcctcggccaatgagaaccagagcacttttccatcaaaccacccttttcagggtttgcaaaggggctacctctccaaatgttactgcaacaggatcagaggtagactggcggaatccgctcgccactttcttctgctttaaaacgagttacgcgctatagctggacgaatgaagaccgagggatctatacggattcgcgcacataatgttgggatggagttggaagactttggctcaaggaaagactagagaatatttcttgaagttgaacccggtggatgcgcgtgtcgcgaatcttggattattattattatcgcctccgtttaacgcgccgcgtttgcctttaactttcttcagcgcttcttctgcctcccgtttggactcaactttgtacaaaaaagcaggctggaccatggctccaaagaagaagcgtaaggtaaatcgatcgaattcaaggcctctcgagcctctagaaggtggcggaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaacacccagctttcttgtacaaagttgatgtagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaattcgcggccgcggcgccaatgcattgggccctcgagcatatgagatctgtcccgatctttctcttctgtgctgtcaatttagagtagtttttgaggtaatttgggtgcattctctgtaaaaacaacaaataaatatttatattgtatgcaataaatgtaacaattacacatacactctctgattacagcattaaagggttagttcacccaaaaatgaaaagatgtcattaatgactcaccctcatgtcgttccaagcccgtaacacctccgttcatcttcggaacacagtttaagatattttagatttagtccgagagctttctgtccctccactgaaaatgtatgtacggtatactgtccatgtccagaaaggtaataaaaacatcaaagtagtccatgtgacatcagtgggttaggtagaattatttgaagcatcgaaaatacattttggtccaaaaataacaaaacctacgactttattcagcattgtagcgtcactgcgaagtcgtgaacgcggattgacaacagacccggaagagaatacaatgccgaataaagtcgtaggttttgttatttttggaccaaaatgtattcgatgcttcaaaaaattctaactaacccactgatgtcacatggactactttgatgtttttattacctttctggacatggacagtataccgtacatacattctcaatggaggcacagaaagctctcggactaaatctaaaatatcttaaactgtgttctgaagatgaacggaggtcttacgggcttggaacgacatgagggcgagtcattaatgacattattttcatttttgggtgaactaaccctttaataaaactgacatttgcagccaattgcacactttataatgttaaatacttgttgattttagaaatacagtagcatttagtgacaatcaaacattaattttatttctgattatattgacaaaaacacaggactgtaaggctgacatgacagggtgaattgatttcattacattagttttgatggtaacacttttacaataaggttcgttagttaactacattagttaacatgaactaataatgaactgcacgtatacatgttaatttcaacatttactaatactttatatcttgttaacattagttaatgcactgtgaactaacatgaacaaacaatgaacatctgtatttctattaactaacgttaacaaagattaacagatacagtaacaaatgtattgctcatggttatttaatgttagttaatacatgaactaatcaacctcattgtaaagtgttaccgttttgacaatacaacatgtttggaaaaaaatttctcgcttggttttcttgtgttttgcatgaattcaaacacaatgagagcacggaatgataatgattattcaaagctgcaataggcgccaaatttcccgtggacctaacgttaccaattacaaatgcattttagcattttattacgtgagccttatattgtcaatcgcatgcattagtgaaaataggcctactacacatataaaaggcgcgccatcaacgtcaaaacatgcacacccaaaaaaaaaaaaaaaaaaaaaaaaaaaaactatcagtaaaacaaaatacttaatgtacttacctcaatatgcttccttaggtttgatggcgaacttttgaaggccgatatttctttattaagcgggagacagaggacacatttcatcttgaatgaatctttatttacaccacttaaagaaaagaattcgcgaagatacggccacgggtgctcttgatcctgtggctgattttggactgtgctgctcgcagctgctgatgaatcacatacttcctccattttcttccactgattgactgttataatttccctaatttccaggtcaaggtgctgtgcattgtggtaatagatgtgacatgacgtcacttccaaaggaccaatgaacatgtctgaccaatttcatataatgtgaaaacgattttcataggcagaataaataacatttaaattaaactgggcatcagcgcaattcaattggtttggtaatagcaagggaaaatagaatgaagtgatctccaaaaaataagtactttttgactgtaaataaaattgtaaggagtaaaaagtacttttttttctaaaaaaatgtaattaagtaaaagtaaaagtattgatttttaattgtactcaagtaaagtaaaaatccccaaaaataatacttaagtacagtaatcaagtaaaattactcaagtactttacacctctggttcttgaccccctaccttcagcaagcccagcagatccactagttctagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtg

SEQ ID NO: 18 - DNA, 7795 bp, Artificial Sequence
  Description of Artificial Sequence Synthetic plasmid
ccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtacaggtcattgcacaacaccagaaatgccctctgatctgcaaaagacgtgaatatctgttcagacacccatatccactctgttccacacaggtcagaggtttgtccaggagttcttgacagaggtgtaaaaagtactcaaaaattttactcaagtgaaagtacaagtacttagggaaaattttactcaattaaaagtaaaagtatctggctagaatcttacttgagtaaaagtaaaaaagtactccattaaaattgtacttgagtattaaggaagtaaaagtaaaagcaagaaagaaaactagagattcttgtttaagcttttaatctcaaaaaacattaaatgaaatgcatacaaggttttatcctgctttagaactgtttgtatttaattatcaaactataagacagacaatctaatgccagtacacgctactcaaagttgtaaaacctcagatttaacttcagtagaagctgattctcaaaattgttagtgtcaagcctagctcttttggggctgaaaagcaatcctgcagtgctgaaaagcctctcacaggcagccgatgcgggaagaggtgtattagtcttgatagagaggctgcaaatagcaggaaacgtgagcagagactccctggtgtctgaaacacaggccagatgggccgatatcaactttgtatagaaaagttgaccacccatgttcacccatattttgatcttttttcaagtaatttgaaacattaagaagactctagcatttaatatgctttatgaaaacaggacgtcatatgttttatcatcatttaacaatcagtacatgttattcatgtttgtaaaataaaaattagtcaagaaataatttgatgttttaatactctcatcaattattttatcggctctacgtttttgtaagtacgcatcagaccagcgaagattaatctgtaaagtctccctctgcatccgactgtcagcataaaagaaatgtgtggctgcagtatcaatagatggcctgtgtgatagcgtgttaaaatacgagctgagctattgggaatgtgcggagcggagcagcagtgcagtgaaagagctgctccagcacttccccctcccggcgttgatgtgaggggctccgcctgggtggtctccctctctctccactttggcgagctgttgcttagcagctaataataggagatgtttgcaaagtaatttgcctcttcctcggccaatgagaaccagagcacttttccatcaaaccacccttttcagggtttgcaaaggggctacctctccaaatgttactgcaacaggatcagaggtagactggcggaatccgctcgccactttcttctgctttaaaacgagttacgcgctatagctggacgaatgaagaccgagggatctatacggattcgcgcacataatgttgggatggagttggaagactttggctcaaggaaagactagagaatatttcttgaagttgaacccggtggatgcgcgtgtcgcgaatcttggattattattattatcgcctccgtttaacgcgccgcgtttgcctttaactttcttcagcgcttcttctgcctcccgtttggactcaactttgtacaaaaaagcaggctacaccatggctccaaagaagaagcgtaaggtaatggtgagcaagggcgaggaggacaacatggccatcatcaaggagttcatgcgcttcaaggtgcacatggagggctccgtgaacggccgcgagttcgagatcgagggcgagggcgagggccgcccctacgagggcacccagaccgccaagctgaaggtgaccaagggcggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgccgaggtcaagaccacctacaaggccaagaagcccgtgcagctgcccggcgcctacaacgtcaacatcaagctggacatcacctcccacaacgaggactacaccatcgtggaacagtacgagcgcgccgagggccgccactccaccggcggcatggacgagctgtacaagtaagacccagctttcttgtacaaagttgatgtagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaattcgcggccgcggcgccaatgcattgggccctcgagcatatgagatctgtcccgatctttctcttctgtgctgtcaatttagagtagtttttgaggtaatttgggtgcattctctgtaaaaacaacaaataaatatttatattgtatgcaataaatgtaacaattacacatacactctctgattacagcattaaagggttagttcacccaaaaatgaaaagatgtcattaatgactcaccctcatgtcgttccaagcccgtaacacctccgttcatcttcggaacacagtttaagatattttagatttagtccgagagctttctgtccctccactgaaaatgtatgtacggtatactgtccatgtccagaaaggtaataaaaacatcaaagtagtccatgtgacatcagtgggttaggtagaattatttgaagcatcgaaaatacattttggtccaaaaataacaaaacctacgactttattcagcattgtagcgtcactgcgaagtcgtgaacgcggattgacaacagacccggaagagaatacaatgccgaataaagtcgtaggttttgttatttttggaccaaaatgtattcgatgcttcaaaaaattctaactaacccactgatgtcacatggactactttgatgtttttattacctttctggacatggacagtataccgtacatacattctcaatggaggcacagaaagctctcggactaaatctaaaatatcttaaactgtgttctgaagatgaacggaggtcttacgggcttggaacgacatgagggcgagtcattaatgacattattttcatttttgggtgaactaaccctttaataaaactgacatttgcagccaattgcacactttataatgttaaatacttgttgattttagaaatacagtagcatttagtgacaatcaaacattaattttatttctgattatattgacaaaaacacaggactgtaaggctgacatgacagggtgaattgatttcattacattagttttgatggtaacacttttacaataaggttcgttagttaactacattagttaacatgaactaataatgaactgcacgtatacatgttaatttcaacatttactaatactttatatcttgttaacattagttaatgcactgtgaactaacatgaacaaacaatgaacatctgtatttctattaactaacgttaacaaagattaacagatacagtaacaaatgtattgctcatggttatttaatgttagttaatacatgaactaatcaacctcattgtaaagtgttaccgttttgacaatacaacatgtttggaaaaaaatttctcgcttggttttcttgtgttttgcatgaattcaaacacaatgagagcacggaatgataatgattattcaaagctgcaataggcgccaaatttcccgtggacctaacgttaccaattacaaatgcattttagcattttattacgtgagccttatattgtcaatcgcatgcattagtgaaaataggcctactacacatataaaaggcgcgccatcaacgtcaaaacatgcacacccaaaaaaaaaaaaaaaaaaaaaaaaaaaaactatcagtaaaacaaaatacttaatgtacttacctcaatatgcttccttaggtttgatggcgaacttttgaaggccgatatttctttattaagcgggagacagaggacacatttcatcttgaatgaatctttatttacaccacttaaagaaaagaattcgcgaagatacggccacgggtgctcttgatcctgtggctgattttggactgtgctgctcgcagctgctgatgaatcacatacttcctccattttcttccactgattgactgttataatttccctaatttccaggtcaaggtgctgtgcattgtggtaatagatgtgacatgacgtcacttccaaaggaccaatgaacatgtctgaccaatttcatataatgtgaaaacgattttcataggcagaataaataacatttaaattaaactgggcatcagcgcaattcaattggtttggtaatagcaagggaaaatagaatgaagtgatctccaaaaaataagtactttttgactgtaaataaaattgtaaggagtaaaaagtacttttttttctaaaaaaatgtaattaagtaaaagtaaaagtattgatttttaattgtactcaagtaaagtaaaaatccccaaaaataatacttaagtacagtaatcaagtaaaattactcaagtactttacacctctggttcttgaccccctaccttcagcaagcccagcagatccactagttctagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtg

SEQ ID NO: 19 - DNA, 7852 bp, Artificial Sequence
  Description of Artificial Sequence Synthetic plasmid
ccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtacaggtcattgcacaacaccagaaatgccctctgatctgcaaaagacgtgaatatctgttcagacacccatatccactctgttccacacaggtcagaggtttgtccaggagttcttgacagaggtgtaaaaagtactcaaaaattttactcaagtgaaagtacaagtacttagggaaaattttactcaattaaaagtaaaagtatctggctagaatcttacttgagtaaaagtaaaaaagtactccattaaaattgtacttgagtattaaggaagtaaaagtaaaagcaagaaagaaaactagagattcttgtttaagcttttaatctcaaaaaacattaaatgaaatgcatacaaggttttatcctgctttagaactgtttgtatttaattatcaaactataagacagacaatctaatgccagtacacgctactcaaagttgtaaaacctcagatttaacttcagtagaagctgattctcaaaattgttagtgtcaagcctagctcttttggggctgaaaagcaatcctgcagtgctgaaaagcctctcacaggcagccgatgcgggaagaggtgtattagtcttgatagagaggctgcaaatagcaggaaacgtgagcagagactccctggtgtctgaaacacaggccagatgggccgatatcaactttgtatagaaaagttgaccacccatgttcacccatattttgatcttttttcaagtaatttgaaacattaagaagactctagcatttaatatgctttatgaaaacaggacgtcatatgttttatcatcatttaacaatcagtacatgttattcatgtttgtaaaataaaaattagtcaagaaataatttgatgttttaatactctcatcaattattttatcggctctacgtttttgtaagtacgcatcagaccagcgaagattaatctgtaaagtctccctctgcatccgactgtcagcataaaagaaatgtgtggctgcagtatcaatagatggcctgtgtgatagcgtgttaaaatacgagctgagctattgggaatgtgcggagcggagcagcagtgcagtgaaagagctgctccagcacttccccctcccggcgttgatgtgaggggctccgcctgggtggtctccctctctctccactttggcgagctgttgcttagcagctaataataggagatgtttgcaaagtaatttgcctcttcctcggccaatgagaaccagagcacttttccatcaaaccacccttttcagggtttgcaaaggggctacctctccaaatgttactgcaacaggatcagaggtagactggcggaatccgctcgccactttcttctgctttaaaacgagttacgcgctatagctggacgaatgaagaccgagggatctatacggattcgcgcacataatgttgggatggagttggaagactttggctcaaggaaagactagagaatatttcttgaagttgaacccggtggatgcgcgtgtcgcgaatcttggattattattattatcgcctccgtttaacgcgccgcgtttgcctttaactttcttcagcgcttcttctgcctcccgtttggactcaactttgtacaaaaaagcaggctggaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggaggaggaagatctaagctgaaccctcctgatgagagtggccccggctgcatgagctgcaagtgtgtgctctcctgagacccagctttcttgtacaaagttgatgtagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaattcgcggccgcggcgccaatgcattgggccctcgagcatatgagatctgtcccgatctttctcttctgtgctgtcaatttagagtagtttttgaggtaatttgggtgcattctctgtaaaaacaacaaataaatatttatattgtatgcaataaatgtaacaattacacatacactctctgattacagcattaaagggttagttcacccaaaaatgaaaagatgtcattaatgactcaccctcatgtcgttccaagcccgtaacacctccgttcatcttcggaacacagtttaagatattttagatttagtccgagagctttctgtccctccactgaaaatgtatgtacggtatactgtccatgtccagaaaggtaataaaaacatcaaagtagtccatgtgacatcagtgggttaggtagaattatttgaagcatcgaaaatacattttggtccaaaaataacaaaacctacgactttattcagcattgtagcgtcactgcgaagtcgtgaacgcggattgacaacagacccggaagagaatacaatgccgaataaagtcgtaggttttgttatttttggaccaaaatgtattcgatgcttcaaaaaattctaactaacccactgatgtcacatggactactttgatgtttttattacctttctggacatggacagtataccgtacatacattctcaatggaggcacagaaagctctcggactaaatctaaaatatcttaaactgtgttctgaagatgaacggaggtcttacgggcttggaacgacatgagggcgagtcattaatgacattattttcatttttgggtgaactaaccctttaataaaactgacatttgcagccaattgcacactttataatgttaaatacttgttgattttagaaatacagtagcatttagtgacaatcaaacattaattttatttctgattatattgacaaaaacacaggactgtaaggctgacatgacagggtgaattgatttcattacattagttttgatggtaacacttttacaataaggttcgttagttaactacattagttaacatgaactaataatgaactgcacgtatacatgttaatttcaacatttactaatactttatatcttgttaacattagttaatgcactgtgaactaacatgaacaaacaatgaacatctgtatttctattaactaacgttaacaaagattaacagatacagtaacaaatgtattgctcatggttatttaatgttagttaatacatgaactaatcaacctcattgtaaagtgttaccgttttgacaatacaacatgtttggaaaaaaatttctcgcttggttttcttgtgttttgcatgaattcaaacacaatgagagcacggaatgataatgattattcaaagctgcaataggcgccaaatttcccgtggacctaacgttaccaattacaaatgcattttagcattttattacgtgagccttatattgtcaatcgcatgcattagtgaaaataggcctactacacatataaaaggcgcgccatcaacgtcaaaacatgcacacccaaaaaaaaaaaaaaaaaaaaaaaaaaaaactatcagtaaaacaaaatacttaatgtacttacctcaatatgcttccttaggtttgatggcgaacttttgaaggccgatatttctttattaagcgggagacagaggacacatttcatcttgaatgaatctttatttacaccacttaaagaaaagaattcgcgaagatacggccacgggtgctcttgatcctgtggctgattttggactgtgctgctcgcagctgctgatgaatcacatacttcctccattttcttccactgattgactgttataatttccctaatttccaggtcaaggtgctgtgcattgtggtaatagatgtgacatgacgtcacttccaaaggaccaatgaacatgtctgaccaatttcatataatgtgaaaacgattttcataggcagaataaataacatttaaattaaactgggcatcagcgcaattcaattggtttggtaatagcaagggaaaatagaatgaagtgatctccaaaaaataagtactttttgactgtaaataaaattgtaaggagtaaaaagtacttttttttctaaaaaaatgtaattaagtaaaagtaaaagtattgatttttaattgtactcaagtaaagtaaaaatccccaaaaataatacttaagtacagtaatcaagtaaaattactcaagtactttacacctctggttcttgaccccctaccttcagcaagcccagcagatccactagttctagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtg

SEQ ID NO: 20 - DNA, 7828 bp, Artificial Sequence
  Description of Artificial Sequence Synthetic plasmid
ccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtacaggtcattgcacaacaccagaaatgccctctgatctgcaaaagacgtgaatatctgttcagacacccatatccactctgttccacacaggtcagaggtttgtccaggagttcttgacagaggtgtaaaaagtactcaaaaattttactcaagtgaaagtacaagtacttagggaaaattttactcaattaaaagtaaaagtatctggctagaatcttacttgagtaaaagtaaaaaagtactccattaaaattgtacttgagtattaaggaagtaaaagtaaaagcaagaaagaaaactagagattcttgtttaagcttttaatctcaaaaaacattaaatgaaatgcatacaaggttttatcctgctttagaactgtttgtatttaattatcaaactataagacagacaatctaatgccagtacacgctactcaaagttgtaaaacctcagatttaacttcagtagaagctgattctcaaaattgttagtgtcaagcctagctcttttggggctgaaaagcaatcctgcagtgctgaaaagcctctcacaggcagccgatgcgggaagaggtgtattagtcttgatagagaggctgcaaatagcaggaaacgtgagcagagactccctggtgtctgaaacacaggccagatgggccgatatcaactttgtatagaaaagttgaccacccatgttcacccatattttgatcttttttcaagtaatttgaaacattaagaagactctagcatttaatatgctttatgaaaacaggacgtcatatgttttatcatcatttaacaatcagtacatgttattcatgtttgtaaaataaaaattagtcaagaaataatttgatgttttaatactctcatcaattattttatcggctctacgtttttgtaagtacgcatcagaccagcgaagattaatctgtaaagtctccctctgcatccgactgtcagcataaaagaaatgtgtggctgcagtatcaatagatggcctgtgtgatagcgtgttaaaatacgagctgagctattgggaatgtgcggagcggagcagcagtgcagtgaaagagctgctccagcacttccccctcccggcgttgatgtgaggggctccgcctgggtggtctccctctctctccactttggcgagctgttgcttagcagctaataataggagatgtttgcaaagtaatttgcctcttcctcggccaatgagaaccagagcacttttccatcaaaccacccttttcagggtttgcaaaggggctacctctccaaatgttactgcaacaggatcagaggtagactggcggaatccgctcgccactttcttctgctttaaaacgagttacgcgctatagctggacgaatgaagaccgagggatctatacggattcgcgcacataatgttgggatggagttggaagactttggctcaaggaaagactagagaatatttcttgaagttgaacccggtggatgcgcgtgtcgcgaatcttggattattattattatcgcctccgtttaacgcgccgcgtttgcctttaactttcttcagcgcttcttctgcctcccgtttggactcaactttgtacaaaaaagcaggctccaccatggtgagcaagggcgaggaggacaacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgccgaggtcaagaccacctacaaggccaagaagcccgtgcagctgcccggcgcctacaacgtcaacatcaagctggacatcacctcccacaacgaggactacaccatcgtggaacagtacgagcgcgccgagggccgccactccaccggcggcatggacgagctgtacaagaagctgaaccctcctgatgagagtggccccggctgcatgagctgcaagtgtgtgctctcctgagacccagctttcttgtacaaagttgatgtagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaattcgcggccgcggcgccaatgcattgggccctcgagcatatgagatctgtcccgatctttctcttctgtgctgtcaatttagagtagtttttgaggtaatttgggtgcattctctgtaaaaacaacaaataaatatttatattgtatgcaataaatgtaacaattacacatacactctctgattacagcattaaagggttagttcacccaaaaatgaaaagatgtcattaatgactcaccctcatgtcgttccaagcccgtaacacctccgttcatcttcggaacacagtttaagatattttagatttagtccgagagctttctgtccctccactgaaaatgtatgtacggtatactgtccatgtccagaaaggtaataaaaacatcaaagtagtccatgtgacatcagtgggttaggtagaattatttgaagcatcgaaaatacattttggtccaaaaataacaaaacctacgactttattcagcattgtagcgtcactgcgaagtcgtgaacgcggattgacaacagacccggaagagaatacaatgccgaataaagtcgtaggttttgttatttttggaccaaaatgtattcgatgcttcaaaaaattctaactaacccactgatgtcacatggactactttgatgtttttattacctttctggacatggacagtataccgtacatacattctcaatggaggcacagaaagctctcggactaaatctaaaatatcttaaactgtgttctgaagatgaacggaggtcttacgggcttggaacgacatgagggcgagtcattaatgacattattttcatttttgggtgaactaaccctttaataaaactgacatttgcagccaattgcacactttataatgttaaatacttgttgattttagaaatacagtagcatttagtgacaatcaaacattaattttatttctgattatattgacaaaaacacaggactgtaaggctgacatgacagggtgaattgatttcattacattagttttgatggtaacacttttacaataaggttcgttagttaactacattagttaacatgaactaataatgaactgcacgtatacatgttaatttcaacatttactaatactttatatcttgttaacattagttaatgcactgtgaactaacatgaacaaacaatgaacatctgtatttctattaactaacgttaacaaagattaacagatacagtaacaaatgtattgctcatggttatttaatgttagttaatacatgaactaatcaacctcattgtaaagtgttaccgttttgacaatacaacatgtttggaaaaaaatttctcgcttggttttcttgtgttttgcatgaattcaaacacaatgagagcacggaatgataatgattattcaaagctgcaataggcgccaaatttcccgtggacctaacgttaccaattacaaatgcattttagcattttattacgtgagccttatattgtcaatcgcatgcattagtgaaaataggcctactacacatataaaaggcgcgccatcaacgtcaaaacatgcacacccaaaaaaaaaaaaaaaaaaaaaaaaaaaaactatcagtaaaacaaaatacttaatgtacttacctcaatatgcttccttaggtttgatggcgaacttttgaaggccgatatttctttattaagcgggagacagaggacacatttcatcttgaatgaatctttatttacaccacttaaagaaaagaattcgcgaagatacggccacgggtgctcttgatcctgtggctgattttggactgtgctgctcgcagctgctgatgaatcacatacttcctccattttcttccactgattgactgttataatttccctaatttccaggtcaaggtgctgtgcattgtggtaatagatgtgacatgacgtcacttccaaaggaccaatgaacatgtctgaccaatttcatataatgtgaaaacgattttcataggcagaataaataacatttaaattaaactgggcatcagcgcaattcaattggtttggtaatagcaagggaaaatagaatgaagtgatctccaaaaaataagtactttttgactgtaaataaaattgtaaggagtaaaaagtacttttttttctaaaaaaatgtaattaagtaaaagtaaaagtattgatttttaattgtactcaagtaaagtaaaaatccccaaaaataatacttaagtacagtaatcaagtaaaattactcaagtactttacacctctggttcttgaccccctaccttcagcaagcccagcagatccactagttctagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtg

SEQ ID NO: 21 - DNA, 8991 bp, Artificial Sequence
  Description of Artificial Sequence Synthetic plasmid
ccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtacaggtcattgcacaacaccagaaatgccctctgatctgcaaaagacgtgaatatctgttcagacacccatatccactctgttccacacaggtcagaggtttgtccaggagttcttgacagaggtgtaaaaagtactcaaaaattttactcaagtgaaagtacaagtacttagggaaaattttactcaattaaaagtaaaagtatctggctagaatcttacttgagtaaaagtaaaaaagtactccattaaaattgtacttgagtattaaggaagtaaaagtaaaagcaagaaagaaaactagagattcttgtttaagcttttaatctcaaaaaacattaaatgaaatgcatacaaggttttatcctgctttagaactgtttgtatttaattatcaaactataagacagacaatctaatgccagtacacgctactcaaagttgtaaaacctcagatttaacttcagtagaagctgattctcaaaattgttagtgtcaagcctagctcttttggggctgaaaagcaatcctgcagtgctgaaaagcctctcacaggcagccgatgcgggaagaggtgtattagtcttgatagagaggctgcaaatagcaggaaacgtgagcagagactccctggtgtctgaaacacaggccagatgggccgatatcaactttgtatagaaaagttgctgtcgtttgatatccatgcttcgttcttttggagcttacctttacacaagcagtgacgggctgtggccgtttatgtttaggggctgtgtgatggcagatttctggacagcgcatggtcatctccccagtttatttatccaaggcctctagacctcggtccggacctccctcagtttgttaggcttaaaggggccccctaccgcatggagcaattcctcagattccacattagctcccattcccagctgcccttaccctcatcaaattaaacaatttcgagaaatgtgctcaacgctgccacagtatacaagcagacgtacccttgtgctttcccttagtgtcaatggtgctatatgctaagacatttaaatttgaactaatattttgccttccactgaattgcagtgaaaaaataatcaaatgacaggtggaccagatgcatgacatgggcttcatatcatattttctgtagggtatttgttgatttggcatatattttgtcattataagcatgcttaggtttctcaaacctttgcattgttctacaagtcagataattgattgagtcacatttggccatttacaattgaaaaaatcatctggaatagtcttgataaggagtctaatctgaaaagccattactctgaaacatcataaaataaccttaagtgttaagtaaaatcaacgtgttgtccaattcaaaatgtgtattatttctgtaatattttgaacatttttacaaagaatgacaaataaaacatcttaagattacctgaaatttgaaatatttatagaaataaattgatcaaagttttaattttctcatataatctaacaaaaatgtgttgtcaagaatagttttttggacaagctcagcaaaggatttatatgggtaaaaggctttttttttctggccatgaatagtgactatagtatttttaccatgatttacacaagacgcccattaagaaataagtcagacattaatttactgatatattaaaacattattttaaggaccatagtgcagcctcaaaatgctaattcattgccatttaaagtctttatattgtactttagccatttttatttacaagattttgatttgattttgatttattttttttacacttattttaaaaaaaaacttgttcacccatattttgatcttttttcaagtaatttgaaacattaagaagactctagcatttaatatgctttatgaaaacaggacgtcatatgttttatcatcatttaacaatcagtacatgttattcatgtttgtaaaataaaaattagtcaagaaataatttgatgttttaatactctcatcaattattttatcggctctacgtttttgtaagtacgcatcagaccagcgaagattaatctgtaaagtctccctctgcatccgactgtcagcataaaagaaatgtgtggctgcagtatcaatagatggcctgtgtgatagcgtgttaaaatacgagctgagctattgggaatgtgcggagcggagcagcagtgcagtgaaagagctgctccagcacttccccctcccggcgttgatgtgaggggctccgcctgggtggtctccctctctctccactttggcgagctgttgcttagcagctaataataggagatgtttgcaaagtaatttgcctcttcctcggccaatgagaaccagagcacttttccatcaaaccacccttttcagggtttgcaaaggggctacctctccaaatgttactgcaacaggatcagaggtagactggcggaatccgctcgccactttcttctgctttaaaacgagttacgcgctatagctggacgaatgaagaccgagggatctatacggattcgcgcacataatgttgggatggagttggaagactttggctcaaggaaagactagagaatatttcttgaagttgaacccggtggatgcgcgtgtcgcgaatcttggattattattattatcgcctccgtttaacgcgccgcgtttgcctttaactttcttcagcgcttcttctgcctcccgtttggactcaactttgtacaaaaaagcaggctggaccatggctccaaagaagaagcgtaaggtaaatcgatcgaattcaaggcctctcgagcctctagaaggtggcggaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaacacccagctttcttgtacaaagttgatgtagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaattcgcggccgcggcgccaatgcattgggccctcgagcatatgagatctgtcccgatctttctcttctgtgctgtcaatttagagtagtttttgaggtaatttgggtgcattctctgtaaaaacaacaaataaatatttatattgtatgcaataaatgtaacaattacacatacactctctgattacagcattaaagggttagttcacccaaaaatgaaaagatgtcattaatgactcaccctcatgtcgttccaagcccgtaacacctccgttcatcttcggaacacagtttaagatattttagatttagtccgagagctttctgtccctccactgaaaatgtatgtacggtatactgtccatgtccagaaaggtaataaaaacatcaaagtagtccatgtgacatcagtgggttaggtagaattatttgaagcatcgaaaatacattttggtccaaaaataacaaaacctacgactttattcagcattgtagcgtcactgcgaagtcgtgaacgcggattgacaacagacccggaagagaatacaatgccgaataaagtcgtaggttttgttatttttggaccaaaatgtattcgatgcttcaaaaaattctaactaacccactgatgtcacatggactactttgatgtttttattacctttctggacatggacagtataccgtacatacattctcaatggaggcacagaaagctctcggactaaatctaaaatatcttaaactgtgttctgaagatgaacggaggtcttacgggcttggaacgacatgagggcgagtcattaatgacattattttcatttttgggtgaactaaccctttaataaaactgacatttgcagccaattgcacactttataatgttaaatacttgttgattttagaaatacagtagcatttagtgacaatcaaacattaattttatttctgattatattgacaaaaacacaggactgtaaggctgacatgacagggtgaattgatttcattacattagttttgatggtaacacttttacaataaggttcgttagttaactacattagttaacatgaactaataatgaactgcacgtatacatgttaatttcaacatttactaatactttatatcttgttaacattagttaatgcactgtgaactaacatgaacaaacaatgaacatctgtatttctattaactaacgttaacaaagattaacagatacagtaacaaatgtattgctcatggttatttaatgttagttaatacatgaactaatcaacctcattgtaaagtgttaccgttttgacaatacaacatgtttggaaaaaaatttctcgcttggttttcttgtgttttgcatgaattcaaacacaatgagagcacggaatgataatgattattcaaagctgcaataggcgccaaatttcccgtggacctaacgttaccaattacaaatgcattttagcattttattacgtgagccttatattgtcaatcgcatgcattagtgaaaataggcctactacacatataaaaggcgcgccatcaacgtcaaaacatgcacacccaaaaaaaaaaaaaaaaaaaaaaaaaaaaactatcagtaaaacaaaatacttaatgtacttacctcaatatgcttccttaggtttgatggcgaacttttgaaggccgatatttctttattaagcgggagacagaggacacatttcatcttgaatgaatctttatttacaccacttaaagaaaagaattcgcgaagatacggccacgggtgctcttgatcctgtggctgattttggactgtgctgctcgcagctgctgatgaatcacatacttcctccattttcttccactgattgactgttataatttccctaatttccaggtcaaggtgctgtgcattgtggtaatagatgtgacatgacgtcacttccaaaggaccaatgaacatgtctgaccaatttcatataatgtgaaaacgattttcataggcagaataaataacatttaaattaaactgggcatcagcgcaattcaattggtttggtaatagcaagggaaaatagaatgaagtgatctccaaaaaataagtactttttgactgtaaataaaattgtaaggagtaaaaagtacttttttttctaaaaaaatgtaattaagtaaaagtaaaagtattgatttttaattgtactcaagtaaagtaaaaatccccaaaaataatacttaagtacagtaatcaagtaaaattactcaagtactttacacctctggttcttgaccccctaccttcagcaagcccagcagatccactagttctagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtg

SEQ ID NO: 22 - DNA, 8937 bp, Artificial Sequence; identical to SEQ ID NO: 14
  Description of Artificial Sequence Synthetic plasmid

SEQ ID NO: 23 - DNA, 8994 bp, Artificial Sequence
  Description of Artificial Sequence Synthetic plasmid
ccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtacaggtcattgcacaacaccagaaatgccctctgatctgcaaaagacgtgaatatctgttcagacacccatatccactctgttccacacaggtcagaggtttgtccaggagttcttgacagaggtgtaaaaagtactcaaaaattttactcaagtgaaagtacaagtacttagggaaaattttactcaattaaaagtaaaagtatctggctagaatcttacttgagtaaaagtaaaaaagtactccattaaaattgtacttgagtattaaggaagtaaaagtaaaagcaagaaagaaaactagagattcttgtttaagcttttaatctcaaaaaacattaaatgaaatgcatacaaggttttatcctgctttagaactgtttgtatttaattatcaaactataagacagacaatctaatgccagtacacgctactcaaagttgtaaaacctcagatttaacttcagtagaagctgattctcaaaattgttagtgtcaagcctagctcttttggggctgaaaagcaatcctgcagtgctgaaaagcctctcacaggcagccgatgcgggaagaggtgtattagtcttgatagagaggctgcaaatagcaggaaacgtgagcagagactccctggtgtctgaaacacaggccagatgggccgatatcaactttgtatagaaaagttgctgtcgtttgatatccatgcttcgttcttttggagcttacctttacacaagcagtgacgggctgtggccgtttatgtttaggggctgtgtgatggcagatttctggacagcgcatggtcatctccccagtttatttatccaaggcctctagacctcggtccggacctccctcagtttgttaggcttaaaggggccccctaccgcatggagcaattcctcagattccacattagctcccattcccagctgcccttaccctcatcaaattaaacaatttcgagaaatgtgctcaacgctgccacagtatacaagcagacgtacccttgtgctttcccttagtgtcaatggtgctatatgctaagacatttaaatttgaactaatattttgccttccactgaattgcagtgaaaaaataatcaaatgacaggtggaccagatgcatgacatgggcttcatatcatattttctgtagggtatttgttgatttggcatatattttgtcattataagcatgcttaggtttctcaaacctttgcattgttctacaagtcagataattgattgagtcacatttggccatttacaattgaaaaaatcatctggaatagtcttgataaggagtctaatctgaaaagccattactctgaaacatcataaaataaccttaagtgttaagtaaaatcaacgtgttgtccaattcaaaatgtgtattatttctgtaatattttgaacatttttacaaagaatgacaaataaaacatcttaagattacctgaaatttgaaatatttatagaaataaattgatcaaagttttaattttctcatataatctaacaaaaatgtgttgtcaagaatagttttttggacaagctcagcaaaggatttatatgggtaaaaggctttttttttctggccatgaatagtgactatagtatttttaccatgatttacacaagacgcccattaagaaataagtcagacattaatttactgatatattaaaacattattttaaggaccatagtgcagcctcaaaatgctaattcattgccatttaaagtctttatattgtactttagccatttttatttacaagattttgatttgattttgatttattttttttacacttattttaaaaaaaaacttgttcacccatattttgatcttttttcaagtaatttgaaacattaagaagactctagcatttaatatgctttatgaaaacaggacgtcatatgttttatcatcatttaacaatcagtacatgttattcatgtttgtaaaataaaaattagtcaagaaataatttgatgttttaatactctcatcaattattttatcggctctacgtttttgtaagtacgcatcagaccagcgaagattaatctgtaaagtctccctctgcatccgactgtcagcataaaagaaatgtgtggctgcagtatcaatagatggcctgtgtgatagcgtgttaaaatacgagctgagctattgggaatgtgcggagcggagcagcagtgcagtgaaagagctgctccagcacttccccctcccggcgttgatgtgaggggctccgcctgggtggtctccctctctctccactttggcgagctgttgcttagcagctaataataggagatgtttgcaaagtaatttgcctcttcctcggccaatgagaaccagagcacttttccatcaaaccacccttttcagggtttgcaaaggggctacctctccaaatgttactgcaacaggatcagaggtagactggcggaatccgctcgccactttcttctgctttaaaacgagttacgcgctatagctggacgaatgaagaccgagggatctatacggattcgcgcacataatgttgggatggagttggaagactttggctcaaggaaagactagagaatatttcttgaagttgaacccggtggatgcgcgtgtcgcgaatcttggattattattattatcgcctccgtttaacgcgccgcgtttgcctttaactttcttcagcgcttcttctgcctcccgtttggactcaactttgtacaaaaaagcaggctggaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggaggaggaagatctaagctgaaccctcctgatgagagtggccccggctgcatgagctgcaagtgtgtgctctcctgagacccagctttcttgtacaaagttgatgtagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaattcgcggccgcggcgccaatgcattgggccctcgagcatatgagatctgtcccgatctttctcttctgtgctgtcaatttagagtagtttttgaggtaatttgggtgcattctctgtaaaaacaacaaataaatatttatattgtatgcaataaatgtaacaattacacatacactctctgattacagcattaaagggttagttcacccaaaaatgaaaagatgtcattaatgactcaccctcatgtcgttccaagcccgtaacacctccgttcatcttcggaacacagtttaagatattttagatttagtccgagagctttctgtccctccactgaaaatgtatgtacggtatactgtccatgtccagaaaggtaataaaaacatcaaagtagtccatgtgacatcagtgggttaggtagaattatttgaagcatcgaaaatacattttggtccaaaaataacaaaacctacgactttattcagcattgtagcgtcactgcgaagtcgtgaacgcggattgacaacagacccggaagagaatacaatgccgaataaagtcgtaggttttgttatttttggaccaaaatgtattcgatgcttcaaaaaattctaactaacccactgatgtcacatggactactttgatgtttttattacctttctggacatggacagtataccgtacatacattctcaatggaggcacagaaagctctcggactaaatctaaaatatcttaaactgtgttctgaagatgaacggaggtcttacgggcttggaacgacatgagggcgagtcattaatgacattattttcatttttgggtgaactaaccctttaataaaactgacatttgcagccaattgcacactttataatgttaaatacttgttgattttagaaatacagtagcatttagtgacaatcaaacattaattttatttctgattatattgacaaaaacacaggactgtaaggctgacatgacagggtgaattgatttcattacattagttttgatggtaacacttttacaataaggttcgttagttaactacattagttaacatgaactaataatgaactgcacgtatacatgttaatttcaacatttactaatactttatatcttgttaacattagttaatgcactgtgaactaacatgaacaaacaatgaacatctgtatttctattaactaacgttaacaaagattaacagatacagtaacaaatgtattgctcatggttatttaatgttagttaatacatgaactaatcaacctcattgtaaagtgttaccgttttgacaatacaacatgtttggaaaaaaatttctcgcttggttttcttgtgttttgcatgaattcaaacacaatgagagcacggaatgataatgattattcaaagctgcaataggcgccaaatttcccgtggacctaacgttaccaattacaaatgcattttagcattttattacgtgagccttatattgtcaatcgcatgcattagtgaaaataggcctactacacatataaaaggcgcgccatcaacgtcaaaacatgcacacccaaaaaaaaaaaaaaaaaaaaaaaaaaaaactatcagtaaaacaaaatacttaatgtacttacctcaatatgcttccttaggtttgatggcgaacttttgaaggccgatatttctttattaagcgggagacagaggacacatttcatcttgaatgaatctttatttacaccacttaaagaaaagaattcgcgaagatacggccacgggtgctcttgatcctgtggctgattttggactgtgctgctcgcagctgctgatgaatcacatacttcctccattttcttccactgattgactgttataatttccctaatttccaggtcaaggtgctgtgcattgtggtaatagatgtgacatgacgtcacttccaaaggaccaatgaacatgtctgaccaatttcatataatgtgaaaacgattttcataggcagaataaataacatttaaattaaactgggcatcagcgcaattcaattggtttggtaatagcaagggaaaatagaatgaagtgatctccaaaaaataagtactttttgactgtaaataaaattgtaaggagtaaaaagtacttttttttctaaaaaaatgtaattaagtaaaagtaaaagtattgatttttaattgtactcaagtaaagtaaaaatccccaaaaataatacttaagtacagtaatcaagtaaaattactcaagtactttacacctctggttcttgaccccctaccttcagcaagcccagcagatccactagttctagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtg

SEQ ID NO: 24 - DNA, 8970 bp, Artificial Sequence
  Description of Artificial Sequence Synthetic plasmid
ccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtacaggtcattgcacaacaccagaaatgccctctgatctgcaaaagacgtgaatatctgttcagacacccatatccactctgttccacacaggtcagaggtttgtccaggagttcttgacagaggtgtaaaaagtactcaaaaattttactcaagtgaaagtacaagtacttagggaaaattttactcaattaaaagtaaaagtatctggctagaatcttacttgagtaaaagtaaaaaagtactccattaaaattgtacttgagtattaaggaagtaaaagtaaaagcaagaaagaaaactagagattcttgtttaagcttttaatctcaaaaaacattaaatgaaatgcatacaaggttttatcctgctttagaactgtttgtatttaattatcaaactataagacagacaatctaatgccagtacacgctactcaaagttgtaaaacctcagatttaacttcagtagaagctgattctcaaaattgttagtgtcaagcctagctcttttggggctgaaaagcaatcctgcagtgctgaaaagcctctcacaggcagccgatgcgggaagaggtgtattagtcttgatagagaggctgcaaatagcaggaaacgtgagcagagactccctggtgtctgaaacacaggccagatgggccgatatcaactttgtatagaaaagttgctgtcgtttgatatccatgcttcgttcttttggagcttacctttacacaagcagtgacgggctgtggccgtttatgtttaggggctgtgtgatggcagatttctggacagcgcatggtcatctccccagtttatttatccaaggcctctagacctcggtccggacctccctcagtttgttaggcttaaaggggccccctaccgcatggagcaattcctcagattccacattagctcccattcccagctgcccttaccctcatcaaattaaacaatttcgagaaatgtgctcaacgctgccacagtatacaagcagacgtacccttgtgctttcccttagtgtcaatggtgctatatgctaagacatttaaatttgaactaatattttgccttccactgaattgcagtgaaaaaataatcaaatgacaggtggaccagatgcatgacatgggcttcatatcatattttctgtagggtatttgttgatttggcatatattttgtcattataagcatgcttaggtttctcaaacctttgcattgttctacaagtcagataattgattgagtcacatttggccatttacaattgaaaaaatcatctggaatagtcttgataaggagtctaatctgaaaagccattactctgaaacatcataaaataaccttaagtgttaagtaaaatcaacgtgttgtccaattcaaaatgtgtattatttctgtaatattttgaacatttttacaaagaatgacaaataaaacatcttaagattacctgaaatttgaaatatttatagaaataaattgatcaaagttttaattttctcatataatctaacaaaaatgtgttgtcaagaatagttttttggacaagctcagcaaaggatttatatgggtaaaaggctttttttttctggccatgaatagtgactatagtatttttaccatgatttacacaagacgcccattaagaaataagtcagacattaatttactgatatattaaaacattattttaaggaccatagtgcagcctcaaaatgctaattcattgccatttaaagtctttatattgtactttagccatttttatttacaagattttgatttgattttgatttattttttttacacttattttaaaaaaaaacttgttcacccatattttgatcttttttcaagtaatttgaaacattaagaagactctagcatttaatatgctttatgaaaacaggacgtcatatgttttatcatcatttaacaatcagtacatgttattcatgtttgtaaaataaaaattagtcaagaaataatttgatgttttaatactctcatcaattattttatcggctctacgtttttgtaagtacgcatcagaccagcgaagattaatctgtaaagtctccctctgcatccgactgtcagcataaaagaaatgtgtggctgcagtatcaatagatggcctgtgtgatagcgtgttaaaatacgagctgagctattgggaatgtgcggagcggagcagcagtgcagtgaaagagctgctccagcacttccccctcccggcgttgatgtgaggggctccgcctgggtggtctccctctctctccactttggcgagctgttgcttagcagctaataataggagatgtttgcaaagtaatttgcctcttcctcggccaatgagaaccagagcacttttccatcaaaccacccttttcagggtttgcaaaggggctacctctccaaatgttactgcaacaggatcagaggtagactggcggaatccgctcgccactttcttctgctttaaaacgagttacgcgctatagctggacgaatgaagaccgagggatctatacggattcgcgcacataatgttgggatggagttggaagactttggctcaaggaaagactagagaatatttcttgaagttgaacccggtggatgcgcgtgtcgcgaatcttggattattattattatcgcctccgtttaacgcgccgcgtttgcctttaactttcttcagcgcttcttctgcctcccgtttggactcaactttgtacaaaaaagcaggctccaccatggtgagcaagggcgaggaggacaacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgccgaggtcaagaccacctacaaggccaagaagcccgtgcagctgcccggcgcctacaacgtcaacatcaagctggacatcacctcccacaacgaggactacaccatcgtggaacagtacgagcgcgccgagggccgccactccaccggcggcatggacgagctgtacaagaagctgaaccctcctgatgagagtggccccggctgcatgagctgcaagtgtgtgctctcctgagacccagctttcttgtacaaagttgatgtagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaattcgcggccgcggcgccaatgcattgggccctcgagcatatgagatctgtcccgatctttctcttctgtgctgtcaatttagagtagtttttgaggtaatttgggtgcattctctgtaaaaacaacaaataaatatttatattgtatgcaataaatgtaacaattacacatacactctctgattacagcattaaagggttagttcacccaaaaatgaaaagatgtcattaatgactcaccctcatgtcgttccaagcccgtaacacctccgttcatcttcggaacacagtttaagatattttagatttagtccgagagctttctgtccctccactgaaaatgtatgtacggtatactgtccatgtccagaaaggtaataaaaacatcaaagtagtccatgtgacatcagtgggttaggtagaattatttgaagcatcgaaaatacattttggtccaaaaataacaaaacctacgactttattcagcattgtagcgtcactgcgaagtcgtgaacgcggattgacaacagacccggaagagaatacaatgccgaataaagtcgtaggttttgttatttttggaccaaaatgtattcgatgcttcaaaaaattctaactaacccactgatgtcacatggactactttgatgtttttattacctttctggacatggacagtataccgtacatacattctcaatggaggcacagaaagctctcggactaaatctaaaatatcttaaactgtgttctgaagatgaacggaggtcttacgggcttggaacgacatgagggcgagtcattaatgacattattttcatttttgggtgaactaaccctttaataaaactgacatttgcagccaattgcacactttataatgttaaatacttgttgattttagaaatacagtagcatttagtgacaatcaaacattaattttatttctgattatattgacaaaaacacaggactgtaaggctgacatgacagggtgaattgatttcattacattagttttgatggtaacacttttacaataaggttcgttagttaactacattagttaacatgaactaataatgaactgcacgtatacatgttaatttcaacatttactaatactttatatcttgttaacattagttaatgcactgtgaactaacatgaacaaacaatgaacatctgtatttctattaactaacgttaacaaagattaacagatacagtaacaaatgtattgctcatggttatttaatgttagttaatacatgaactaatcaacctcattgtaaagtgttaccgttttgacaatacaacatgtttggaaaaaaatttctcgcttggttttcttgtgttttgcatgaattcaaacacaatgagagcacggaatgataatgattattcaaagctgcaataggcgccaaatttcccgtggacctaacgttaccaattacaaatgcattttagcattttattacgtgagccttatattgtcaatcgcatgcattagtgaaaataggcctactacacatataaaaggcgcgccatcaacgtcaaaacatgcacacccaaaaaaaaaaaaaaaaaaaaaaaaaaaaactatcagtaaaacaaaatacttaatgtacttacctcaatatgcttccttaggtttgatggcgaacttttgaaggccgatatttctttattaagcgggagacagaggacacatttcatcttgaatgaatctttatttacaccacttaaagaaaagaattcgcgaagatacggccacgggtgctcttgatcctgtggctgattttggactgtgctgctcgcagctgctgatgaatcacatacttcctccattttcttccactgattgactgttataatttccctaatttccaggtcaaggtgctgtgcattgtggtaatagatgtgacatgacgtcacttccaaaggaccaatgaacatgtctgaccaatttcatataatgtgaaaacgattttcataggcagaataaataacatttaaattaaactgggcatcagcgcaattcaattggtttggtaatagcaagggaaaatagaatgaagtgatctccaaaaaataagtactttttgactgtaaataaaattgtaaggagtaaaaagtacttttttttctaaaaaaatgtaattaagtaaaagtaaaagtattgatttttaattgtactcaagtaaagtaaaaatccccaaaaataatacttaagtacagtaatcaagtaaaattactcaagtactttacacctctggttcttgaccccctaccttcagcaagcccagcagatccactagttctagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtg

SEQ ID NO: 25 - DNA, 6985 bp, Artificial Sequence
  Description of Artificial Sequence Synthetic plasmid
ccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtacaggtcattgcacaacaccagaaatgccctctgatctgcaaaagacgtgaatatctgttcagacacccatatccactctgttccacacaggtcagaggtttgtccaggagttcttgacagaggtgtaaaaagtactcaaaaattttactcaagtgaaagtacaagtacttagggaaaattttactcaattaaaagtaaaagtatctggctagaatcttacttgagtaaaagtaaaaaagtactccattaaaattgtacttgagtattaaggaagtaaaagtaaaagcaagaaagaaaactagagattcttgtttaagcttttaatctcaaaaaacattaaatgaaatgcatacaaggttttatcctgctttagaactgtttgtatttaattatcaaactataagacagacaatctaatgccagtacacgctactcaaagttgtaaaacctcagatttaacttcagtagaagctgattctcaaaattgttagtgtcaagcctagctcttttggggctgaaaagcaatcctgcagtgctgaaaagcctctcacaggcagccgatgcgggaagaggtgtattagtcttgatagagaggctgcaaatagcaggaaacgtgagcagagactccctggtgtctgaaacacaggccagatgggccgatatcaactttgtatagaaaagttgaccacccagaccacccagaccacccagaccacccagaccacccacaactttgtacaaaaaagcaggctggaccatggctccaaagaagaagcgtaaggtaaatcgatcgaattcaaggcctctcgagcctctagaaggtggcggaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaacacccagctttcttgtacaaagttgatgtagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaattcgcggccgcggcgccaatgcattgggccctcgagcatatgagatctgtcccgatctttctcttctgtgctgtcaatttagagtagtttttgaggtaatttgggtgcattctctgtaaaaacaacaaataaatatttatattgtatgcaataaatgtaacaattacacatacactctctgattacagcattaaagggttagttcacccaaaaatgaaaagatgtcattaatgactcaccctcatgtcgttccaagcccgtaacacctccgttcatcttcggaacacagtttaagatattttagatttagtccgagagctttctgtccctccactgaaaatgtatgtacggtatactgtccatgtccagaaaggtaataaaaacatcaaagtagtccatgtgacatcagtgggttaggtagaattatttgaagcatcgaaaatacattttggtccaaaaataacaaaacctacgactttattcagcattgtagcgtcactgcgaagtcgtgaacgcggattgacaacagacccggaagagaatacaatgccgaataaagtcgtaggttttgttatttttggaccaaaatgtattcgatgcttcaaaaaattctaactaacccactgatgtcacatggactactttgatgtttttattacctttctggacatggacagtataccgtacatacattctcaatggaggcacagaaagctctcggactaaatctaaaatatcttaaactgtgttctgaagatgaacggaggtcttacgggcttggaacgacatgagggcgagtcattaatgacattattttcatttttgggtgaactaaccctttaataaaactgacatttgcagccaattgcacactttataatgttaaatacttgttgattttagaaatacagtagcatttagtgacaatcaaacattaattttatttctgattatattgacaaaaacacaggactgtaaggctgacatgacagggtgaattgatttcattacattagttttgatggtaacacttttacaataaggttcgttagttaactacattagttaacatgaactaataatgaactgcacgtatacatgttaatttcaacatttactaatactttatatcttgttaacattagttaatgcactgtgaactaacatgaacaaacaatgaacatctgtatttctattaactaacgttaacaaagattaacagatacagtaacaaatgtattgctcatggttatttaatgttagttaatacatgaactaatcaacctcattgtaaagtgttaccgttttgacaatacaacatgtttggaaaaaaatttctcgcttggttttcttgtgttttgcatgaattcaaacacaatgagagcacggaatgataatgattattcaaagctgcaataggcgccaaatttcccgtggacctaacgttaccaattacaaatgcattttagcattttattacgtgagccttatattgtcaatcgcatgcattagtgaaaataggcctactacacatataaaaggcgcgccatcaacgtcaaaacatgcacacccaaaaaaaaaaaaaaaaaaaaaaaaaaaaactatcagtaaaacaaaatacttaatgtacttacctcaatatgcttccttaggtttgatggcgaacttttgaaggccgatatttctttattaagcgggagacagaggacacatttcatcttgaatgaatctttatttacaccacttaaagaaaagaattcgcgaagatacggccacgggtgctcttgatcctgtggctgattttggactgtgctgctcgcagctgctgatgaatcacatacttcctccattttcttccactgattgactgttataatttccctaatttccaggtcaaggtgctgtgcattgtggtaatagatgtgacatgacgtcacttccaaaggaccaatgaacatgtctgaccaatttcatataatgtgaaaacgattttcataggcagaataaataacatttaaattaaactgggcatcagcgcaattcaattggtttggtaatagcaagggaaaatagaatgaagtgatctccaaaaaataagtactttttgactgtaaataaaattgtaaggagtaaaaagtacttttttttctaaaaaaatgtaattaagtaaaagtaaaagtattgatttttaattgtactcaagtaaagtaaaaatccccaaaaataatacttaagtacagtaatcaagtaaaattactcaagtactttacacctctggttcttgaccccctaccttcagcaagcccagcagatccactagttctagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtg

SEQ ID NO: 26 - DNA, 6931 bp, Artificial Sequence
  Description of Artificial Sequence Synthetic plasmid
ccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtacaggtcattgcacaacaccagaaatgccctctgatctgcaaaagacgtgaatatctgttcagacacccatatccactctgttccacacaggtcagaggtttgtccaggagttcttgacagaggtgtaaaaagtactcaaaaattttactcaagtgaaagtacaagtacttagggaaaattttactcaattaaaagtaaaagtatctggctagaatcttacttgagtaaaagtaaaaaagtactccattaaaattgtacttgagtattaaggaagtaaaagtaaaagcaagaaagaaaactagagattcttgtttaagcttttaatctcaaaaaacattaaatgaaatgcatacaaggttttatcctgctttagaactgtttgtatttaattatcaaactataagacagacaatctaatgccagtacacgctactcaaagttgtaaaacctcagatttaacttcagtagaagctgattctcaaaattgttagtgtcaagcctagctcttttggggctgaaaagcaatcctgcagtgctgaaaagcctctcacaggcagccgatgcgggaagaggtgtattagtcttgatagagaggctgcaaatagcaggaaacgtgagcagagactccctggtgtctgaaacacaggccagatgggccgatatcaactttgtatagaaaagttgaccacccagaccacccagaccacccagaccacccagaccacccacaactttgtacaaaaaagcaggctacaccatggctccaaagaagaagcgtaaggtaatggtgagcaagggcgaggaggacaacatggccatcatcaaggagttcatgcgcttcaaggtgcacatggagggctccgtgaacggccgcgagttcgagatcgagggcgagggcgagggccgcccctacgagggcacccagaccgccaagctgaaggtgaccaagggcggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgccgaggtcaagaccacctacaaggccaagaagcccgtgcagctgcccggcgcctacaacgtcaacatcaagctggacatcacctcccacaacgaggactacaccatcgtggaacagtacgagcgcgccgagggccgccactccaccggcggcatggacgagctgtacaagtaagacccagctttcttgtacaaagttgatgtagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaattcgcggccgcggcgccaatgcattgggccctcgagcatatgagatctgtcccgatctttctcttctgtgctgtcaatttagagtagtttttgaggtaatttgggtgcattctctgtaaaaacaacaaataaatatttatattgtatgcaataaatgtaacaattacacatacactctctgattacagcattaaagggttagttcacccaaaaatgaaaagatgtcattaatgactcaccctcatgtcgttccaagcccgtaacacctccgttcatcttcggaacacagtttaagatattttagatttagtccgagagctttctgtccctccactgaaaatgtatgtacggtatactgtccatgtccagaaaggtaataaaaacatcaaagtagtccatgtgacatcagtgggttaggtagaattatttgaagcatcgaaaatacattttggtccaaaaataacaaaacctacgactttattcagcattgtagcgtcactgcgaagtcgtgaacgcggattgacaacagacccggaagagaatacaatgccgaataaagtcgtaggttttgttatttttggaccaaaatgtattcgatgcttcaaaaaattctaactaacccactgatgtcacatggactactttgatgtttttattacctttctggacatggacagtataccgtacatacattctcaatggaggcacagaaagctctcggactaaatctaaaatatcttaaactgtgttctgaagatgaacggaggtcttacgggcttggaacgacatgagggcgagtcattaatgacattattttcatttttgggtgaactaaccctttaataaaactgacatttgcagccaattgcacactttataatgttaaatacttgttgattttagaaatacagtagcatttagtgacaatcaaacattaattttatttctgattatattgacaaaaacacaggactgtaaggctgacatgacagggtgaattgatttcattacattagttttgatggtaacacttttacaataaggttcgttagttaactacattagttaacatgaactaataatgaactgcacgtatacatgttaatttcaacatttactaatactttatatcttgttaacattagttaatgcactgtgaactaacatgaacaaacaatgaacatctgtatttctattaactaacgttaacaaagattaacagatacagtaacaaatgtattgctcatggttatttaatgttagttaatacatgaactaatcaacctcattgtaaagtgttaccgttttgacaatacaacatgtttggaaaaaaatttctcgcttggttttcttgtgttttgcatgaattcaaacacaatgagagcacggaatgataatgattattcaaagctgcaataggcgccaaatttcccgtggacctaacgttaccaattacaaatgcattttagcattttattacgtgagccttatattgtcaatcgcatgcattagtgaaaataggcctactacacatataaaaggcgcgccatcaacgtcaaaacatgcacacccaaaaaaaaaaaaaaaaaaaaaaaaaaaaactatcagtaaaacaaaatacttaatgtacttacctcaatatgcttccttaggtttgatggcgaacttttgaaggccgatatttctttattaagcgggagacagaggacacatttcatcttgaatgaatctttatttacaccacttaaagaaaagaattcgcgaagatacggccacgggtgctcttgatcctgtggctgattttggactgtgctgctcgcagctgctgatgaatcacatacttcctccattttcttccactgattgactgttataatttccctaatttccaggtcaaggtgctgtgcattgtggtaatagatgtgacatgacgtcacttccaaaggaccaatgaacatgtctgaccaatttcatataatgtgaaaacgattttcataggcagaataaataacatttaaattaaactgggcatcagcgcaattcaattggtttggtaatagcaagggaaaatagaatgaagtgatctccaaaaaataagtactttttgactgtaaataaaattgtaaggagtaaaaagtacttttttttctaaaaaaatgtaattaagtaaaagtaaaagtattgatttttaattgtactcaagtaaagtaaaaatccccaaaaataatacttaagtacagtaatcaagtaaaattactcaagtactttacacctctggttcttgaccccctaccttcagcaagcccagcagatccactagttctagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtg

SEQ ID NO: 27 - DNA, 6988 bp, Artificial Sequence
  Description of Artificial Sequence Synthetic plasmid
ccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtacaggtcattgcacaacaccagaaatgccctctgatctgcaaaagacgtgaatatctgttcagacacccatatccactctgttccacacaggtcagaggtttgtccaggagttcttgacagaggtgtaaaaagtactcaaaaattttactcaagtgaaagtacaagtacttagggaaaattttactcaattaaaagtaaaagtatctggctagaatcttacttgagtaaaagtaaaaaagtactccattaaaattgtacttgagtattaaggaagtaaaagtaaaagcaagaaagaaaactagagattcttgtttaagcttttaatctcaaaaaacattaaatgaaatgcatacaaggttttatcctgctttagaactgtttgtatttaattatcaaactataagacagacaatctaatgccagtacacgctactcaaagttgtaaaacctcagatttaacttcagtagaagctgattctcaaaattgttagtgtcaagcctagctcttttggggctgaaaagcaatcctgcagtgctgaaaagcctctcacaggcagccgatgcgggaagaggtgtattagtcttgatagagaggctgcaaatagcaggaaacgtgagcagagactccctggtgtctgaaacacaggccagatgggccgatatcaactttgtatagaaaagttgaccacccagaccacccagaccacccagaccacccagaccacccacaactttgtacaaaaaagcaggctggaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggaggaggaagatctaagctgaaccctcctgatgagagtggccccggctgcatgagctgcaagtgtgtgctctcctgagacccagctttcttgtacaaagttgatgtagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaattcgcggccgcggcgccaatgcattgggccctcgagcatatgagatctgtcccgatctttctcttctgtgctgtcaatttagagtagtttttgaggtaatttgggtgcattctctgtaaaaacaacaaataaatatttatattgtatgcaataaatgtaacaattacacatacactctctgattacagcattaaagggttagttcacccaaaaatgaaaagatgtcattaatgactcaccctcatgtcgttccaagcccgtaacacctccgttcatcttcggaacacagtttaagatattttagatttagtccgagagctttctgtccctccactgaaaatgtatgtacggtatactgtccatgtccagaaaggtaataaaaacatcaaagtagtccatgtgacatcagtgggttaggtagaattatttgaagcatcgaaaatacattttggtccaaaaataacaaaacctacgactttattcagcattgtagcgtcactgcgaagtcgtgaacgcggattgacaacagacccggaagagaatacaatgccgaataaagtcgtaggttttgttatttttggaccaaaatgtattcgatgcttcaaaaaattctaactaacccactgatgtcacatggactactttgatgtttttattacctttctggacatggacagtataccgtacatacattctcaatggaggcacagaaagctctcggactaaatctaaaatatcttaaactgtgttctgaagatgaacggaggtcttacgggcttggaacgacatgagggcgagtcattaatgacattattttcatttttgggtgaactaaccctttaataaaactgacatttgcagccaattgcacactttataatgttaaatacttgttgattttagaaatacagtagcatttagtgacaatcaaacattaattttatttctgattatattgacaaaaacacaggactgtaaggctgacatgacagggtgaattgatttcattacattagttttgatggtaacacttttacaataaggttcgttagttaactacattagttaacatgaactaataatgaactgcacgtatacatgttaatttcaacatttactaatactttatatcttgttaacattagttaatgcactgtgaactaacatgaacaaacaatgaacatctgtatttctattaactaacgttaacaaagattaacagatacagtaacaaatgtattgctcatggttatttaatgttagttaatacatgaactaatcaacctcattgtaaagtgttaccgttttgacaatacaacatgtttggaaaaaaatttctcgcttggttttcttgtgttttgcatgaattcaaacacaatgagagcacggaatgataatgattattcaaagctgcaataggcgccaaatttcccgtggacctaacgttaccaattacaaatgcattttagcattttattacgtgagccttatattgtcaatcgcatgcattagtgaaaataggcctactacacatataaaaggcgcgccatcaacgtcaaaacatgcacacccaaaaaaaaaaaaaaaaaaaaaaaaaaaaactatcagtaaaacaaaatacttaatgtacttacctcaatatgcttccttaggtttgatggcgaacttttgaaggccgatatttctttattaagcgggagacagaggacacatttcatcttgaatgaatctttatttacaccacttaaagaaaagaattcgcgaagatacggccacgggtgctcttgatcctgtggctgattttggactgtgctgctcgcagctgctgatgaatcacatacttcctccattttcttccactgattgactgttataatttccctaatttccaggtcaaggtgctgtgcattgtggtaatagatgtgacatgacgtcacttccaaaggaccaatgaacatgtctgaccaatttcatataatgtgaaaacgattttcataggcagaataaataacatttaaattaaactgggcatcagcgcaattcaattggtttggtaatagcaagggaaaatagaatgaagtgatctccaaaaaataagtactttttgactgtaaataaaattgtaaggagtaaaaagtacttttttttctaaaaaaatgtaattaagtaaaagtaaaagtattgatttttaattgtactcaagtaaagtaaaaatccccaaaaataatacttaagtacagtaatcaagtaaaattactcaagtactttacacctctggttcttgaccccctaccttcagcaagcccagcagatccactagttctagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtg

SEQ ID NO: 28 - DNA, 6964 bp, Artificial Sequence
  Description of Artificial Sequence Synthetic plasmid
ccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtacaggtcattgcacaacaccagaaatgccctctgatctgcaaaagacgtgaatatctgttcagacacccatatccactctgttccacacaggtcagaggtttgtccaggagttcttgacagaggtgtaaaaagtactcaaaaattttactcaagtgaaagtacaagtacttagggaaaattttactcaattaaaagtaaaagtatctggctagaatcttacttgagtaaaagtaaaaaagtactccattaaaattgtacttgagtattaaggaagtaaaagtaaaagcaagaaagaaaactagagattcttgtttaagcttttaatctcaaaaaacattaaatgaaatgcatacaaggttttatcctgctttagaactgtttgtatttaattatcaaactataagacagacaatctaatgccagtacacgctactcaaagttgtaaaacctcagatttaacttcagtagaagctgattctcaaaattgttagtgtcaagcctagctcttttggggctgaaaagcaatcctgcagtgctgaaaagcctctcacaggcagccgatgcgggaagaggtgtattagtcttgatagagaggctgcaaatagcaggaaacgtgagcagagactccctggtgtctgaaacacaggccagatgggccgatatcaactttgtatagaaaagttgaccacccagaccacccagaccacccagaccacccagaccacccacaactttgtacaaaaaagcaggctccaccatggtgagcaagggcgaggaggacaacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgccgaggtcaagaccacctacaaggccaagaagcccgtgcagctgcccggcgcctacaacgtcaacatcaagctggacatcacctcccacaacgaggactacaccatcgtggaacagtacgagcgcgccgagggccgccactccaccggcggcatggacgagctgtacaagaagctgaaccctcctgatgagagtggccccggctgcatgagctgcaagtgtgtgctctcctgagacccagctttcttgtacaaagttgatgtagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaattcgcggccgcggcgccaatgcattgggccctcgagcatatgagatctgtcccgatctttctcttctgtgctgtcaatttagagtagtttttgaggtaatttgggtgcattctctgtaaaaacaacaaataaatatttatattgtatgcaataaatgtaacaattacacatacactctctgattacagcattaaagggttagttcacccaaaaatgaaaagatgtcattaatgactcaccctcatgtcgttccaagcccgtaacacctccgttcatcttcggaacacagtttaagatattttagatttagtccgagagctttctgtccctccactgaaaatgtatgtacggtatactgtccatgtccagaaaggtaataaaaacatcaaagtagtccatgtgacatcagtgggttaggtagaattatttgaagcatcgaaaatacattttggtccaaaaataacaaaacctacgactttattcagcattgtagcgtcactgcgaagtcgtgaacgcggattgacaacagacccggaagagaatacaatgccgaataaagtcgtaggttttgttatttttggaccaaaatgtattcgatgcttcaaaaaattctaactaacccactgatgtcacatggactactttgatgtttttattacctttctggacatggacagtataccgtacatacattctcaatggaggcacagaaagctctcggactaaatctaaaatatcttaaactgtgttctgaagatgaacggaggtcttacgggcttggaacgacatgagggcgagtcattaatgacattattttcatttttgggtgaactaaccctttaataaaactgacatttgcagccaattgcacactttataatgttaaatacttgttgattttagaaatacagtagcatttagtgacaatcaaacattaattttatttctgattatattgacaaaaacacaggactgtaaggctgacatgacagggtgaattgatttcattacattagttttgatggtaacacttttacaataaggttcgttagttaactacattagttaacatgaactaataatgaactgcacgtatacatgttaatttcaacatttactaatactttatatcttgttaacattagttaatgcactgtgaactaacatgaacaaacaatgaacatctgtatttctattaactaacgttaacaaagattaacagatacagtaacaaatgtattgctcatggttatttaatgttagttaatacatgaactaatcaacctcattgtaaagtgttaccgttttgacaatacaacatgtttggaaaaaaatttctcgcttggttttcttgtgttttgcatgaattcaaacacaatgagagcacggaatgataatgattattcaaagctgcaataggcgccaaatttcccgtggacctaacgttaccaattacaaatgcattttagcattttattacgtgagccttatattgtcaatcgcatgcattagtgaaaataggcctactacacatataaaaggcgcgccatcaacgtcaaaacatgcacacccaaaaaaaaaaaaaaaaaaaaaaaaaaaaactatcagtaaaacaaaatacttaatgtacttacctcaatatgcttccttaggtttgatggcgaacttttgaaggccgatatttctttattaagcgggagacagaggacacatttcatcttgaatgaatctttatttacaccacttaaagaaaagaattcgcgaagatacggccacgggtgctcttgatcctgtggctgattttggactgtgctgctcgcagctgctgatgaatcacatacttcctccattttcttccactgattgactgttataatttccctaatttccaggtcaaggtgctgtgcattgtggtaatagatgtgacatgacgtcacttccaaaggaccaatgaacatgtctgaccaatttcatataatgtgaaaacgattttcataggcagaataaataacatttaaattaaactgggcatcagcgcaattcaattggtttggtaatagcaagggaaaatagaatgaagtgatctccaaaaaataagtactttttgactgtaaataaaattgtaaggagtaaaaagtacttttttttctaaaaaaatgtaattaagtaaaagtaaaagtattgatttttaattgtactcaagtaaagtaaaaatccccaaaaataatacttaagtacagtaatcaagtaaaattactcaagtactttacacctctggttcttgaccccctaccttcagcaagcccagcagatccactagttctagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtg

SEQ ID NO: 29 - DNA, 7030 bp, Artificial Sequence
  Description of Artificial Sequence Synthetic plasmid
ccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtacaggtcattgcacaacaccagaaatgccctctgatctgcaaaagacgtgaatatctgttcagacacccatatccactctgttccacacaggtcagaggtttgtccaggagttcttgacagaggtgtaaaaagtactcaaaaattttactcaagtgaaagtacaagtacttagggaaaattttactcaattaaaagtaaaagtatctggctagaatcttacttgagtaaaagtaaaaaagtactccattaaaattgtacttgagtattaaggaagtaaaagtaaaagcaagaaagaaaactagagattcttgtttaagcttttaatctcaaaaaacattaaatgaaatgcatacaaggttttatcctgctttagaactgtttgtatttaattatcaaactataagacagacaatctaatgccagtacacgctactcaaagttgtaaaacctcagatttaacttcagtagaagctgattctcaaaattgttagtgtcaagcctagctcttttggggctgaaaagcaatcctgcagtgctgaaaagcctctcacaggcagccgatgcgggaagaggtgtattagtcttgatagagaggctgcaaatagcaggaaacgtgagcagagactccctggtgtctgaaacacaggccagatgggccgatatcaactttgtatagaaaagttgaccacccagaccacccagaccacccagaccacccagaccacccagaccacccagaccacccagaccacccagaccacccagaccacccacaactttgtacaaaaaagcaggctggaccatggctccaaagaagaagcgtaaggtaaatcgatcgaattcaaggcctctcgagcctctagaaggtggcggaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaacacccagctttcttgtacaaagttgatgtagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaattcgcggccgcggcgccaatgcattgggccctcgagcatatgagatctgtcccgatctttctcttctgtgctgtcaatttagagtagtttttgaggtaatttgggtgcattctctgtaaaaacaacaaataaatatttatattgtatgcaataaatgtaacaattacacatacactctctgattacagcattaaagggttagttcacccaaaaatgaaaagatgtcattaatgactcaccctcatgtcgttccaagcccgtaacacctccgttcatcttcggaacacagtttaagatattttagatttagtccgagagctttctgtccctccactgaaaatgtatgtacggtatactgtccatgtccagaaaggtaataaaaacatcaaagtagtccatgtgacatcagtgggttaggtagaattatttgaagcatcgaaaatacattttggtccaaaaataacaaaacctacgactttattcagcattgtagcgtcactgcgaagtcgtgaacgcggattgacaacagacccggaagagaatacaatgccgaataaagtcgtaggttttgttatttttggaccaaaatgtattcgatgcttcaaaaaattctaactaacccactgatgtcacatggactactttgatgtttttattacctttctggacatggacagtataccgtacatacattctcaatggaggcacagaaagctctcggactaaatctaaaatatcttaaactgtgttctgaagatgaacggaggtcttacgggcttggaacgacatgagggcgagtcattaatgacattattttcatttttgggtgaactaaccctttaataaaactgacatttgcagccaattgcacactttataatgttaaatacttgttgattttagaaatacagtagcatttagtgacaatcaaacattaattttatttctgattatattgacaaaaacacaggactgtaaggctgacatgacagggtgaattgatttcattacattagttttgatggtaacacttttacaataaggttcgttagttaactacattagttaacatgaactaataatgaactgcacgtatacatgttaatttcaacatttactaatactttatatcttgttaacattagttaatgcactgtgaactaacatgaacaaacaatgaacatctgtatttctattaactaacgttaacaaagattaacagatacagtaacaaatgtattgctcatggttatttaatgttagttaatacatgaactaatcaacctcattgtaaagtgttaccgttttgacaatacaacatgtttggaaaaaaatttctcgcttggttttcttgtgttttgcatgaattcaaacacaatgagagcacggaatgataatgattattcaaagctgcaataggcgccaaatttcccgtggacctaacgttaccaattacaaatgcattttagcattttattacgtgagccttatattgtcaatcgcatgcattagtgaaaataggcctactacacatataaaaggcgcgccatcaacgtcaaaacatgcacacccaaaaaaaaaaaaaaaaaaaaaaaaaaaaactatcagtaaaacaaaatacttaatgtacttacctcaatatgcttccttaggtttgatggcgaacttttgaaggccgatatttctttattaagcgggagacagaggacacatttcatcttgaatgaatctttatttacaccacttaaagaaaagaattcgcgaagatacggccacgggtgctcttgatcctgtggctgattttggactgtgctgctcgcagctgctgatgaatcacatacttcctccattttcttccactgattgactgttataatttccctaatttccaggtcaaggtgctgtgcattgtggtaatagatgtgacatgacgtcacttccaaaggaccaatgaacatgtctgaccaatttcatataatgtgaaaacgattttcataggcagaataaataacatttaaattaaactgggcatcagcgcaattcaattggtttggtaatagcaagggaaaatagaatgaagtgatctccaaaaaataagtactttttgactgtaaataaaattgtaaggagtaaaaagtacttttttttctaaaaaaatgtaattaagtaaaagtaaaagtattgatttttaattgtactcaagtaaagtaaaaatccccaaaaataatacttaagtacagtaatcaagtaaaattactcaagtactttacacctctggttcttgaccccctaccttcagcaagcccagcagatccactagttctagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtg

SEQ ID NO: 30 - DNA, 6976 bp, Artificial Sequence
  Description of Artificial Sequence Synthetic plasmid
ccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtacaggtcattgcacaacaccagaaatgccctctgatctgcaaaagacgtgaatatctgttcagacacccatatccactctgttccacacaggtcagaggtttgtccaggagttcttgacagaggtgtaaaaagtactcaaaaattttactcaagtgaaagtacaagtacttagggaaaattttactcaattaaaagtaaaagtatctggctagaatcttacttgagtaaaagtaaaaaagtactccattaaaattgtacttgagtattaaggaagtaaaagtaaaagcaagaaagaaaactagagattcttgtttaagcttttaatctcaaaaaacattaaatgaaatgcatacaaggttttatcctgctttagaactgtttgtatttaattatcaaactataagacagacaatctaatgccagtacacgctactcaaagttgtaaaacctcagatttaacttcagtagaagctgattctcaaaattgttagtgtcaagcctagctcttttggggctgaaaagcaatcctgcagtgctgaaaagcctctcacaggcagccgatgcgggaagaggtgtattagtcttgatagagaggctgcaaatagcaggaaacgtgagcagagactccctggtgtctgaaacacaggccagatgggccgatatcaactttgtatagaaaagttgaccacccagaccacccagaccacccagaccacccagaccacccagaccacccagaccacccagaccacccagaccacccagaccacccacaactttgtacaaaaaagcaggctacaccatggctccaaagaagaagcgtaaggtaatggtgagcaagggcgaggaggacaacatggccatcatcaaggagttcatgcgcttcaaggtgcacatggagggctccgtgaacggccgcgagttcgagatcgagggcgagggcgagggccgcccctacgagggcacccagaccgccaagctgaaggtgaccaagggcggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgccgaggtcaagaccacctacaaggccaagaagcccgtgcagctgcccggcgcctacaacgtcaacatcaagctggacatcacctcccacaacgaggactacaccatcgtggaacagtacgagcgcgccgagggccgccactccaccggcggcatggacgagctgtacaagtaagacccagctttcttgtacaaagttgatgtagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaattcgcggccgcggcgccaatgcattgggccctcgagcatatgagatctgtcccgatctttctcttctgtgctgtcaatttagagtagtttttgaggtaatttgggtgcattctctgtaaaaacaacaaataaatatttatattgtatgcaataaatgtaacaattacacatacactctctgattacagcattaaagggttagttcacccaaaaatgaaaagatgtcattaatgactcaccctcatgtcgttccaagcccgtaacacctccgttcatcttcggaacacagtttaagatattttagatttagtccgagagctttctgtccctccactgaaaatgtatgtacggtatactgtccatgtccagaaaggtaataaaaacatcaaagtagtccatgtgacatcagtgggttaggtagaattatttgaagcatcgaaaatacattttggtccaaaaataacaaaacctacgactttattcagcattgtagcgtcactgcgaagtcgtgaacgcggattgacaacagacccggaagagaatacaatgccgaataaagtcgtaggttttgttatttttggaccaaaatgtattcgatgcttcaaaaaattctaactaacccactgatgtcacatggactactttgatgtttttattacctttctggacatggacagtataccgtacatacattctcaatggaggcacagaaagctctcggactaaatctaaaatatcttaaactgtgttctgaagatgaacggaggtcttacgggcttggaacgacatgagggcgagtcattaatgacattattttcatttttgggtgaactaaccctttaataaaactgacatttgcagccaattgcacactttataatgttaaatacttgttgattttagaaatacagtagcatttagtgacaatcaaacattaattttatttctgattatattgacaaaaacacaggactgtaaggctgacatgacagggtgaattgatttcattacattagttttgatggtaacacttttacaataaggttcgttagttaactacattagttaacatgaactaataatgaactgcacgtatacatgttaatttcaacatttactaatactttatatcttgttaacattagttaatgcactgtgaactaacatgaacaaacaatgaacatctgtatttctattaactaacgttaacaaagattaacagatacagtaacaaatgtattgctcatggttatttaatgttagttaatacatgaactaatcaacctcattgtaaagtgttaccgttttgacaatacaacatgtttggaaaaaaatttctcgcttggttttcttgtgttttgcatgaattcaaacacaatgagagcacggaatgataatgattattcaaagctgcaataggcgccaaatttcccgtggacctaacgttaccaattacaaatgcattttagcattttattacgtgagccttatattgtcaatcgcatgcattagtgaaaataggcctactacacatataaaaggcgcgccatcaacgtcaaaacatgcacacccaaaaaaaaaaaaaaaaaaaaaaaaaaaaactatcagtaaaacaaaatacttaatgtacttacctcaatatgcttccttaggtttgatggcgaacttttgaaggccgatatttctttattaagcgggagacagaggacacatttcatcttgaatgaatctttatttacaccacttaaagaaaagaattcgcgaagatacggccacgggtgctcttgatcctgtggctgattttggactgtgctgctcgcagctgctgatgaatcacatacttcctccattttcttccactgattgactgttataatttccctaatttccaggtcaaggtgctgtgcattgtggtaatagatgtgacatgacgtcacttccaaaggaccaatgaacatgtctgaccaatttcatataatgtgaaaacgattttcataggcagaataaataacatttaaattaaactgggcatcagcgcaattcaattggtttggtaatagcaagggaaaatagaatgaagtgatctccaaaaaataagtactttttgactgtaaataaaattgtaaggagtaaaaagtacttttttttctaaaaaaatgtaattaagtaaaagtaaaagtattgatttttaattgtactcaagtaaagtaaaaatccccaaaaataatacttaagtacagtaatcaagtaaaattactcaagtactttacacctctggttcttgaccccctaccttcagcaagcccagcagatccactagttctagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtg

SEQ ID NO: 31 - DNA, 7033 bp, Artificial Sequence
  Description of Artificial Sequence Synthetic plasmid
ccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtacaggtcattgcacaacaccagaaatgccctctgatctgcaaaagacgtgaatatctgttcagacacccatatccactctgttccacacaggtcagaggtttgtccaggagttcttgacagaggtgtaaaaagtactcaaaaattttactcaagtgaaagtacaagtacttagggaaaattttactcaattaaaagtaaaagtatctggctagaatcttacttgagtaaaagtaaaaaagtactccattaaaattgtacttgagtattaaggaagtaaaagtaaaagcaagaaagaaaactagagattcttgtttaagcttttaatctcaaaaaacattaaatgaaatgcatacaaggttttatcctgctttagaactgtttgtatttaattatcaaactataagacagacaatctaatgccagtacacgctactcaaagttgtaaaacctcagatttaacttcagtagaagctgattctcaaaattgttagtgtcaagcctagctcttttggggctgaaaagcaatcctgcagtgctgaaaagcctctcacaggcagccgatgcgggaagaggtgtattagtcttgatagagaggctgcaaatagcaggaaacgtgagcagagactccctggtgtctgaaacacaggccagatgggccgatatcaactttgtatagaaaagttgaccacccagaccacccagaccacccagaccacccagaccacccagaccacccagaccacccagaccacccagaccacccagaccacccacaactttgtacaaaaaagcaggctggaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggaggaggaagatctaagctgaaccctcctgatgagagtggccccggctgcatgagctgcaagtgtgtgctctcctgagacccagctttcttgtacaaagttgatgtagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaattcgcggccgcggcgccaatgcattgggccctcgagcatatgagatctgtcccgatctttctcttctgtgctgtcaatttagagtagtttttgaggtaatttgggtgcattctctgtaaaaacaacaaataaatatttatattgtatgcaataaatgtaacaattacacatacactctctgattacagcattaaagggttagttcacccaaaaatgaaaagatgtcattaatgactcaccctcatgtcgttccaagcccgtaacacctccgttcatcttcggaacacagtttaagatattttagatttagtccgagagctttctgtccctccactgaaaatgtatgtacggtatactgtccatgtccagaaaggtaataaaaacatcaaagtagtccatgtgacatcagtgggttaggtagaattatttgaagcatcgaaaatacattttggtccaaaaataacaaaacctacgactttattcagcattgtagcgtcactgcgaagtcgtgaacgcggattgacaacagacccggaagagaatacaatgccgaataaagtcgtaggttttgttatttttggaccaaaatgtattcgatgcttcaaaaaattctaactaacccactgatgtcacatggactactttgatgtttttattacctttctggacatggacagtataccgtacatacattctcaatggaggcacagaaagctctcggactaaatctaaaatatcttaaactgtgttctgaagatgaacggaggtcttacgggcttggaacgacatgagggcgagtcattaatgacattattttcatttttgggtgaactaaccctttaataaaactgacatttgcagccaattgcacactttataatgttaaatacttgttgattttagaaatacagtagcatttagtgacaatcaaacattaattttatttctgattatattgacaaaaacacaggactgtaaggctgacatgacagggtgaattgatttcattacattagttttgatggtaacacttttacaataaggttcgttagttaactacattagttaacatgaactaataatgaactgcacgtatacatgttaatttcaacatttactaatactttatatcttgttaacattagttaatgcactgtgaactaacatgaacaaacaatgaacatctgtatttctattaactaacgttaacaaagattaacagatacagtaacaaatgtattgctcatggttatttaatgttagttaatacatgaactaatcaacctcattgtaaagtgttaccgttttgacaatacaacatgtttggaaaaaaatttctcgcttggttttcttgtgttttgcatgaattcaaacacaatgagagcacggaatgataatgattattcaaagctgcaataggcgccaaatttcccgtggacctaacgttaccaattacaaatgcattttagcattttattacgtgagccttatattgtcaatcgcatgcattagtgaaaataggcctactacacatataaaaggcgcgccatcaacgtcaaaacatgcacacccaaaaaaaaaaaaaaaaaaaaaaaaaaaaactatcagtaaaacaaaatacttaatgtacttacctcaatatgcttccttaggtttgatggcgaacttttgaaggccgatatttctttattaagcgggagacagaggacacatttcatcttgaatgaatctttatttacaccacttaaagaaaagaattcgcgaagatacggccacgggtgctcttgatcctgtggctgattttggactgtgctgctcgcagctgctgatgaatcacatacttcctccattttcttccactgattgactgttataatttccctaatttccaggtcaaggtgctgtgcattgtggtaatagatgtgacatgacgtcacttccaaaggaccaatgaacatgtctgaccaatttcatataatgtgaaaacgattttcataggcagaataaataacatttaaattaaactgggcatcagcgcaattcaattggtttggtaatagcaagggaaaatagaatgaagtgatctccaaaaaataagtactttttgactgtaaataaaattgtaaggagtaaaaagtacttttttttctaaaaaaatgtaattaagtaaaagtaaaagtattgatttttaattgtactcaagtaaagtaaaaatccccaaaaataatacttaagtacagtaatcaagtaaaattactcaagtactttacacctctggttcttgaccccctaccttcagcaagcccagcagatccactagttctagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtg

SEQ ID NO: 32 - DNA, 7009 bp, Artificial Sequence
  Description of Artificial Sequence Synthetic plasmid
ccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtacaggtcattgcacaacaccagaaatgccctctgatctgcaaaagacgtgaatatctgttcagacacccatatccactctgttccacacaggtcagaggtttgtccaggagttcttgacagaggtgtaaaaagtactcaaaaattttactcaagtgaaagtacaagtacttagggaaaattttactcaattaaaagtaaaagtatctggctagaatcttacttgagtaaaagtaaaaaagtactccattaaaattgtacttgagtattaaggaagtaaaagtaaaagcaagaaagaaaactagagattcttgtttaagcttttaatctcaaaaaacattaaatgaaatgcatacaaggttttatcctgctttagaactgtttgtatttaattatcaaactataagacagacaatctaatgccagtacacgctactcaaagttgtaaaacctcagatttaacttcagtagaagctgattctcaaaattgttagtgtcaagcctagctcttttggggctgaaaagcaatcctgcagtgctgaaaagcctctcacaggcagccgatgcgggaagaggtgtattagtcttgatagagaggctgcaaatagcaggaaacgtgagcagagactccctggtgtctgaaacacaggccagatgggccgatatcaactttgtatagaaaagttgaccacccagaccacccagaccacccagaccacccagaccacccagaccacccagaccacccagaccacccagaccacccagaccacccacaactttgtacaaaaaagcaggctccaccatggtgagcaagggcgaggaggacaacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgccgaggtcaagaccacctacaaggccaagaagcccgtgcagctgcccggcgcctacaacgtcaacatcaagctggacatcacctcccacaacgaggactacaccatcgtggaacagtacgagcgcgccgagggccgccactccaccggcggcatggacgagctgtacaagaagctgaaccctcctgatgagagtggccccggctgcatgagctgcaagtgtgtgctctcctgagacccagctttcttgtacaaagttgatgtagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaattcgcggccgcggcgccaatgcattgggccctcgagcatatgagatctgtcccgatctttctcttctgtgctgtcaatttagagtagtttttgaggtaatttgggtgcattctctgtaaaaacaacaaataaatatttatattgtatgcaataaatgtaacaattacacatacactctctgattacagcattaaagggttagttcacccaaaaatgaaaagatgtcattaatgactcaccctcatgtcgttccaagcccgtaacacctccgttcatcttcggaacacagtttaagatattttagatttagtccgagagctttctgtccctccactgaaaatgtatgtacggtatactgtccatgtccagaaaggtaataaaaacatcaaagtagtccatgtgacatcagtgggttaggtagaattatttgaagcatcgaaaatacattttggtccaaaaataacaaaacctacgactttattcagcattgtagcgtcactgcgaagtcgtgaacgcggattgacaacagacccggaagagaatacaatgccgaataaagtcgtaggttttgttatttttggaccaaaatgtattcgatgcttcaaaaaattctaactaacccactgatgtcacatggactactttgatgtttttattacctttctggacatggacagtataccgtacatacattctcaatggaggcacagaaagctctcggactaaatctaaaatatcttaaactgtgttctgaagatgaacggaggtcttacgggcttggaacgacatgagggcgagtcattaatgacattattttcatttttgggtgaactaaccctttaataaaactgacatttgcagccaattgcacactttataatgttaaatacttgttgattttagaaatacagtagcatttagtgacaatcaaacattaattttatttctgattatattgacaaaaacacaggactgtaaggctgacatgacagggtgaattgatttcattacattagttttgatggtaacacttttacaataaggttcgttagttaactacattagttaacatgaactaataatgaactgcacgtatacatgttaatttcaacatttactaatactttatatcttgttaacattagttaatgcactgtgaactaacatgaacaaacaatgaacatctgtatttctattaactaacgttaacaaagattaacagatacagtaacaaatgtattgctcatggttatttaatgttagttaatacatgaactaatcaacctcattgtaaagtgttaccgttttgacaatacaacatgtttggaaaaaaatttctcgcttggttttcttgtgttttgcatgaattcaaacacaatgagagcacggaatgataatgattattcaaagctgcaataggcgccaaatttcccgtggacctaacgttaccaattacaaatgcattttagcattttattacgtgagccttatattgtcaatcgcatgcattagtgaaaataggcctactacacatataaaaggcgcgccatcaacgtcaaaacatgcacacccaaaaaaaaaaaaaaaaaaaaaaaaaaaaactatcagtaaaacaaaatacttaatgtacttacctcaatatgcttccttaggtttgatggcgaacttttgaaggccgatatttctttattaagcgggagacagaggacacatttcatcttgaatgaatctttatttacaccacttaaagaaaagaattcgcgaagatacggccacgggtgctcttgatcctgtggctgattttggactgtgctgctcgcagctgctgatgaatcacatacttcctccattttcttccactgattgactgttataatttccctaatttccaggtcaaggtgctgtgcattgtggtaatagatgtgacatgacgtcacttccaaaggaccaatgaacatgtctgaccaatttcatataatgtgaaaacgattttcataggcagaataaataacatttaaattaaactgggcatcagcgcaattcaattggtttggtaatagcaagggaaaatagaatgaagtgatctccaaaaaataagtactttttgactgtaaataaaattgtaaggagtaaaaagtacttttttttctaaaaaaatgtaattaagtaaaagtaaaagtattgatttttaattgtactcaagtaaagtaaaaatccccaaaaataatacttaagtacagtaatcaagtaaaattactcaagtactttacacctctggttcttgaccccctaccttcagcaagcccagcagatccactagttctagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtg

SEQ ID NO: 33 - DNA, 7119 bp, Artificial Sequence
  Description of Artificial Sequence Synthetic plasmid
ccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtacaggtcattgcacaacaccagaaatgccctctgatctgcaaaagacgtgaatatctgttcagacacccatatccactctgttccacacaggtcagaggtttgtccaggagttcttgacagaggtgtaaaaagtactcaaaaattttactcaagtgaaagtacaagtacttagggaaaattttactcaattaaaagtaaaagtatctggctagaatcttacttgagtaaaagtaaaaaagtactccattaaaattgtacttgagtattaaggaagtaaaagtaaaagcaagaaagaaaactagagattcttgtttaagcttttaatctcaaaaaacattaaatgaaatgcatacaaggttttatcctgctttagaactgtttgtatttaattatcaaactataagacagacaatctaatgccagtacacgctactcaaagttgtaaaacctcagatttaacttcagtagaagctgattctcaaaattgttagtgtcaagcctagctcttttggggctgaaaagcaatcctgcagtgctgaaaagcctctcacaggcagccgatgcgggaagaggtgtattagtcttgatagagaggctgcaaatagcaggaaacgtgagcagagactccctggtgtctgaaacacaggccagatgggccgatatcaactttgtatagaaaagttgaccacccagaccacccagaccacccagaccacccagaccacccagaccacccagaccacccagaccacccagaccacccagaccacccagaccacccagaccacccagaccacccagaccacccagaccacccagaccacccagaccacccagaccacccagaccacccagaccacccacaactttgtacaaaaaagcaggctggaccatggctccaaagaagaagcgtaaggtaaatcgatcgaattcaaggcctctcgagcctctagaaggtggcggaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaacccagctttcttgtacaaagttgatgtagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaattcgcggccgcggcgccaatgcattgggccctcgagcatatgagatctgtcccgatctttctcttctgtgctgtcaatttagagtagtttttgaggtaatttgggtgcattctctgtaaaaacaacaaataaatatttatattgtatgcaataaatgtaacaattacacatacactctctgattacagcattaaagggttagttcacccaaaaatgaaaagatgtcattaatgactcaccctcatgtcgttccaagcccgtaacacctccgttcatcttcggaacacagtttaagatattttagatttagtccgagagctttctgtccctccactgaaaatgtatgtacggtatactgtccatgtccagaaaggtaataaaaacatcaaagtagtccatgtgacatcagtgggttaggtagaattatttgaagcatcgaaaatacattttggtccaaaaataacaaaacctacgactttattcagcattgtagcgtcactgcgaagtcgtgaacgcggattgacaacagacccggaagagaatacaatgccgaataaagtcgtaggttttgttatttttggaccaaaatgtattcgatgcttcaaaaaattctaactaacccactgatgtcacatggactactttgatgtttttattacctttctggacatggacagtataccgtacatacattctcaatggaggcacagaaagctctcggactaaatctaaaatatcttaaactgtgttctgaagatgaacggaggtcttacgggcttggaacgacatgagggcgagtcattaatgacattattttcatttttgggtgaactaaccctttaataaaactgacatttgcagccaattgcacactttataatgttaaatacttgttgattttagaaatacagtagcatttagtgacaatcaaacattaattttatttctgattatattgacaaaaacacaggactgtaaggctgacatgacagggtgaattgatttcattacattagttttgatggtaacacttttacaataaggttcgttagttaactacattagttaacatgaactaataatgaactgcacgtatacatgttaatttcaacatttactaatactttatatcttgttaacattagttaatgcactgtgaactaacatgaacaaacaatgaacatctgtatttctattaactaacgttaacaaagattaacagatacagtaacaaatgtattgctcatggttatttaatgttagttaatacatgaactaatcaacctcattgtaaagtgttaccgttttgacaatacaacatgtttggaaaaaaatttctcgcttggttttcttgtgttttgcatgaattcaaacacaatgagagcacggaatgataatgattattcaaagctgcaataggcgccaaatttcccgtggacctaacgttaccaattacaaatgcattttagcattttattacgtgagccttatattgtcaatcgcatgcattagtgaaaataggcctactacacatataaaaggcgcgccatcaacgtcaaaacatgcacacccaaaaaaaaaaaaaaaaaaaaaaaaaaaaactatcagtaaaacaaaatacttaatgtacttacctcaatatgcttccttaggtttgatggcgaacttttgaaggccgatatttctttattaagcgggagacagaggacacatttcatcttgaatgaatctttatttacaccacttaaagaaaagaattcgcgaagatacggccacgggtgctcttgatcctgtggctgattttggactgtgctgctcgcagctgctgatgaatcacatacttcctccattttcttccactgattgactgttataatttccctaatttccaggtcaaggtgctgtgcattgtggtaatagatgtgacatgacgtcacttccaaaggaccaatgaacatgtctgaccaatttcatataatgtgaaaacgattttcataggcagaataaataacatttaaattaaactgggcatcagcgcaattcaattggtttggtaatagcaagggaaaatagaatgaagtgatctccaaaaaataagtactttttgactgtaaataaaattgtaaggagtaaaaagtacttttttttctaaaaaaatgtaattaagtaaaagtaaaagtattgatttttaattgtactcaagtaaagtaaaaatccccaaaaataatacttaagtacagtaatcaagtaaaattactcaagtactttacacctctggttcttgaccccctaccttcagcaagcccagcagatccactagttctagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtg

SEQ ID NO: 34 - DNA, 7066 bp, Artificial Sequence
  Description of Artificial Sequence Synthetic plasmid
ccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtacaggtcattgcacaacaccagaaatgccctctgatctgcaaaagacgtgaatatctgttcagacacccatatccactctgttccacacaggtcagaggtttgtccaggagttcttgacagaggtgtaaaaagtactcaaaaattttactcaagtgaaagtacaagtacttagggaaaattttactcaattaaaagtaaaagtatctggctagaatcttacttgagtaaaagtaaaaaagtactccattaaaattgtacttgagtattaaggaagtaaaagtaaaagcaagaaagaaaactagagattcttgtttaagcttttaatctcaaaaaacattaaatgaaatgcatacaaggttttatcctgctttagaactgtttgtatttaattatcaaactataagacagacaatctaatgccagtacacgctactcaaagttgtaaaacctcagatttaacttcagtagaagctgattctcaaaattgttagtgtcaagcctagctcttttggggctgaaaagcaatcctgcagtgctgaaaagcctctcacaggcagccgatgcgggaagaggtgtattagtcttgatagagaggctgcaaatagcaggaaacgtgagcagagactccctggtgtctgaaacacaggccagatgggccgatatcaactttgtatagaaaagttgaccacccagaccacccagaccacccagaccacccagaccacccagaccacccagaccacccagaccacccagaccacccagaccacccagaccacccagaccacccagaccacccagaccacccagaccacccagaccacccagaccacccagaccacccagaccacccagaccacccacaactttgtacaaaaaagcaggctacaccatggctccaaagaagaagcgtaaggtaatggtgagcaagggcgaggaggacaacatggccatcatcaaggagttcatgcgcttcaaggtgcacatggagggctccgtgaacggccgcgagttcgagatcgagggcgagggcgagggccgcccctacgagggcacccagaccgccaagctgaaggtgaccaagggcggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgccgaggtcaagaccacctacaaggccaagaagcccgtgcagctgcccggcgcctacaacgtcaacatcaagctggacatcacctcccacaacgaggactacaccatcgtggaacagtacgagcgcgccgagggccgccactccaccggcggcatggacgagctgtacaagtaagacccagctttcttgtacaaagttgatgtagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaattcgcggccgcggcgccaatgcattgggccctcgagcatatgagatctgtcccgatctttctcttctgtgctgtcaatttagagtagtttttgaggtaatttgggtgcattctctgtaaaaacaacaaataaatatttatattgtatgcaataaatgtaacaattacacatacactctctgattacagcattaaagggttagttcacccaaaaatgaaaagatgtcattaatgactcaccctcatgtcgttccaagcccgtaacacctccgttcatcttcggaacacagtttaagatattttagatttagtccgagagctttctgtccctccactgaaaatgtatgtacggtatactgtccatgtccagaaaggtaataaaaacatcaaagtagtccatgtgacatcagtgggttaggtagaattatttgaagcatcgaaaatacattttggtccaaaaataacaaaacctacgactttattcagcattgtagcgtcactgcgaagtcgtgaacgcggattgacaacagacccggaagagaatacaatgccgaataaagtcgtaggttttgttatttttggaccaaaatgtattcgatgcttcaaaaaattctaactaacccactgatgtcacatggactactttgatgtttttattacctttctggacatggacagtataccgtacatacattctcaatggaggcacagaaagctctcggactaaatctaaaatatcttaaactgtgttctgaagatgaacggaggtcttacgggcttggaacgacatgagggcgagtcattaatgacattattttcatttttgggtgaactaaccctttaataaaactgacatttgcagccaattgcacactttataatgttaaatacttgttgattttagaaatacagtagcatttagtgacaatcaaacattaattttatttctgattatattgacaaaaacacaggactgtaaggctgacatgacagggtgaattgatttcattacattagttttgatggtaacacttttacaataaggttcgttagttaactacattagttaacatgaactaataatgaactgcacgtatacatgttaatttcaacatttactaatactttatatcttgttaacattagttaatgcactgtgaactaacatgaacaaacaatgaacatctgtatttctattaactaacgttaacaaagattaacagatacagtaacaaatgtattgctcatggttatttaatgttagttaatacatgaactaatcaacctcattgtaaagtgttaccgttttgacaatacaacatgtttggaaaaaaatttctcgcttggttttcttgtgttttgcatgaattcaaacacaatgagagcacggaatgataatgattattcaaagctgcaataggcgccaaatttcccgtggacctaacgttaccaattacaaatgcattttagcattttattacgtgagccttatattgtcaatcgcatgcattagtgaaaataggcctactacacatataaaaggcgcgccatcaacgtcaaaacatgcacacccaaaaaaaaaaaaaaaaaaaaaaaaaaaaactatcagtaaaacaaaatacttaatgtacttacctcaatatgcttccttaggtttgatggcgaacttttgaaggccgatatttctttattaagcgggagacagaggacacatttcatcttgaatgaatctttatttacaccacttaaagaaaagaattcgcgaagatacggccacgggtgctcttgatcctgtggctgattttggactgtgctgctcgcagctgctgatgaatcacatacttcctccattttcttccactgattgactgttataatttccctaatttccaggtcaaggtgctgtgcattgtggtaatagatgtgacatgacgtcacttccaaaggaccaatgaacatgtctgaccaatttcatataatgtgaaaacgattttcataggcagaataaataacatttaaattaaactgggcatcagcgcaattcaattggtttggtaatagcaagggaaaatagaatgaagtgatctccaaaaaataagtactttttgactgtaaataaaattgtaaggagtaaaaagtacttttttttctaaaaaaatgtaattaagtaaaagtaaaagtattgatttttaattgtactcaagtaaagtaaaaatccccaaaaataatacttaagtacagtaatcaagtaaaattactcaagtactttacacctctggttcttgaccccctaccttcagcaagcccagcagatccactagttctagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtg

SEQ ID NO: 35 - DNA, 7123 bp, Artificial Sequence
  Description of Artificial Sequence Synthetic plasmid
ccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtacaggtcattgcacaacaccagaaatgccctctgatctgcaaaagacgtgaatatctgttcagacacccatatccactctgttccacacaggtcagaggtttgtccaggagttcttgacagaggtgtaaaaagtactcaaaaattttactcaagtgaaagtacaagtacttagggaaaattttactcaattaaaagtaaaagtatctggctagaatcttacttgagtaaaagtaaaaaagtactccattaaaattgtacttgagtattaaggaagtaaaagtaaaagcaagaaagaaaactagagattcttgtttaagcttttaatctcaaaaaacattaaatgaaatgcatacaaggttttatcctgctttagaactgtttgtatttaattatcaaactataagacagacaatctaatgccagtacacgctactcaaagttgtaaaacctcagatttaacttcagtagaagctgattctcaaaattgttagtgtcaagcctagctcttttggggctgaaaagcaatcctgcagtgctgaaaagcctctcacaggcagccgatgcgggaagaggtgtattagtcttgatagagaggctgcaaatagcaggaaacgtgagcagagactccctggtgtctgaaacacaggccagatgggccgatatcaactttgtatagaaaagttgaccacccagaccacccagaccacccagaccacccagaccacccagaccacccagaccacccagaccacccagaccacccagaccacccagaccacccagaccacccagaccacccagaccacccagaccacccagaccacccagaccacccagaccacccagaccacccagaccacccacaactttgtacaaaaaagcaggctggaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggaggaggaagatctaagctgaaccctcctgatgagagtggccccggctgcatgagctgcaagtgtgtgctctcctgagacccagctttcttgtacaaagttgatgtagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaattcgcggccgcggcgccaatgcattgggccctcgagcatatgagatctgtcccgatctttctcttctgtgctgtcaatttagagtagtttttgaggtaatttgggtgcattctctgtaaaaacaacaaataaatatttatattgtatgcaataaatgtaacaattacacatacactctctgattacagcattaaagggttagttcacccaaaaatgaaaagatgtcattaatgactcaccctcatgtcgttccaagcccgtaacacctccgttcatcttcggaacacagtttaagatattttagatttagtccgagagctttctgtccctccactgaaaatgtatgtacggtatactgtccatgtccagaaaggtaataaaaacatcaaagtagtccatgtgacatcagtgggttaggtagaattatttgaagcatcgaaaatacattttggtccaaaaataacaaaacctacgactttattcagcattgtagcgtcactgcgaagtcgtgaacgcggattgacaacagacccggaagagaatacaatgccgaataaagtcgtaggttttgttatttttggaccaaaatgtattcgatgcttcaaaaaattctaactaacccactgatgtcacatggactactttgatgtttttattacctttctggacatggacagtataccgtacatacattctcaatggaggcacagaaagctctcggactaaatctaaaatatcttaaactgtgttctgaagatgaacggaggtcttacgggcttggaacgacatgagggcgagtcattaatgacattattttcatttttgggtgaactaaccctttaataaaactgacatttgcagccaattgcacactttataatgttaaatacttgttgattttagaaatacagtagcatttagtgacaatcaaacattaattttatttctgattatattgacaaaaacacaggactgtaaggctgacatgacagggtgaattgatttcattacattagttttgatggtaacacttttacaataaggttcgttagttaactacattagttaacatgaactaataatgaactgcacgtatacatgttaatttcaacatttactaatactttatatcttgttaacattagttaatgcactgtgaactaacatgaacaaacaatgaacatctgtatttctattaactaacgttaacaaagattaacagatacagtaacaaatgtattgctcatggttatttaatgttagttaatacatgaactaatcaacctcattgtaaagtgttaccgttttgacaatacaacatgtttggaaaaaaatttctcgcttggttttcttgtgttttgcatgaattcaaacacaatgagagcacggaatgataatgattattcaaagctgcaataggcgccaaatttcccgtggacctaacgttaccaattacaaatgcattttagcattttattacgtgagccttatattgtcaatcgcatgcattagtgaaaataggcctactacacatataaaaggcgcgccatcaacgtcaaaacatgcacacccaaaaaaaaaaaaaaaaaaaaaaaaaaaaactatcagtaaaacaaaatacttaatgtacttacctcaatatgcttccttaggtttgatggcgaacttttgaaggccgatatttctttattaagcgggagacagaggacacatttcatcttgaatgaatctttatttacaccacttaaagaaaagaattcgcgaagatacggccacgggtgctcttgatcctgtggctgattttggactgtgctgctcgcagctgctgatgaatcacatacttcctccattttcttccactgattgactgttataatttccctaatttccaggtcaaggtgctgtgcattgtggtaatagatgtgacatgacgtcacttccaaaggaccaatgaacatgtctgaccaatttcatataatgtgaaaacgattttcataggcagaataaataacatttaaattaaactgggcatcagcgcaattcaattggtttggtaatagcaagggaaaatagaatgaagtgatctccaaaaaataagtactttttgactgtaaataaaattgtaaggagtaaaaagtacttttttttctaaaaaaatgtaattaagtaaaagtaaaagtattgatttttaattgtactcaagtaaagtaaaaatccccaaaaataatacttaagtacagtaatcaagtaaaattactcaagtactttacacctctggttcttgaccccctaccttcagcaagcccagcagatccactagttctagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtg

SEQ ID NO: 36 - DNA, 7099 bp, Artificial Sequence
  Description of Artificial Sequence Synthetic plasmid
ccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtacaggtcattgcacaacaccagaaatgccctctgatctgcaaaagacgtgaatatctgttcagacacccatatccactctgttccacacaggtcagaggtttgtccaggagttcttgacagaggtgtaaaaagtactcaaaaattttactcaagtgaaagtacaagtacttagggaaaattttactcaattaaaagtaaaagtatctggctagaatcttacttgagtaaaagtaaaaaagtactccattaaaattgtacttgagtattaaggaagtaaaagtaaaagcaagaaagaaaactagagattcttgtttaagcttttaatctcaaaaaacattaaatgaaatgcatacaaggttttatcctgctttagaactgtttgtatttaattatcaaactataagacagacaatctaatgccagtacacgctactcaaagttgtaaaacctcagatttaacttcagtagaagctgattctcaaaattgttagtgtcaagcctagctcttttggggctgaaaagcaatcctgcagtgctgaaaagcctctcacaggcagccgatgcgggaagaggtgtattagtcttgatagagaggctgcaaatagcaggaaacgtgagcagagactccctggtgtctgaaacacaggccagatgggccgatatcaactttgtatagaaaagttgaccacccagaccacccagaccacccagaccacccagaccacccagaccacccagaccacccagaccacccagaccacccagaccacccagaccacccagaccacccagaccacccagaccacccagaccacccagaccacccagaccacccagaccacccagaccacccagaccacccacaactttgtacaaaaaagcaggctccaccatggtgagcaagggcgaggaggacaacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgccgaggtcaagaccacctacaaggccaagaagcccgtgcagctgcccggcgcctacaacgtcaacatcaagctggacatcacctcccacaacgaggactacaccatcgtggaacagtacgagcgcgccgagggccgccactccaccggcggcatggacgagctgtacaagaagctgaaccctcctgatgagagtggccccggctgcatgagctgcaagtgtgtgctctcctgagacccagctttcttgtacaaagttgatgtagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaattcgcggccgcggcgccaatgcattgggccctcgagcatatgagatctgtcccgatctttctcttctgtgctgtcaatttagagtagtttttgaggtaatttgggtgcattctctgtaaaaacaacaaataaatatttatattgtatgcaataaatgtaacaattacacatacactctctgattacagcattaaagggttagttcacccaaaaatgaaaagatgtcattaatgactcaccctcatgtcgttccaagcccgtaacacctccgttcatcttcggaacacagtttaagatattttagatttagtccgagagctttctgtccctccactgaaaatgtatgtacggtatactgtccatgtccagaaaggtaataaaaacatcaaagtagtccatgtgacatcagtgggttaggtagaattatttgaagcatcgaaaatacattttggtccaaaaataacaaaacctacgactttattcagcattgtagcgtcactgcgaagtcgtgaacgcggattgacaacagacccggaagagaatacaatgccgaataaagtcgtaggttttgttatttttggaccaaaatgtattcgatgcttcaaaaaattctaactaacccactgatgtcacatggactactttgatgtttttattacctttctggacatggacagtataccgtacatacattctcaatggaggcacagaaagctctcggactaaatctaaaatatcttaaactgtgttctgaagatgaacggaggtcttacgggcttggaacgacatgagggcgagtcattaatgacattattttcatttttgggtgaactaaccctttaataaaactgacatttgcagccaattgcacactttataatgttaaatacttgttgattttagaaatacagtagcatttagtgacaatcaaacattaattttatttctgattatattgacaaaaacacaggactgtaaggctgacatgacagggtgaattgatttcattacattagttttgatggtaacacttttacaataaggttcgttagttaactacattagttaacatgaactaataatgaactgcacgtatacatgttaatttcaacatttactaatactttatatcttgttaacattagttaatgcactgtgaactaacatgaacaaacaatgaacatctgtatttctattaactaacgttaacaaagattaacagatacagtaacaaatgtattgctcatggttatttaatgttagttaatacatgaactaatcaacctcattgtaaagtgttaccgttttgacaatacaacatgtttggaaaaaaatttctcgcttggttttcttgtgttttgcatgaattcaaacacaatgagagcacggaatgataatgattattcaaagctgcaataggcgccaaatttcccgtggacctaacgttaccaattacaaatgcattttagcattttattacgtgagccttatattgtcaatcgcatgcattagtgaaaataggcctactacacatataaaaggcgcgccatcaacgtcaaaacatgcacacccaaaaaaaaaaaaaaaaaaaaaaaaaaaaactatcagtaaaacaaaatacttaatgtacttacctcaatatgcttccttaggtttgatggcgaacttttgaaggccgatatttctttattaagcgggagacagaggacacatttcatcttgaatgaatctttatttacaccacttaaagaaaagaattcgcgaagatacggccacgggtgctcttgatcctgtggctgattttggactgtgctgctcgcagctgctgatgaatcacatacttcctccattttcttccactgattgactgttataatttccctaatttccaggtcaaggtgctgtgcattgtggtaatagatgtgacatgacgtcacttccaaaggaccaatgaacatgtctgaccaatttcatataatgtgaaaacgattttcataggcagaataaataacatttaaattaaactgggcatcagcgcaattcaattggtttggtaatagcaagggaaaatagaatgaagtgatctccaaaaaataagtactttttgactgtaaataaaattgtaaggagtaaaaagtacttttttttctaaaaaaatgtaattaagtaaaagtaaaagtattgatttttaattgtactcaagtaaagtaaaaatccccaaaaataatacttaagtacagtaatcaagtaaaattactcaagtactttacacctctggttcttgaccccctaccttcagcaagcccagcagatccactagttctagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtg

SEQ ID NO: 37 - DNA, 11352 bp, Artificial Sequence
  Description of Artificial Sequence Synthetic plasmid
ccgctctagaactagtggatcccccgggctgcagcccgggggatccttcaggggtgtcgcttggttatttccaaaaatcaaattaattttattaaactattagaacgagcatgttttgtctatatgctacagaagataaaaaataataggagttaacagttataaaacaacacatttgtttctattgattgttgaccacactggggtctcattaagttagattaaagacacactaactgggtcaaaagcagcagattgatttcatagcaccagggtaaactttctaacacttttacggcaatcatatacattaaaattaaatacagaccacgactgaacaaggaggatgatctccaatattaaacaaagagacttgtgcctatttctctgagggtaaacatgacctctcaagttagcaagttgtttttaacactacaaaaatagttaagacctgcaatcccagaataaagtattggttttaaccaatcaatatagtacagtaaacatccatttgttttgttgaaacgttaaacaaatctgaccaaagctattagcttatataaaacaggtttgccttctatgtagctgaaaacaccacaggcccgattttgctactgtgtaaaacatttcagcaagatttttttattgcatttttttttactgaatcgttcaaacattttatcattttagtttgttcattcattgcaactggaaaaacaacacatcacacaaccgcacatatttcagcaataagtacaataaaacactcaaataaaaaaaacattttaaatctctttgtatttttgaccgctgtttcgcgtaatttcacggtaaaactctggaaatctccactacattcctctcagcggctcctctcaatgacagctgaagaagtgacggcggctgcctgctgtgttttgattggtcgaattcactggaggcttccagaacagtgtagagtctgaacgggtgcgcgctctgctgtatttaaagggcgaaagagagaccgcagagaaactcaaccgaagagaagcgacttgacaaagaagaaaagagcagcctgacaggacttttccccgacgaggtgtttattcgctctatttaagaatctactgtaaggtaagtctcaatatattgtactctattggctaatcagaattatatagagattatatgtacttaatgtcaaaaaatcaactttgtatatgtaatctttttacatgtggactgcctatgttcatcttattttaggtctactagaaaattatatttcccgttttcacaataaggattttaaaaaaagcaatgaacagacgggcatttactttatgttgctgacattattttatatgagcataataaccataaatactagcaaatgtcctaaatgaatttgtgttaatgttgtctacaaaagaaaattagcgttttacttgtacaactaataataacttggttattaagagaatttcacttgttgactagaaaaatcctttcataatgaaacaattgcaccgataaattgtataaatataaaattaattctaattgtttttttttttcctgcaggaattcgatatcaagcttgatttaggtgacactatagaatacaagctacttgttctttttgcaggatccatggcgtctggcagtatggcaaccagtgaggaagagcggagtctccgggaatgcgagctctatgtgcagaagcacaatatccaggccctgctgaaggactccatcgtgcagctgtgcactacgcggcccgagaggcccatggcattccttcgggaatactttgagaggttggagaaggaggaggcaagacagattcagtgtctacagaaaaccggcatccgtactgactcgagggaggacgagatctctcctccaccccccaatccagtggtgaagggccgacggcgccgaggtgctatcagtgctgaagtttacactgaggaggatgctgcctcctacgttagaaaggttattccaaaagattataagacaatggctgctttagccaaggccatcgaaaagaatgtgctgttttcacaccttgatgataacgagagaagtgacatttttgatgctatgtttccagtctcctttattgctggagagacggttattcagcaaggtgatgaaggggataacttctatgtgatcgatcaaggagaaatggatgtctatgtcaataatgaatgggcaaccagtgttggggaaggagggagctttgaagagctggctttgatttatggaacacccagagcagccactgtcaaagcaaagacaaacgtgaaactgtggggcatcgaccgagacagctaccgaagaatcctcatgggaagcactctgcgaaagaggaagatgtatgaagaattccttagtaaagtgtctattttagagtctctggacaagtgggagcgtctcacagtagccgatgcattggagcctgtccagtttgaagatggacagaagatcgtggtgcaaggagagccgggggatgagttcttcatcattttagagggcacagctgctgtgctgcagcgtcggtcagaaaacgaagaatttgttgaagtgggacgactggggccttctgattattttgatgaaattgccctgctgatgaatcatcctcgggctgccactgtggttgcccggggccctttgaagtgcgttaagttggaccggcctcggtttgaacgcgtccttggcccgtgctcagacatcctcaagcggaacatccagcagtacaacagcttcgtgtccctgtccgtccatcgattgatgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactgaaaactacctgtccatgacaacacttgtcactactttcacttatggtgttcaatgcttttcaagatacccagatcatatgaaacggcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaataagaattcaaggcctctcgagcctctagaactatagtgagtcgtattacgtagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaattcgcggccctcgagcatatgagatctgtcccgatctttctcttctgtgctgtcaatttagagtagtttttgaggtaatttgggtgcattctctgtaaaaacaacaaataaatatttatattgtatgcaataaatgtaacaattacacatacactctctgattacagcattaaagggttagttcacccaaaaatgaaaagatgtcattaatgactcaccctcatgtcgttccaagcccgtaacacctccgttcatcttcggaacacagtttaagatattttagatttagtccgagagctttctgtccctccactgaaaatgtatgtacggtatactgtccatgtccagaaaggtaataaaaacatcaaagtagtccatgtgacatcagtgggttaggtagaattatttgaagcatcgaaaatacattttggtccaaaaataacaaaacctacgactttattcagcattgtagcgtcactgcgaagtcgtgaacgcggattgacaacagacccggaagagaatacaatgccgaataaagtcgtaggttttgttatttttggaccaaaatgtattcgatgcttcaaaaaattctaactaacccactgatgtcacatggactactttgatgtttttattacctttctggacatggacagtataccgtacatacattctcaatggaggcacagaaagctctcggactaaatctaaaatatcttaaactgtgttctgaagatgaacggaggtcttacgggcttggaacgacatgagggcgagtcattaatgacattattttcatttttgggtgaactaaccctttaataaaactgacatttgcagccaattgcacactttataatgttaaatacttgttgattttagaaatacagtagcatttagtgacaatcaaacattaattttatttctgattatattgacaaaaacacaggactgtaaggctgacatgacagggtgaattgatttcattacattagttttgatggtaacacttttacaataaggttcgttagttaactacattagttaacatgaactaataatgaactgcacgtatacatgttaatttcaacatttactaatactttatatcttgttaacattagttaatgcactgtgaactaacatgaacaaacaatgaacatctgtatttctattaactaacgttaacaaagattaacagatacagtaacaaatgtattgctcatggttatttaatgttagttaatacatgaactaatcaacctcattgtaaagtgttaccgttttgacaatacaacatgtttggaaaaaaatttctcgcttggttttcttgtgttttgcatgaattcaaacacaatgagagcacggaatgataatgattattcaaagctgcaataggcgccaaatttcccgtggacctaacgttaccaattacaaatgcattttagcattttattacgtgagccttatattgtcaatcgcatgcattagtgaaaataggcctactacacatataaaaggcgcgccatcaacgtcaaaacatgcacacccaaaaaaaaaaaaaaaaaaaaaaaaaaaaactatcagtaaaacaaaatacttaatgtacttacctcaatatgcttccttaggtttgatggcgaacttttgaaggccgatatttctttattaagcgggagacagaggacacatttcatcttgaatgaatctttatttacaccacttaaagaaaagaattcgcgaagatacggccacgggtgctcttgatcctgtggctgattttggactgtgctgctcgcagctgctgatgaatcacatacttcctccattttcttccactgattgactgttataatttccctaatttccaggtcaaggtgctgtgcattgtggtaatagatgtgacatgacgtcacttccaaaggaccaatgaacatgtctgaccaatttcatataatgtgaaaacgattttcataggcagaataaataacatttaaattaaactgggcatcagcgcaattcaattggtttggtaatagcaagggaaaatagaatgaagtgatctccaaaaaataagtactttttgactgtaaataaaattgtaaggagtaaaaagtacttttttttctaaaaaaatgtaattaagtaaaagtaaaagtattgatttttaattgtactcaagtaaagtaaaaatccccaaaaataatacttaagtacagtaatcaagtaaaattactcaagtactttacacctctggttcttgaccccctaccttcagcaagcccagcagatccactagttctagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgtccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccaaatagtaggaattacccacctgtacaagtgctgaaaacttggatgaataagcccgtttgccattttctactgctattttaaatcttttctgtatttgtctgcatttgtctttacccttgaataaatgttttagttgttttttttcaattatgactgtgtttaacagacattattacaataatatgtaatagtagtacacactattattatgtaatagtacttgttgactgtatttgagaactggaccgagtgagtgttacgtcacccattcaaaatgacttacttctggctccaacaaaatgaagttaattcagttgccatttttcactgtatggacatcgccgtgttggagctagacgtcgccaataagcaagaaagagccgagatgcgtcgagtctgagtcaccgttcctatggcaacccctctaaccaatcagaagtaagcttgttggaagtccacagcctaccacttgaaagcgggctgcacaaaatctgtcaaacgttttgaacgttggatgtgagagcacatacttttattaaggcatctggttggtcagtttataatatcaacaacttgggctacagaaaagaaaagttattacagaaattatgattaacaagtacatgttaaataaagattttaatatgaatgccaccactggagcattcatgccatttggagcttcttcctgtttggatcactagaaggaggaggtcactcattacagttctcatatacagtcgttggttggttggttggttggttggttggtagattgattgattgattgattgattgattgattgattgattgattgattgattgattgattgattgattgattgattgattgattgattgattgattgattgattggtagtcaaaataagaaataatttccacagattcattacagaaatgattaaatgcatacataaaaaactggggggggggggatacaacaacacacttaagtcacatttgcctacgtaaagaaaagtaaagaaaatcaatagctatattttacatctcctttttttgctgtcttttaattagccttgttttgctgttatcttgattgaaactgtaacttcttcacctgcttcttttctttgtaggttttgccagcccagaaacaggcatggctgtgcaaggccagagcaccatgcacaatgcgctgcatgtcttcatgaacggctcaatgtcctcagtccagggctcagccaacgaccccatcttcctccttcaccatgctttcattgacaggtaacaaacacgtcatgacattagactgcacagtttttgacaaagttcatacaatctgttgtttatagctgctacaattagtgaagtttgtgaatgtacttggatgagcagcgaaagatcaattgagatcaattgttagagtttggttgccctgcagagcaaagaacaaaaaataatctggtggctttactgcgtgaggttattattggtggaatagaaacacaaaacataattgcatttatttgtttaattttttatcttatcttaactttcatcttgcatatttgtttcttacatcatttctagcatctttgagcgctggctaagaactcatcagcctccccggtccatctacccacgtaccaatgcaccaattggccacaatgacggctactacatggtgccattccttcctctttataggaatggagactacctcctgtccaacaaggctcttggatacgagtacgcctacctgttggacccaggtcattgcacaacaccagaaatgccctctgatctgcaaaagacgtgaatatctgttcagacacccatatccactctgttccacacaggtcagaggtttgtccaggagttcttgacagaggtgtaaaaagtactcaaaaattttactcaagtgaaagtacaagtacttagggaaaattttactcaattaaaagtaaaagtatctggctagaatcttacttgagtaaaagtaaaaaagtactccattaaaattgtacttgagtattaaggaagtaaaagtaaaagcaagaaagaaaactagagattcttgtttaagcttttaatctcaaaaaacattaaatgaaatgcatacaaggttttatcctgctttagaactgtttgtatttaattatcaaactataagacagacaatctaatgccagtacacgctactcaaagttgtaaaacctcagatttaacttcagtagaagctgattctcaaaattgttagtgtcaagcctagctcttttggggctgaaaagcaatcctgcagtgctgaaaagcctctcacaggcagccgatgcgggaagaggtgtattagtcttgatagagaggctgcaaatagcaggaaacgtgagcagagactccctggtgtctgaaacacaggccagatggg

SEQ ID NO: 38 - DNA, 12529 bp, Artificial Sequence
  Description of Artificial Sequence Synthetic plasmid
atccttcaggggtgtcgcttggttatttccaaaaatcaaattaattttattaaactattagaacgagcatgttttgtctatatgctacagaagataaaaaataataggagttaacagttataaaacaacacatttgtttctattgattgttgaccacactggggtctcattaagttagattaaagacacactaactgggtcaaaagcagcagattgatttcatagcaccagggtaaactttctaacacttttacggcaatcatatacattaaaattaaatacagaccacgactgaacaaggaggatgatctccaatattaaacaaagagacttgtgcctatttctctgagggtaaacatgacctctcaagttagcaagttgtttttaacactacaaaaatagttaagacctgcaatcccagaataaagtattggttttaaccaatcaatatagtacagtaaacatccatttgttttgttgaaacgttaaacaaatctgaccaaagctattagcttatataaaacaggtttgccttctatgtagctgaaaacaccacaggcccgattttgctactgtgtaaaacatttcagcaagatttttttattgcatttttttttactgaatcgttcaaacattttatcattttagtttgttcattcattgcaactggaaaaacaacacatcacacaaccgcacatatttcagcaataagtacaataaaacactcaaataaaaaaaacattttaaatctctttgtatttttgaccgctgtttcgcgtaatttcacggtaaaactctggaaatctccactacattcctctcagcggctcctctcaatgacagctgaagaagtgacggcggctgcctgctgtgttttgattggtcgaattcactggaggcttccagaacagtgtagagtctgaacgggtgcgcgctctgctgtatttaaagggcgaaagagagaccgcagagaaactcaaccgaagagaagcgacttgacaaagaagaaaagagcagcctgacaggacttttccccgacgaggtgtttattcgctctatttaagaatctactgtaaggtaagtctcaatatattgtactctattggctaatcagaattatatagagattatatgtacttaatgtcaaaaaatcaactttgtatatgtaatctttttacatgtggactgcctatgttcatcttattttaggtctactagaaaattatatttcccgttttcacaataaggattttaaaaaaagcaatgaacagacgggcatttactttatgttgctgacattattttatatgagcataataaccataaatactagcaaatgtcctaaatgaatttgtgttaatgttgtctacaaaagaaaattagcgttttacttgtacaactaataataacttggttattaagagaatttcacttgttgactagaaaaatcctttcataatgaaacaattgcaccgataaattgtataaatataaaattaattctaattgtttttttttttcctgcagtcgacggtaccgcggatggagaccacaagtcccacctccacagagaagaaggaattgaagccgtcagtcctggatggcagtagcttcacagaccttcccaaaaagccctcacccaccacagcatccagagctcctcatctgttctccaccttccacacaccaatcccgatcgacatgcggcatcatgaaggaaggtaccactacgagccgcaccctctgcaccccatgcatggacctcatggattagctggcagtccagttatctctgatatctctctgatccgactgtcgcctcacgccgcagccactggagaatcccccttcaaccctccacacccgtatgtcaatccacacatggagcattacctgcgctccgtccacagcagccccacgctctccatgatctctgctgcccgaggactgagccctgcagaattgacccatgagcatctgaaagagcgcagtttgtttggcctccctcctccgcctcctggagcaaacccttcagagtactatcacctcatcgccagccaccgtagcccttatggagacctgctcatgcagacgggagctgcggccgctcatcttcctgactacatgagcccagtggacatgtctcgtttcccgagtcccagactgacgccgagggtgagcaggaagcgagcattgtccatctctccagtctcggatgccagcatcgacctgcagaccatgatccgcacctcacccaactctctggtggcctacatcaataactcacgctccagctcggcagccagcagctcttacggccacctctccgtcggggccatcagtccatccttcacgttcccacaccccatcacaccggttgcataccaccagctgttgtcccagcagagaggtctgaatgcatttggacacactcctcctctgctgcagccttcacctgccctcagcagcagacagcagacactcgtggctgcagccgctctcaacaacaccaccagcagcagcaccagcagcagcactgcagacaccagcaccgagtccagccagaacgctgggggagacccggccgtgagcagcaccgtcaaccccatgatcttcaagcggtccaaggtgaagaccgaagcagaggggcctcatcccatctctcctggttctcaggatcacttggagttacgagaggagcttgataaagatgaatgcaagcaggagcccgaggccgtgtatgaaaccaactgccactgggaaggatgtgccaaagagtacgacacgcaggaccagctcgttcatcacatcagtaatgaccacatccatggagagaagaaggagtttgtgtgtcgatgggtggagtgctctcgagaacagaagcctttcaaggcccagtacatgcttgtggtccacatgcgtcgacacactggggagaagccccataagtgcacatttgaaggttgctcgaaggcttattctcgactggaaaatctcaaaactcacttgaggtcccacacaggggagaaaccttacgtatgcgagcacgagggctgcaacaaggctttttccaacgcttcagaccgggccaagcaccagaaccgcacgcactccaatgagaaaccatatgtgtgtaagatcccaggttgcacaaaacgttatacagaccccagttcactcaggaagcatgtgaagactgttcacggtccagaagcacatgtcactaagaagcagcgcggcgatgcaccgccgaaacctcacccccctaaagggaatggagaaaatgaggcccacacaaagcatgtgagggggagaacagatgggtcaggagaggccaacagcaccaccagaggcgtggaggactgccaacatgtcaagacgatcaagactgaaaacacagtgatgtatcagtccagccctggtggtcagtcgtcatgtagcagtgagccatcgccacttggcagtgccaccaacaatgacagtggagtagagatggccatgcacagtggaggcagtttgggggacctgagtgctttagatgacacacctgtggtcgattctaccggttctccaggtacttctgctggagttggtctccagctgcgtaaaaacagggcgggactactacagcttgagcacatcaagaaggaaaaactaaaaactgtacgagactcctgctcctgggcaaatgcaccacctcaagtccgaaataccaagctgcctccaataccctctattgactcactgctggacgctccaaatatggggactcaaatgtctgtacctccaacccagcatttaggagacttgtcttcatatgagatgacaatgcttaatcagctacatgagcgcagagatagctccaccagcacaatgagctcagcatatataagccgtcggtcctctggcatttcaccttgctactccagtcgtcgctcaagtgaggcttcccaatttggagtccgcaacaacaatgtcagctcagcagactcctatgacccgatctccacagatctgtcacgtcgttccagtgaagcaagccaatgtggtggtacaggtgggctcccaagtttactaaatctcacaccagctcaacactacagtcttaaggcgaaatatgctgcagccactgggggtgcaccaccaacacctctgcccaacatggatcgcatgagccttaaaactagaatggccatgtacatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtataaatagcggccgcgggcccgggatctgtcccgatctttctcttctgtgctgtcaatttagagtagtttttgaggtaatttgggtgcattctctgtaaaaacaacaaataaatatttatattgtatgcaataaatgtaacaattacacatacactctctgattacagcattaaagggttagttcacccaaaaatgaaaagatgtcattaatgactcaccctcatgtcgttccaagcccgtaacacctccgttcatcttcggaacacagtttaagatattttagatttagtccgagagctttctgtccctccactgaaaatgtatgtacggtatactgtccatgtccagaaaggtaataaaaacatcaaagtagtccatgtgacatcagtgggttaggtagaattatttgaagcatcgaaaatacattttggtccaaaaataacaaaacctacgactttattcagcattgtagcgtcactgcgaagtcgtgaacgcggattgacaacagacccggaagagaatacaatgccgaataaagtcgtaggttttgttatttttggaccaaaatgtattcgatgcttcaaaaaattctaactaacccactgatgtcacatggactactttgatgtttttattacctttctggacatggacagtataccgtacatacattctcaatggaggcacagaaagctctcggactaaatctaaaatatcttaaactgtgttctgaagatgaacggaggtcttacgggcttggaacgacatgagggcgagtcattaatgacattattttcatttttgggtgaactaaccctttaataaaactgacatttgcagccaattgcacactttataatgttaaatacttgttgattttagaaatacagtagcatttagtgacaatcaaacattaattttatttctgattatattgacaaaaacacaggactgtaaggctgacatgacagggtgaattgatttcattacattagttttgatggtaacacttttacaataaggttcgttagttaactacattagttaacatgaactaataatgaactgcacgtatacatgttaatttcaacatttactaatactttatatcttgttaacattagttaatgcactgtgaactaacatgaacaaacaatgaacatctgtatttctattaactaacgttaacaaagattaacagatacagtaacaaatgtattgctcatggttatttaatgttagttaatacatgaactaatcaacctcattgtaaagtgttaccgttttgacaatacaacatgtttggaaaaaaatttctcgcttggttttcttgtgttttgcatgaattcaaacacaatgagagcacggaatgataatgattattcaaagctgcaataggcgccaaatttcccgtggacctaacgttaccaattacaaatgcattttagcattttattacgtgagccttatattgtcaatcgcatgcattagtgaaaataggcctactacacatataaaaggcgcgccatcaacgtcaaaacatgcacacccaaaaaaaaaaaaaaaaaaaaaaaaaaaaactatcagtaaaacaaaatacttaatgtacttacctcaatatgcttccttaggtttgatggcgaacttttgaaggccgatatttctttattaagcgggagacagaggacacatttcatcttgaatgaatctttatttacaccacttaaagaaaagaattcgcgaagatacggccacgggtgctcttgatcctgtggctgattttggactgtgctgctcgcagctgctgatgaatcacatacttcctccattttcttccactgattgactgttataatttccctaatttccaggtcaaggtgctgtgcattgtggtaatagatgtgacatgacgtcacttccaaaggaccaatgaacatgtctgaccaatttcatataatgtgaaaacgattttcataggcagaataaataacatttaaattaaactgggcatcagcgcaattcaattggtttggtaatagcaagggaaaatagaatgaagtgatctccaaaaaataagtactttttgactgtaaataaaattgtaaggagtaaaaagtacttttttttctaaaaaaatgtaattaagtaaaagtaaaagtattgatttttaattgtactcaagtaaagtaaaaatccccaaaaataatacttaagtacagtaatcaagtaaaattactcaagtactttacacctctggttcttgaccccctaccttcagcaagcccagcagatccactagttctagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgtccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccaaatagtaggaattacccacctgtacaagtgctgaaaacttggatgaataagcccgtttgccattttctactgctattttaaatcttttctgtatttgtctgcatttgtctttacccttgaataaatgttttagttgttttttttcaattatgactgtgtttaacagacattattacaataatatgtaatagtagtacacactattattatgtaatagtacttgttgactgtatttgagaactggaccgagtgagtgttacgtcacccattcaaaatgacttacttctggctccaacaaaatgaagttaattcagttgccatttttcactgtatggacatcgccgtgttggagctagacgtcgccaataagcaagaaagagccgagatgcgtcgagtctgagtcaccgttcctatggcaacccctctaaccaatcagaagtaagcttgttggaagtccacagcctaccacttgaaagcgggctgcacaaaatctgtcaaacgttttgaacgttggatgtgagagcacatacttttattaaggcatctggttggtcagtttataatatcaacaacttgggctacagaaaagaaaagttattacagaaattatgattaacaagtacatgttaaataaagattttaatatgaatgccaccactggagcattcatgccatttggagcttcttcctgtttggatcactagaaggaggaggtcactcattacagttctcatatacagtcgttggttggttggttggttggttggttggtagattgattgattgattgattgattgattgattgattgattgattgattgattgattgattgattgattgattgattgattgattgattgattgattgattgattggtagtcaaaataagaaataatttccacagattcattacagaaatgattaaatgcatacataaaaaactggggggggggggatacaacaacacacttaagtcacatttgcctacgtaaagaaaagtaaagaaaatcaatagctatattttacatctcctttttttgctgtcttttaattagccttgttttgctgttatcttgattgaaactgtaacttcttcacctgcttcttttctttgtaggttttgccagcccagaaacaggcatggctgtgcaaggccagagcaccatgcacaatgcgctgcatgtcttcatgaacggctcaatgtcctcagtccagggctcagccaacgaccccatcttcctccttcaccatgctttcattgacaggtaacaaacacgtcatgacattagactgcacagtttttgacaaagttcatacaatctgttgtttatagctgctacaattagtgaagtttgtgaatgtacttggatgagcagcgaaagatcaattgagatcaattgttagagtttggttgccctgcagagcaaagaacaaaaaataatctggtggctttactgcgtgaggttattattggtggaatagaaacacaaaacataattgcatttatttgtttaattttttatcttatcttaactttcatcttgcatatttgtttcttacatcatttctagcatctttgagcgctggctaagaactcatcagcctccccggtccatctacccacgtaccaatgcaccaattggccacaatgacggctactacatggtgccattccttcctctttataggaatggagactacctcctgtccaacaaggctcttggatacgagtacgcctacctgttggacccaggtcattgcacaacaccagaaatgccctctgatctgcaaaagacgtgaatatctgttcagacacccatatccactctgttccacacaggtcagaggtttgtccaggagttcttgacagaggtgtaaaaagtactcaaaaattttactcaagtgaaagtacaagtacttagggaaaattttactcaattaaaagtaaaagtatctggctagaatcttacttgagtaaaagtaaaaaagtactccattaaaattgtacttgagtattaaggaagtaaaagtaaaagcaagaaagaaaactagagattcttgtttaagcttttaatctcaaaaaacattaaatgaaatgcatacaaggttttatcctgctttagaactgtttgtatttaattatcaaactataagacagacaatctaatgccagtacacgctactcaaagttgtaaaacctcagatttaacttcagtagaagctgattctcaaaattgttagtgtcaagcctagctcttttggggctgaaaagcaatcctgcagtgctgaaaagcctctcacaggcagccgatgcgggaagaggtgtattagtcttgatagagaggctgcaaatagcaggaaacgtgagcagagactccctggtgtctgaaacacaggccagatgggccctcgagcatatgag

(19) United States
(12) Patent Application Publication
(10) Pub. No.: US 2008/0127358 A1
(43) Pub. Date: May 29, 2008

(54) Compositions and methods for modulating hedgehog signaling

(76) Inventor: Rolf Karlstrom, Amherst, MA (US)

Correspondence Address: FOLEY HOAG, LLP;PATENT GROUP, WORLD TRADE CENTER WEST, 155 SEAPORT BLVD, BOSTON, MA, 02110, US

(21) Appl. No.: 11/796,131

(22) Filed: Apr. 26, 2007

Related U.S. Application Data
(60) Provisional application No. 60/795,209, filed on Apr. 26, 2006.

(51) Int. Cl. G01N 33/00; C12N 15/64; C12N 5/00; C07H 21/04; H05B 1/00; C12Q 1/02; A01K 67/027; C12Q 1/68

(52) U.S. Cl. 800/3; 435/320.1; 536/23.5; 435/325; 435/29; 800/20; 435/6; 219/229

(57) ABSTRACT

Provided herein are nucleic acid constructs encoding Hh regulating proteins, which are proteins that activate or inhibit Hh signaling. Exemplary Hh regulating proteins are N- or C-terminally truncated forms of Gli proteins or a dominant negative form of PKA. The Hh regulating proteins may be linked to a heterologous protein, e.g., green fluorescent protein (GFP), and may further be under the control of an inducible promoter. These constructs may be used, e.g., for modulating Hh signaling, and therefore, e.g., cell proliferation and differentiation. Also provided herein are Hh reporter construct, comprising, e.g., a promoter or promoter element that is controlled by a Gli protein. Cells and organisms comprising the nucleic acids are also described, as well as the use of these in assays for identifying agents that can be used for treating or preventing Hh associated diseases, such as cancer. They may also be used in assays for determining the toxicity of an agent or a sample.

### RELATED APPLICATIONS

The present application claims priority to U.S. Provisional Patent Application Ser. No. 60/795,209 filed on Apr. 26, 2006, the entire content of which is specifically incorporated herein by reference.

### BACKGROUND

The hedgehog (hh) gene was first discovered over 20 years ago by Nusslein-Volhard and Wieschaus in a screen for mutations affecting Drosophila development (Nusslein-Volhard and Wieschaus, 1980). During embryogenesis, secreted Hh molecules can act as classic morphogens to influence cellular differentiation and/or proliferation (Placzek, 1995; Hammerschmidt et al., 1997; Dodd et al., 1998; Ingham, 1998b). Hh signaling has since been implicated in the development of a wide range of tissues in many different animals including; fly eyes, segments and wing discs, as well as the vertebrate spinal cord, lungs, limbs, heart, and pancreas, to name a few (Ingham and McMahon, 2001). Mutational analysis in mouse and zebrafish has established the essential role of sonic hedgehog (shh) in cell differentiation during vertebrate embryonic development. Mouse mutants lacking shh have severe ventral forebrain defects, lack a floor plate and motor neurons in the neural tube, and have reduced sclerotomal tissue in the somites (Chiang et al., 1996). Zebrafish Hh pathway mutants have a range of midline defects including; loss or reduction of the ventral forebrain and pituitary, axon guidance defects, absence of slow muscle fibers, and pancreas defects (Chen et al., 2001; dilorio et al., 2002; Karlstrom et al., 1996; Karlstrom et al., 1999; Karlstrom et al., 2003; Sbrogna et al., 2003; Schauerte et al., 1998; Ungos et al., 2003; Varga et al., 2001). The list of tissues and organs that require Hh signaling is continually growing. To date, in vivo studies implicate Hh in at least 24 distinct areas of the embryo (Ingham and McMahon, 2001). Despite this work, the exact role, source, and timing of Hh signaling in the pituitary and forebrain have yet to be determined. The adenohypophysis has a limited number off well-defined cell types, making it an ideal tissue for the study of the Hh response in the anterior neural tube. This proposal focuses on understanding the largely unanswered question of how Hh signals guide cell differentiation in the developing forebrain and pituitary gland.

The importance of Hh signaling in human development is underscored by the large number of human developmental disorders that arise from defects in Hh signaling (Ming et al., 1988). Holoprosencephaly (HPE) is a common disorder that affects development of the ventral hypothalamus in the region of the pituitary. HPE is in fact the most common human developmental disorder, affecting up to 1 in 250 conceptuses and 1 in 16,000 live births (Muenke and Cohen, 2000). HPE results in midline defects in the face and ventral brain and include defects in the pituitary gland such as hypopituitarism. Mutations in 8 different genes have now been linked to human HPE. Four of these genes are in the Hh signaling pathway (Shh, Patched, DispatchedA and now Gli2 (Roessler et al., 2003)), two are involved in nodal signaling (TGIF and Cripto/Tdgf1) and two are transcription factors involved in forebrain patterning (six3 and zic2) (rev. in Hayhurst and McConnell, 2003). HPE-like midline defects seen in Smith-Lemli-Opitz syndrome are linked to defects in cholesterol biosynthesis (Kelley and Herman, 2001), with Hh signaling being a major contributor to the observed phenotypes (Cooper et al., 2003). Mutations in gli3 are associated with at least three distinct human developmental syndromes including Greig cephalopolysyndactyly syndrome (GCPS) (Wild et al., 1997), Pallister-Hall syndrome (PHS) (Biesecker, 1997; Kang et al., 1997) and postaxial polydactyly type A1 (PAP-A) (Radhakrishna et al., 1997).

In addition to these Hh related birth defects, several of the most common forms of cancer involve activating mutations in the Hh signaling pathway. A variety of new evidence suggests Gli mediated Hh signaling plays a central role in brain tumors (Ruiz i Altaba et al., 2004). Sporadic basal cell carcinoma (BCC), a common malignant cancer in fair-skinned adults, is associated with mutations that activate the Hh pathway (Ingham, 1998; Xie et al., 1998). Overexpression of the Hh responsive transcription factor gli1 in frogs (Dahmane et al., 1997) and fish (R. Karlstrom unpublished results) can lead to tumors that express endogenous gli1, suggesting a positive feedback loop. If, as seems likely, the Hh responsive transcription factors of the Gli family can act as causative agents in cell transformation, then mutations in any component of the Hh pathway that lead to activation of gli1 could lead to tumorigenesis.

Over 25 gene products have been identified as playing a role in relaying Hh signals (Ingham and McMahon, 2001; Nybakken and Perrimon, 2002). Although the nature of the Hedgehog response is dependent on the responding cell type and species, the mechanism by which that response is generated seems to be remarkably conserved throughout metazoan species. Whenever tested, Drosophila Hh signaling genes can substitute for their vertebrate homologues and vice versa. However, several of the genes that regulate Hh reception in vertebrates have not been found in Drosophila, indicating that vertebrate Hh signaling may have some unique features which cannot be studied using the Drosophila model system.

After translation, Hh proteins are processed and secreted through action of the Dispatched transmembrane protein (Burke et al., 1999; Lee and Treisman, 2001). In responding cells, Hh binds and inhibits the receptor protein Patched (Ptc), acting to release the surface protein Smoothened (Smo) from Ptc inhibition. Smo acts to transduce the Hh signal within the cell in conjunction with the serine-threonine kinase Fused (Fu). The signaling pathway converges on the zinc-finger-containing transcription factors of the Gli family (Ci in Drosophila), which regulate downstream genes including many transcription factors, ptc, and shh itself.

Drosophila Cubitus interruptus (Ci) is homologous to the vertebrate Gli proteins and acts to transduce Hh signals. The Ci protein is primarily found in the cytoplasm where it is associated with microtubules in a complex that contains the kinesin-like molecule Costal-2 and the kinases Fused and Supressor of Fused. Activity of the Ci seems to be regulated primarily at the post-translational level (Aza-Blanc et al., 1997). In the absence of Hh signals, proteolytic cleavage of full length Ci produces a 75 kD N-terminal fragment that represses Hh target genes. This fragment contains the DNA binding region of Ci, but lacks the C-terminal activation domain. C-terminal epitopes (representing full length Ci) are predominantly localized in the cytoplasm and are only found near sources of hedgehog, whereas N-terminal epitopes (representing Ci repressor isoform) are nuclear and are found wherever Ci mRNA is expressed. The activator form of Ci also requires Hh regulated phosphorylation and active transport to the nucleus (Wang and Holmgren, 2000). The working model is that hedgehog signaling represses the cleavage of Ci, resulting in an increase in the Ci activator isoform and a corresponding reduction in the Ci repressor isoform.

Vertebrate Gli expression appears to be regulated at both the transcriptional and post-translation levels, and Glis can in turn also act as transcriptional activators and/or repressors of Hh target genes. We showed that in zebrafish Gli1 is an essential activator of Hh signaling, while Gli2 plays a more minor role as an activator or repressor of Hh signaling (Karlstrom et al., 2003). The situation is reversed in mouse, where Gli2 is the major activator of the Hh response, with Gli1 playing a non-essential role (Matise et al., 1998a; Park et al., 2000). In mouse, Gli1 can completely substitute for Gli2 function in neural patterning, showing Gli1 can also activate Hh signaling (Bai and Joyner, 2001). Work on Gli3 has shown both activator and repressor functions for both zebrafish and mouse Gli3. Gli3 is a major repressor of Hh signaling in the dorsal spinal cord (e.g. Ruiz i Altaba, 1998; Shin et al., 1999; Tole et al., 2000), but recent data also indicates that Gli3 can play a role in activating Hh response (Bai et al., 2004).

### SUMMARY

Provided herein are nucleic acids comprising a nucleotide sequence encoding (i) a Hedgehog (Hh) regulating protein; (ii) a heterologous peptide; wherein the nucleotide sequence is operably linked to an inducible promoter. The Hh regulating protein may be a Gli or PKA protein or a biologically active portion thereof. The Hh regulating protein may be a dominant repressor of Gli mediated Hh signaling. The dominant repressor of Gli mediated Hh signaling may be a C-terminally truncated Gli2 protein. The Hh regulating protein may be an activator of Hh signaling. The activator of Hh signaling may be an N-terminally truncated form of Gli2 or Gli3. The activator of Hh signaling may also be a dominant negative form of protein kinase A (PKA). A heterologous protein may be an enzyme, e.g., green fluorescent protein (GFP). An inducible promoter may be a heat-shock promoter, e.g., the promoter of HSP70.

Also provided herein are vectors comprising a nucleic acid described herein, as well as cells comprising one or more nucleic acid or vector described herein. A cell may be a zebrafish cell. Further provided are organisms comprising one or more nucleic acid or vector described herein. An organism may be a zebrafish. In one embodiment, a zebrafish comprises a nucleic acid that encodes a Hh regulatory protein from a zebrafish.

Further provided herein are cells and organisms, e.g., a zebrafish or cell thereof, comprising a nucleic acid comprising (i) a nucleotide sequence comprising one or more Hh regulatory elements that are regulated by the regulatory protein, and (ii) a nucleotide sequence encoding a reporter protein. The one or more Hh regulatory elements may be binding sites for Gli1. The one or more Hh regulatory elements may be the DNA regulator region that regulates Patched (Ptc) expression. The reporter protein may be GFP or luciferase.

Also provided are methods, e.g., methods for modulating Hh signaling response in a cell. A method may comprise contacting a cell comprising nucleic acids comprising a nucleotide sequence encoding (i) a Hedgehog (Hh) regulating protein; (ii) a heterologous peptide; wherein the nucleotide sequence is operably linked to an inducible promoter, with an agent, or subjecting the cell to a condition, that induces the inducible promoter. A method may comprise subjecting a cell or organism to a heat-shock at a temperature and for a time sufficient to induce the expression of the heat-shock promoter. Methods for modulating Hh signaling response in an organism may comprise contacting an organism comprising a nucleic acid described herein with an agent, or subjecting the organism to a condition that induces the inducible promoter.

A method for identifying an agent that modulates Hh signaling may comprise (a) contacting a cell comprising a nucleic acid comprising a nucleotide sequence encoding (i) a Hedgehog (Hh) regulating protein; (ii) a heterologous peptide; wherein the nucleotide sequence is operably linked to an inducible promoter, with a test agent; (b) inducing the inducible promoter; and (c) determining the level of Hh signaling; wherein a different level of Hh signaling in a cell contacted with a test agent relative to a cell that was not contacted with a test agent indicates that the test agent is an agent that modulates Hh signaling. A method for identifying an agent that modulates Hh signaling may also comprise (a) contacting an organism comprising a nucleic acid comprising a nucleotide sequence encoding (i) a Hedgehog (Hh) regulating protein; (ii) a heterologous peptide; wherein the nucleotide sequence is operably linked to an inducible promoter, with a test agent; (b) inducing the inducible promoter; and (c) determining the level of Hh signaling; wherein a different level of Hh signaling in a cell contacted with a test agent relative to a cell that was not contacted with a test agent indicates that the test agent is an agent that modulates Hh signaling. A method may comprise initiating step (a) before or after step (b) or the two steps may be initiated essentially at the same time. A method may further comprise contacting the test agent with a cell or organism that is a model for a disease that is associated with an abnormal Hh regulation, e.g., cancer.

Also provided are methods for determining whether an agent modulates Hh signaling in a cell. A method may comprise (a) contacting a cell with a nucleic acid comprising a nucleotide sequence encoding (i) a Hedgehog (Hh) regulating protein; (ii) a heterologous peptide; wherein the nucleotide sequence is operably linked to an inducible promoter, with an agent; (b) inducing the inducible promoter; and (c) determining the level of Hh signaling; wherein a different level of Hh signaling in a cell contacted with the agent relative to a cell that was not contacted with an agent indicates that the agent modulates Hh signaling in a cell. A method for determining whether an agent modulates Hh signaling in an organism may also comprise (a) contacting an organism comprising a nucleic acid comprising a nucleotide sequence encoding (i) a Hedgehog (Hh) regulating protein; (ii) a heterologous peptide; wherein the nucleotide sequence is operably linked to an inducible promoter, with an agent; (b) inducing the inducible promoter; and (c) determining the level of Hh signaling; wherein a different level of Hh signaling in a cell contacted with the agent relative to a cell that was not contacted with the agent indicates that the agent modulates Hh signaling in an organism. The agent may be an environmental sample, and the method may be for determining whether the agent is toxic to a cell or organism, wherein an agent is toxic to a cell or organism if the agent modulates Hh signaling.

A method for determining whether an agent modulates Hh signaling in a cell or organism may comprise (a) contacting a cell or organism comprising a nucleic acid comprising (i) a nucleotide sequence comprising one or more Hh regulatory elements that are regulated by the regulatory protein, and (ii) a nucleotide sequence encoding a reporter protein, with an agent; and (b) determining the level of expression of the reporter protein, wherein a different level of expression of the reporter protein in a cell or organism that was contacted with the agent relative to a cell or organism that was not contacted with the agent indicates that the agent modulates Hh signaling in a cell or organism. The agent may be an environmental sample, and the method may be for determining whether the agent is toxic to a cell or organism, wherein an agent is toxic to a cell or organism if the agent modulates Hh signaling.

Also provided are local heating tools. A heating probe may comprise: an uncoiled portion of an igniter wire having a length of about 3 cm and bent to form a heating element; a J-type thermocouple attached to an end of the igniter wire; a controller connected to the heating element and thermocouple for limiting the temperature of the heating element based on heat sensed by the thermocouple; a micropipette tip for attaching the heating element and thermocouple to a syringe, wherein wiring from the heating element and thermocouple pass through the syringe to the controller.

### BRIEF DESCRIPTION OF THE FIGURES

FIGS. 1-38 set forth the nucleotide sequences of the vectors in Table 1 and 2.

FIG. 39 shows the use of the HSP70 promoter to drive transgene expression in zebrafish. A,B) Embryos were injected at the 2 cell stage with the HS-GFP plasmid construct, heatshocked at the 20 hr stage for 30 min., and photographed 2 hours later. A) This frontal view shows an embryo with several cells (arrows) in the region of the POC expressing the Sema3D-GFP fusion protein. B) This ventral view shows another embryo with 2 Sema3D-GFP expressing cells, one near the POC (arrow) and one near the AC (arrowhead) C) Global heatshock of the HS-GFP transgenic line activates GFP expression throughout the embryo (top). D) Diagram showing the wire heating element generating a local heatshock. E) Placing the heated tungsten wire near embryonic tissue has proven to be a surprisingly simple and accurate way to induce local expression of the transgene, in this case in the telencephalon (arrow).

FIG. 40 shows cell autonomous activation and repression of Hh signaling. A) A luciferase reporter assay carried out in a C17 neuronal cell line shows the Gli2DRGFP fusion protein effectively blocks Gli1 mediated gene expression (light blue). B-D) Embryos injected at the 2 cell stage with HSP70 promoter containing plasmid constructs encoding the Gli2DR protein fused to GFP (Gli2DR/GFP) (B,C) or a dominant negative PKA GFP fusion protein (DNPKA/GFP) (D). B) GFP expression was first seen at 30 min., was maximal between 1 hour and 10 hours after heatshock. C) nk2.2 was regionally absent in injected/heat-shocked embryos, indicating the Gli2-DR/GFP protein blocks transcription of this Hh target gene. The loss of nk2.2 coincided with GFP labeling of the Gli2DR/GFP fusion protein (inset). D) Conversely, heat-shock induced DNPKA/GFP fusion protein led to ectopic expression of nk2.2. Inset shows GFP fusion protein expression in the eye 8 hours after heatshock.

### DETAILED DESCRIPTION

### Definitions

As used herein, the following terms and phrases shall have the meanings set forth below. Unless defined otherwise, all technical and scientific terms used herein have the same meaning as commonly understood to one of ordinary skill in the art.

The singular forms “a,” “an,” and “the” include plural reference unless the context clearly dictates otherwise.

The term “agent” is used herein to denote a chemical compound, a mixture of chemical compounds, a biological macromolecule (such as a nucleic acid, an antibody, a protein or portion thereof, e.g., a peptide), or an extract made from biological materials such as bacteria, plants, fungi, or animal (particularly mammalian) cells or tissues. The activity of such agents may render it suitable as a “therapeutic agent” which is a biologically, physiologically, or pharmacologically active substance (or substances) that acts locally or systemically in a subject.

The term “isolated” in reference to a polypeptide or polynucleotide of the invention means substantially separated from the components of its natural environment. Preferably, an isolated polypeptide or polynucleotide is a composition that consists of at least eighty percent of the polypeptide or polynucleotide identified by sequence on a weight basis as compared to components of its natural environment; more preferably, such composition consists of at least ninety-five percent of the polypeptide or polynucleotide identified by sequence on a weight basis as compared to components of its natural environment; and still more preferably, such composition consists of at least ninety-nine percent of the polypeptide or polynucleotide identified by sequence on a weight basis as compared to components of its natural environment. Most preferably, an isolated polypeptide or polynucleotide is a homogeneous composition that can be resolved as a single spot after conventional separation by two-dimensional gel electrophoresis based on molecular weight and isoelectric point. Protocols for such analysis by conventional two-dimensional gel electrophoresis are well known to one of ordinary skill in the art, e.g. Hames and Rickwood, Editors, Gel Electrophoresis of Proteins: A Practical Approach (IRL Press, Oxford, 1981); Scopes, Protein Purification (Springer-Verlag, New York, 1982); Rabilloud, Editor, Proteome Research: Two-Dimensional Gel Electrophoresis and Identification Methods (Springer-Verlag, Berlin, 2000).

The term “percent identical” refers to sequence identity between two amino acid sequences or between two nucleotide sequences. Identity can each be determined by comparing a position in each sequence which may be aligned for purposes of comparison. When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology, similarity, or identity refers to a function of the number of identical or similar amino acids at positions shared by the compared sequences. Expression as a percentage of homology, similarity, or identity refers to a function of the number of identical or similar amino acids at positions shared by the compared sequences. Various alignment algorithms and/or programs may be used, including FASTA, BLAST, or ENTREZ. FASTA and BLAST are available as a part of the GCG sequence analysis package (University of Wisconsin, Madison, Wis.), and can be used with, e.g., default settings. ENTREZ is available through the National Center for Biotechnology Information, National Library of Medicine, National Institutes of Health, Bethesda, Md. In one embodiment, the percent identity of two sequences can be determined by the GCG program with a gap weight of 1, e.g., each amino acid gap is weighted as if it were a single amino acid or nucleotide mismatch between the two sequences.

Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Preferably, an alignment program that permits gaps in the sequence is utilized to align the sequences. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. An alternative search strategy uses MPSRCH software, which runs on a MASPAR computer. MPSRCH uses a Smith-Waterman algorithm to score sequences on a massively parallel computer. This approach improves ability to pick up distantly related matches, and is especially tolerant of small gaps and nucleotide sequence errors. Nucleic acid-encoded amino acid sequences can be used to search both protein and DNA databases.

The terms “polynucleotide”, and “nucleic acid” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified, such as by conjugation with a labeling component. The term “recombinant” polynucleotide means a polynucleotide of genomic, cDNA, semisynthetic, or synthetic origin which either does not occur in nature or is linked to another polynucleotide in a normatural arrangement.

A “patient,” “individual,” “subject” or “host” refers to either a human or a non-human animal.

The term “substantially homologous” when used in connection with amino acid sequences, refers to sequences which are substantially identical to or similar in sequence with each other, giving rise to a homology of conformation and thus to retention, to a useful degree, of one or more biological (including immunological) activities. The term is not intended to imply a common evolution of the sequences.

The term “modulation” is art-recognized and refers to up regulation (i.e., activation or stimulation), down regulation (i.e., inhibition or suppression) of a response, or the two in combination or apart.

“Small molecule” as used herein, is meant to refer to a composition, which has a molecular weight of less than about 5 kD and most preferably less than about 4 kD. Small molecules can be nucleic acids, peptides, polypeptides, peptidomimetics, carbohydrates, lipids or other organic (carbon-containing) or inorganic molecules. Many pharmaceutical companies have extensive libraries of chemical and/or biological mixtures, often fungal, bacterial, or algal extracts, which can be screened with any of the assays described herein.

The terms “systemic administration,” “administered systemically,” “peripheral administration” and “administered peripherally” are art-recognized and refer to the administration of a subject composition, therapeutic or other material other than directly into the central nervous system, such that it enters the patient's system and, thus, is subject to metabolism and other like processes.

The terms “parenteral administration” and “administered parenterally” are art-recognized and refer to modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intra-articulare, subcapsular, subarachnoid, intraspinal, and intrasternal injection and infusion.

“Transcriptional regulatory sequence” is a generic term used to refer to DNA sequences, such as initiation signals, enhancers, and promoters, which induce or control transcription of protein coding sequences with which they are operable linked. Transcription of a recombinant genes may be under the control of a promoter sequence (or other transcriptional regulatory sequence) which controls the expression of the recombinant gene in a cell-type which expression is intended. It will also be understood that the recombinant gene can be under the control of transcriptional regulatory sequences which are the same or which are different from those sequences which control transcription of the naturally-occurring forms of genes as described herein.

A “vector” is a self-replicating nucleic acid molecule that transfers an inserted nucleic acid molecule into and/or between host cells. The term includes vectors that function primarily for insertion of a nucleic acid molecule into a cell, replication of vectors that function primarily for the replication of nucleic acid, and expression vectors that function for transcription and/or translation of the DNA or RNA. Also included are vectors that provide more than one of the above functions. As used herein, “expression vectors” are defined as polynucleotides which, when introduced into an appropriate host cell, can be transcribed and translated into a polypeptide(s). An “expression system” usually connotes a suitable host cell comprised of an expression vector that can function to yield a desired expression product.

“Treating” a condition or disease refers to curing as well as ameliorating at least one symptom of the condition or disease or preventing the condition or disease from worsening.

The term “therapeutic agent” is art-recognized and refers to any chemical moiety that is a biologically, physiologically, or pharmacologically active substance that acts locally or systemically in a subject. The term also means any substance intended for use in the diagnosis, cure, mitigation, treatment or prevention of disease or in the enhancement of desirable physical or mental development and/or conditions in an animal or human.

The term “therapeutic effect” is art-recognized and refers to a local or systemic effect in animals, particularly mammals, and more particularly humans caused by a pharmacologically active substance. The phrase “therapeutically-effective amount” means that amount of such a substance that produces some desired local or systemic effect at a reasonable benefit/risk ratio applicable to any treatment. The therapeutically effective amount of such substance will vary depending upon the subject and disease or condition being treated, the weight and age of the subject, the severity of the disease or condition, the manner of administration and the like, which can readily be determined by one of ordinary skill in the art. For example, certain compositions described herein may be administered in a sufficient amount to produce a desired effect at a reasonable benefit/risk ratio applicable to such treatment.

### Nucleic Acids Encoding Hh Activator or Repressor Proteins

Provided herein are nucleic acids and host cells and organisms containing one or more nucleic acids, e.g., heterologous or non-naturally occurring nucleic acids, which encode proteins that modulate the expression of Hh target genes. These nucleic acids may be used to artificially activate or inactivate the Hh signaling response within responding cells, e.g., in an organism. A nucleic acid may encode a protein, such as a transcription factor, or portion thereof, that is known to regulate the transcription of Hh target genes. Such proteins are referred to herein as “Hh regulatory proteins.” In one embodiment, the Hh regulatory protein is a Gli protein. In another embodiment, the Hh regulatory protein is protein kinase A (PKA).

A Gli protein may be a Gli1, 2 or 3 protein. Gli proteins are found in various vertebrate species, e.g., human, mouse, zebrafish, Drosophila, and any of these Gli proteins may be used. The nucleotide and amino acid sequences of Gli proteins may be found in GenBank, e.g., under GenBank Accession numbers set forth in the Table below:

| Name | Gene ID | Nucleotide seq. | Amino Acid seq. |
|---|---|---|---|
| Gli1 (zebrafish) | 352930 | NM_178296 | NP_840081 |
| | | AY173030 | |
| Gli2a (zebrafish) | 30154 | NM_130967 | NP_571042 |
| | | AF085746 | |
| Gli2b (zebrafish) | 548610 | NM_001015069 | NP_001015069 |
| | | AY928397 | |
| Gli3 (zebrafish) | 403042 | NM_205728 | NP_991291 |
| | | AY377429 | |
| PKA (mouse) | | AK005039 | BAB23766.1 |

Exemplary Gli proteins that may be used are truncated Gli proteins. Whereas full length Gli2 is a weak activator, it has been shown that truncation of Gli2 after the DNA binding domain (zinc finger domain) results in a strong activator of the Hh response, whereas truncation of Gli2 before the DNA binding domain results in a strong repressor of the Hh response.

A preferred Gli protein is a C-terminally truncated Gli2 protein, such as a C-terminally truncated zebrafish Gli2 protein. Such a protein has been shown to be a potent dominant repressor of Gli mediated Hh signaling (Karlstrom et al. (2003) Development 130: 1549) (“GLI2DR”). The C-terminal truncation may remove all or part of the first activation domain (A1), which is located at about amino acids 642-1183 of mouse Gli2; all or part of the second activation domain (A2), which is located at about amino acids 1184-1544 of mouse Gli2; or least about amino acids 1452-1544 of mouse Gli2, which is a domain that is required for activation. An exemplary zebrafish C-terminally truncated Gli2 protein is encoded by nucleotides 364-3021 of the nucleotide sequence set forth in GenBank Accession No. AF085746 and comprises the DNA binding zinc finger domain that is encoded by about nucleotides 1560 to 2060 of the same nucleotide sequence.

Another preferred Gli protein is an N-terminally truncated form of Gli2 or Gli3, such as an N-terminally truncated form of zebrafish Gli2 or Gli3. Such proteins have been shown to act as activators of Hh signaling (Sasaki et al. (1999) Development 126:3915). Activation of Hh signaling at the level of Gli transcription factors has been achieved in chicken using the truncated Gli3 construct (Stamataki et al. (2005) Genes Dev. 19:626). The N-terminal truncation may remove all or part of the repression (N) domain, which is located at about amino acids 1-279 of mouse Gli2. An exemplary zebrafish N-terminally truncated Gli3 protein is encoded by about nucleotides 1800-5086 of the nucleotide sequence set forth in GenBank Accession No. AY377429 and includes the DNA binding zinc finger domain that is located at about nucleotides 1840-2320 of the same nucleotide sequence.

Yet another preferred Hh regulatory protein that may be used for modulating the Hh signaling response in cells is a dominant negative form of the kinase PKA (DNPKA), e.g., from zebrafish, which has been shown to activate Hh signaling within a cell (Hammerschmidt et al. (1996) Genes Dev. 10:647). An exemplary dominant negative form of mouse PKA is encoded by about nucleotides 134-1276 of the nucleotide sequence set forth in GenBank Accession No. AK005039.

Exemplary Hh regulatory proteins are those encoded by the vectors set forth in Table 1, or proteins comprising, consisting essentially of, or consisting of Hh regulatory proteins encoded by these vectors.

Variants of naturally-occurring Hh regulatory proteins or portions thereof may also be used. Exemplary variants or homologs are biologically active variants, i.e., variants that have a biological activity, e.g., to activate or repress Hh signaling, e.g., by a factor of 50%, 2 fold, 3 fold, 5 fold, 10 fold, 30 fold, 60 fold, 100 fold or more.

A variant of a polypeptide may be a polypeptide having the amino acid sequence of the polypeptide in which one or more amino acid residues (e.g., 1, 2, 3, 4, 5, 10, 15, 20, 30, 40, 50 or more amino acids) are altered, e.g., substituted, deleted or added. A variant may have “conservative” changes, wherein a substituted amino acid has similar structural or chemical properties (e.g., replacement of leucine with isoleucine). A variant may have “nonconservative” changes (e.g., replacement of glycine with tryptophan). Analogous minor variations may also include amino acid deletions or insertions, or both (e.g., of 1, 2, 3, 4, 5, 10, 15, 20, 30, 40, 50 or more amino acids). Guidance in determining which amino acid residues may be substituted, inserted, or deleted without abolishing biological activity may be found using computer programs well known in the art, for example, LASERGENE software (DNASTAR).

Variant nucleic acids that may be used include, for example, “allelic,” “splice,” “species,” or “polymorphic” variants. Polymorphic variants may also encompass “single nucleotide polymorphisms” (SNPs) in which the polynucleotide sequence varies by one base.

Hh regulatory proteins may differ from their corresponding naturally occurring protein or a fragment thereof, by the addition of 1, 2, 3, 4, 5, 6, 7, 8, 9, 10, 15, 20, 30, 40, 50 or more amino acids, e.g., consecutive amino acids, at the C-terminus, N-terminus, and/or within the protein. Where a Hh regulatory protein is a fragment of a naturally occurring Hh protein, the fragment may comprise additional amino acid stretches that are heterologous to the naturally occurring protein, i.e., that do not occur at the same place in the naturally-occurring protein. Proteins comprising a portion of a naturally occurring protein may be proteins that comprise a portion of the naturally occurring protein provided (or with the proviso that) the protein does not comprise the full length naturally occurring protein.

Variants may also be proteins that are at least about 70%, 80%, 90%, 95%, 97%, 98% or 99% identical to a wild-type protein or portion thereof, such as the portions described herein. Variants may also be proteins that are encoded by nucleic acids that are at least about 70%, 80%, 90%, 95%, 97%, 98% or 99% identical to a nucleic acid encoding a wild-type protein or portion thereof, such as the portions described herein.

Yet other variants are those that are encoded by a nucleic acid that hybridizes, preferably under stringent hybridization conditions, to a nucleic acid encoding a wild-type protein and/or a protein described herein or a portion thereof, such as those described herein. Stringent hybridization conditions may include hybridization and a wash in 0.2×SSC at 65° C. Nucleic acids that hybridize under high stringency conditions of 0.2 to 1×SSC at 65° C. followed by a wash at 0.2×SSC at 65° C. to a nucleic acid described herein or a portion thereof can be used. Nucleic acids that hybridize under low stringency conditions of 6×SSC at room temperature followed by a wash at 2×SSC at room temperature to nucleic acid described herein or a portion thereof can be used. Other hybridization conditions include 3×SSC at 40 or 50° C., followed by a wash in 1 or 2×SSC at 20, 30, 40, 50, 60, or 65° C. Hybridizations can be conducted in the presence of formaldehyde, e.g., 10%, 20%, 30% 40% or 50%, which further increases the stringency of hybridization. Theory and practice of nucleic acid hybridization is described, e.g., in S. Agrawal (ed.) Methods in Molecular Biology, volume 20; and Tijssen (1993) Laboratory Techniques in biochemistry and molecular biology-hybridization with nucleic acid probes, e.g., part I chapter 2 “Overview of principles of hybridization and the strategy of nucleic acid probe assays,” Elsevier, New York provide a basic guide to nucleic acid hybridization.

Also provided are nucleic acids encoding proteins that are variants of proteins encoded by the nucleic acids set forth in Table 1, e.g., proteins having a particular homology or similarity to those encoded by the nucleic acids in Table 1, and proteins that are encoded by nucleic acids that have a particular homology or similarity or hybridize to those in Table 1 or portions thereof. A portion may be any of the elements of the nucleic acids, such as the elements that are set forth in the Figures.

Hh regulatory proteins may further be linked or fused to a heterologous protein or peptide (generally referred to herein as “heterologous protein”), such as a protein that allows the Hh regulatory protein to be detected and/or quantitated. A heterologous protein may be any protein that may be detected, such as by the use of an antibody binding specifically to the protein. A heterologous protein may also be an enzyme that provides a product that can be detected. In one embodiment a heterologous protein is green fluorescent protein (GFP) or Sema3D-GFP (Halloran). Different color fluorescent proteins may also be used. Other heterologous proteins that may be used include a His tag, such as a hexaHis tag. A His tag may be added in addition to another heterologous protein. Other useful epitope tags include myc-epitopes (e.g., see Ellison et al. (1991) J Biol Chem 266:21150-21157) which includes a 10-residue sequence from c-myc, as well as the pFLAG system (International Biotechnologies, Inc.) or the pEZZ-protein A system (Pharmacia, NJ).

A heterologous protein may be linked directly or indirectly to an Hh regulating protein. In one embodiment, a heterologous protein is linked at the C-terminus of a Hh regulating protein. In another embodiment, it is linked at the N-terminus. In certain embodiments, it may even be located within an Hh regulating protein.

A nucleic acid encoding a Hh regulatory protein may be operably linked to one or more transcriptional regulatory elements, such as a promoter and an enhancer. In a preferred embodiment, the promoter is an inducible promoter. A preferred inducible promoter is a heat inducible promoter, such as a heat-shock inducible promoter controlling the expression of a heat-shock gene. An exemplary heat-shock inducible promoter is that of HSP70. The zebrafish HSP70 promoter is set forth in GenBank Accession No. AF158020 (Halloran (2000) infra and Xiao et al. (2003) J. Neurosc. 23:4190). In one embodiment, about 1.5 kb of DNA upstream of the putative ATG translation start site of this HSP70 promoter sequence is cloned upstream of a coding sequence of interest. Exemplary promoters are those set forth in the vectors of Table 1. A person of skill in the art will recognize that longer and shorter portions of this promoter may also be used, provided that it still drives transcription and is heat inducible. Raising the temperature of the environment to about 37° C., e.g., for about 30-60 minutes, will result in the induction of the heat-shock promoter and expression of genes linked downstream thereof.

Other inducible promoters that may be used include the glutathione receptor promoter, e.g., the human glutathione receptor promoter, which is inducible by dexamethasone (Tribulo et al. (2003)). A promoter may comprise the site to which the ligand binding domain of the human glucocorticoid receptor (amino acid residues 512-777) binds.

Nucleic acids encoding Hh regulatory proteins may further comprise additional nucleotide sequences, such as additional regulatory nucleotide sequences. They may also be encompassed within a plasmid or a vector, such as an expression vector. They may also comprise elements facilitating the integration of a nucleic acid into a genome, such as a zebrafish genome. An exemplary DNA based integration system is the Tol2-transposon based system (Kawakami (2004) Methods Cell Biol. 77:201). Thus, a nucleic acid may comprise one or more transposons, e.g., Tol2 transposons.

Nucleic acids may also comprise elements that allow expression of more than one protein. For example, nucleic acids may be multicistronic vectors or vectors comprising one or more IRES (internal ribosome entry site). Di-, tri-, and quattrocistronic expression vectors may allow the simultaneous, coordinated, and adjustable expression of 2, 3 or 4.

Exemplary vectors are those set forth in Table 1. Constructs for use may also comprise combinations of the various elements set forth in the figures pertaining to the vectors set forth in Table 1.

### Hh Reporter Nucleic Acids

Also provided herein are nucleic acids comprising a reporter gene operably linked to a transcriptional response element that is indicative of Hh signaling, e.g., an Hh regulatory element. An Hh regulatory element may be a 5′ or 3′ regulatory region of a gene that is regulated by an Hh regulatory protein, e.g., a Gli. An Hh regulatory element may be the promoter and/or enhancer of such genes, or portions thereof. For example an Hh regulatory element may be a discrete fragment of a promoter or enhancer. In one embodiment, an Hh regulatory element is a binding site for a transcription factor, such as a Gli protein (see, e.g., Sasaki et al. (1997) infra). A know consensus sequence of human Gli-binding site is 5′ GACCACCCA 3′ and a consensus sequence of mouse Gli-binding site is 5′GAACACCCA3′ (Sasaki et al. (1997) infra). An exemplary binding site for a Gli protein is present in the 3′ region of the gene encoding Hepatocyte Nuclear Factor-3β (HNF-3β), such as the mouse HNF-3β (Sasaki et al. (1997) infra). The DNA regulator region that regulates Patched (Ptc) expression (Ptc promoter region) can also be used to monitor Hh activation.

Hh regulatory elements may also comprise more than one transcriptional control element. For example, an Hh regulatory element may comprise 2, 3, 4, 5, 6, 7, 8, 9, 10 or more transcriptional control elements, such as transcription factor binding sites. In one embodiment, a nucleic acid comprises more than one Gli binding site, e.g., 8 Gli binding sites.

A reporter gene may be any nucleic acid encoding a protein that can be detected, e.g., luciferase and GFP (and any others further described herein).

The Hh reporter nucleic acid may further be linked to other DNA elements, such as other regulatory elements. They may also be part of a plasmid or vector.

Exemplary reporter constructs are set forth in Table 2. Nucleic acids that are variants of the nucleic acids in Table 2 may also be used. Variants may be nucleic acids having a particular homology or similarity to a nucleic acid in Table 2 or an element thereof, such as the elements set forth in the Figures pertaining to these constructs. Reporter constructs may also comprise combinations of the various elements set forth in these figures.

### Transgenic Cells and Organisms

Also provided herein are cells and organisms, such as isolated cells and organisms, comprising one or more of the nucleic acids described herein. In certain embodiments, a cell or organism may comprise a nucleic acid encoding a Hh regulating protein and a Hh reporter nucleic acid. A cell may be a eukaryotic or a prokaryotic cell. Exemplary eukaryotic cells include vertebrate and mammalian cells, such as human, mouse, rat, non-human simian, ovine, bovine, equine, feline, canine, yeast, and zebrafish cells. A prokaryotic cell may be a bacterial cell. A cell may also be a plant cell. Other cells include progenitor and stem cells, such as embryonic stem cells and adult stem cells.

An organism may be a unicellular or a multicellular organism, such as those described above. A preferred organism is a vertebrate, e.g., a zebrafish.

Methods for introducing one or more nucleic acids, e.g., those described herein, into a cell or organism are well known in the art. For example, the following method may be used to create transgenic zebrafish lines (Lin et al. (2000) Methods Mol. Biol. 136:375). The HSP70-Gli2DR-GFP cassette may be placed into a meganuclease site containing vector (J. Wittbrodt) to increase incorporation into the genome (Thermes et al. (2002) Mech. Dev. 118:91). This vector may then be injected at the 1 cell stage, optionally with the enzyme SceI, and fish may be heat-shocked at 24 hours to identify embryos with a high percentage of cells expressing the plasmid gene. These embryos may be grown to maturity, out-crossed to wild-type adults. Inducible GFP expression may be monitored in these embryos. Those embryos with GFP expression will be grown to adulthood and used to establish a transgenic line. If heatshock expression of Gli2DR-GFP or DNPKA-GFP or other Hh regulating protein is lethal, un-heat shocked siblings from positive clutches may be grown and their progeny will be heatshocked to identify founding adults. Alternatively, and in combination, PCR amplification of vector DNA sequences from pooled embryo DNA may be used to identify germline transformed adults, which may then be out-crossed to establish the transgenic line.

In certain embodiments, instead of using a cell or organism comprising a nucleic acid described herein that is stably integrated in the genome of the cell or organism, cells and organisms transiently expressing the nucleic acid may also be used. Such cells and organisms may be obtained by injecting into them the nucleic acid of interest. When using an organism for example, a nucleic acid of interest, such as a plasmid, may be administered (e.g., injected) into an embryo, such as an early stage embryo.

Zebrafish lines can be stored at the Zebrafish International Resource Center (ZIRC) in Eugene Oreg. (http://zebrafish.org/zirc/home/guide.php) as either live fish or frozen sperm, or both.

### Exemplary Methods

Provided herein are methods for modulating Hh signaling in a cell or organism and thereby modulate events resulting from modulation of Hh signaling, e.g., cell proliferation and differentiation. A method may comprise contacting a cell or organism with, or administering into the cell or organism, a Hh regulating protein or a nucleic acid encoding such. The Hh regulating protein may further be linked to a heterologous protein, e.g., allowing the detection of the Hh regulating protein. In one embodiment, the nucleic acid encoding the Hh regulating protein comprises or is operably linked to a nucleic acid comprising an inducible promoter, and the method comprises contacting the cell or organism with, or subjecting the cell or organism to a condition, that induces the inducible promoter. When an inducible promoter is a heat inducible promoter, e.g., a heat-shock promoter, the method may comprise subjecting the cell or organism to heat, e.g. a heat-shock, at a temperature and for a time sufficient to induce the expression of the heat-inducible promoter.

The time of heat induction will depend on the tool or condition used to heat the cell or organism. If the condition is incubation of the cell or organism at an inducing temperature, e.g., about 37° C., induction may be for about, or for at least about, 10 minutes, 20 minutes, 30 minutes, 40 minutes, 50 minutes, 60 minutes, 70 minutes or more. For example, zebrafish may be heat induced by placing them in a 37° C. waterbath for about 30-60 minutes. Heat induction of an organism or at least some cells of an organism may also be conducted using a tungsten wire.

In one embodiment, embryos, e.g., zebrafish embryos, e.g., 15-24 hour embryos, will be anesthetized in tricaine as needed and mounted in about 0.1% agarose. A tungsten wire electrode will be heated to the point at which it just melts the agarose, then will be brought to a desired position on the embryo, e.g., by using a Narishige micromanipulator. Exposure may be for a few minutes, e.g., 1, 2, 3, 4, 5, or 10 minutes, preferably 3 minutes, using approximately 0.5, 1, 2, 3, or 5 Volts, preferably 1 Volt. Exposure for 3 minutes using approximately 1 Volt has been shown to cause local expression of GFP in a heat-shock promoter-GFP transgenic zebrafish cell line. After heating, embryos may be incubated for a certain amount of time, e.g., about 30 minutes, 1 hour, 2 hours or 3 hours and expression of the heterologous protein, e.g., GFP, will be monitored, e.g., with a fluorescent dissecting microscope or compound microscope, as needed.

Another method of heat inducing a heat inducible promoter is localized laser generated heatshock to drive expression in small numbers of cells in a desired region (Halloran et al. (2000) Development 127:1953). A laser may be a helium laser, e.g., mounted on a Zeiss compound microscope. Embryos, e.g., 14-20 somite embryos, may be anesthetized in tricaine, mounted in methylcellulose, and placed under a cover lip. Laser light from a MicroPoint nitrogen laser using a Coumarin 440 dye cube (Photonic Instruments, Arlington Heights, Ill.) may be focused on the cells of interest through a 40× objective, activating the HSP70 promoter in, e.g., 2-10 cells. A 2 minute burst of 4 ns laser pulses delivered at a frequency of 3-4 Hz has been reported to effectively activate the HSP70 promoter without inducing cell death (Halloran et al., 2000). Thus, this heat inducing method as well as others, e.g., using an electrode can be used to locally and temporally induce transcription from a heat inducible promoter, e.g., a heatshock promoter.

A local heatshock tool may also be used. 1. A heating probe may comprise: an uncoiled portion of an igniter wire having a length of about 3 cm and bent to form a heating element; a J-type thermocouple attached to an end of the igniter wire; a controller connected to the heating element and thermocouple for limiting the temperature of the heating element based on heat sensed by the thermocouple; a micropipette tip for attaching the heating element and thermocouple to a syringe, wherein wiring from the heating element and thermocouple pass through the syringe to the controller. The igniter wire may be an NiCr wire. The heating probe may further comprise an additional NiCr wire element coiled about the heating element for operation of the driver circuitry components at a safe operating area. The additional NiCr wire element may be 40 AWG and have a length of about 1.5 mm. The heating element may be 35 AWG.

Although zebrafish in which a nucleic acid encoding a Hh regulating protein is stably integrated (transgenic zebrafish), are preferably used, zebrafish which are mosaic for a nucleic acid encoding a Hh regulating protein may also be used. A mosaic zebrafish may be obtained by injecting into a zebrafish embryo the nucleic acid. For example, a nucleic acid, such as in a circular plasmid, may be injected into a zebrafish embryo at a two cell stage. In one embodiment, a nucleic acid encoding a Hh regulating protein is injected at a 2 cell stage zebrafish embryo, heat-shocked for about 30-60 minutes at 37° C. at the 10 hour stage, and expression of the heterologous protein, e.g., GFP, is monitored about 1 hour later using a fluorescent dissecting microscope or a compound microscope as needed. Embryos with expression of the heterologous protein may then be fixed, e.g., for about 20 hours, and labeled using a marker of Hh signaling, e.g., an nk2.2, in situ probe to determine whether Hh signaling has been modulated. If the heterologous protein is GFP, fluorescence may be detected or an anti-GFP antibody (e.g., from Clontech) may be used to label expressing cells. An in vivo assay of function may be to inject the nucleic acid at the two cell stage, heatshock at 15-18 hours, then assay Hh signaling marker, e.g., nk2.2, expression by in situ hybridization at 24 hours.

Inducing the expression of an Hh regulating protein in a cell or organism, such as temporally and/or spatially inducing the expression of a Hh regulating protein in an organism may be used to determine the effect of a modulation of Hh signaling in particular cells. For example, a cell or an organism, in particular a transgenic organism, comprising a nucleic acid encoding a Hh regulating protein may be used as a model, e.g., an animal model, of an Hh associated disease, e.g., a disease that is caused by or associated with an abnormal Hh signaling. Such cell or animal models may be used, e.g., for identifying agents that modulate Hh signaling and to identify agents that are toxic to cells and organisms due to their modulation of Hh signaling.

Also provided herein are methods for identifying an agent that modulates Hh signaling. A method may comprise contacting a cell or organism comprising a nucleic acid encoding a Hh regulating protein with a test agent and determining the level of Hh signaling, wherein a different level of Hh signaling in a cell or organism that was contacted with the agent relative to a cell or organism that was not contacted with the agent indicates that the agent modulates Hh signaling. The method may also comprise determining the level of expression of the Hh regulating protein. An Hh regulating protein may be linked or fused to a heterologous protein, and the method may comprise detecting the level of expression of the heterologous protein instead of, or in addition to, the level of expression of the Hh regulating protein.

In the method described in the previous paragraph, the nucleic acid encoding the Hh regulating protein may also be operably linked to an inducible promoter. In this case, a method may comprise one or more of the following steps, not necessarily in the order provided here: (i) contacting the cell or organism with a test agent; (ii) inducing the inducible promoter; and (iii) determining the level of Hh signaling; wherein a different level of Hh signaling in a cell or organism contacted with a test agent relative to a cell or organism that was not contacted with a test agent indicates that the test agent is an agent that modulates Hh signaling. Contacting the cell or organism with the test agent may be initiated before, after or at about the same time as inducing the inducible promoter. Contacting the cell or organism with the test agent may be terminated before, after or at about the same time as inducing the inducible promoter. Contacting the cell or organism with the test agent may be conducted before, after or during about the same time as inducing the inducible promoter.

In a preferred embodiment, a method is a screening method for identifying agents that may be used for treating or preventing Hh associated diseases, such as cancer or other hyperproliferating diseases. A method may comprise contacting a cell or organism comprising a nucleic acid encoding a Hh regulatory protein that is an activator and monitoring Hh signaling. An activator may be an N-terminally truncated form of Gli2 or Gli3 or a dominant negative form of PKA. The assay thus uses cells and organisms in which Hh signaling is abnormally high and can be used to identify molecules that reduce Hh signaling.

Methods for identifying an agent that modulates Hh signaling may also comprising using a cell or organism comprising a nucleic acid encoding a Hh reporter construct with a test agent and determining the level of the reporter construct, wherein a different level of reporter expression in a cell or organism that was contacted with the agent relative to a cell or organism that was not contacted with the agent indicates that the agent modulates Hh signaling.

A test agent may be any molecule or composition comprising more than one type of molecule, e.g., a small molecule. Thus, in one embodiment, a cell or organism is contacted with a single molecule or type of molecule to determine whether that molecule modulates Hh signaling. In another embodiment, a cell or organism is contacted with a mixture of different molecules or types of molecules to determine whether one or more of these molecules modulate Hh signaling. If a mixture of different molecules is found to modulate Hh signaling, then the mixture can be fractioned and individual fractions assayed to determine whether one or more of these modulate Hh signaling. Fractionation can be repeated several times, e.g., until an individual molecule that modulates Hh signaling is identified.

A test agent may be a molecule from a library. Accordingly, methods described herein may comprise contacting a cell or organism with a member or a library or with a pool of members from a library, e.g., a pool of about 3, 5, 10, 20 or more members.

Also provided herein are methods for determining whether a sample, such as an environmental sample, is toxic to a subject. A method may comprise contacting the sample with a cell or organism comprising a nucleic acid encoding a Hh regulating protein or reporter construct with the sample, and determining the level of Hh signaling in the cell or organism. A different level of Hh signaling in a cell or organism contacted with the sample relative to a cell or organism that was not contacted with the sample indicates that the sample is toxic to a subject. In a preferred embodiment, the Hh regulating protein is a repressor, such as a C-terminally truncated Gli2 protein. A toxic sample may be a sample that upregulates Hh signaling.

Monitoring Hh signaling can be conducted in a variety of ways. For example, when using a system in which Hh signaling is activated in a cell or organism, such as by using Hh regulating proteins that are activators, e.g., N-terminally truncated Gli2 and Gli3 proteins, this may result in hyperproliferation of the cell or cells in the organism. In this situation, one may identify agents that reduce Hh signaling by looking for those that reduce proliferation of the cell or cells in the organism. Assays for determining the proliferation rate of cells and organisms containing such are well known in the art. An exemplary assay comprises using a radioactive molecule, e.g., tritiated thymidine, that is specifically incorporated into proliferating cells. When using a system in which a tumor is caused by the expression of an Hh regulating protein, i.e., an activator in this case, tumor growth can be measured as an assay for measuring Hh signaling. Tumor growth can be measured by methods known in the art.

In another embodiment, Hh signaling is determined by measuring the expression of a reporter gene that is under the control of a promoter (or regulatory region) that is responsive to Hh signaling. In one embodiment, a cell or organism used in a method described herein using a nucleic acid encoding a Hh regulating protein further comprises a reporter construct, comprising a promoter that is responsive to Hh signaling operably linked to a reporter gene. Modulation of the reporter gene expression will reflect or correlate with modulation of Hh signaling. A promoter that is responsive to Hh signaling may be a nucleic acid comprising Gli binding sites, as further described herein. Other promoters include the nk2.2 and Ptc promoters. Any reporter gene may be used, such as those further described herein.

Compounds identified herein as inhibiting Hh signaling as well as nucleic acids encoding repressor Hh regulating proteins may be used for treating or preventing any disease or disorder that is caused by or associated with an abnormally high Hh signaling. Exemplary diseases include hyperproliferative diseases, such as cancer, e.g., basal cell carcinoma and medulloblastoma. Other diseases and conditions that may be treated or prevented are those described, e.g., in U.S. Patent application publication number 2006/0020020.

On the other hand, compounds identified herein as stimulating Hh signaling as well as nucleic acids encoding activator Hh regulating proteins may be used for treating or preventing any disease or disorder that is caused by or associated with an abnormally low Hh signaling. Exemplary diseases or conditions are set forth, e.g., in U.S. Patent application publication number 2006/0020020.

Transgenic lines that allow the conditional up- and down-regulation of Hh signals at any time in development will also be extremely useful for characterizing newly found drugs that affect Hh signals. Additionally, they will help identify novel genes that might be defective in human disease.

The idea to use transgenic zebrafish lines for drug discovery is particularly compelling (MacRae and Peterson, 2003; Stern and Zon, 2003). One of the advantages of this system is that compounds can be tested simultaneously for their effectiveness and for their toxicity. Several zebrafish labs have recently published small-molecule chemical screens using transgenic lines, identifying compounds that affect developmental events and progression of disease models (Anderson et al., 2007; Murphey et al., 2006; Peterson et al., 2004). Whole organism chemical screens are particularly important, as they allow chemicals to be assessed for toxicity and non-specific effects. Hedgehog/Gli signaling is involved in common birth defects and cancers, and chemical screens have identified several promising agents that affect Hh signals (Berman et al., 2002). However a rapid method of monitoring effects on Hh signaling at any age is lacking. Thus our transgenic zebrafish lines that report Hh activity (i.e. Glibs-GFP) will be extremely useful for the identification and characterization of compounds that affect different aspects of the pathway and might thus be used to treat Hh signal dysfunction in human cancers such as basal cell carcinoma and medulloblastoma. Anderson, C., Bartlett, S. J., Gansner, J. M., Wilson, D., He, L., Gitlin, J. D., Kelsh, R. N. and Dowden, J. (2007). Chemical genetics suggests a critical role for lysyl oxidase in zebrafish notochord morphogenesis. Mol Biosyst 3, 51-9. Berman, D. M., Karhadkar, S. S., Hallahan, A. R., Pritchard, J. I., Eberhart, C. G., Watkins, D. N., Chen, J. K., Cooper, M. K., Taipale, J., Olson, J. M. et al. (2002). Medulloblastoma growth inhibition by hedgehog pathway blockade. Science 297, 1559-61. MacRae, C. A. and Peterson, R. T. (2003). Zebrafish-based small molecule discovery. Chem Biol 10, 901-8. Murphey, R. D., Stern, H. M., Straub, C. T. and Zon, L. I. (2006). A chemical genetic screen for cell cycle inhibitors in zebrafish embryos. Chem Biol Drug Des 68, 213-9. Peterson, R. T., Shaw, S. Y., Peterson, T. A., Milan, D. J., Zhong, T. P., Schreiber, S. L., MacRae, C. A. and Fishman, M. C. (2004). Chemical suppression of a genetic mutation in a zebrafish model of aortic coarctation. Nat Biotechnol 22, 595-9. Stern, H. M. and Zon, L. I. (2003). Cancer genetics and drug discovery in the zebrafish. Nat Rev Cancer 3, 533-9.

Also provided herein are kits comprising one or more reagents described herein. A kit may provide components for purposes of regulating Hh signaling or for conducting screening assays or toxicity assays.

The present description is further illustrated by the following examples, which should not be construed as limiting in any way. The contents of all cited references (including literature references, issued patents, published patent applications and GenBank Accession numbers as cited throughout this application) are hereby expressly incorporated by reference. When definitions of terms in documents that are incorporated by reference herein conflict with those used herein, the definitions used herein govern.

The practice of the present methods will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2^{nd} Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

### EXAMPLES

### Example 1

### Transgenic Zebrafish Lines Allowing Cell-Autonomous Temporal and Spatial Manipulation of Hh Signaling in a Vertebrate

During embryonic development, cell-cell communication is critical to the formation of diverse cellular tissues, for the patterning of these tissues, and for the regulation of cell proliferation. Small secreted proteins of the Hedgehog (Hh) family are among a number of important molecules that guide embryonic development. These same molecules continue to regulate cell proliferation into the adult organism and may be important for stem cell maintenance and differentiation. Mis-regulation of Hedgehog signaling is implicated in a large number of human diseases, including skin cancer (basal cell carcinoma), brain cancers (medulloblastoma), as well as a number of relatively common birth defects (holoprosencephaly). Understanding how Hh signaling is regulated in the developing embryo is an important step toward understanding how mis-regulation can lead to human diseases, and this information may have thus have profound implications for human health.

Our lab studies how cells respond to hedgehog signals in the vertebrate embryo, using the zebrafish as a model experimental organism. We have examined how Hh responsive transcription factors of the Gli family interpret Hh signals, resulting in proper induction and patterning of the embryonic nervous system and pituitary gland. In order to understand this process, we are developing genetic tools (transgenic zebrafish lines) that allow us to artificially activate or inactivate the Hh signaling response within responding cells in the intact organism. These tools will provide a new means to understand Hh signaling in living embryos and adults, and will allow us to study the results of cell-autonomous mis-regulation of Hh at any time in the life of a vertebrate.

We have taken advantage of our knowledge of the Hh signaling system to design three genetic constructs that allow us to regulate Hh signaling both positively and negatively within responding cells. We have shown that a C-terminally truncated zebrafish Gli2 protein acts as a potent dominant repressor of Gli mediated Hh signaling (Karlstrom et al., 2003). Conversely, others showed some time ago that N-terminally truncated forms of Gli2 and Gli3 act as activators of Hh signaling (Sasaki et al., 1999) and that a dominant negative form of the kinase PKA can activate Hh signaling within a cell (Hammerschmidt et al., 1996). Activation of Hh signaling at the level of gli transcription factors has been achieved in chicken using the truncated Gli3 construct (Stamataki et al., 2005). In order to understand how Hh signaling acts throughout the life of an organism, it would be ideal to be able to turn Hh signaling on or off at specific times and in specific locations, then monitor the effects on cell proliferation and differentiation. We are doing this in zebrafish by using the heat-shock inducible promoter HSP70 to drive the expression of these genes. Raising the temperature of embryos or adults to 37° C. for 30-60 minutes effectively activates the expression of genes downstream of this regulatory element. In order to monitor the expression of these genes, we have made fusion proteins that include the fluorescent GFP molecule. Now, by applying a mild heat-shock, we can turn any of these genes on in the entire organism and monitor the level of expression by GFP fluorescence. We have also generated a local heatshock device that allows us to heatshock a small region of the organism, allowing spatial as well as temporal control of expression.

Set forth below is a table (Table 1) listing the constructs that have been made:

| Construct name | Fig. | SEQ ID NO |
|---|---|---|
| hsp70.Gli3Act.eGFP | 1 | 1 |
| hsp70.Gli3Act.IRES.nls.eGFP | 2 | 3 |
| hsp70.Gli3Act.mCherry | 3 | 5 |
| hsp70.Gli3act474.eGFP | 4 | 7 |
| hsp70.Gli3act474.IRES.nls.eGFP | 5 | 9 |
| hsp70.Gli3Act474.mCherry | 6 | 11 |
| hsp70.Gli1Act.eGFP | 7 | 13 |
| hsp70.Gli1Act.IRES.nls.eGFP | 8 | 15 |
| hsp70.Gli1Act.mCherry | 9 | 17 |
| hsp.Gli2Act.eGFP | 10 | 19 |
| hsp.Gli2.IRES.nls.GFP | 11 | 21 |
| hsp70.Gli2Act.mcherry | 12 | 23 |
| hsp.dnpka.GFP | 37 | 49 |
| hsp70.Gli2DR.eGFP | 38 | 51 |

Set forth below are explanations of the names and constructs:

hsp70.Gli3act.eGFP: hsp70: heat shock promoter 70; Gli3 Act: Gli3 activactor contains mRNA sequence 1754-5062 (5045 nucleotide has been changed from T to A to avoid stop code); eGFP:enhanced green fluorescent protein.

hsp70.Gli3act.IRES.nlsGFP: hsp70: heat shock promoter 70; IRES:encephalomyocarditis (EMCV) IRES (Internal ribosom entry site) sequence; nlsGFP: nucleus GFP; Gli3 Act: Gli3 activactor contains mRNA sequence 1754-5062 (CDs,386-5047; 5045 nucleotide has been changed from T to A to avoid stop code)

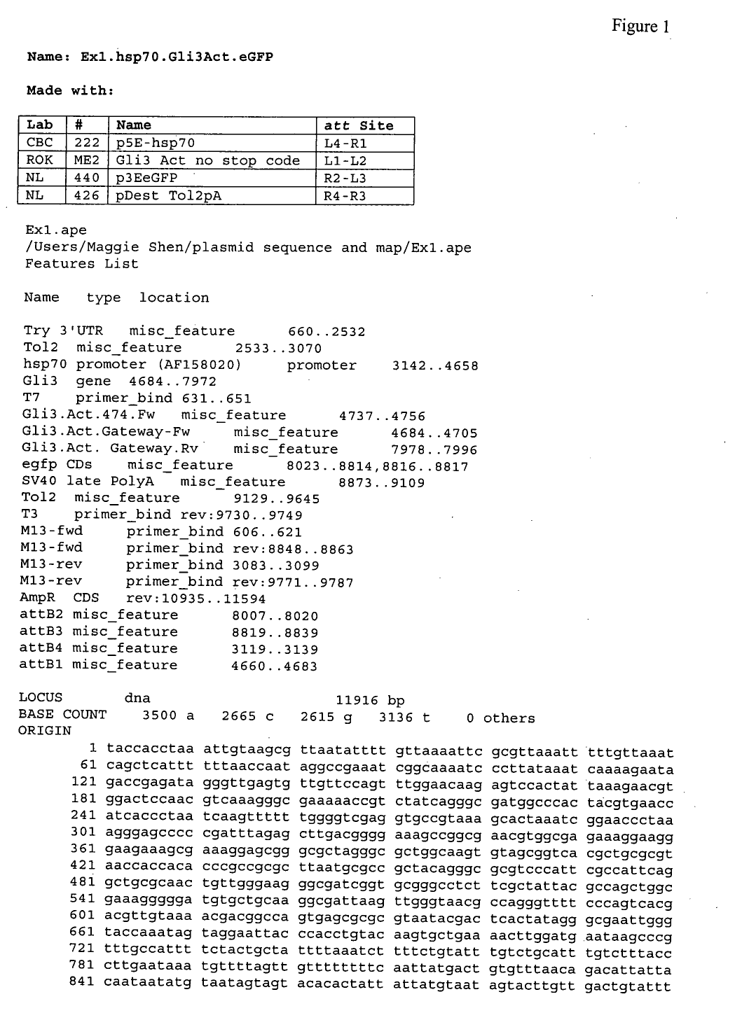

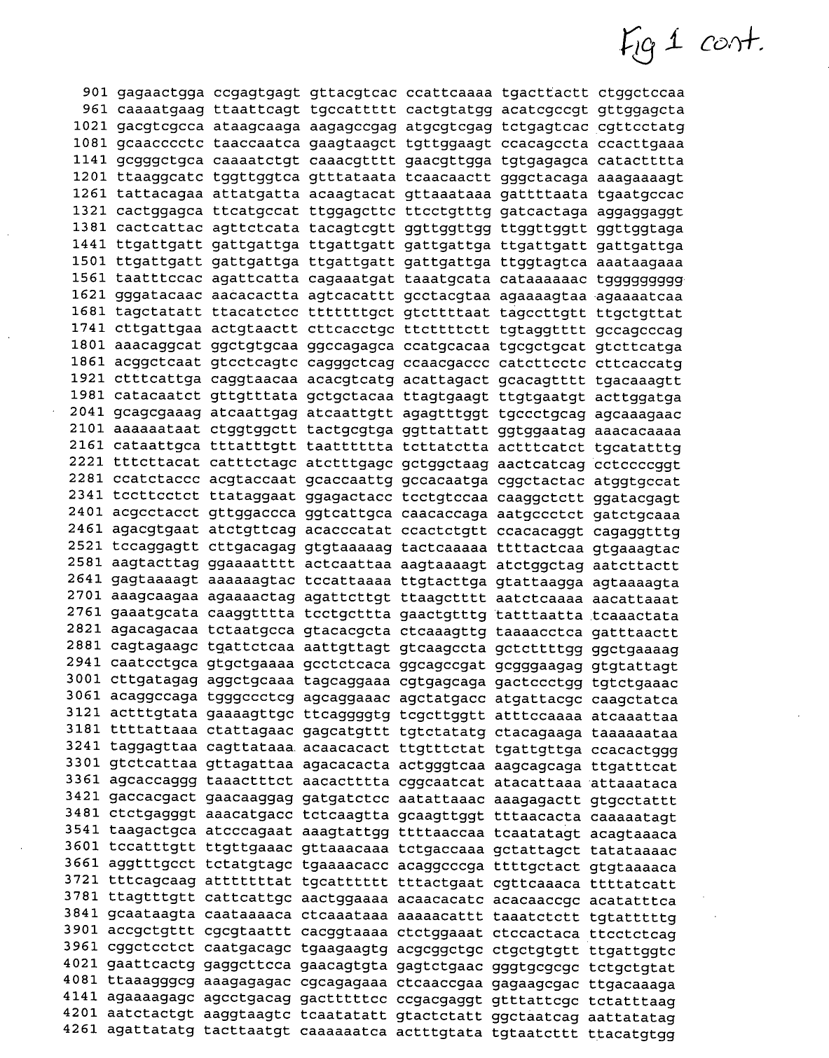

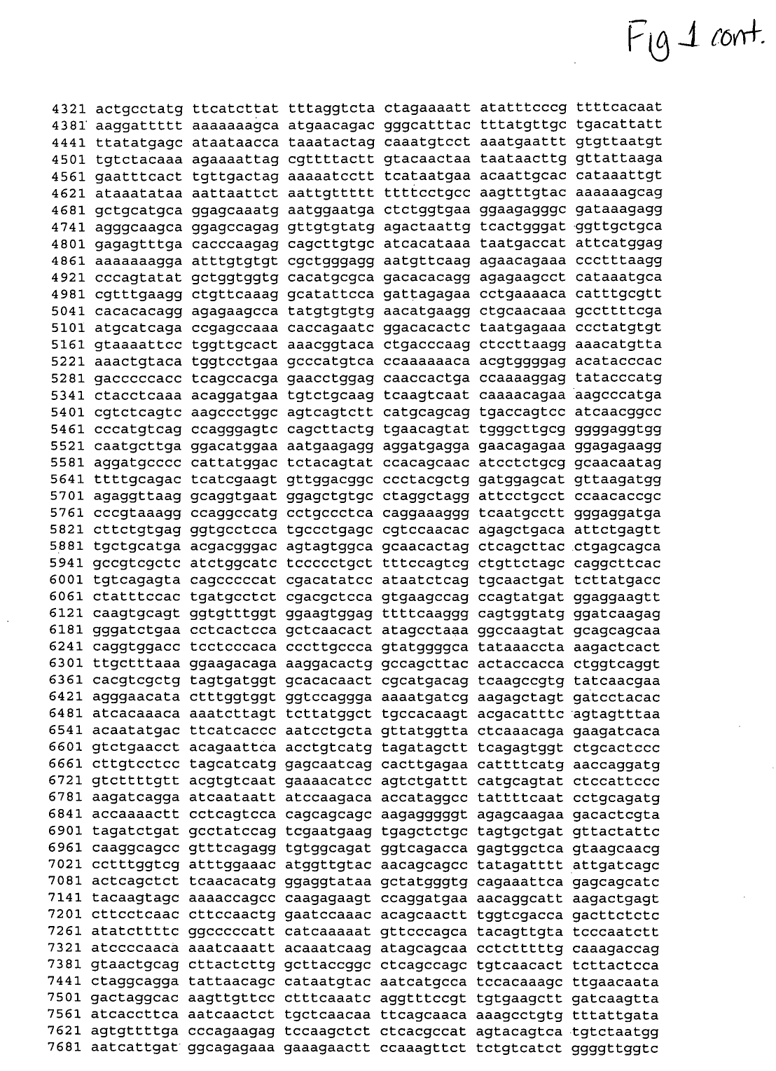

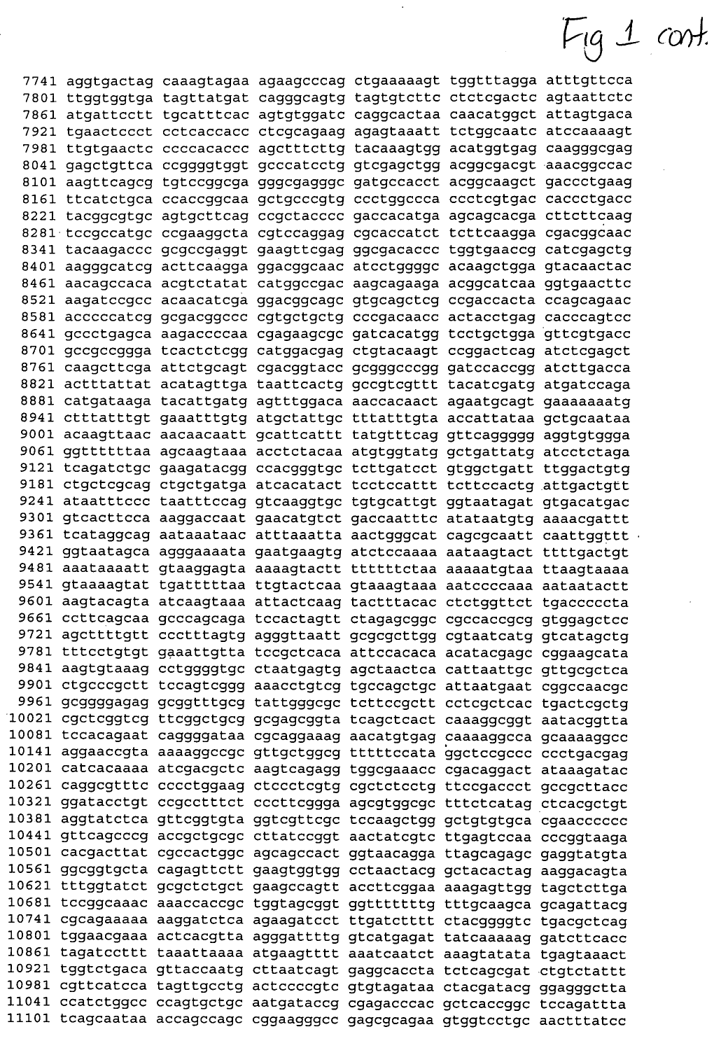

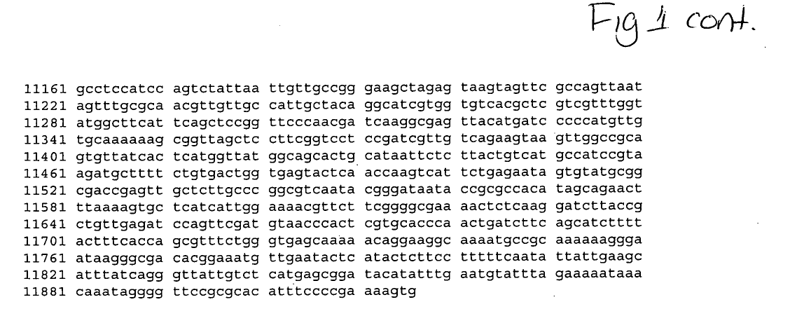

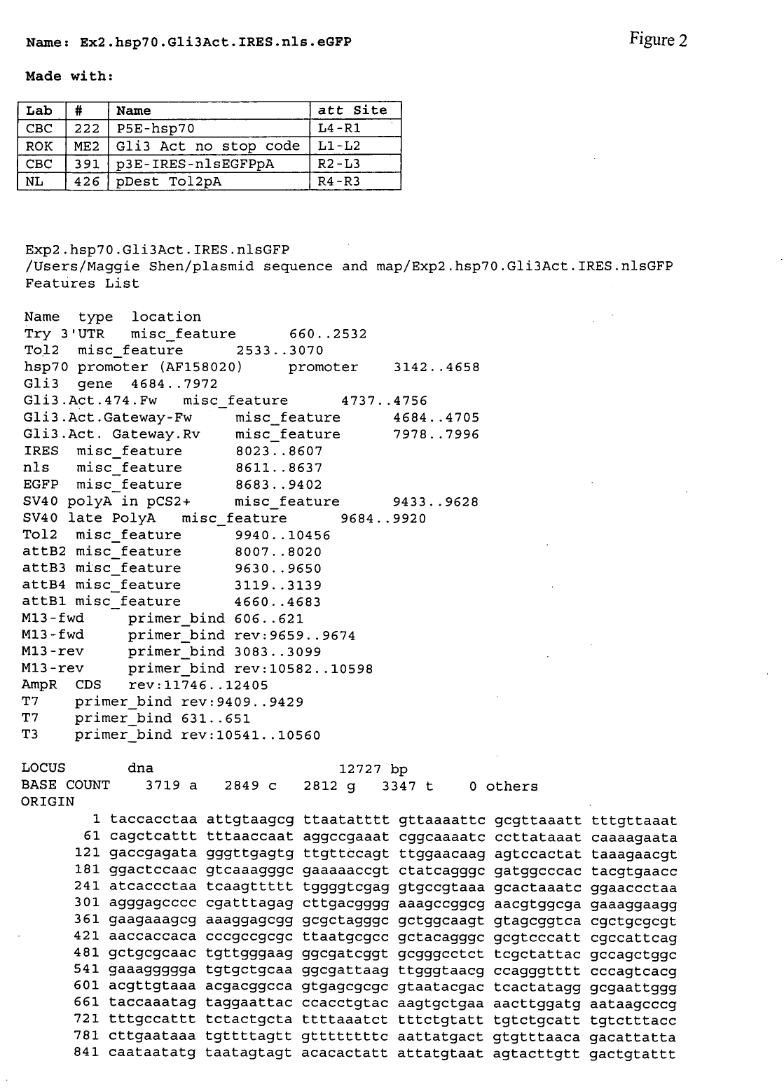

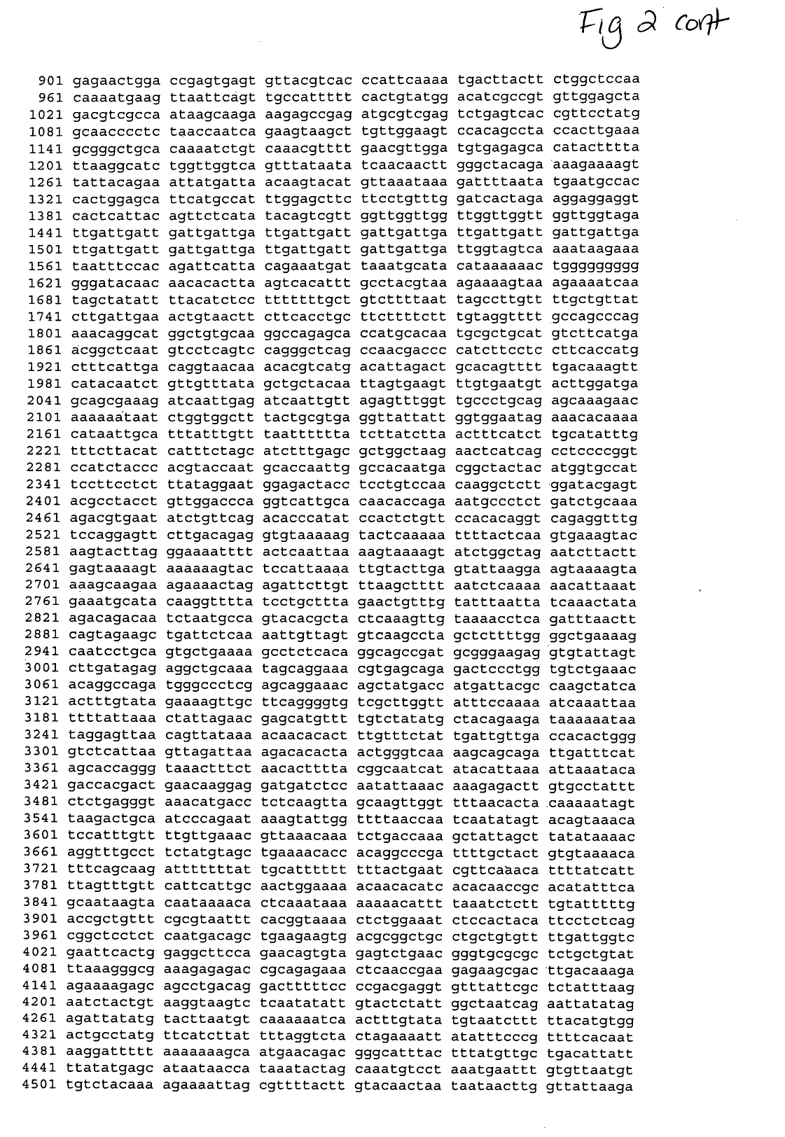

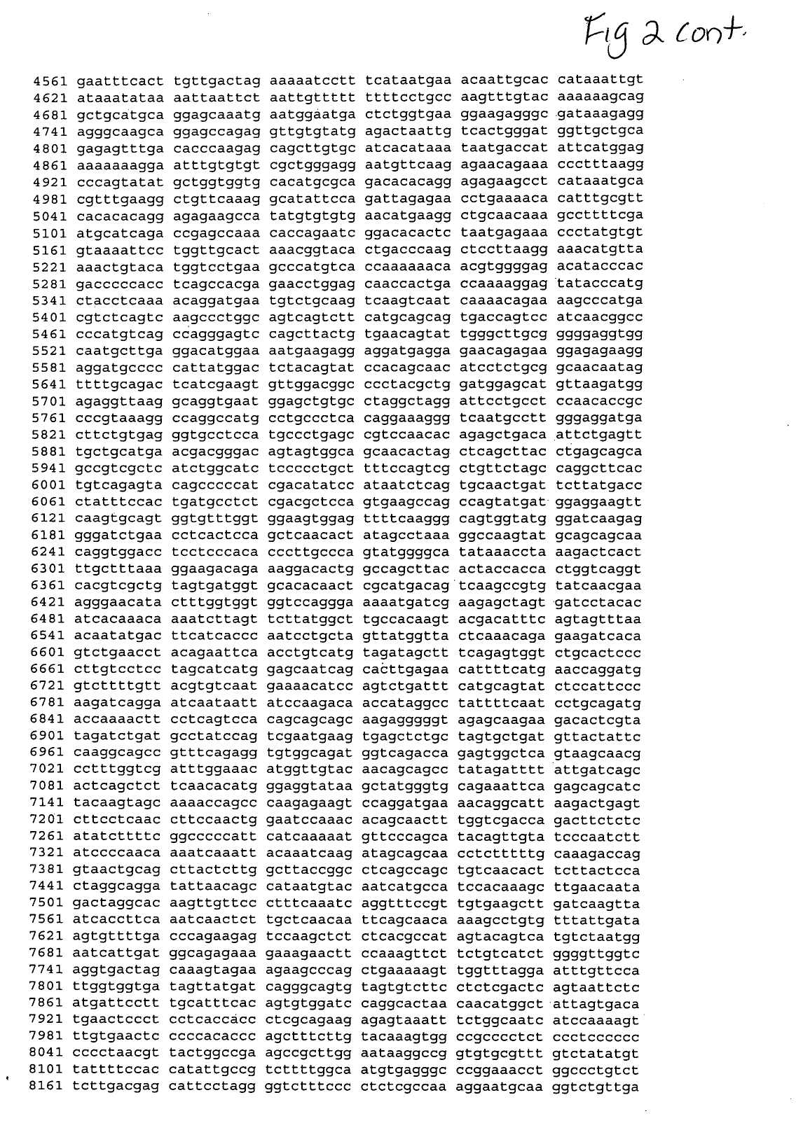

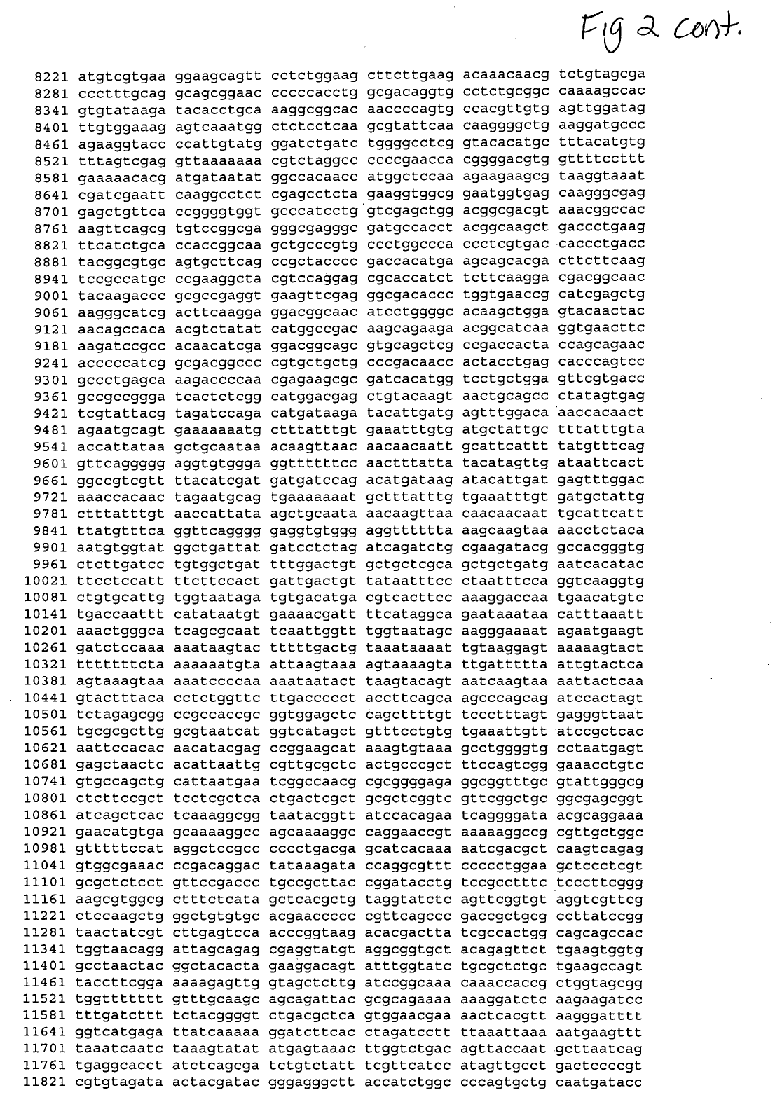

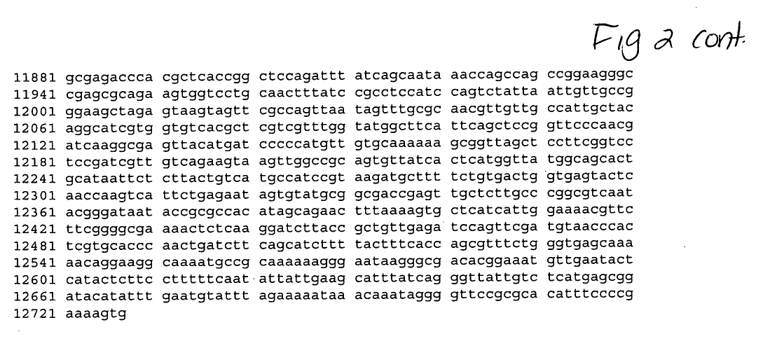

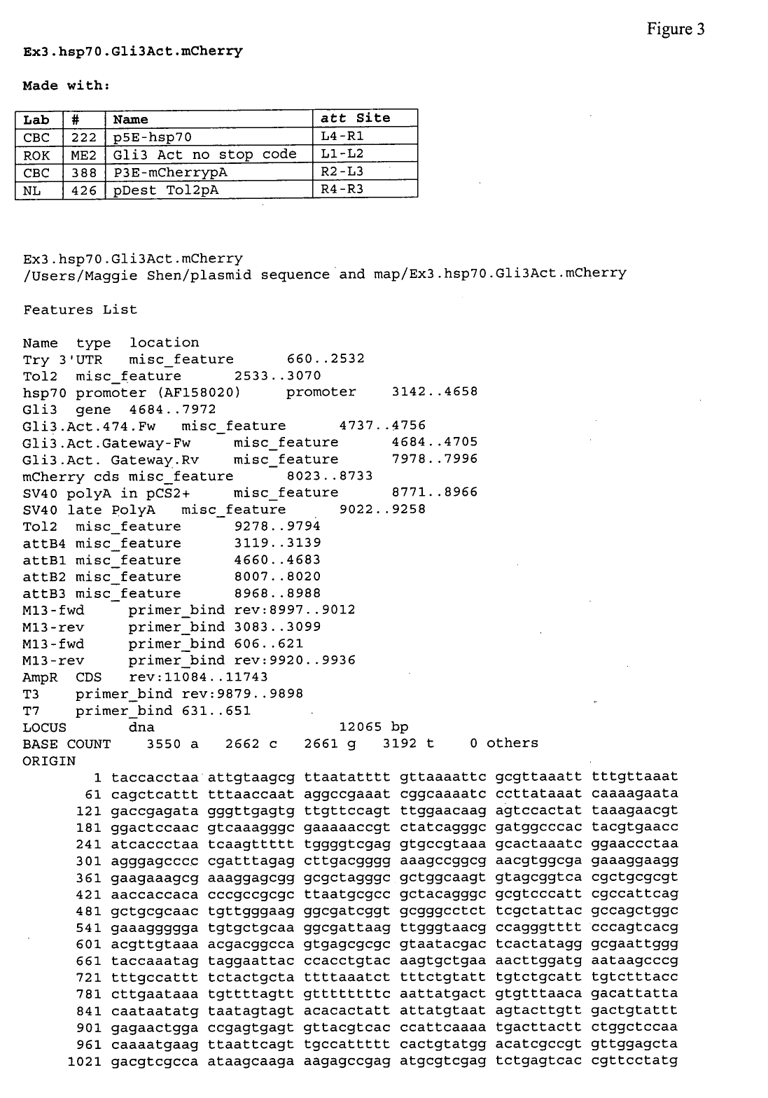

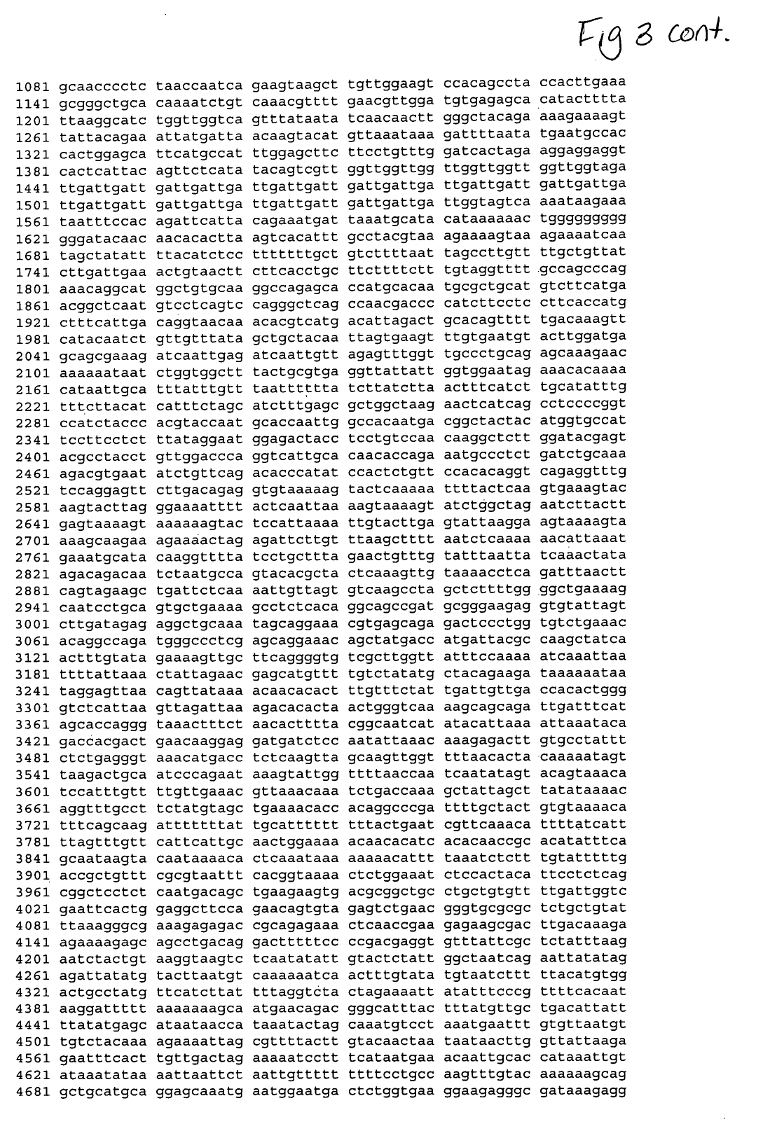

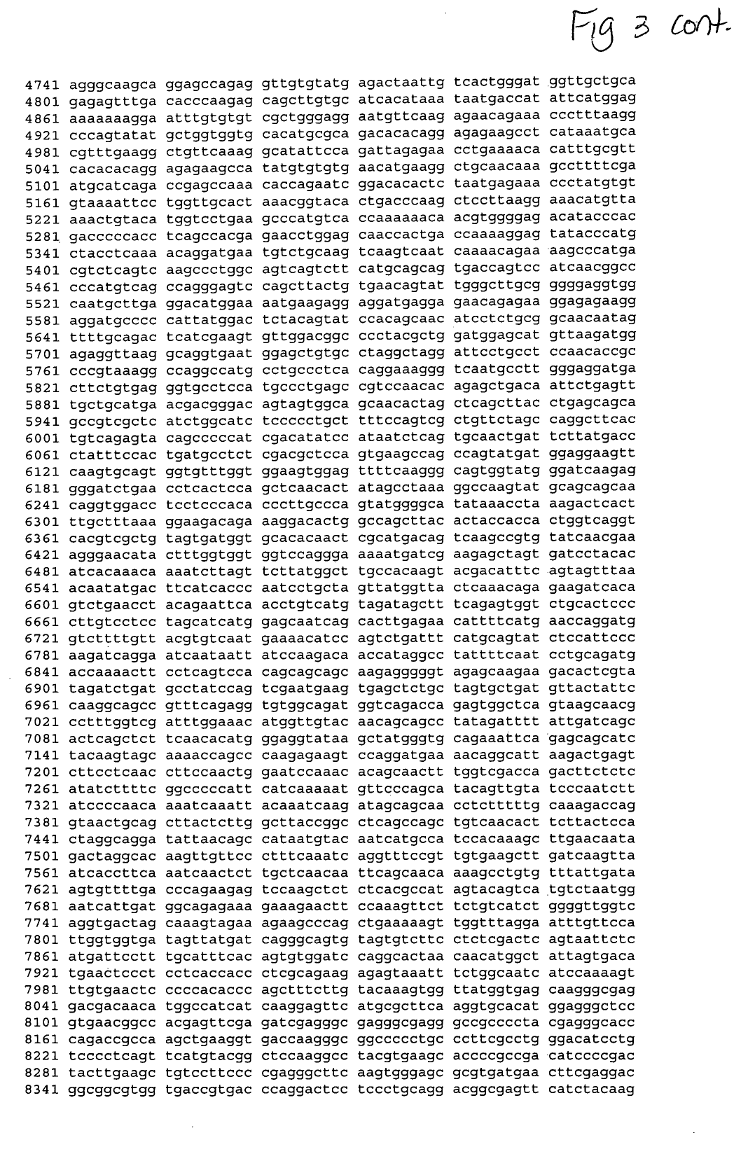

FIG. 3 has the same Gli3 construct as FIGS. 1 and 2, and has mCherry; mcherry: monomeric Cherry, which is synthetic construct monomeric red fluorescent protein gene.

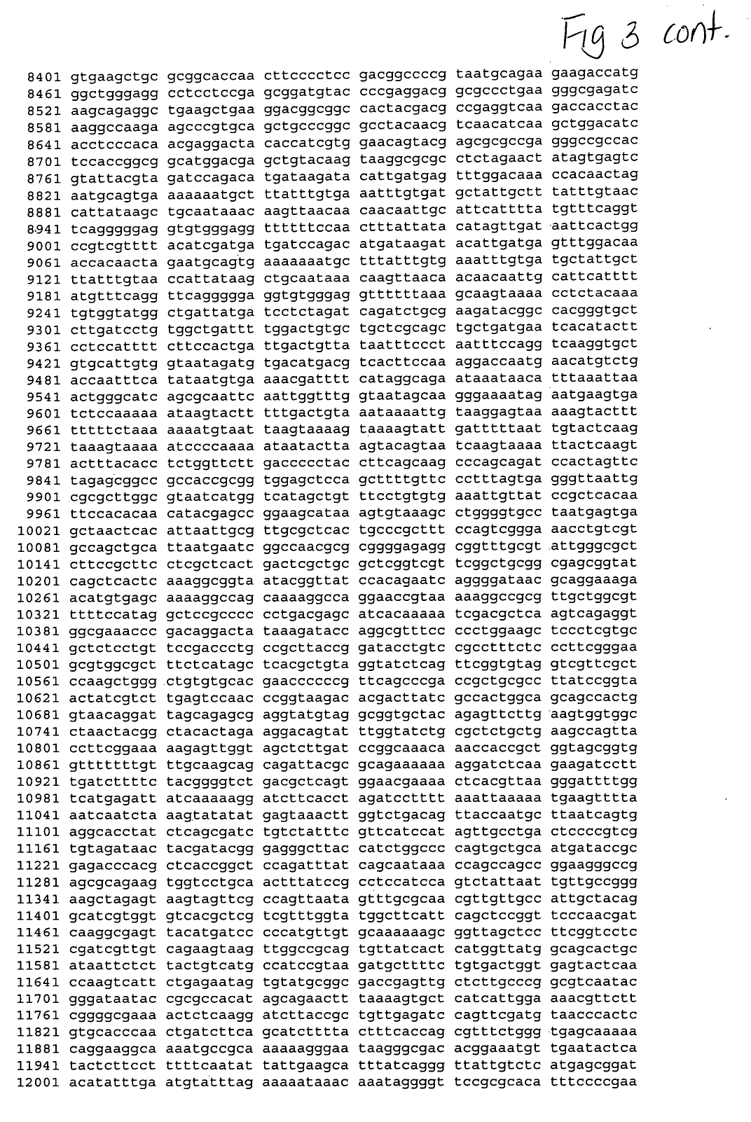

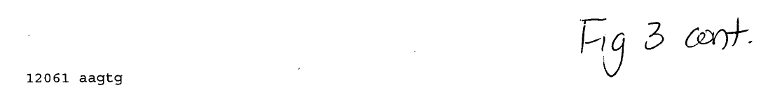

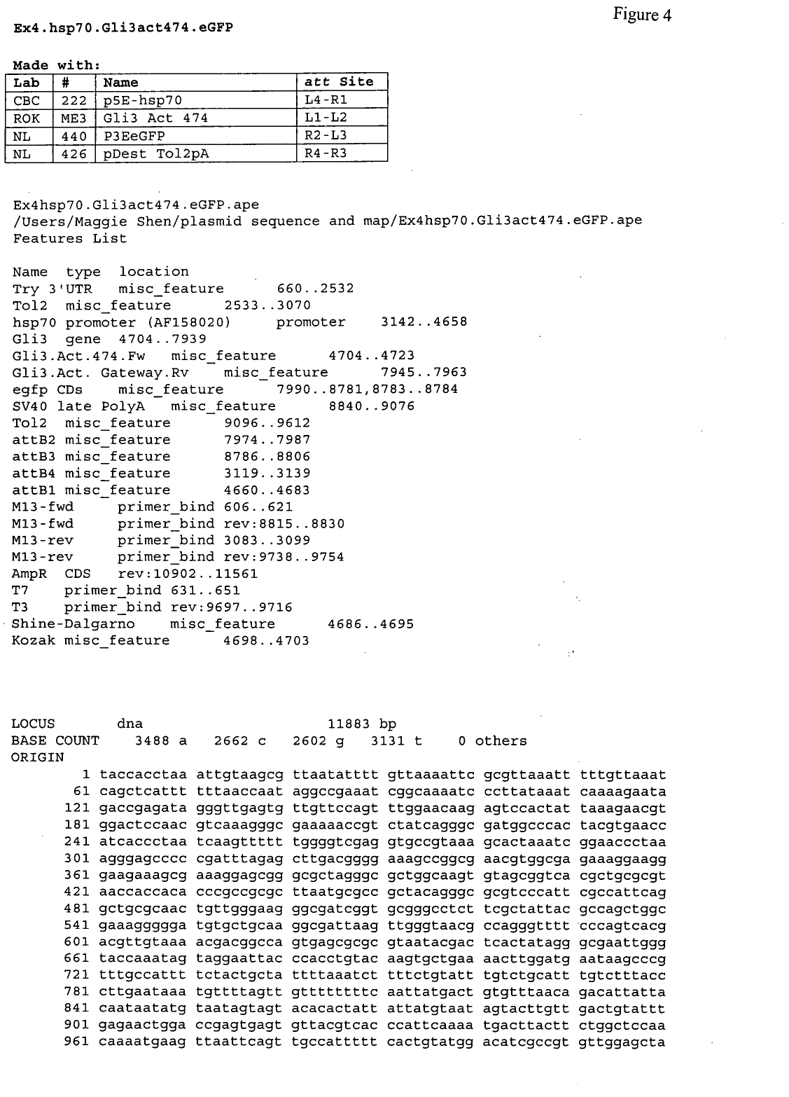

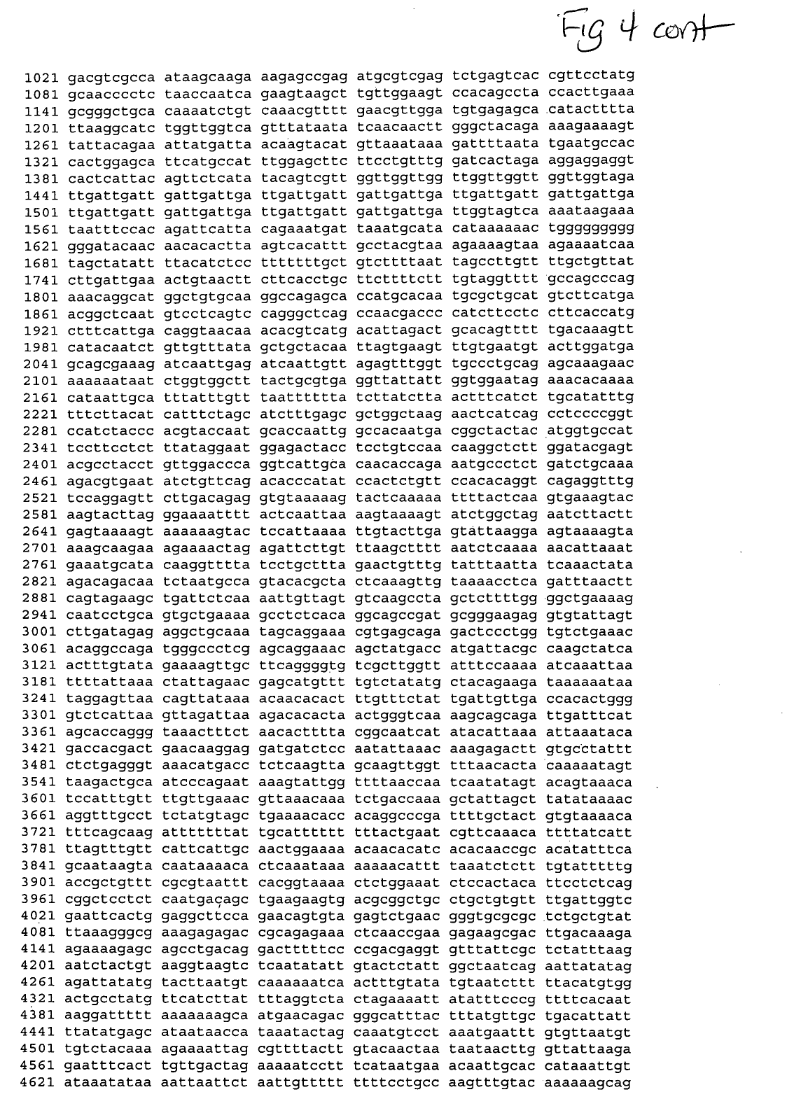

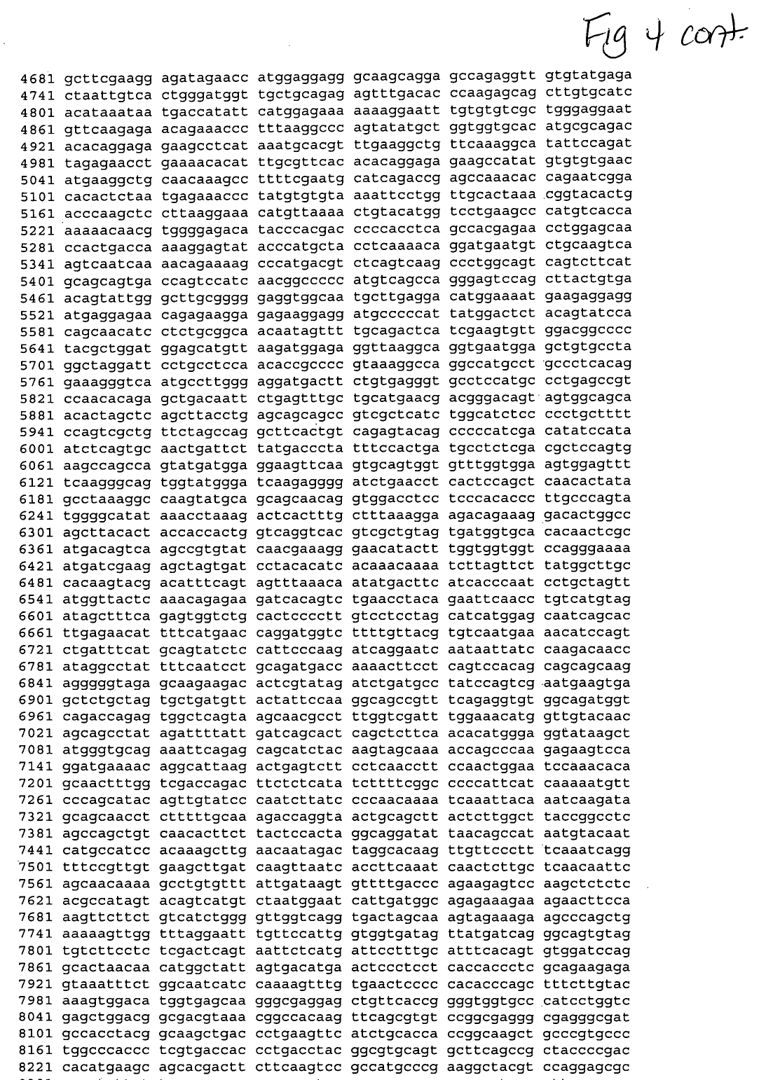

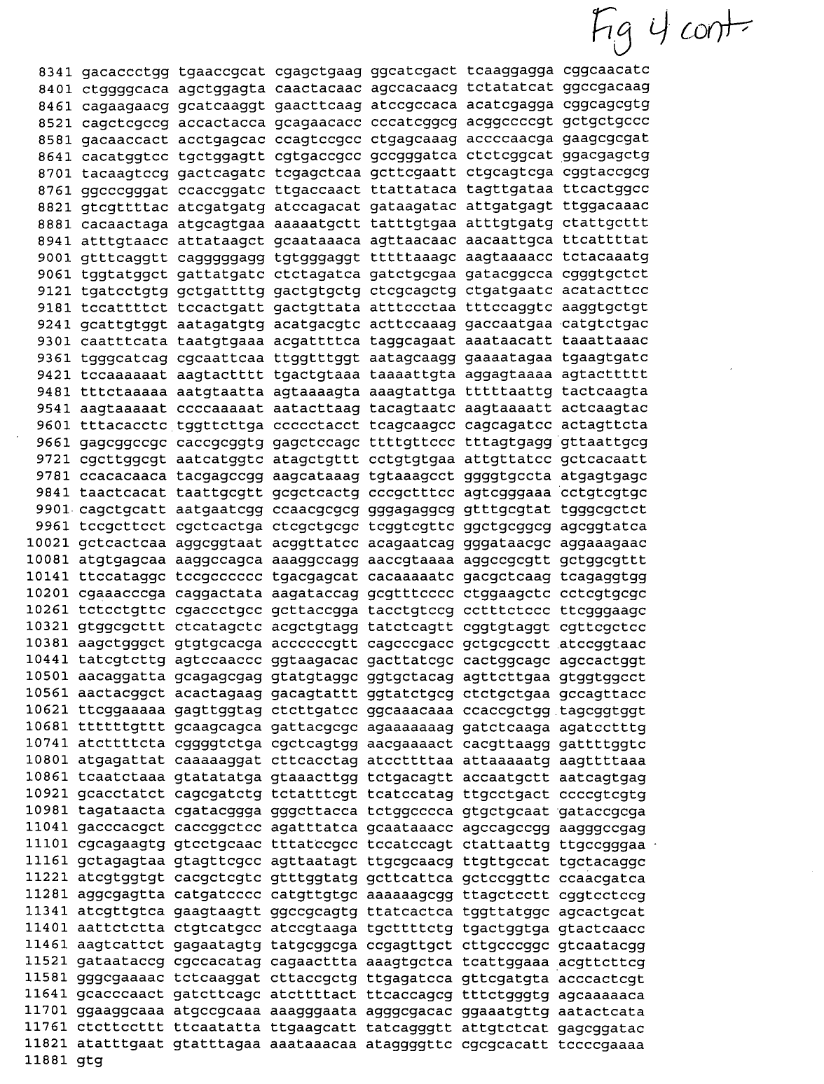

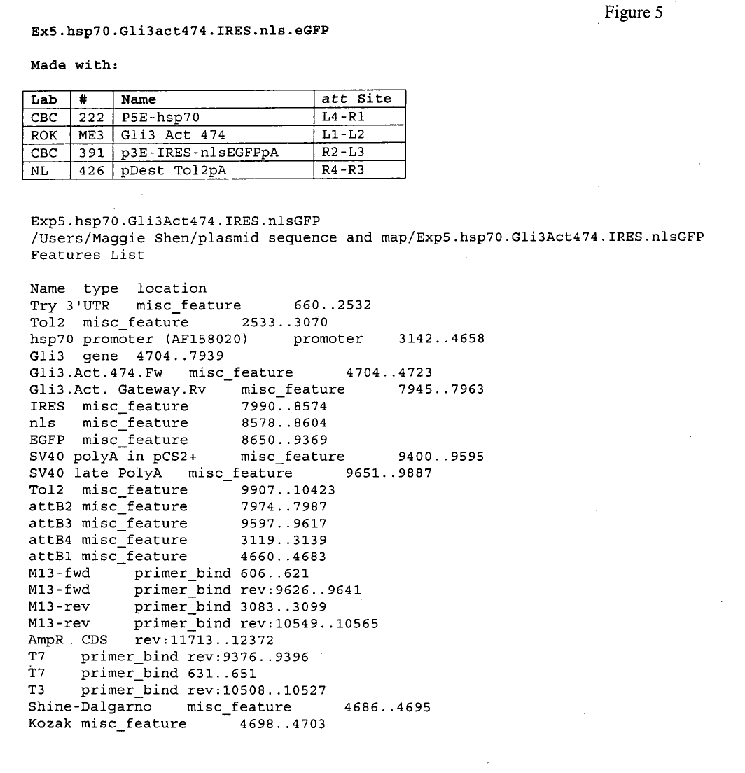

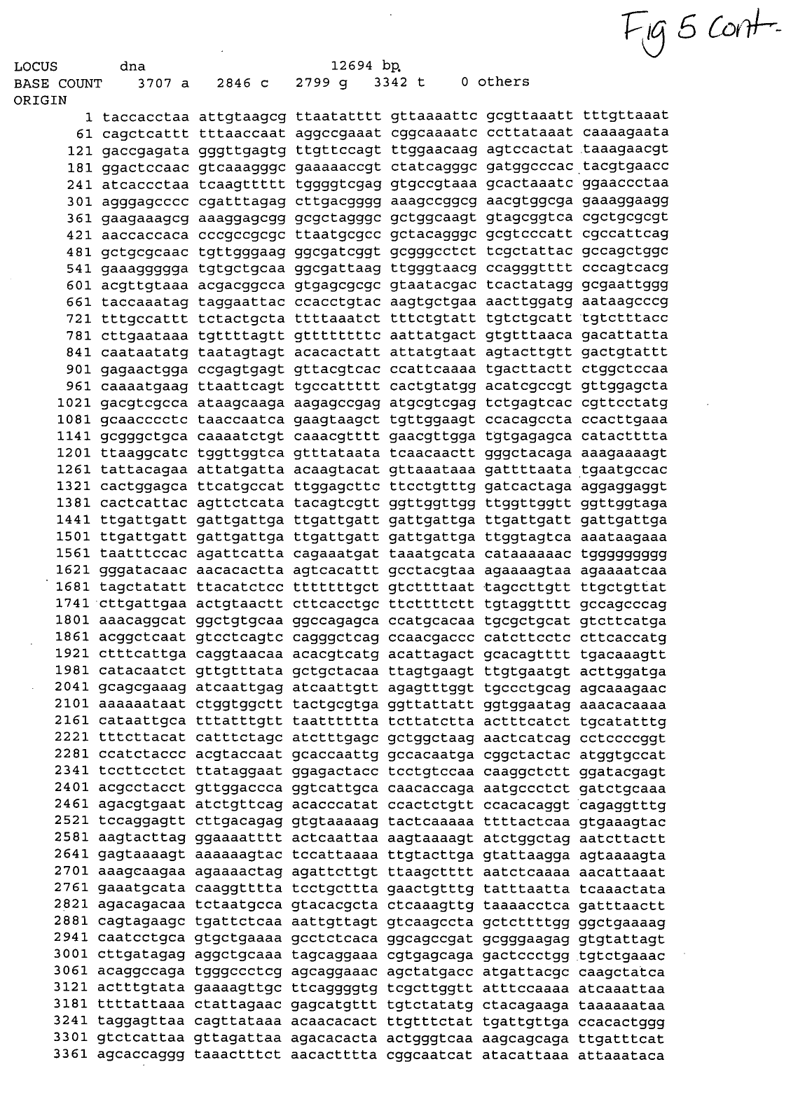

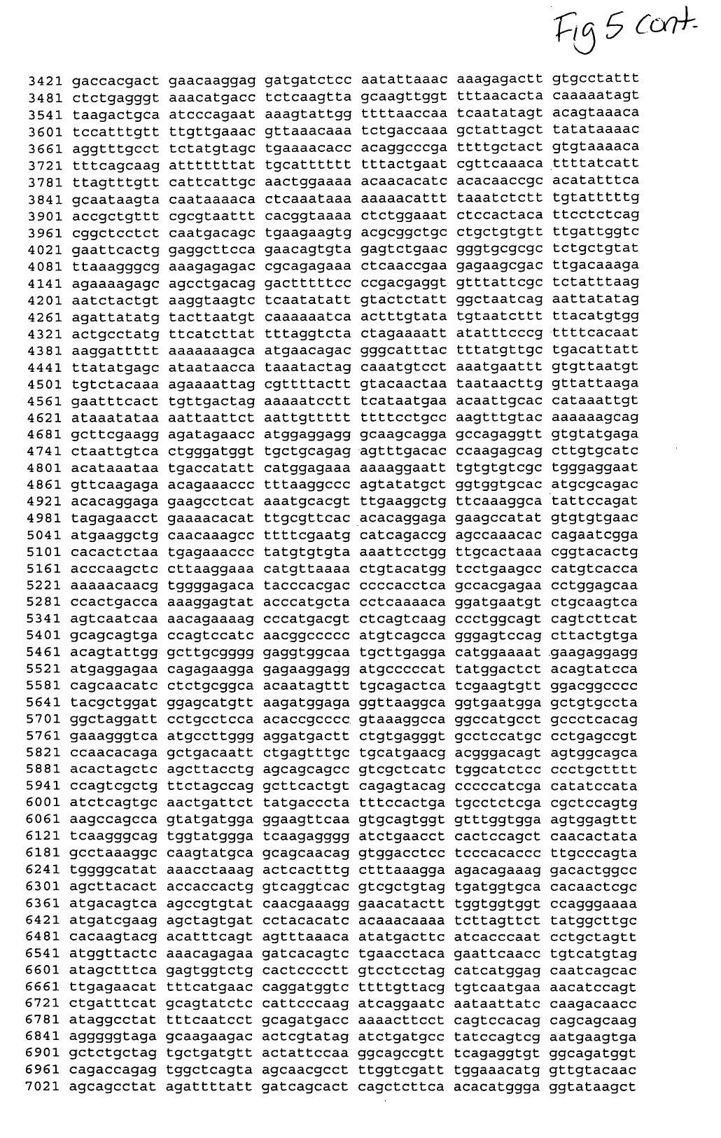

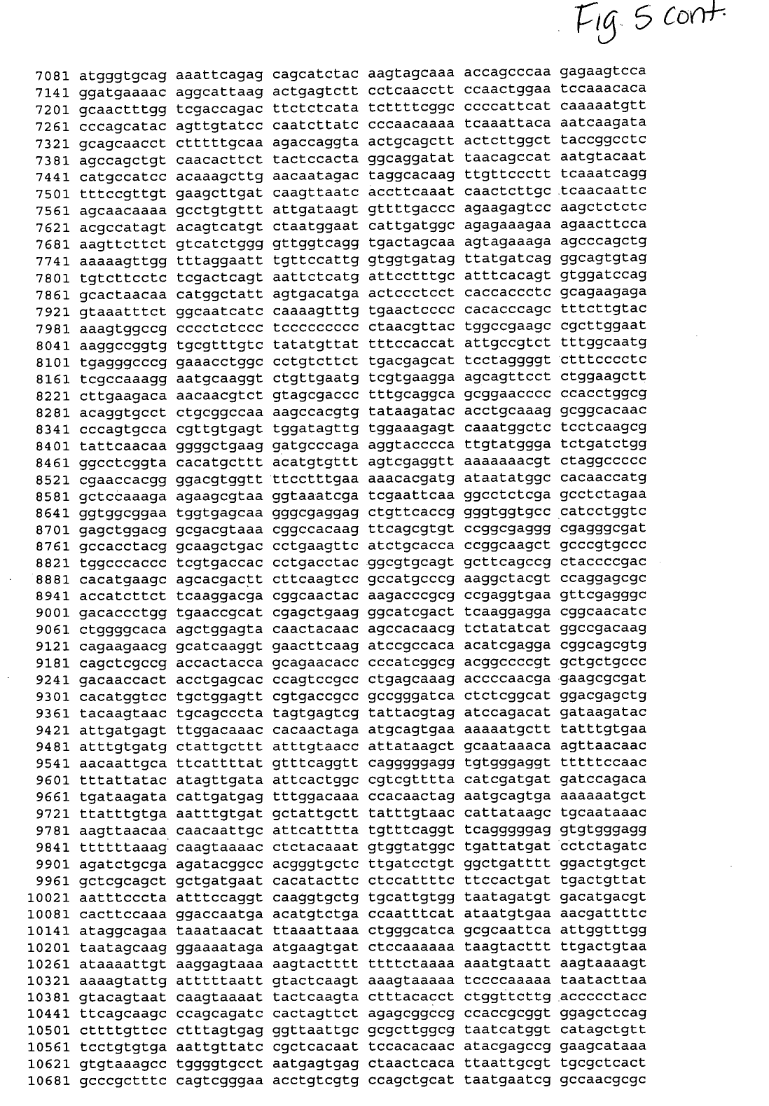

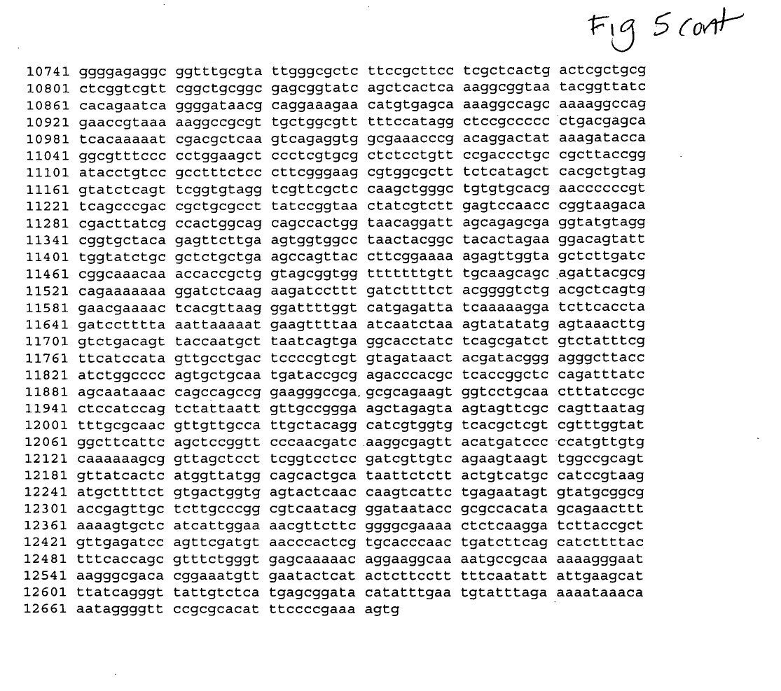

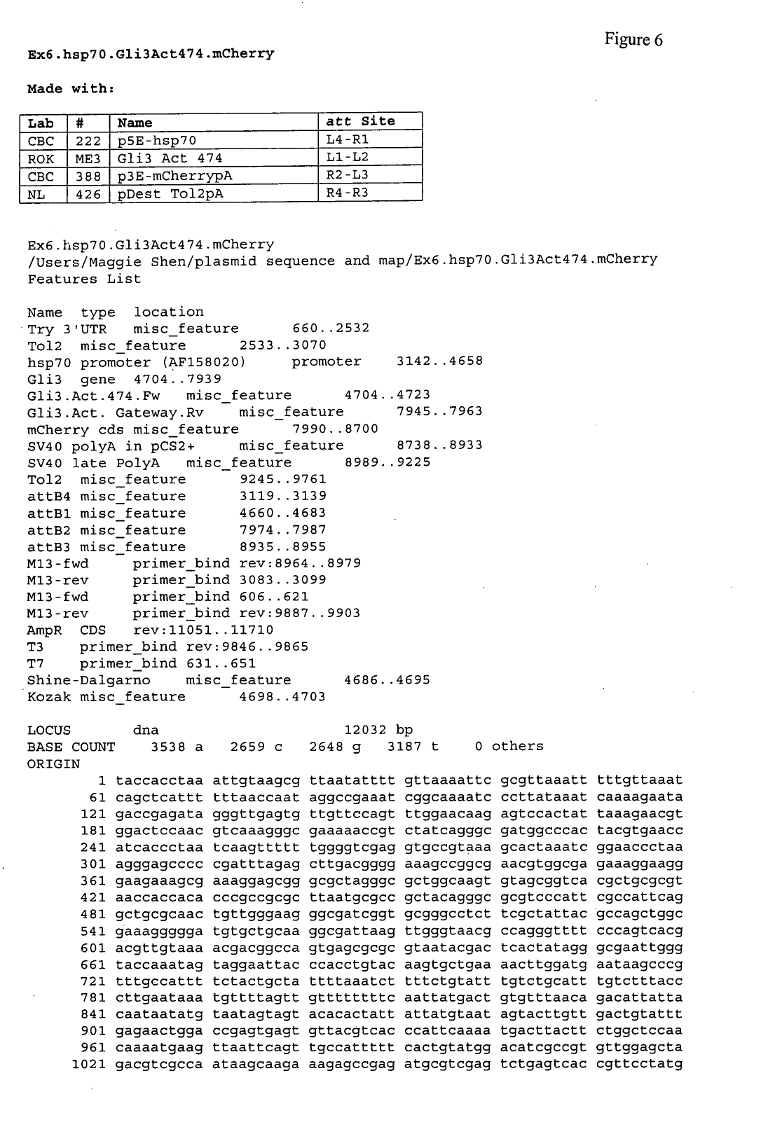

FIGS. 4-6: Gli3act474: Gli3 activactor contains mRNA sequence 1805-5062 (CDs, 386-5047; 5045 nucleotide has been changed from T to A to avoid stop code), so the first amino acid will be number 474 amino acid of the zfGli3.

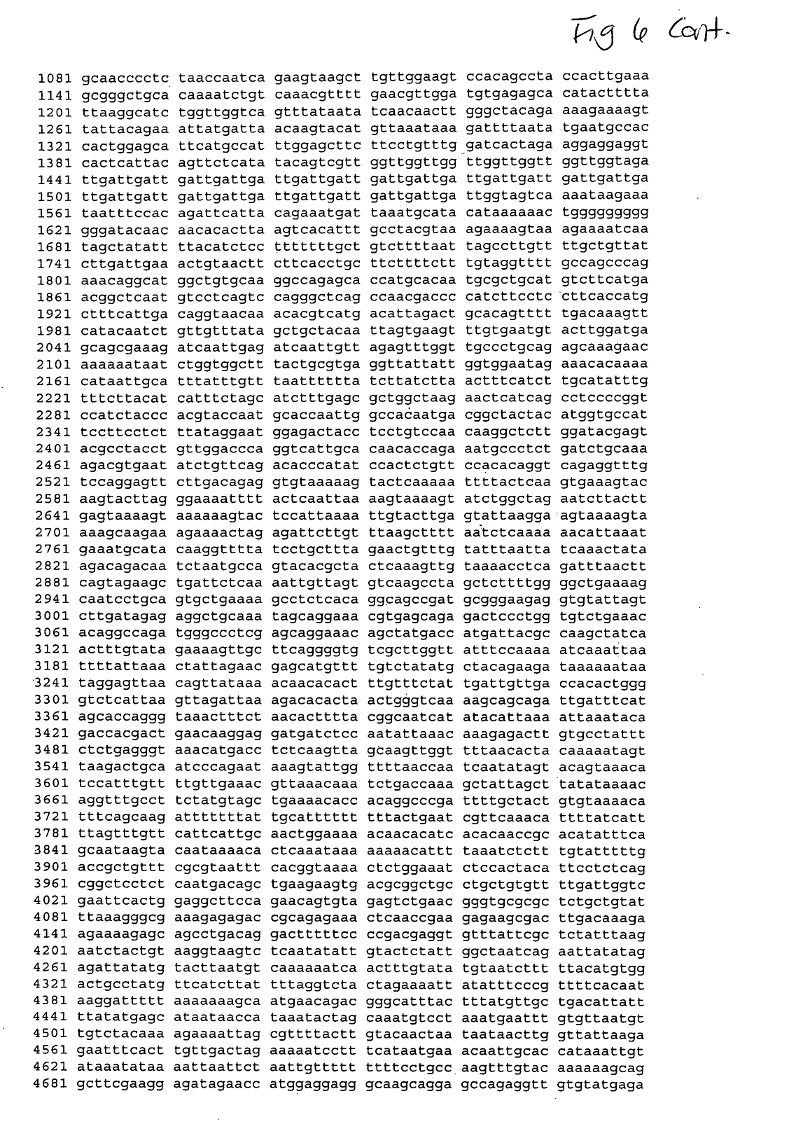

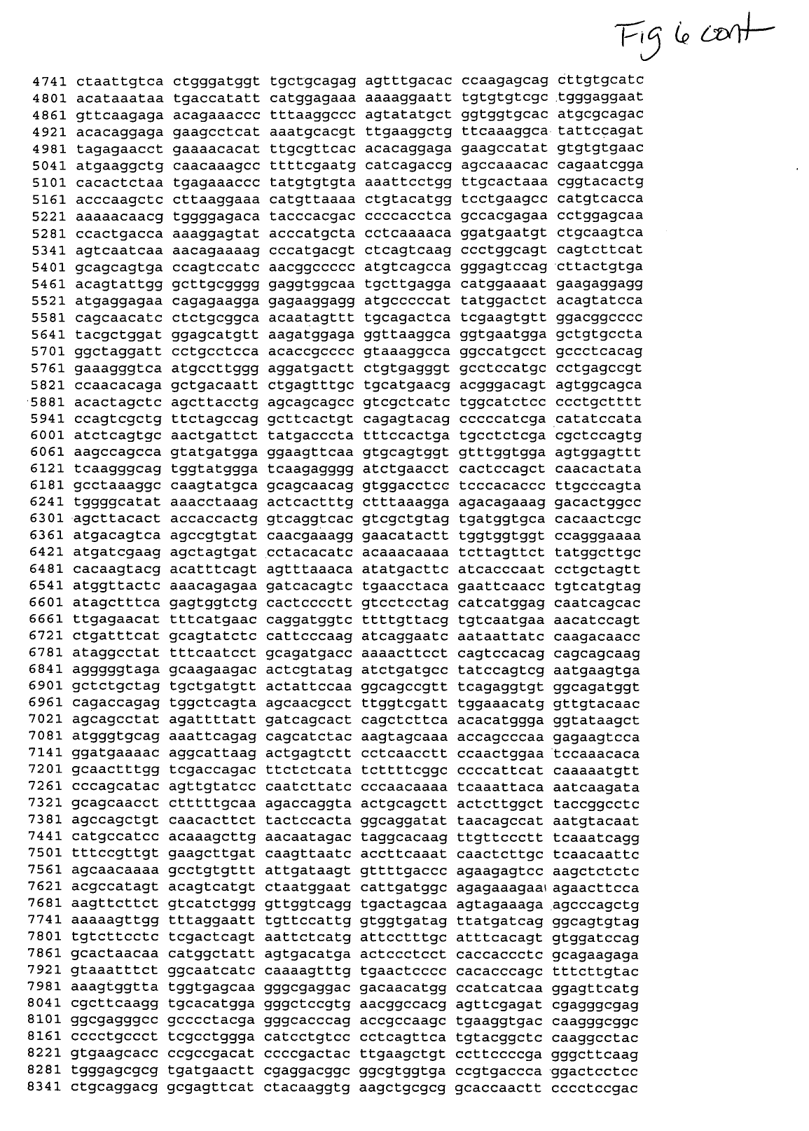

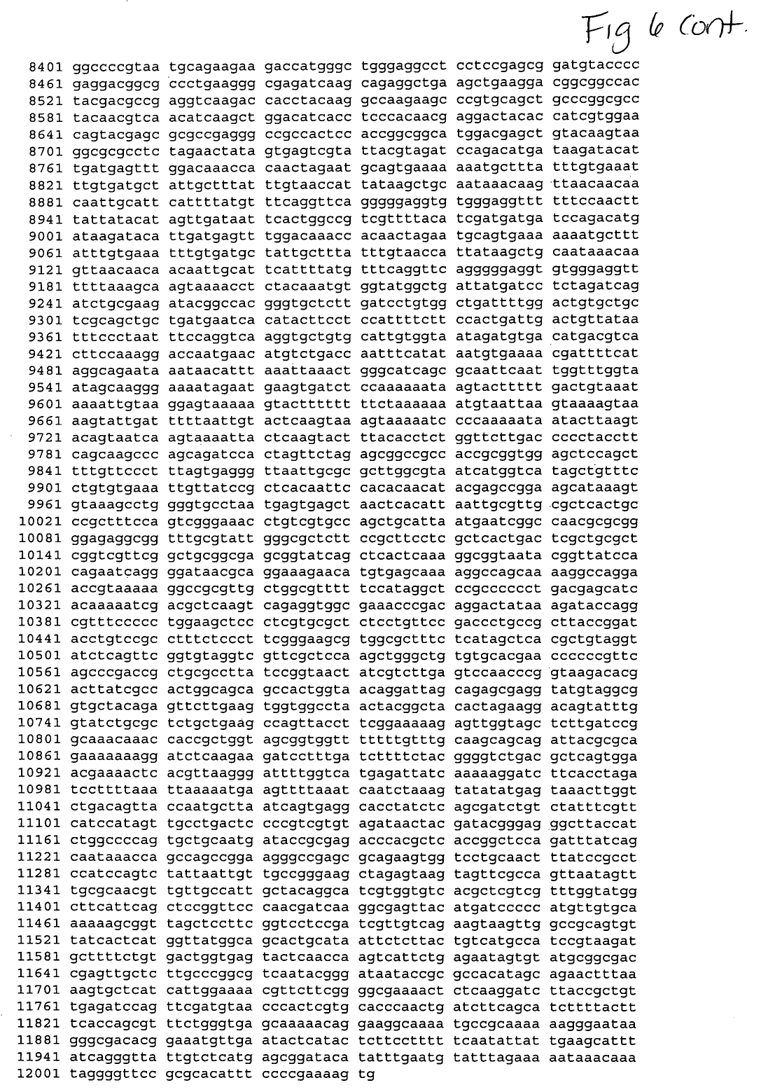

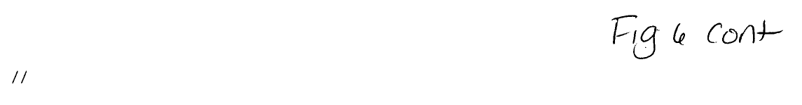

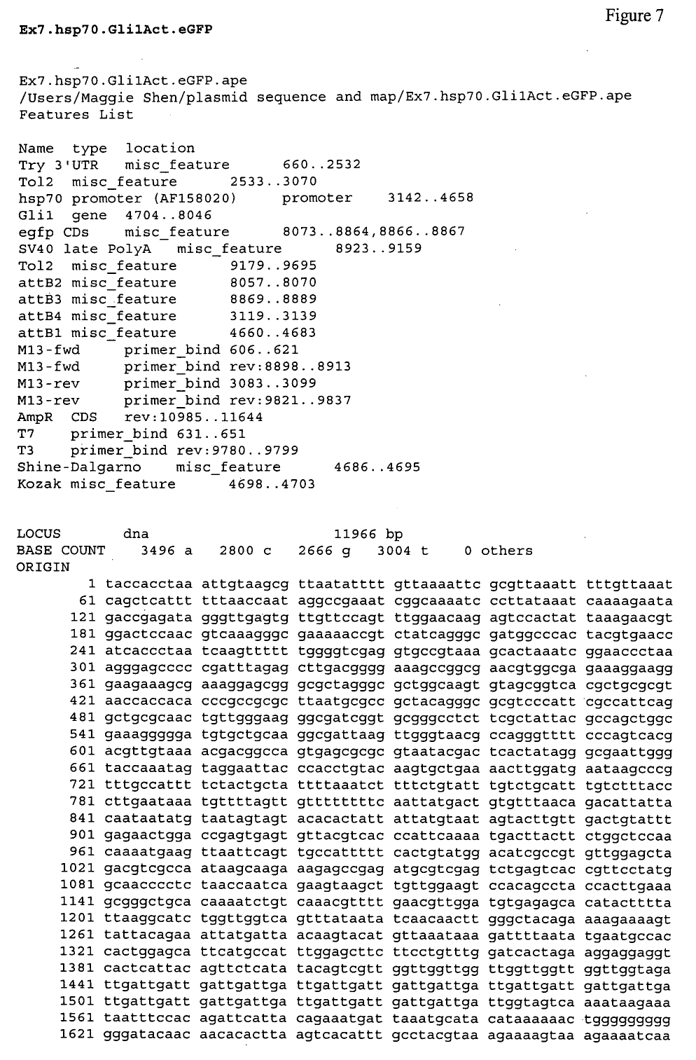

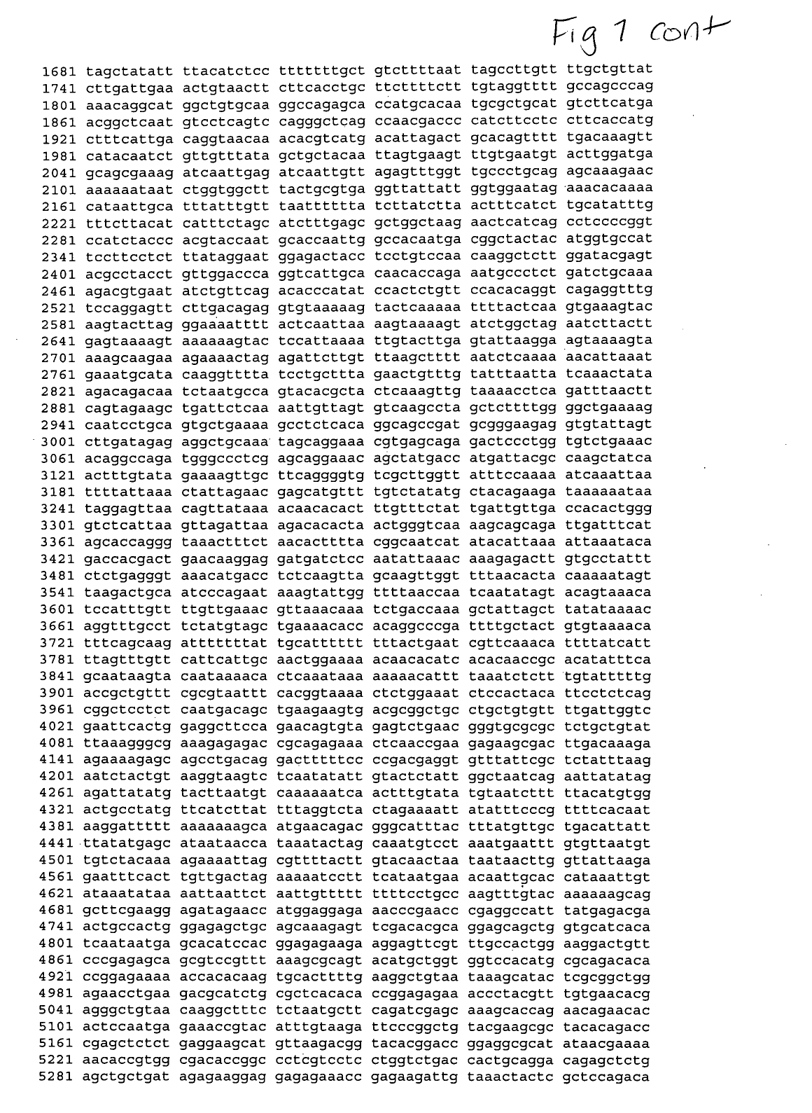

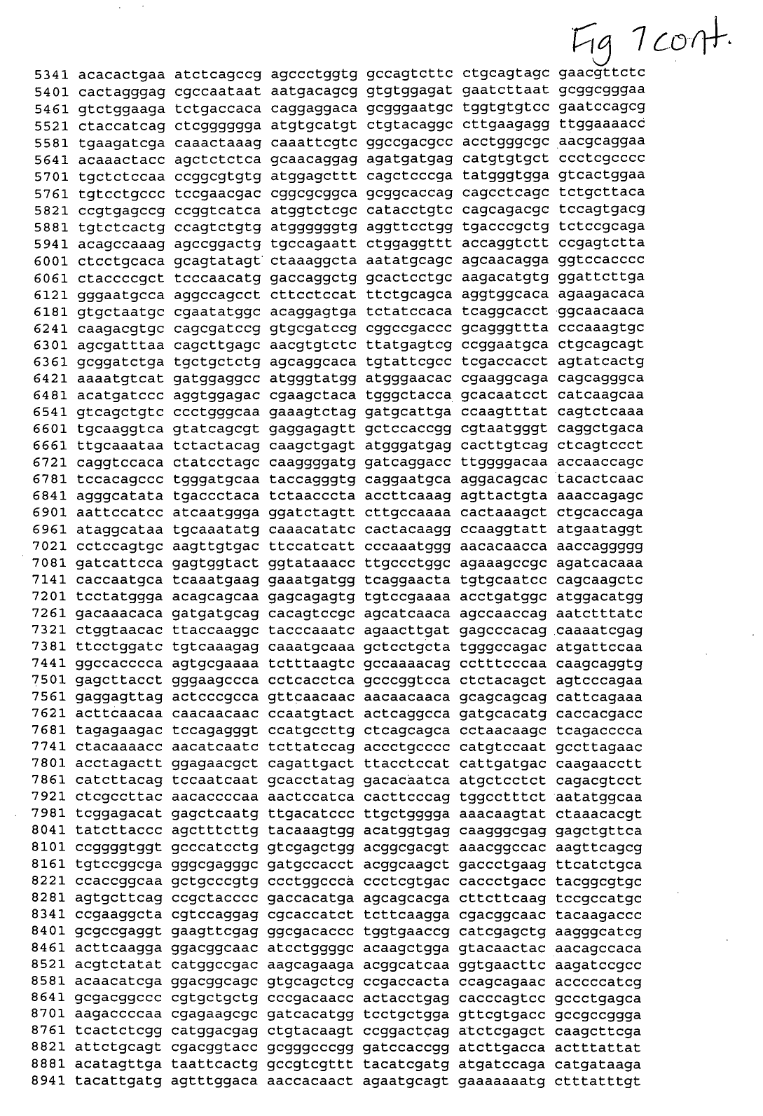

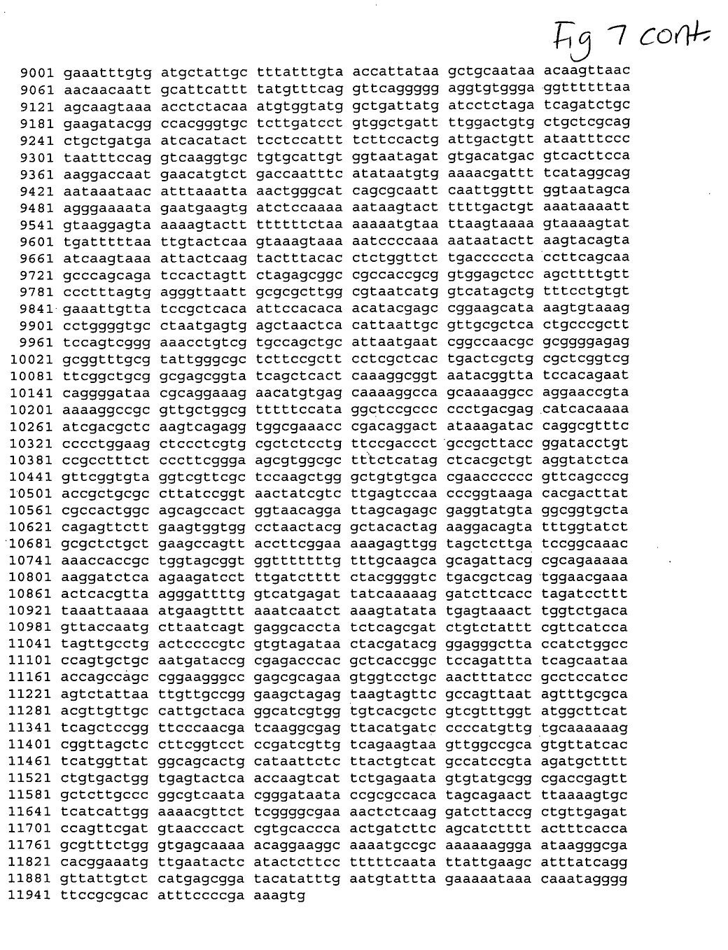

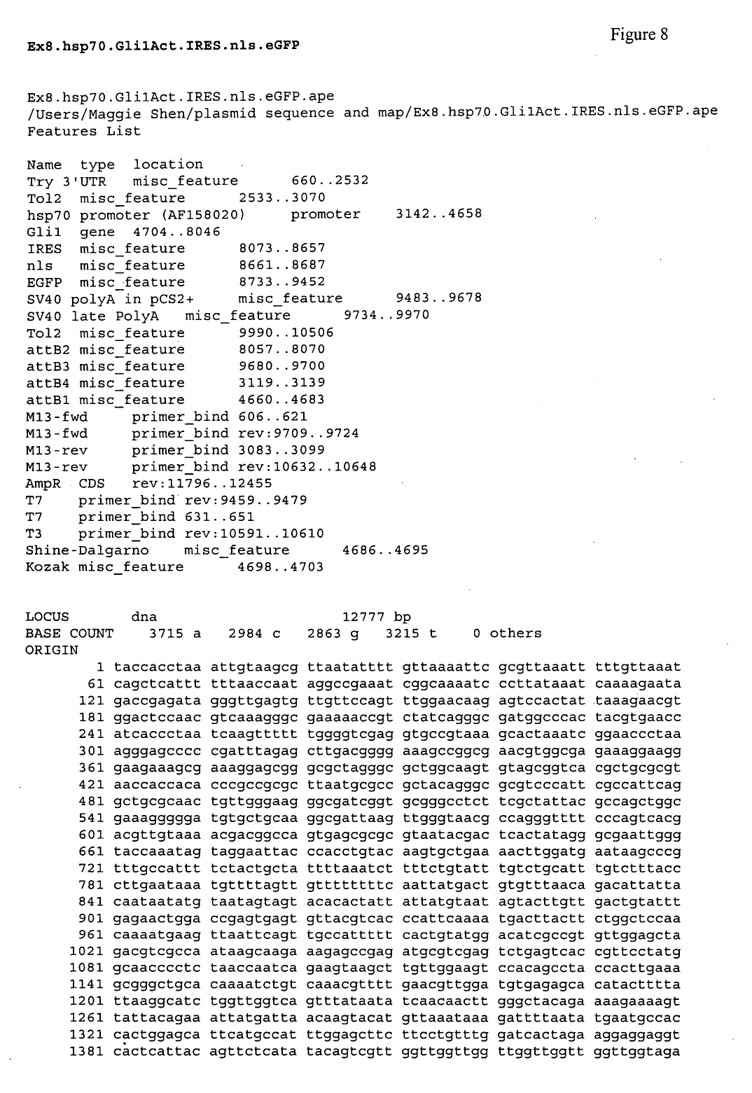

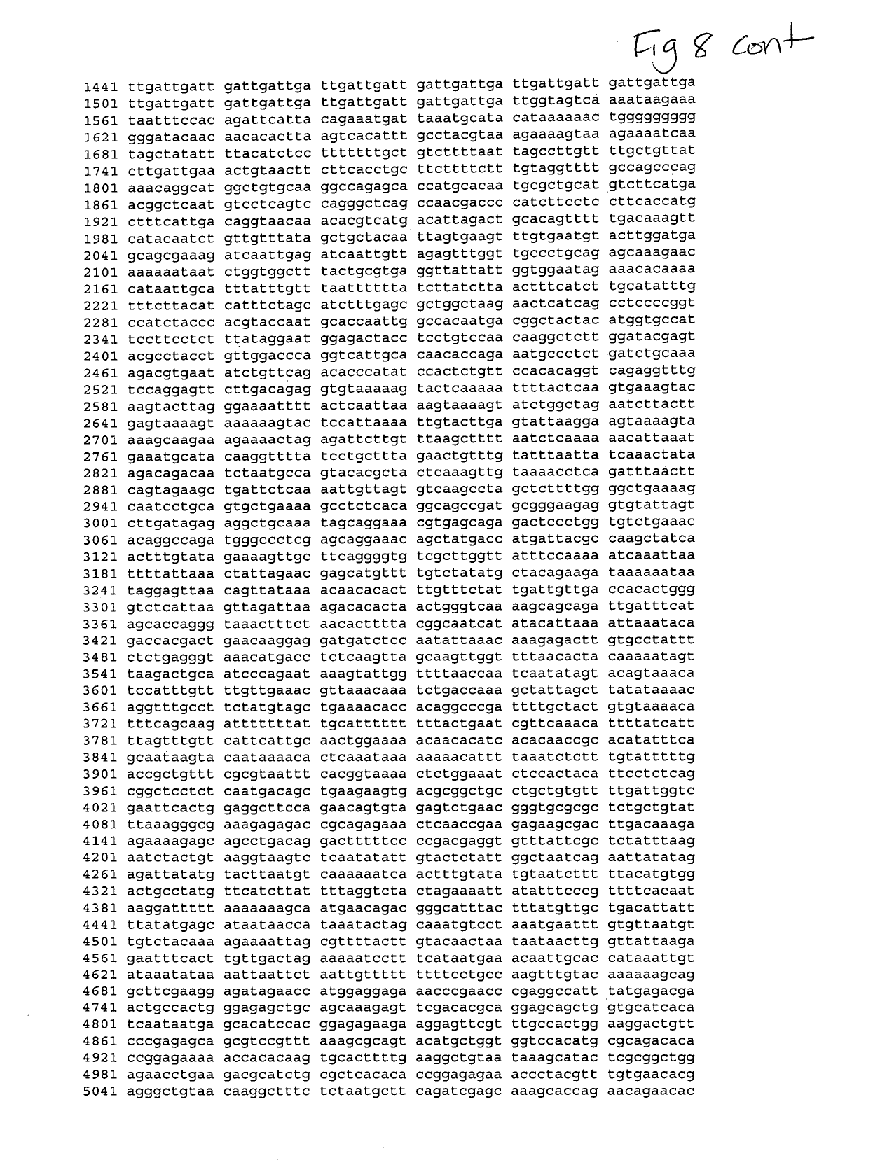

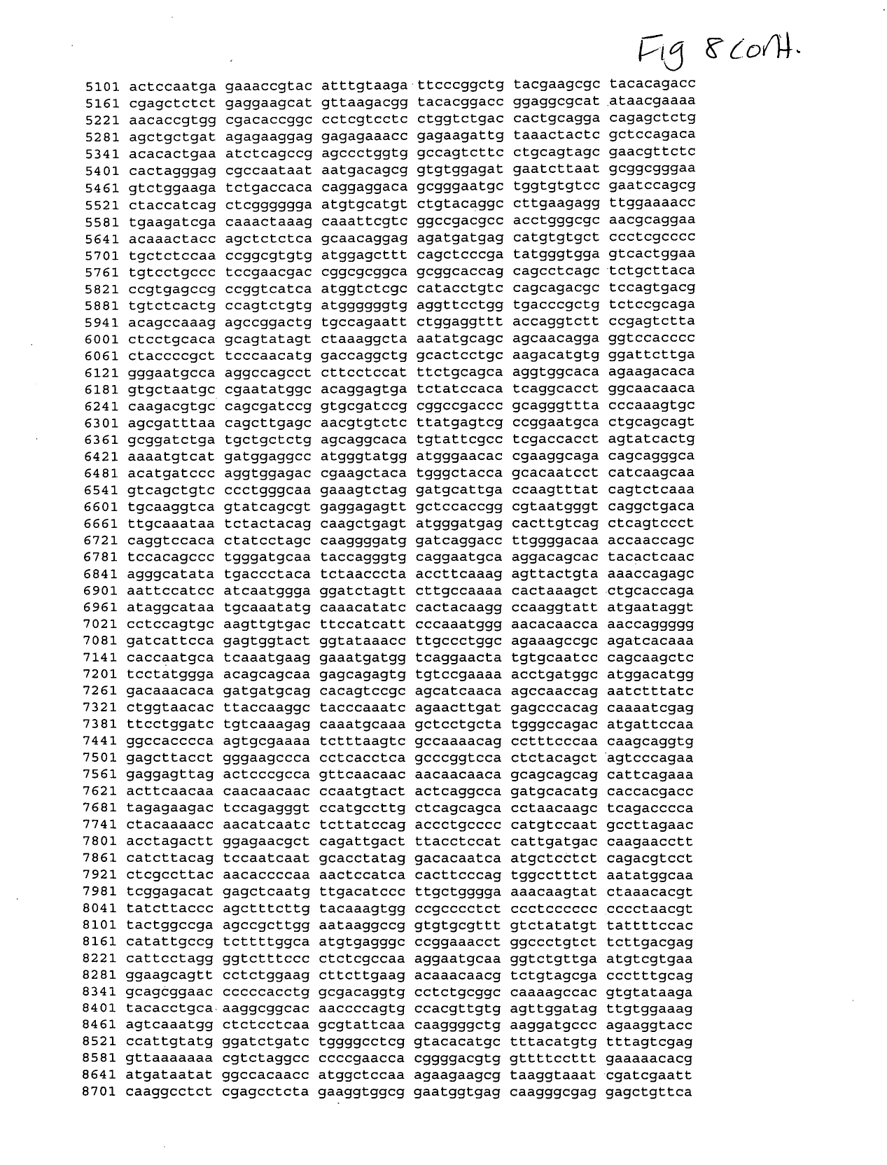

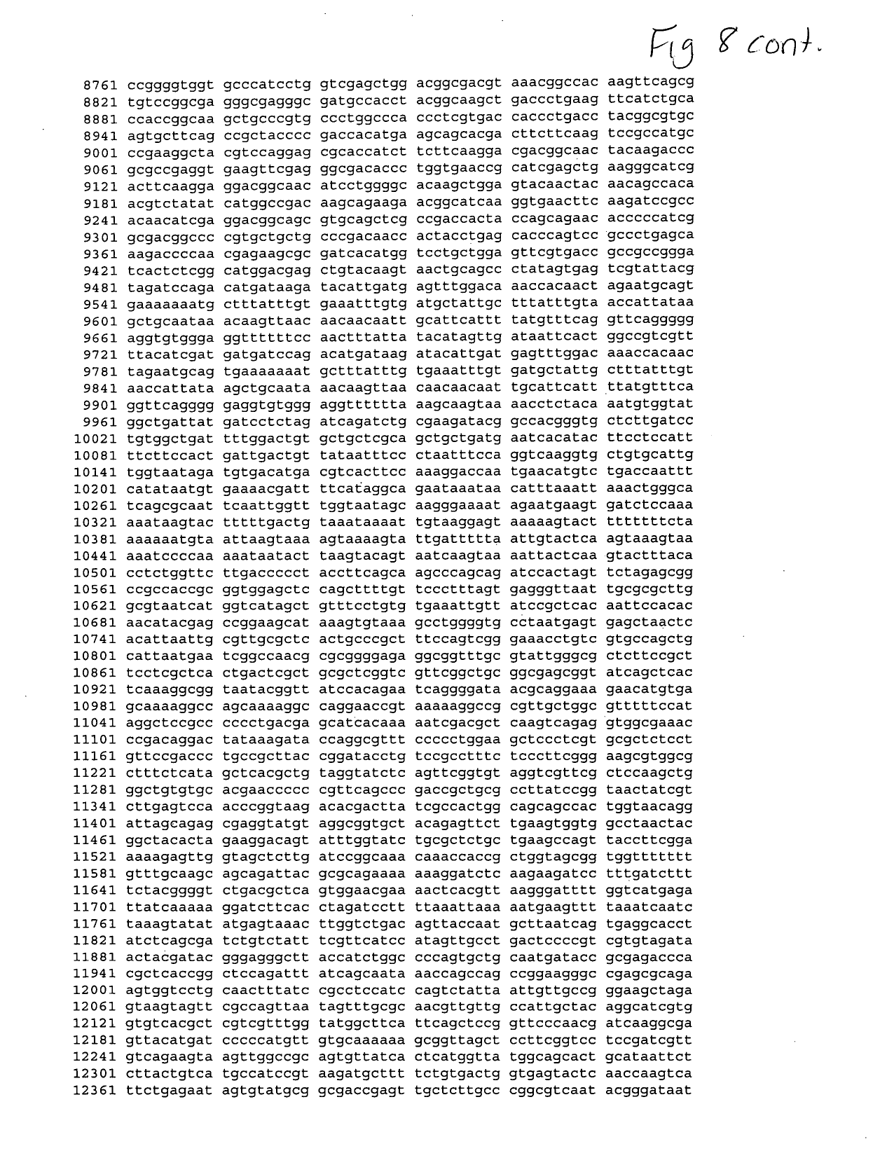

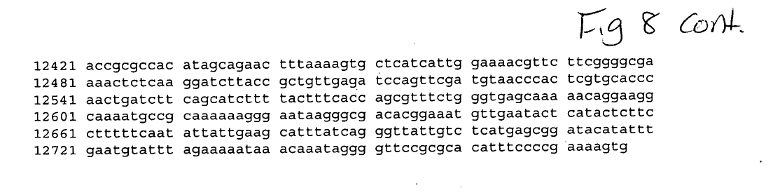

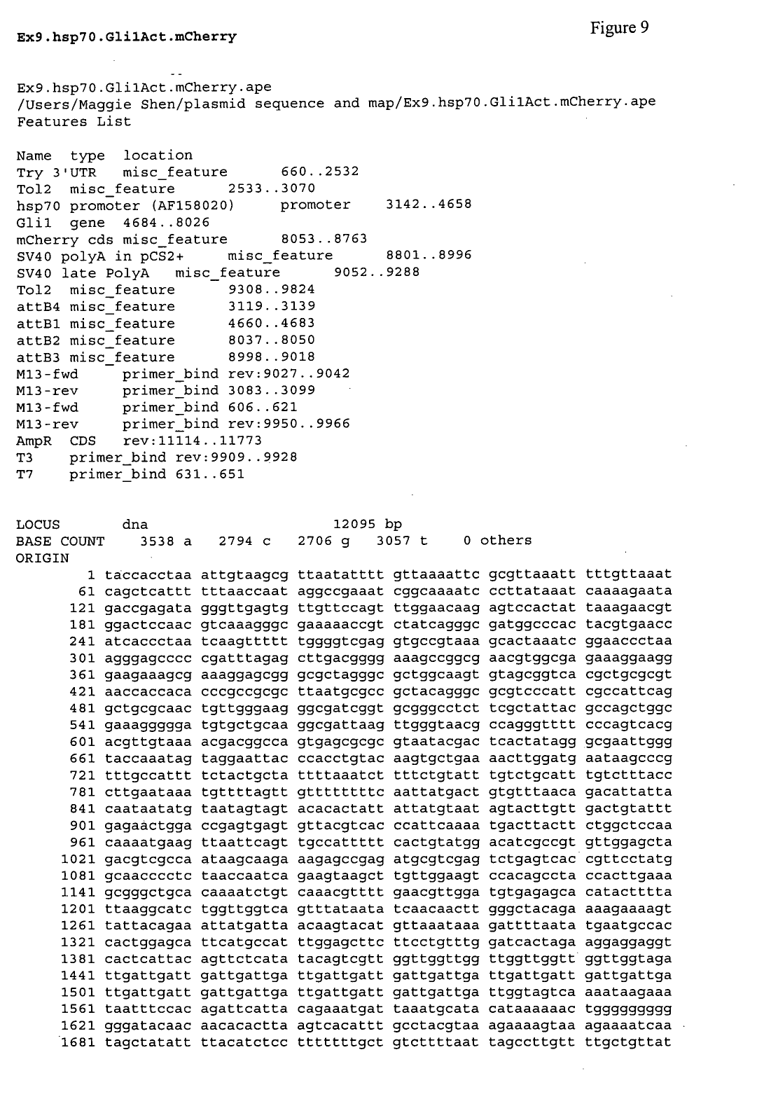

FIGS. 7-9: Gli1Act: Gli1 activator, contains Gli1 mRNA sequence 1189-4531.

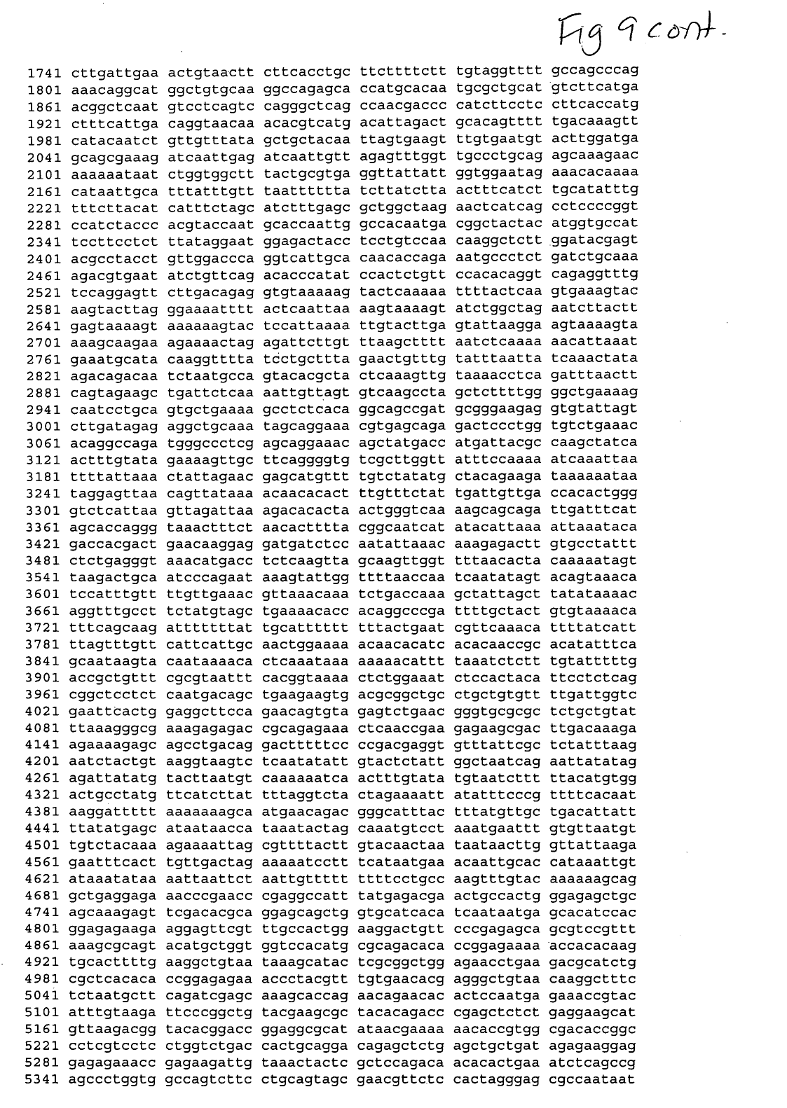

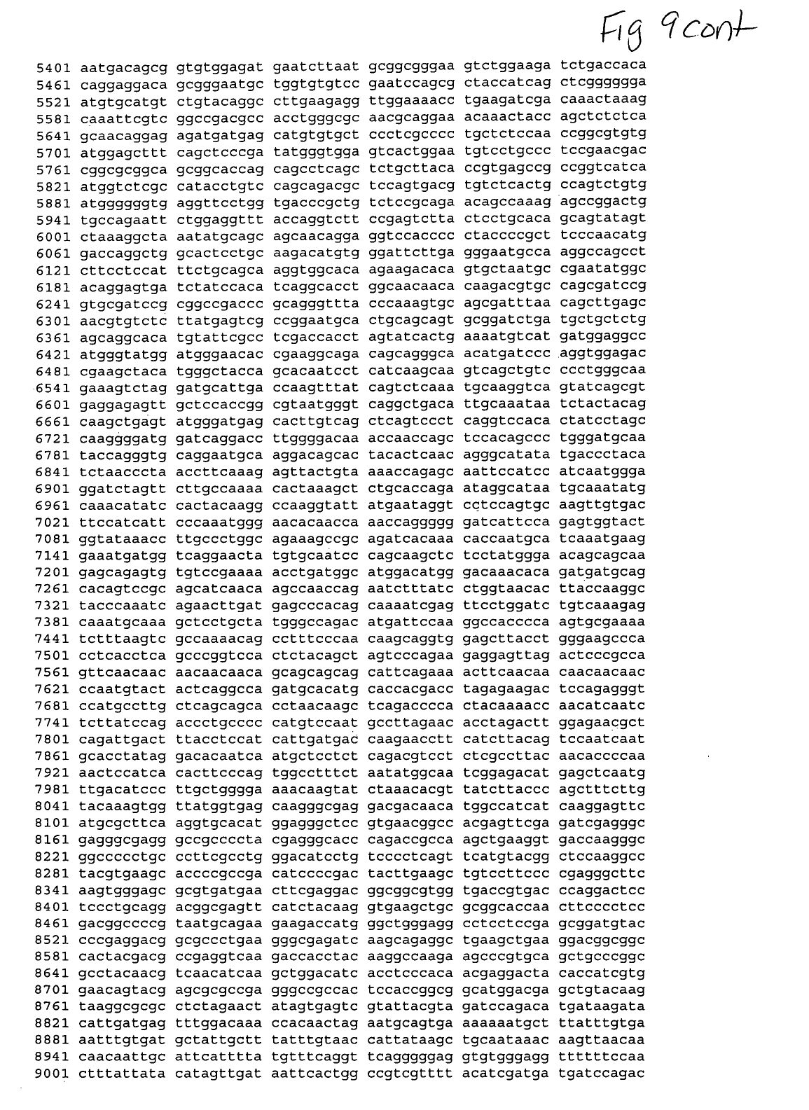

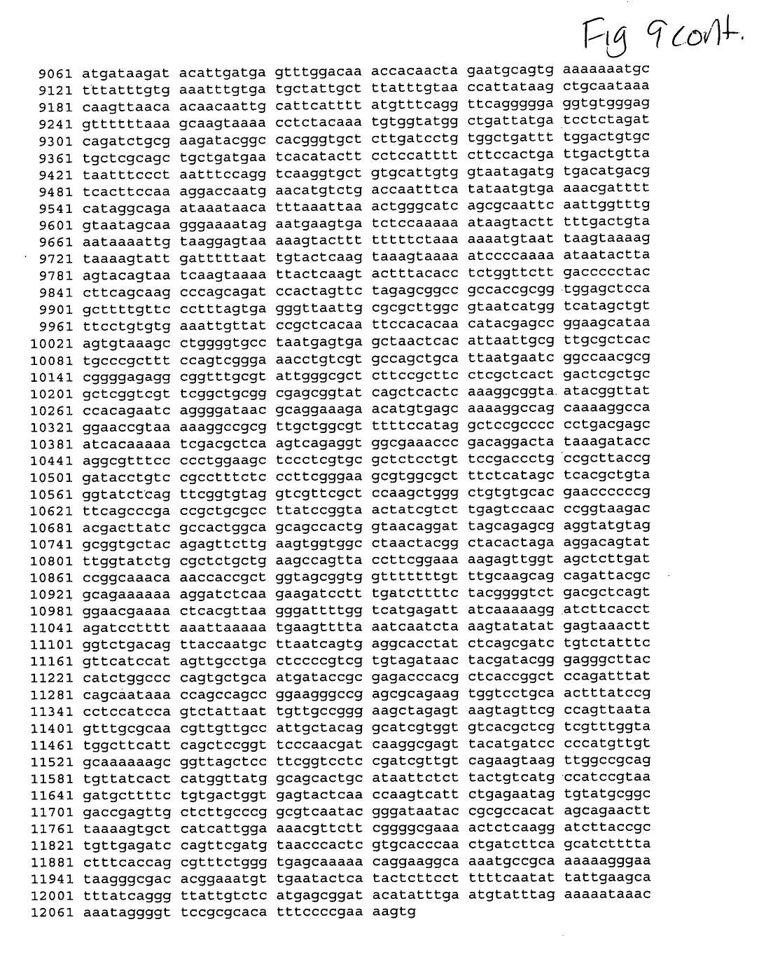

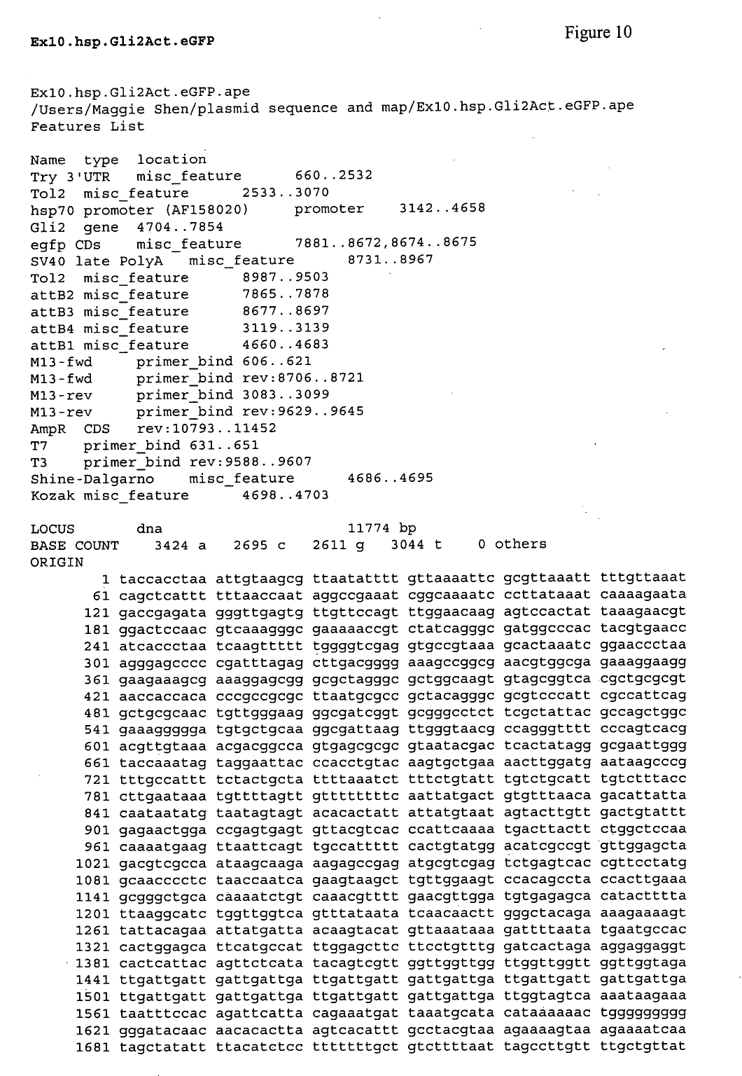

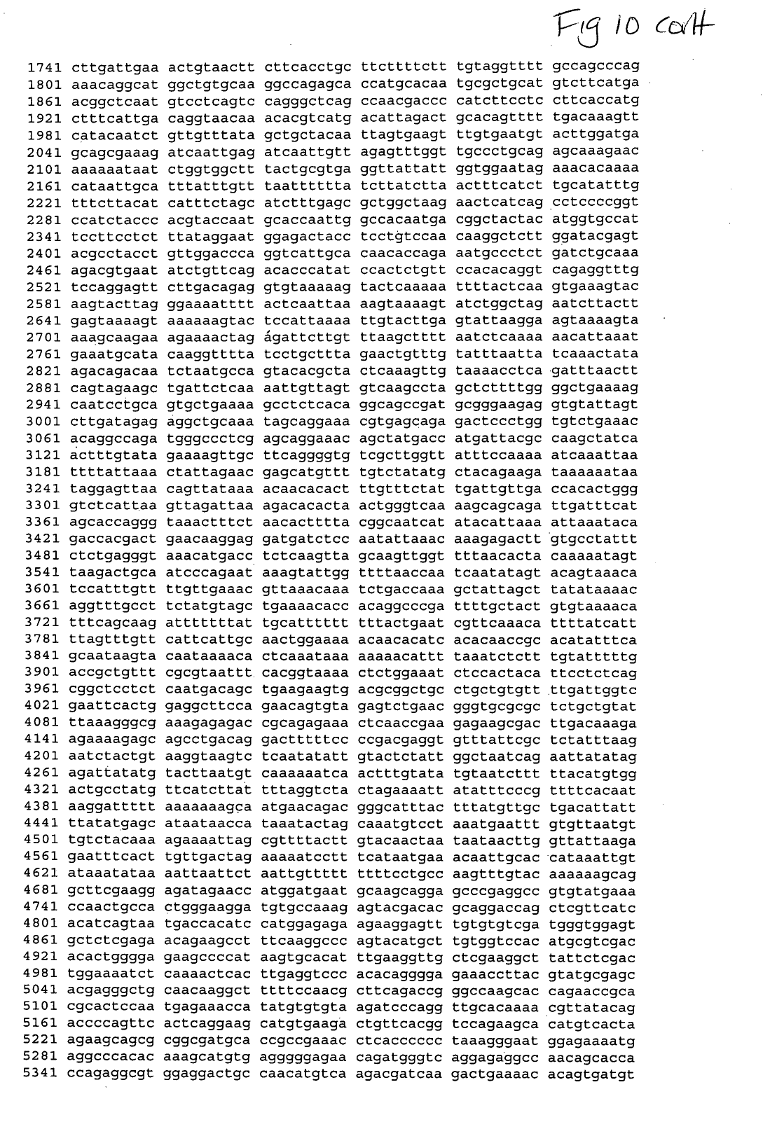

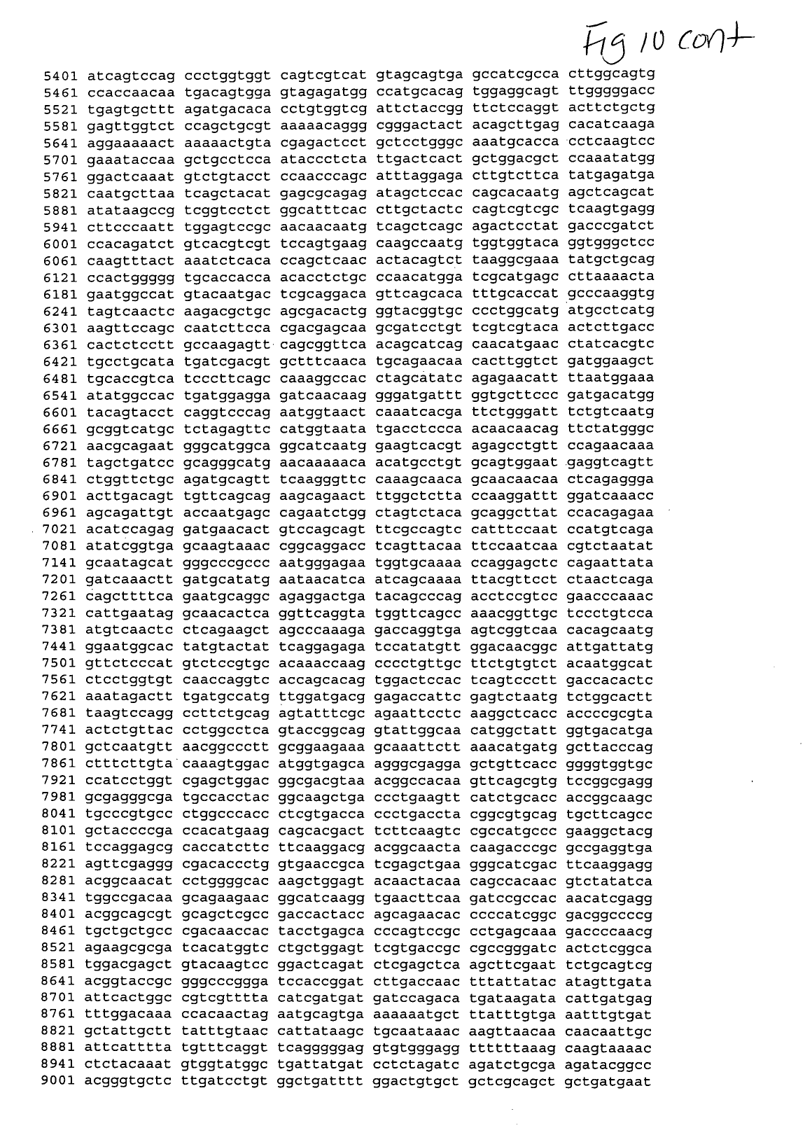

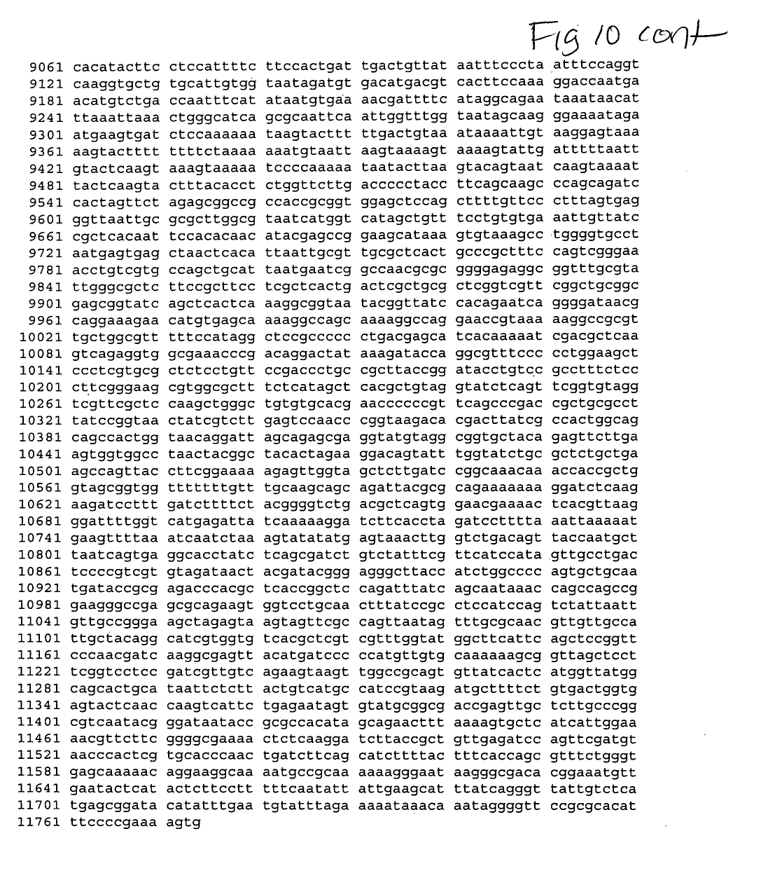

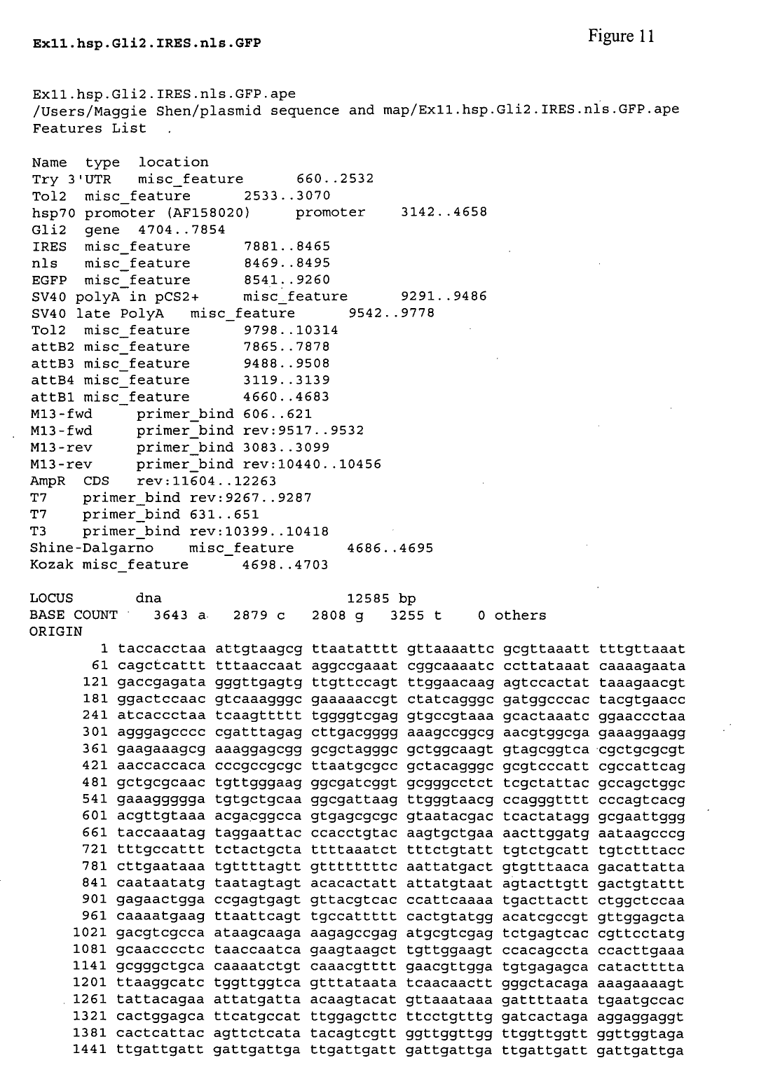

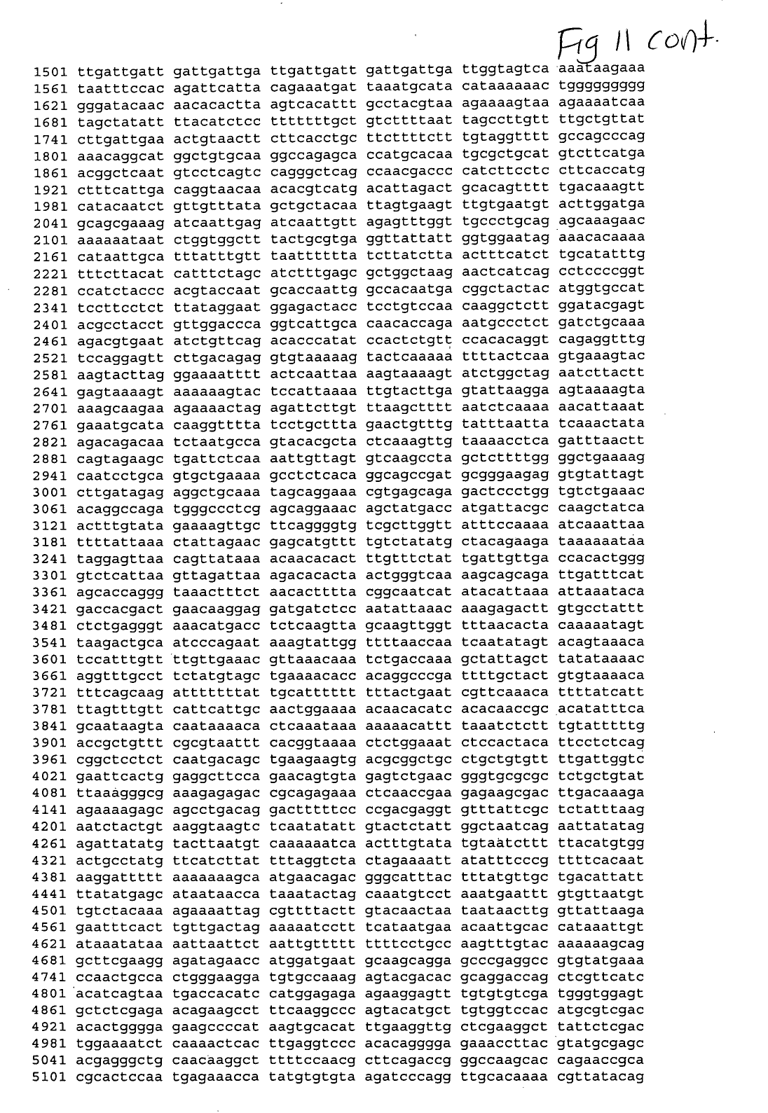

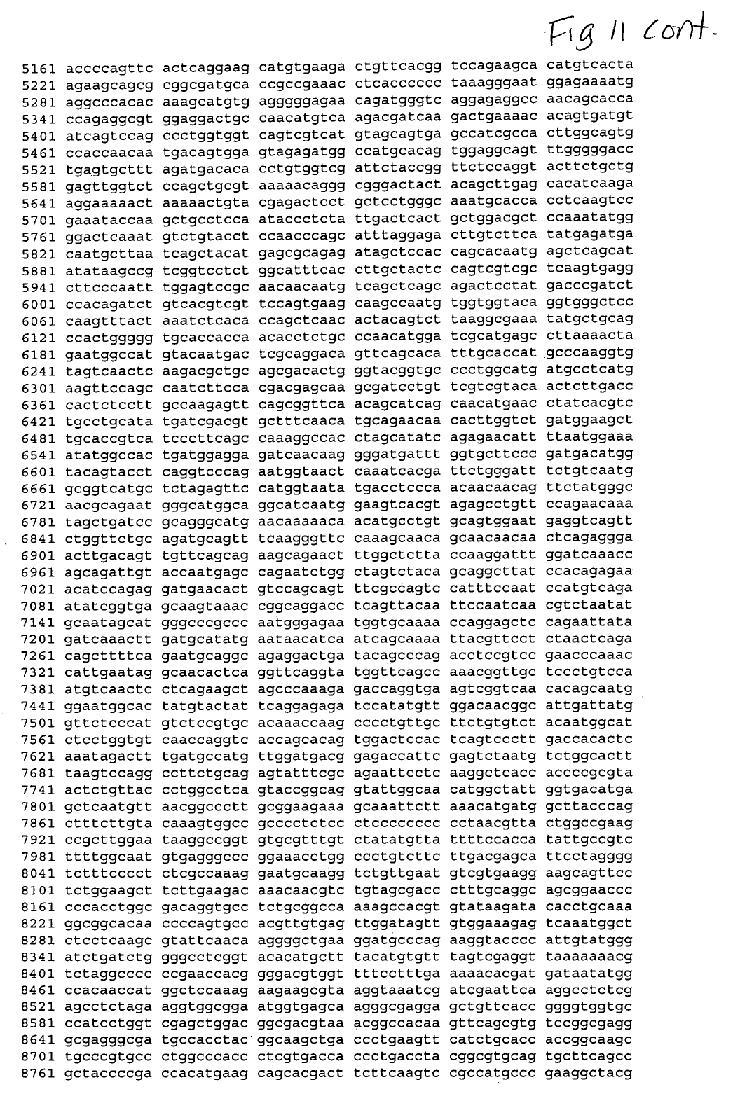

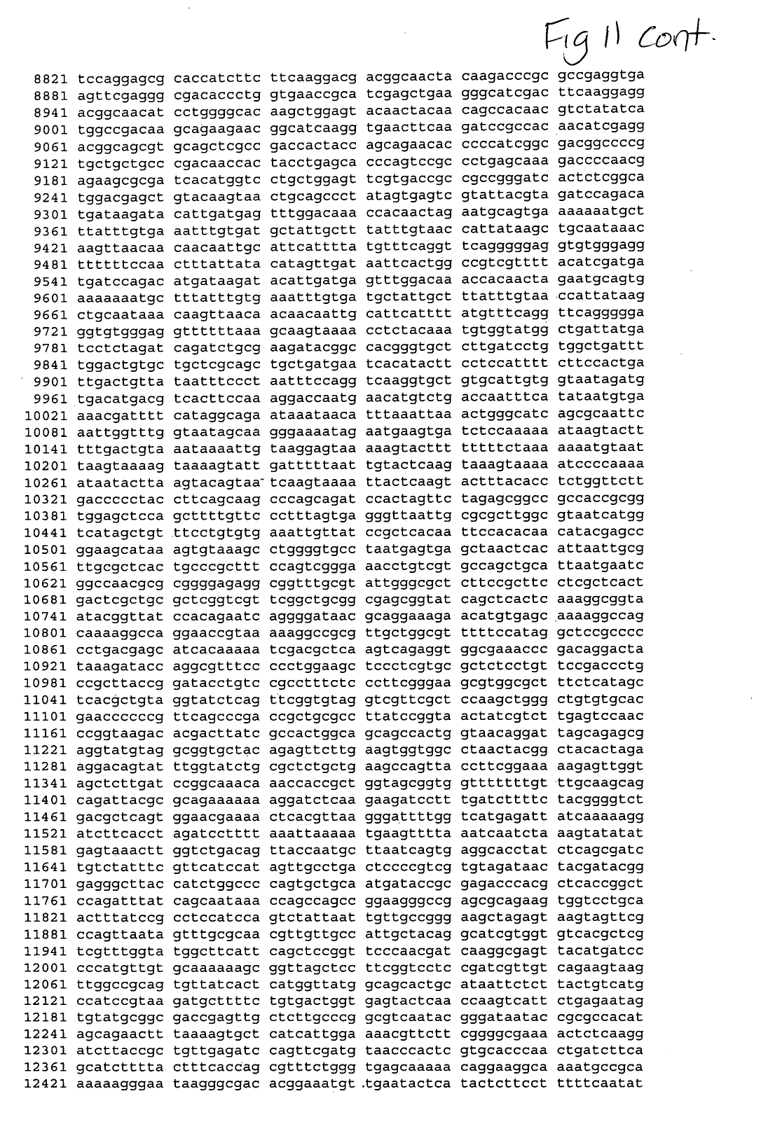

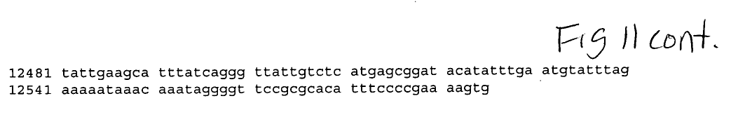

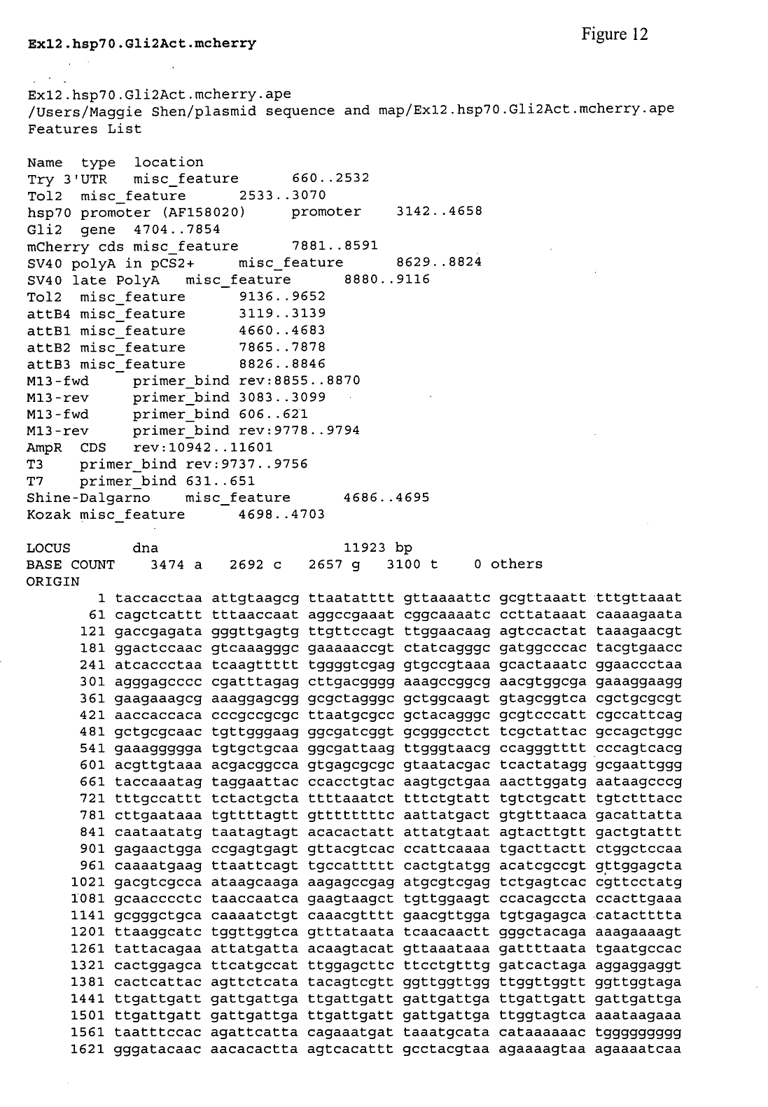

FIGS. 10-12: Gli2Act: Gli2 activator, contains Gli2 mRNA sequence 1531-4681.

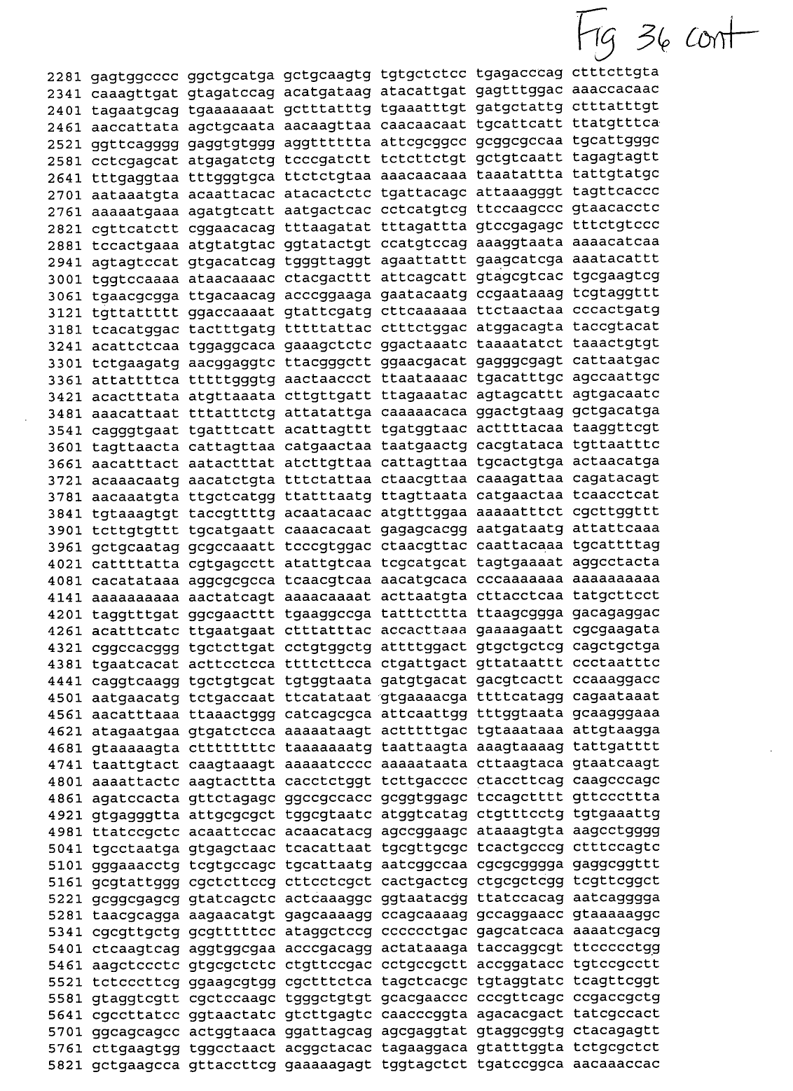

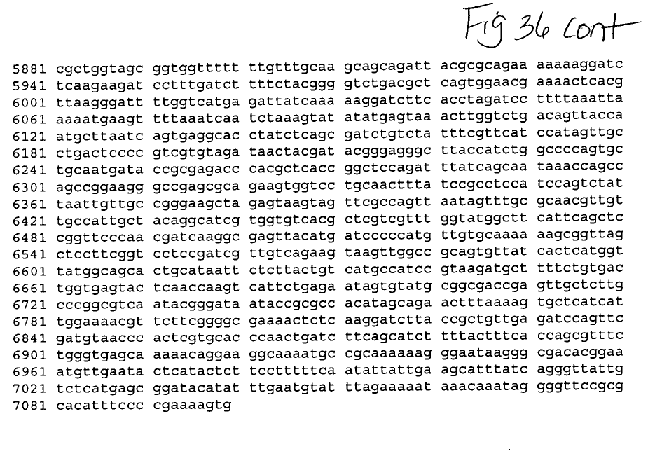

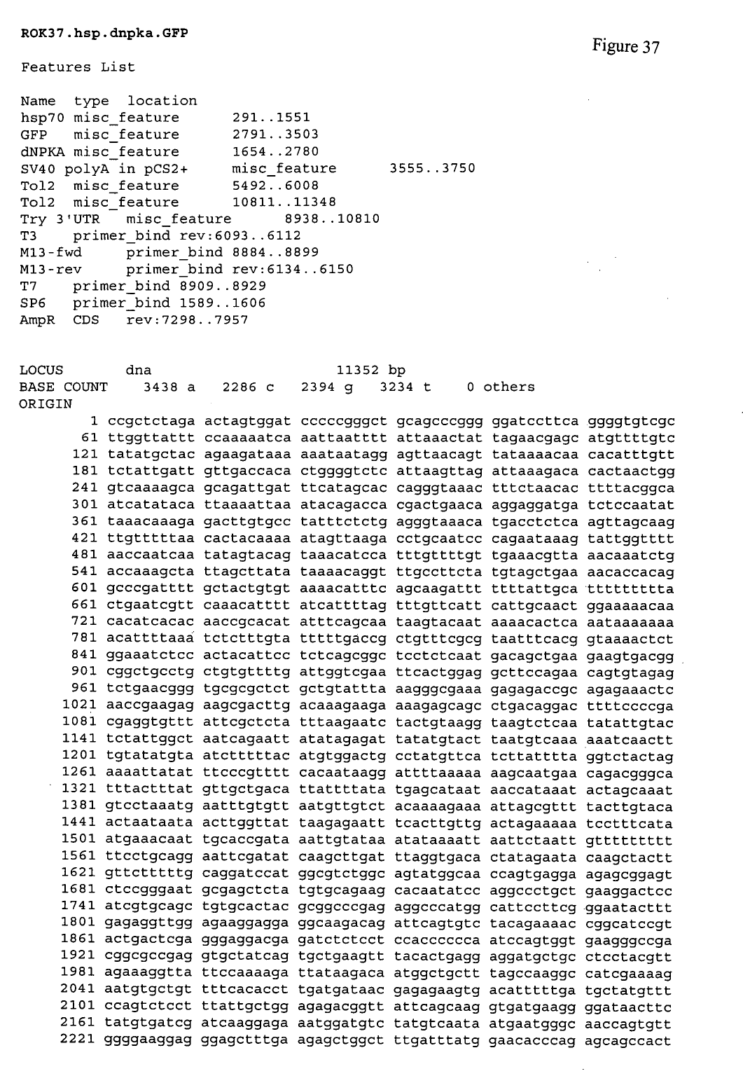

FIG. 37. hsp.dnpka.GFP:hsp: heat shock promoter; dnpka: dominate negative protein kinase A.

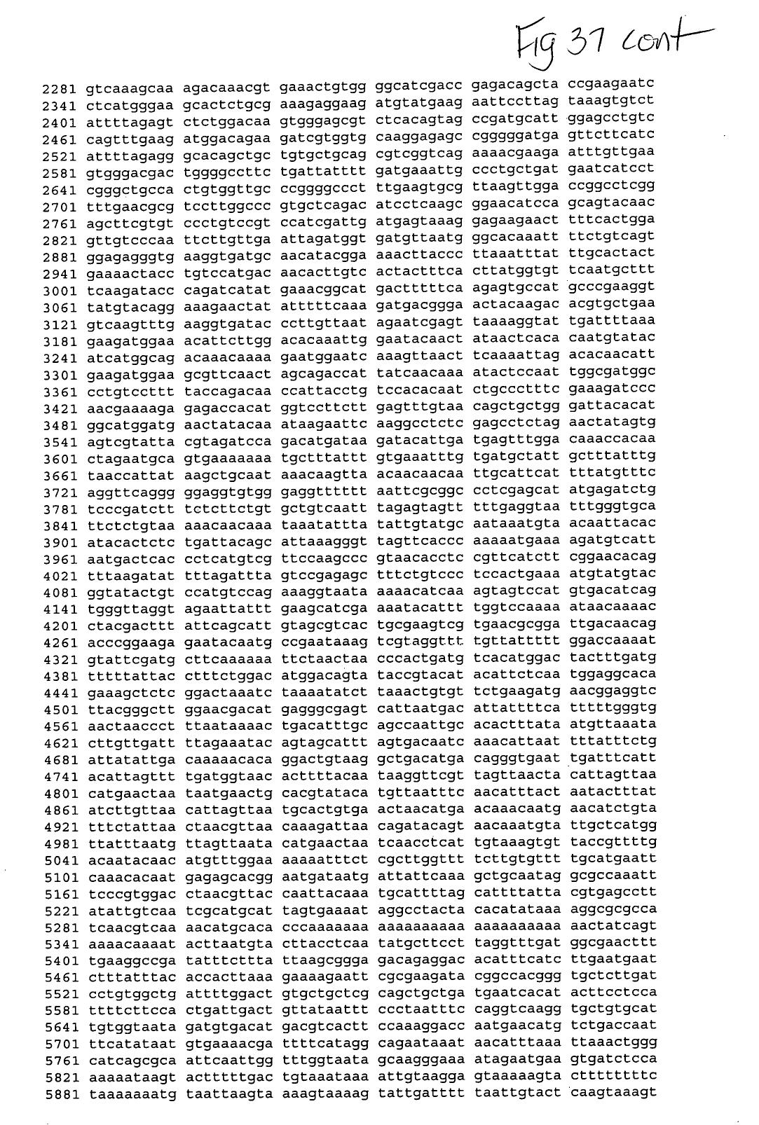

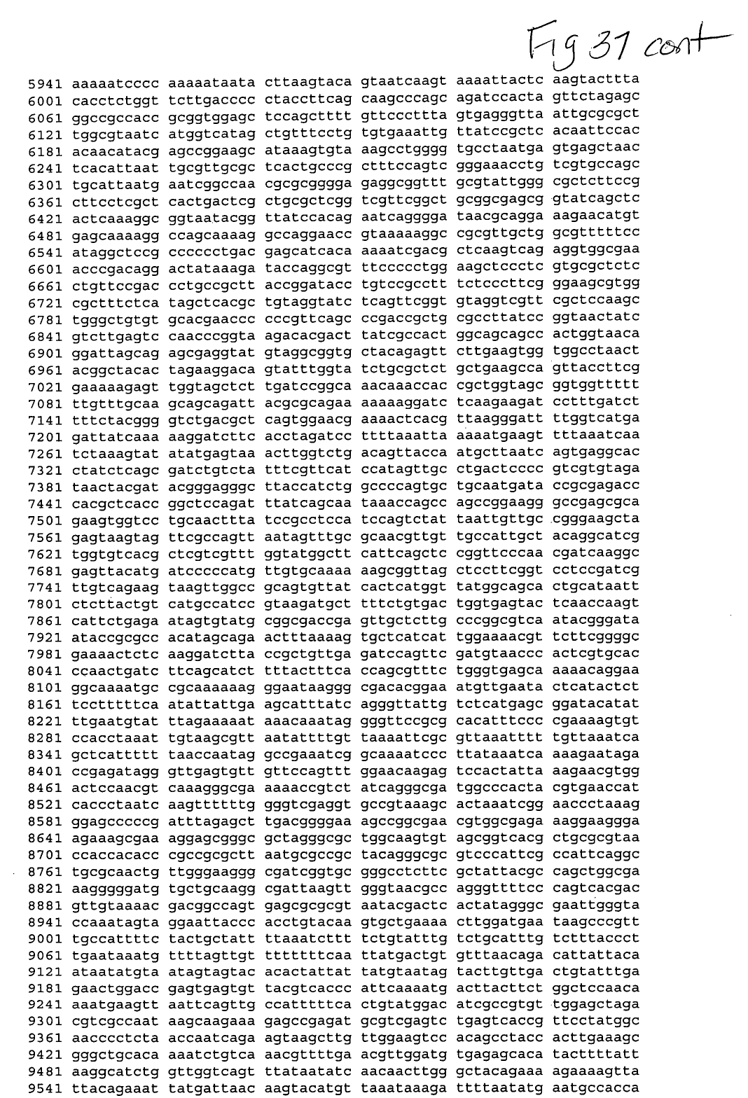

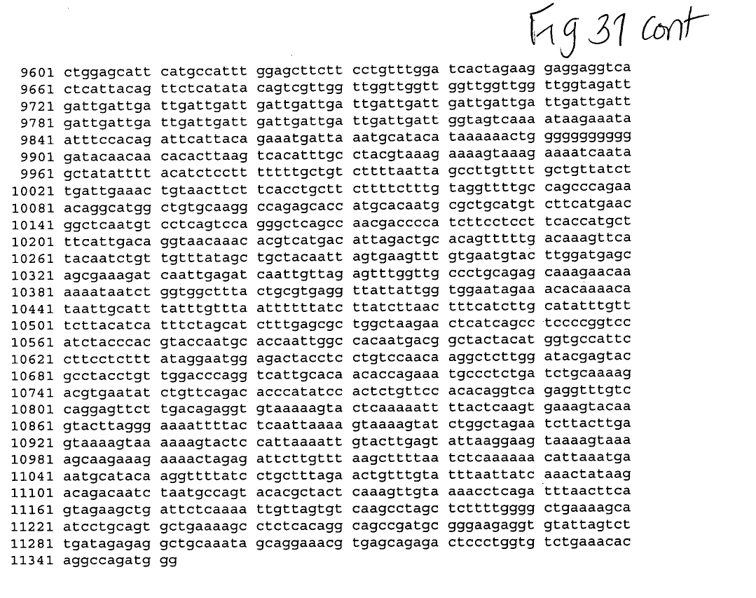

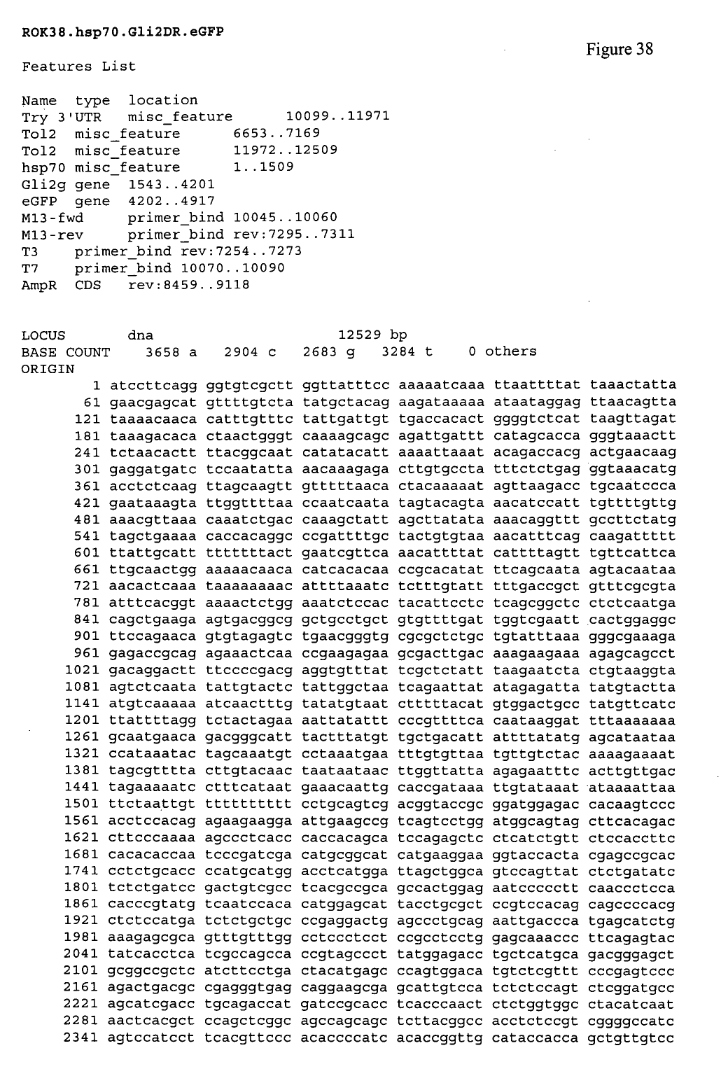

FIG. 38: hsp70.Gli2DR.eGFP: Gli2 DR: Gli2 dominate repressor.

A hsp70-gli2DR-GFP line has been established and functions as planned to cell-autonomously block Hh signaling at any age in the life of the organism. This line can be used to monitor the effects of loss of Hh signaling at different ages on vertebrate development. This analysis can be extended to the regulation of neural stem cell fates in larvae and adults in the near future.

A hsp70-dnPKA-GFP line has also been established and GFP expression has been observed. It is currently been tested to determine whether Hh signaling is occurring.

The hsp70-gli3act-GFP constructs have been made and injected into zebrafish embryos. GFP is clearly expressed after heat shock, and we are now testing for Hh activation.

Using an IRES-GFP plasmid may be helpful to avoid potential problems created by fusing GFP to the gli sequence. Using of a monomeric cherry fluorescent protein may be useful in avoiding potential problems that may be caused by multimerization of a fusion protein.

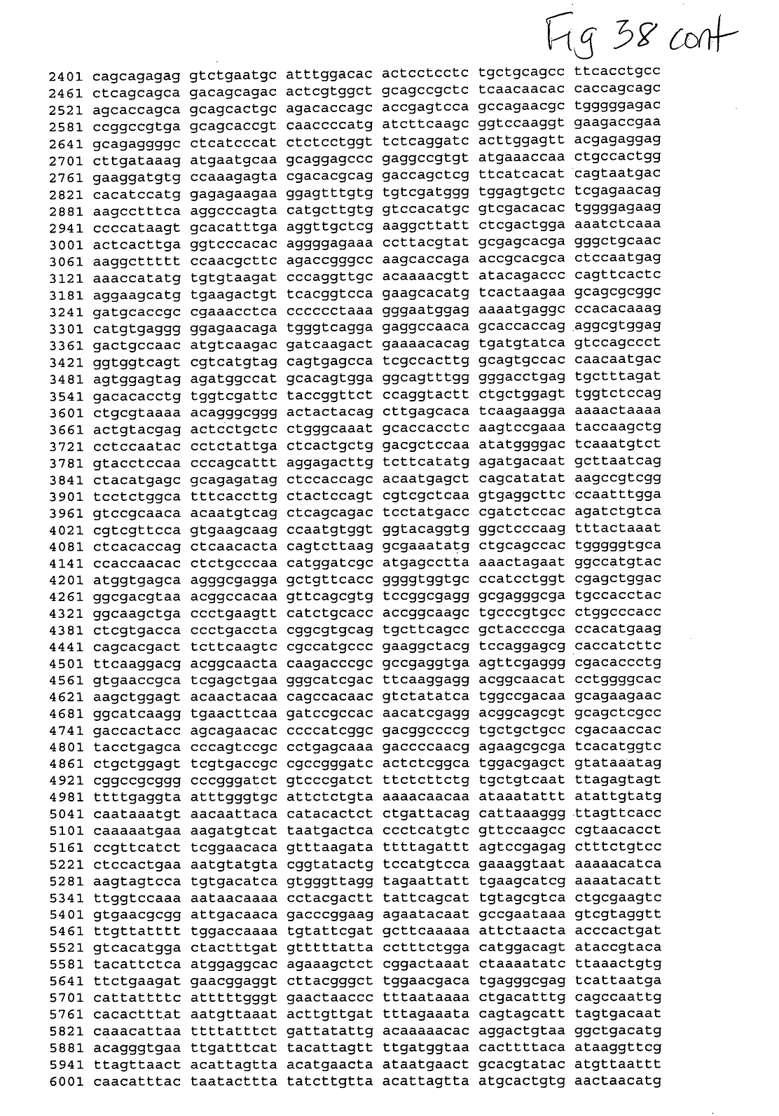

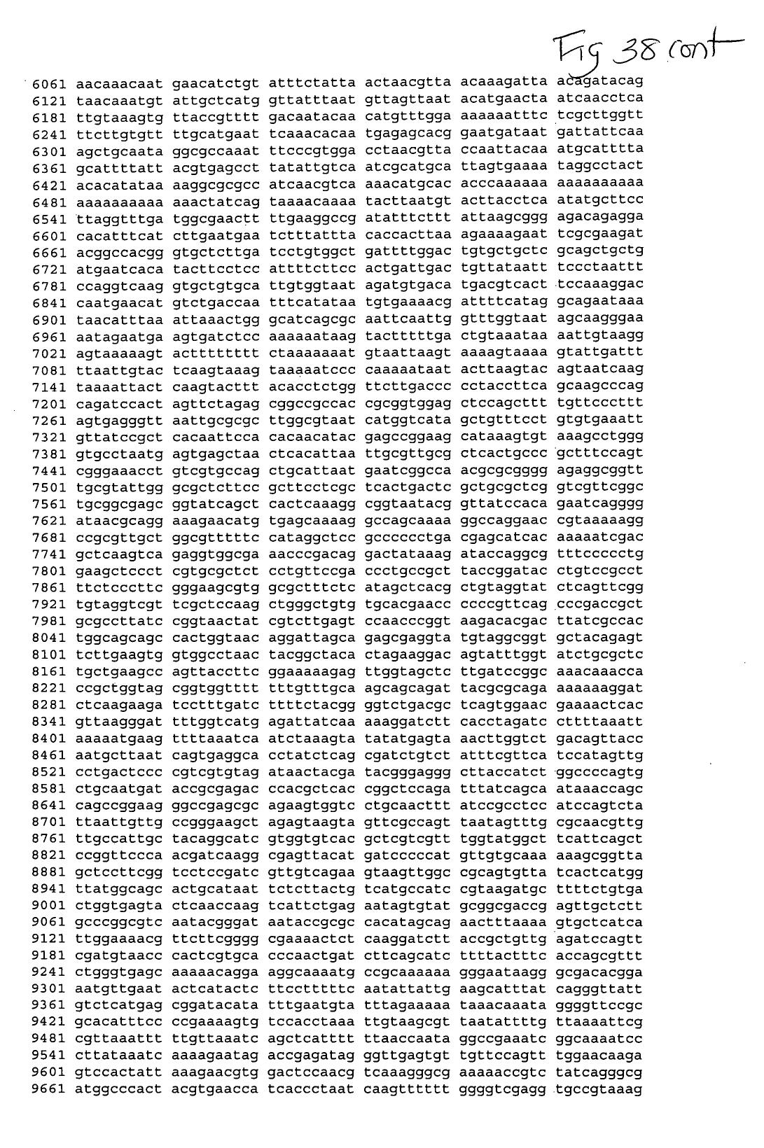

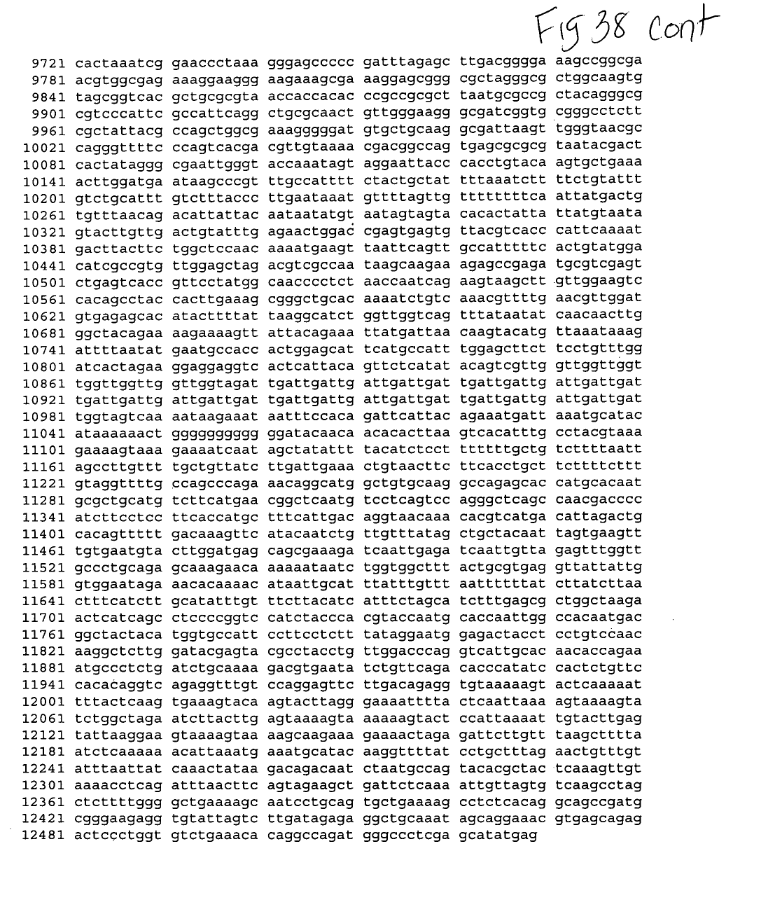

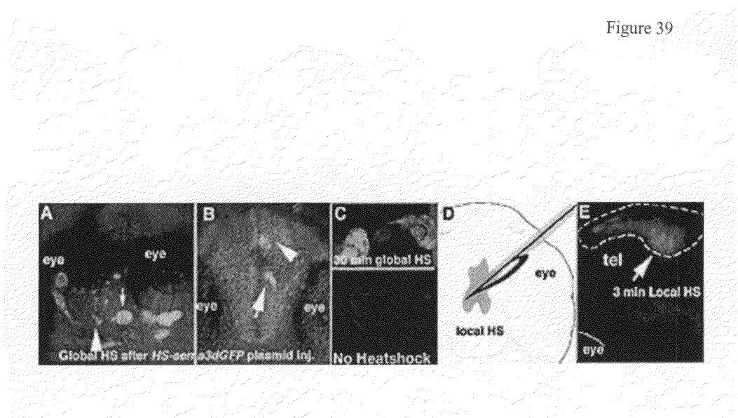

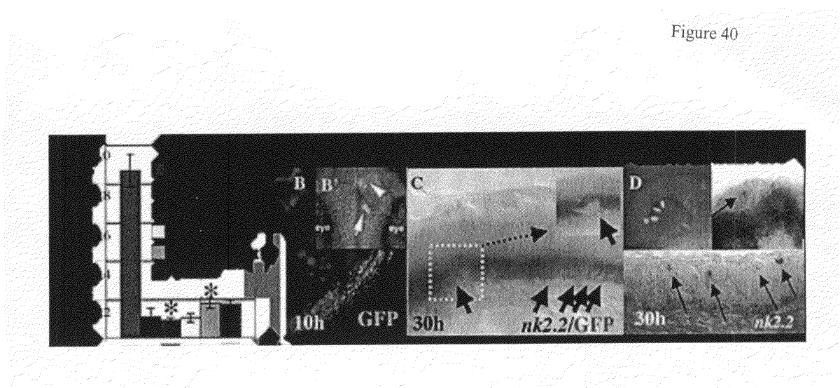

The use of HSP70 promoter to drive transgene expression in zebrafish is shown in FIGS. 39 and 40.

These transgenic lines will allow us to induce Hh signaling at any stage and any place in the organism. Since over-activation of Hh in adults leads to cancer in humans, our lines will allow us to better understand how Hh activation leads to tumors in different tissues, and will allow us to test reagents that might block Hh mediated tumorigenesis. These transgenic lines could thus form an important model for human skin and brain cancer, and allow rapid and efficient testing of anti-cancer agents. In addition, these lines may allow us to determine whether chemicals present in the environment, including human generated waste products, might activate Hh signaling and thus be potential causes of cancer.

### Example 2

### Hh Signaling Reporter Constructs

In a related project, we are developing a zebrafish transgenic Hh reporter line that will allow us to visualize Hh signal activation in living embryos. This line will take advantage of known Hh regulatory elements. Tandem DNA elements that contain binding sites for human Gli1 (hGli1-BS) provide a good reporter for Hh activation (Sasaki et al., 1997) and we have used a GliBS-luciferase reporter to monitor Hh activation by zebrafish Gli gene function in cell culture (Karlstrom et al., 2003; Tyurina et al., 2005) The DNA regulator region that regulates Ptc expression (ptc-promoter region) can also be used to monitor Hh activation in vitro. We are building transgenic lines in which hGli-BS, zebrafish Gli-BS, and ptc-promoter elements are used to drive GFP expression, in living embryos. With these lines, we will be able to visually monitor whether and where Hh signaling is activated throughout the living embryo and adult in normal and various manipulated conditions. This will be a powerful experimental tool for our analysis of Hh mis-regulation, and will allow a powerful assay for chemical compounds that might activate or block Hh signaling. It could thus be used to screen for pharmaceuticals or for environmental agents that activate or block Hh signaling.

The gli binding site (GLIbs) GFP and RFP constructs needed to create the Hh-reporter line are under construction. We will also take advantage of two new fluorescent molecules to increase the utility of this line. One is a “timer” gfp that switches colors after a few hours, allowing newly synthesized protein to be distinguished from older protein. A second is the kaede protein, which changes color following illumination.

Exemplary vectors that can be used are set forth in Table 2 below:

| Construct name | Fig. | SEQ ID NO |
|---|---|---|
| ptc1prmoter.500bp.nlsGFP | 13 | 25 |
| Ptc1promoter.500bp.nlsCherry | 14 | 26 |
| Ptc1promoter.500bp.mEGFP | 15 | 27 |
| Ptc1promoter.500bp.mmCherry | 16 | 28 |
| ptc1promoter.1kb.nlsGFP | 17 | 29 |
| ptc1promoter.1kb.nlsCherry | 18 | 30 |
| Ptc1promoter1kb.mEGFP | 19 | 31 |
| Ptc1promoter.1kb.mmcherry | 20 | 32 |
| ptc1promoter.2kb.nlsGFP | 21 | 33 |
| ptc1promoter.2kb.nlsCherry | 22 | 34 |
| Ptc1promoter.2kb.mEGFP | 23 | 35 |
| Ptc1promoter.2kb.mmCherry | 24 | 36 |
| 5hGBs.nlsGFP | 25 | 37 |
| 5hGBs.nlsChery | 26 | 38 |
| 5hGBs.mEGFP | 27 | 39 |
| 5hGBs.mmCherry | 28 | 40 |
| 10GBs.nlsGFP | 29 | 41 |
| 10hGBs.nlsCherry | 30 | 42 |
| 10hGBs.mEGFP | 31 | 43 |
| 10hGBs.mmCherry | 32 | 44 |
| 20GBs.nlsGFP | 33 | 45 |
| 20hGBs.nlsCherry | 34 | 46 |
| 20hGBs.mEGFP | 35 | 47 |
| 20hGBs.mmCherry | 36 | 48 |

Set forth below are explanations regarding the constructs:

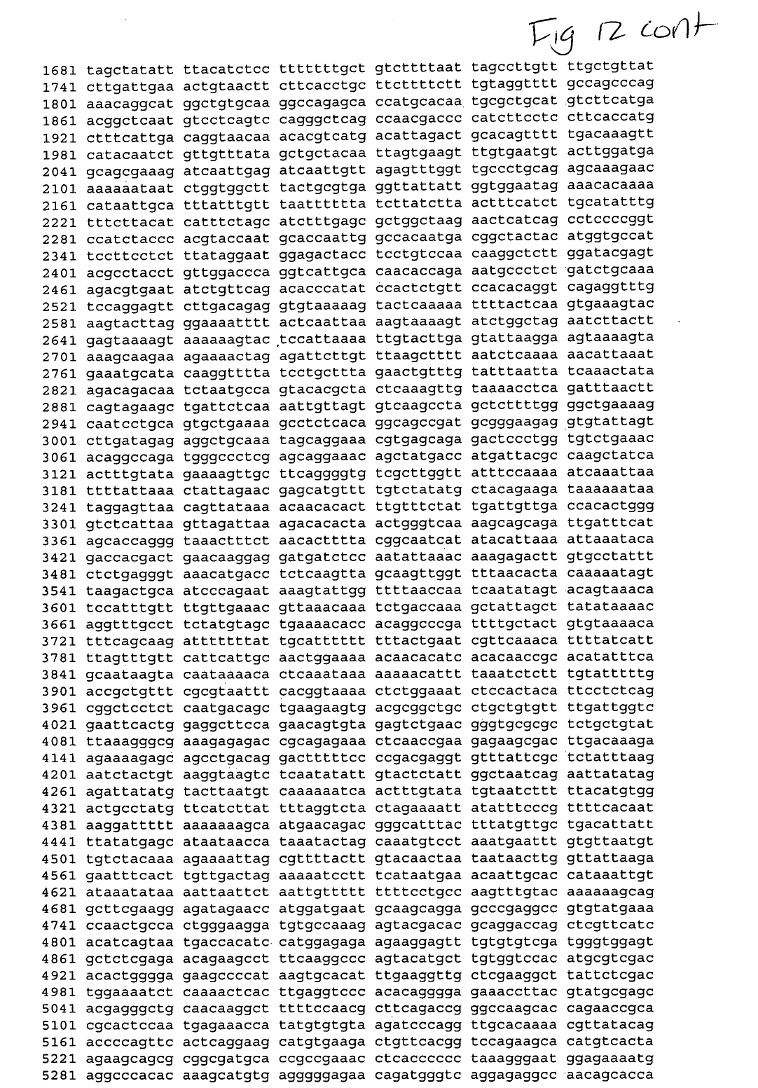

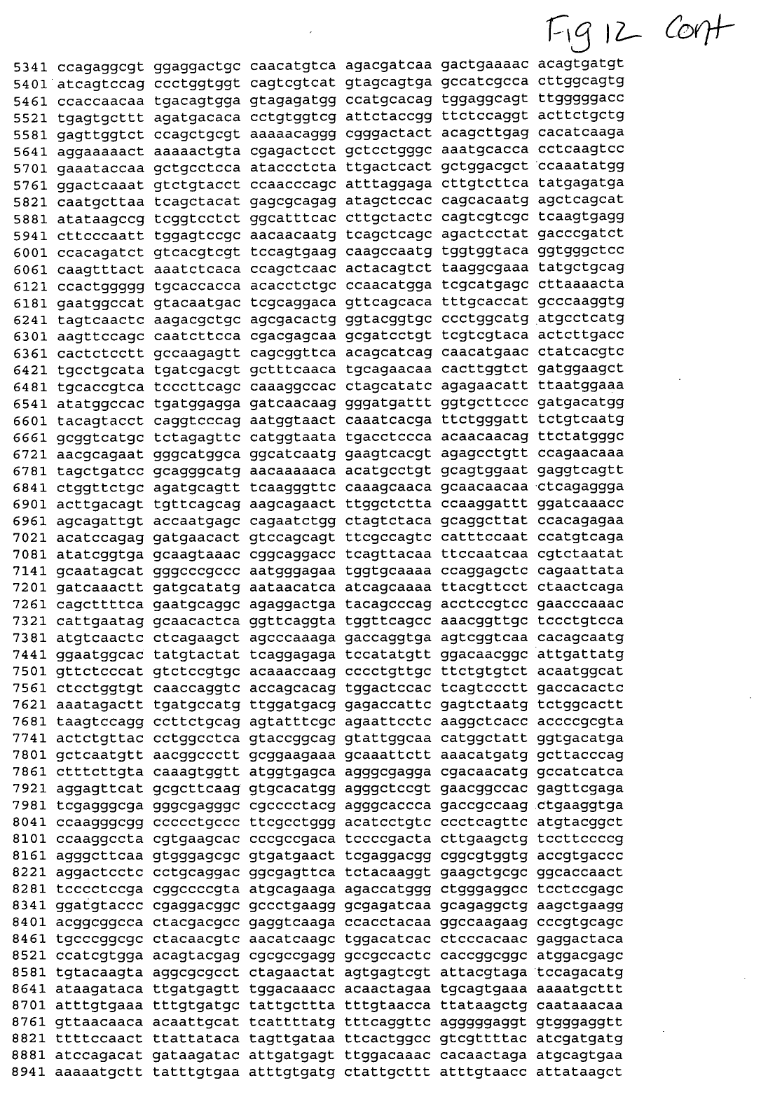

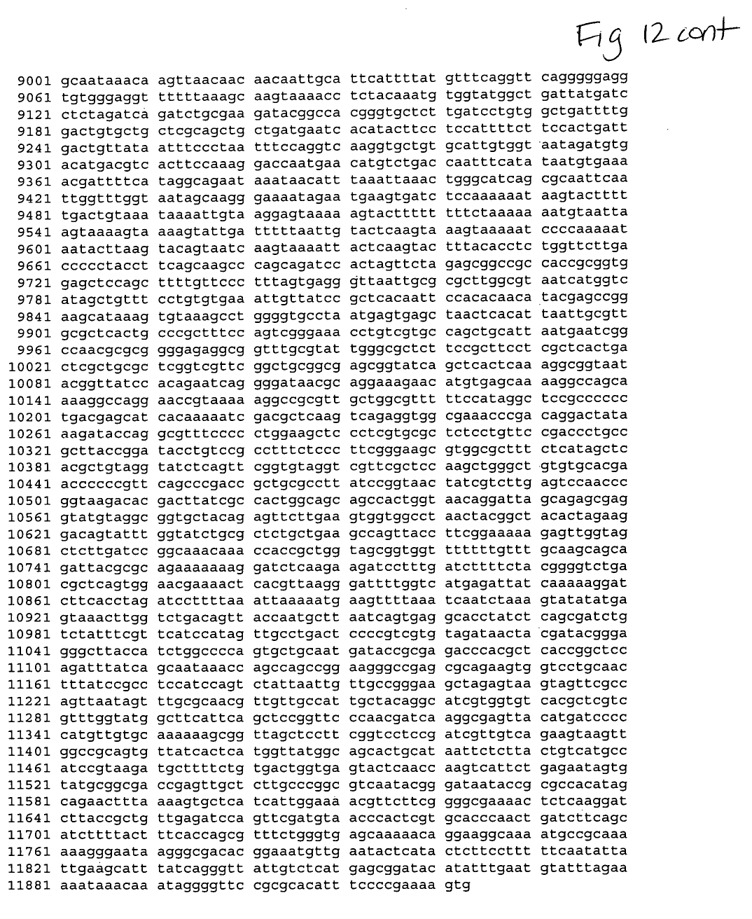

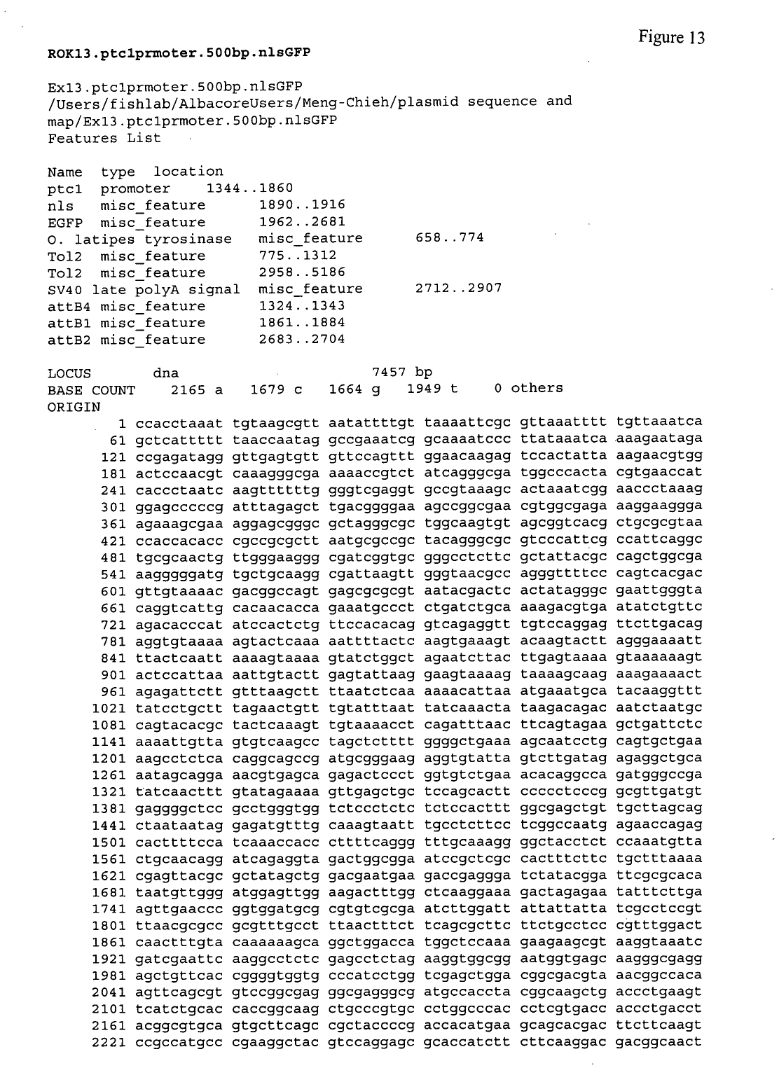

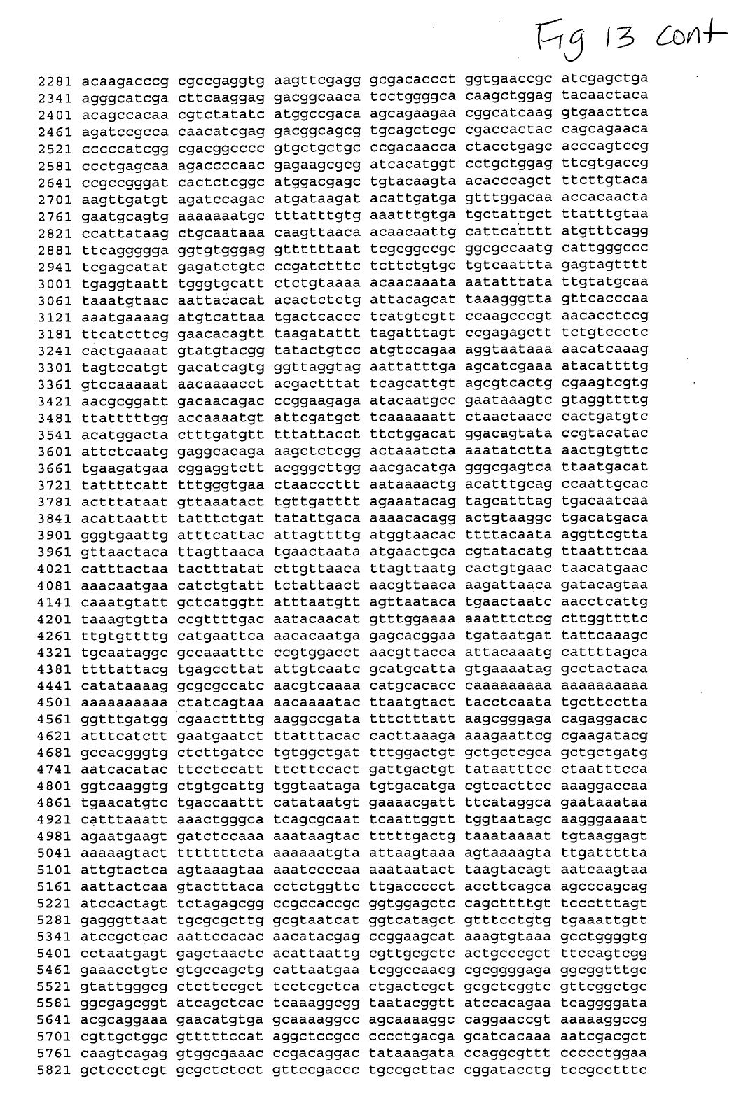

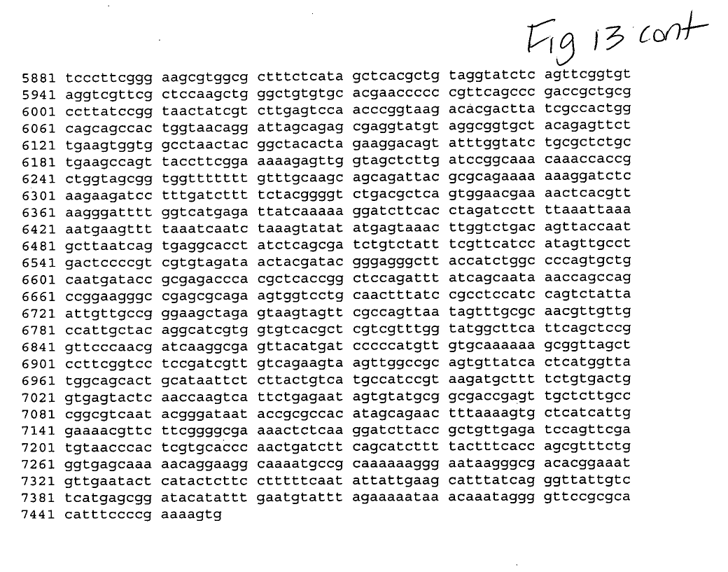

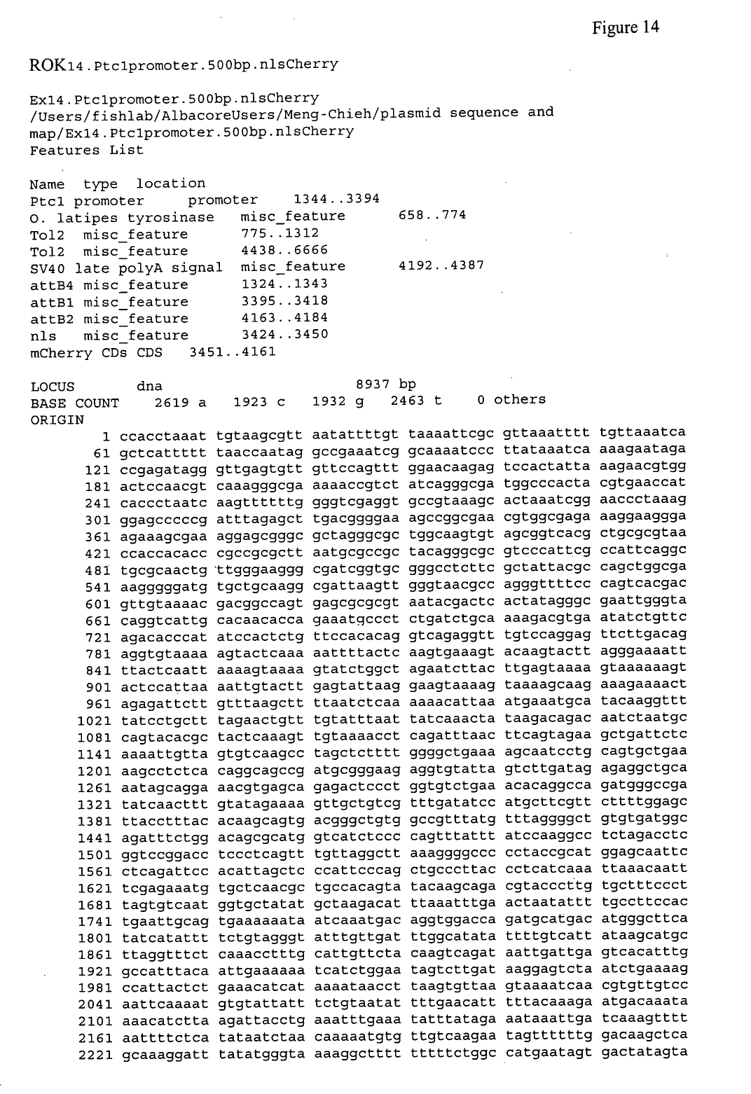

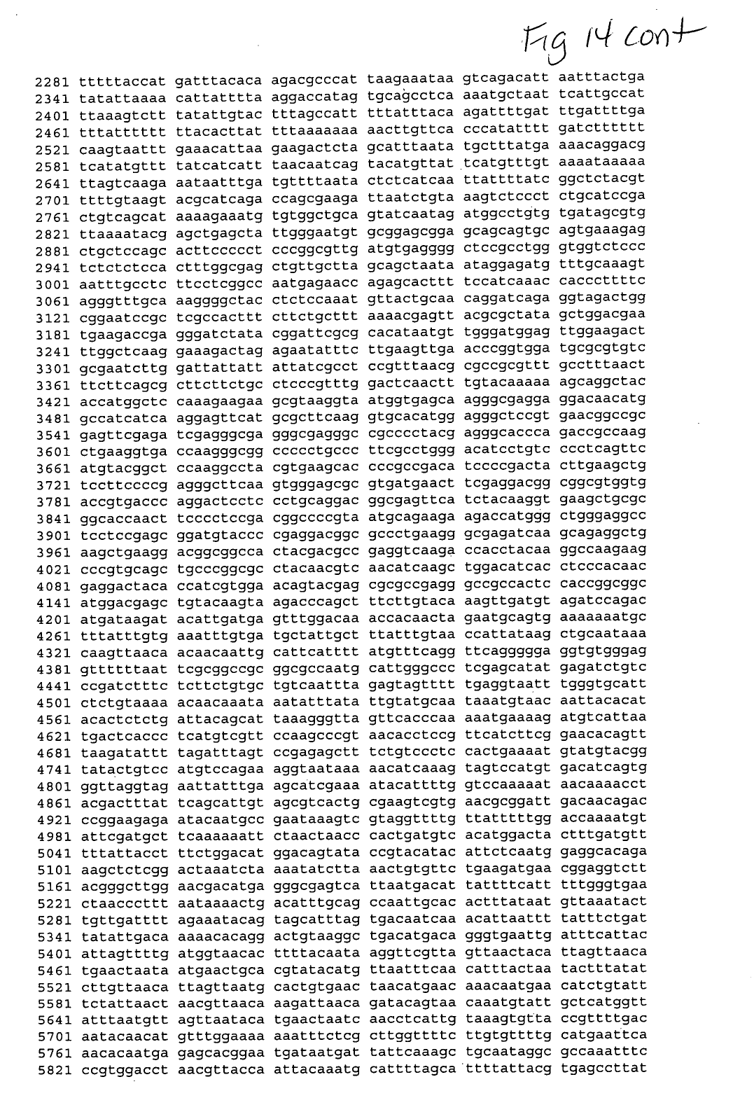

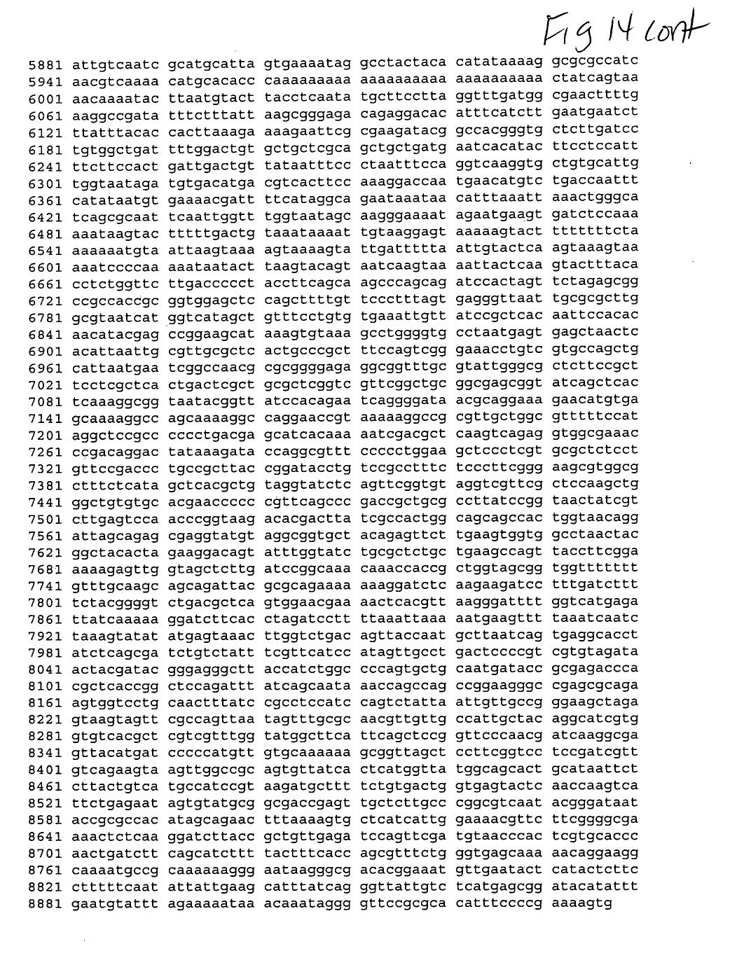

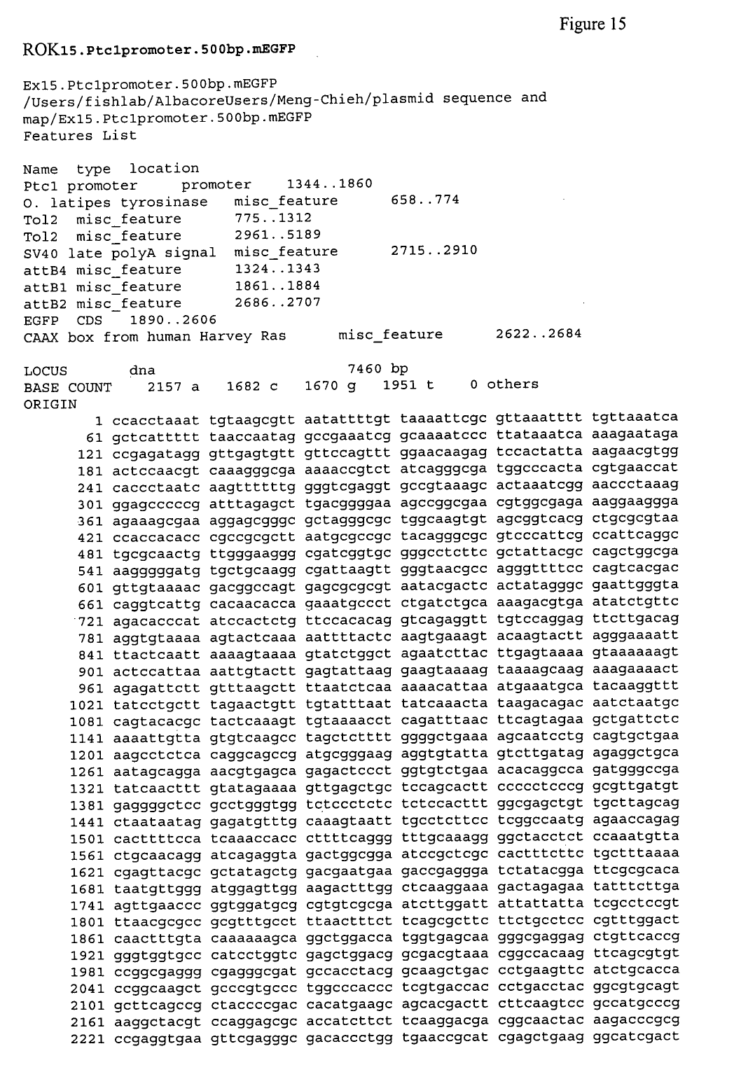

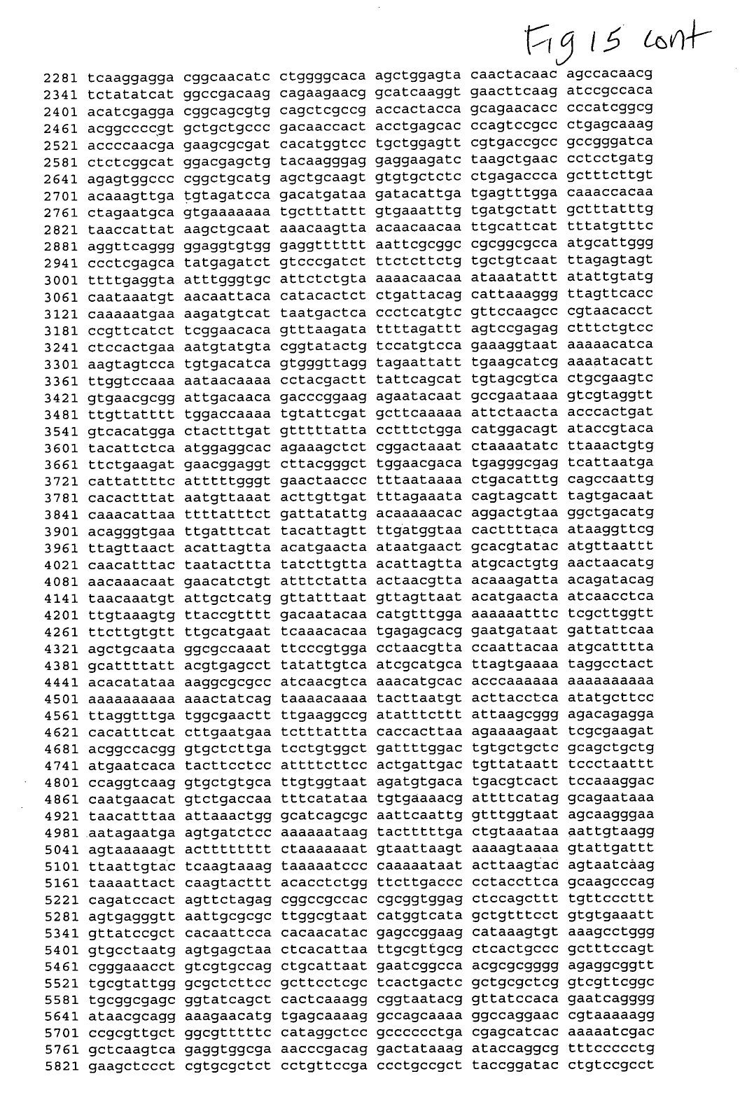

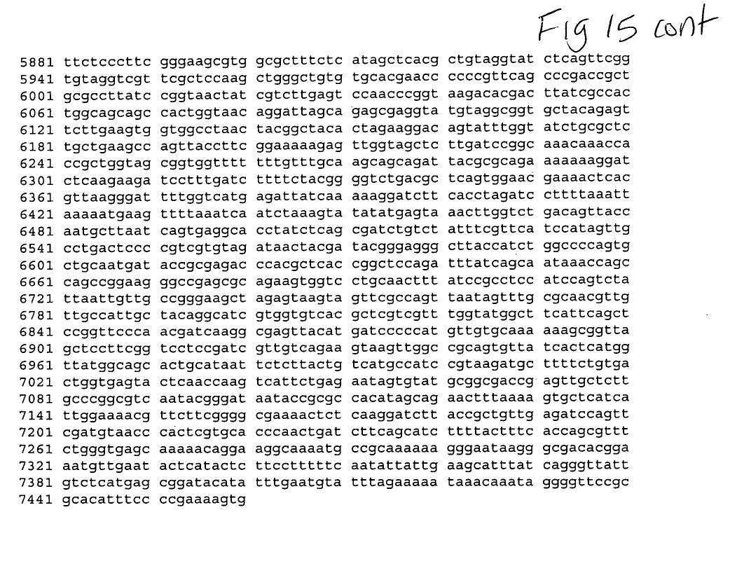

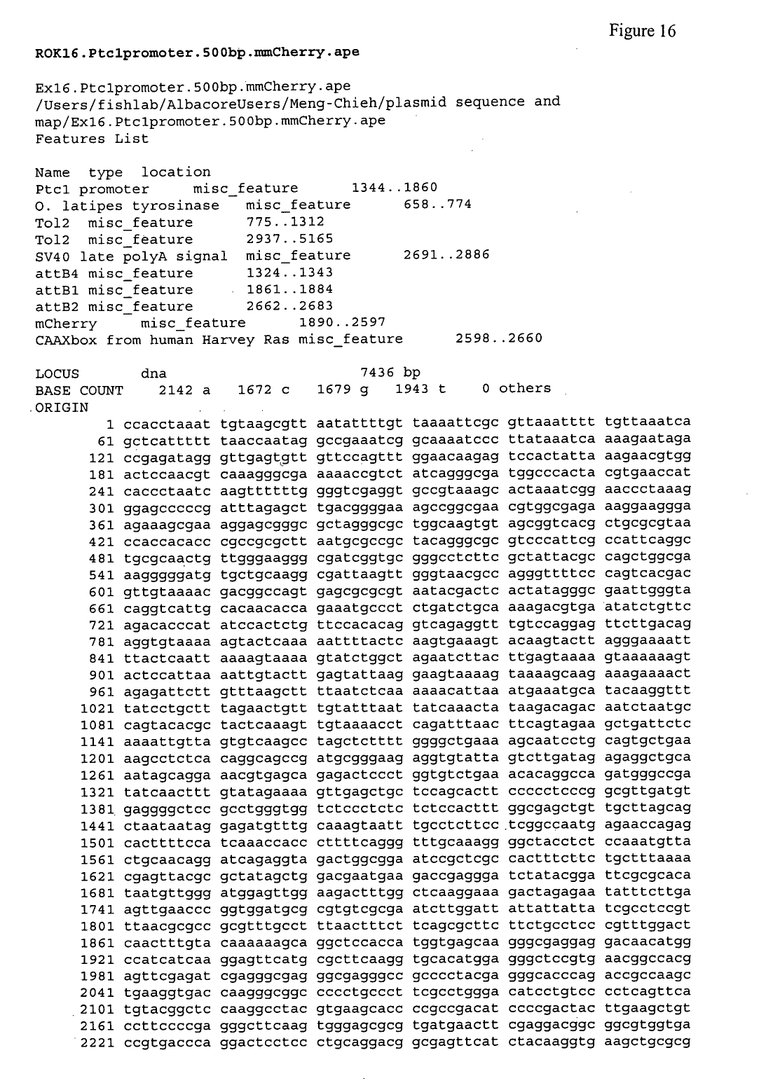

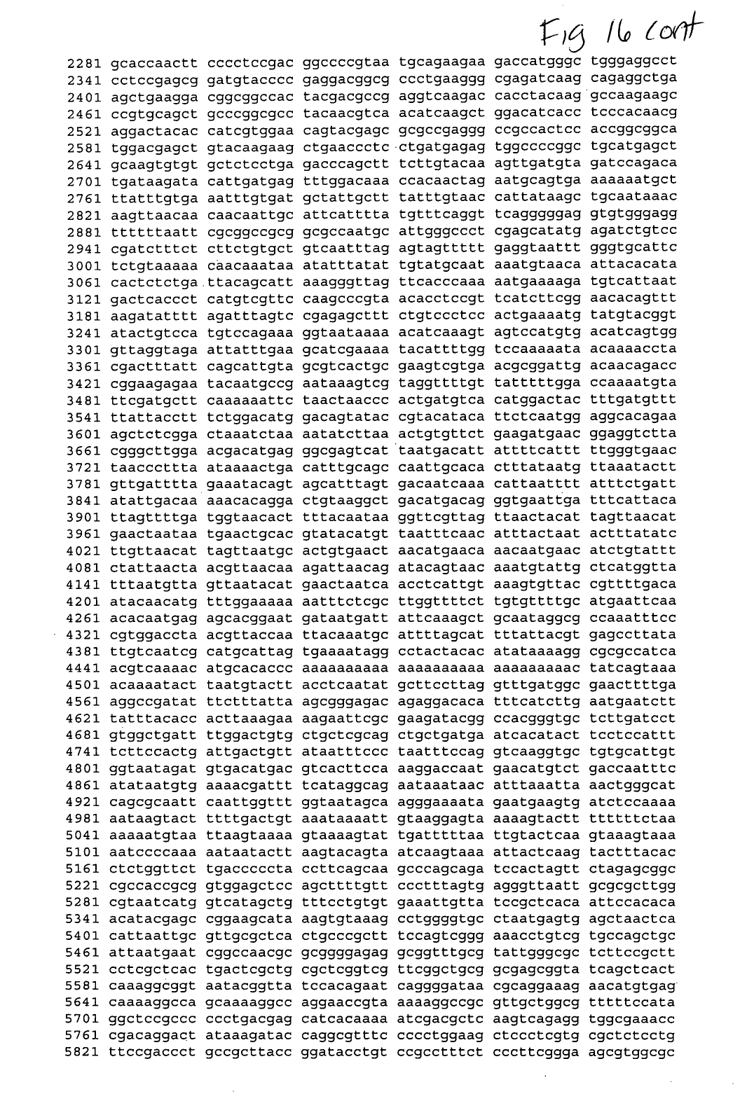

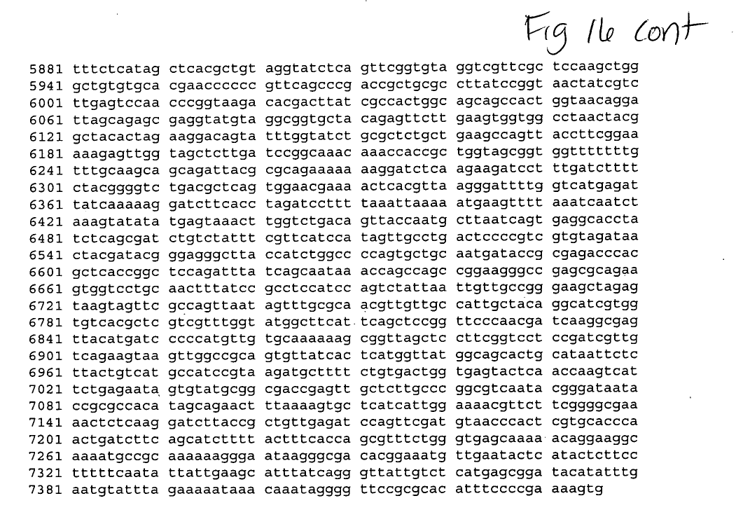

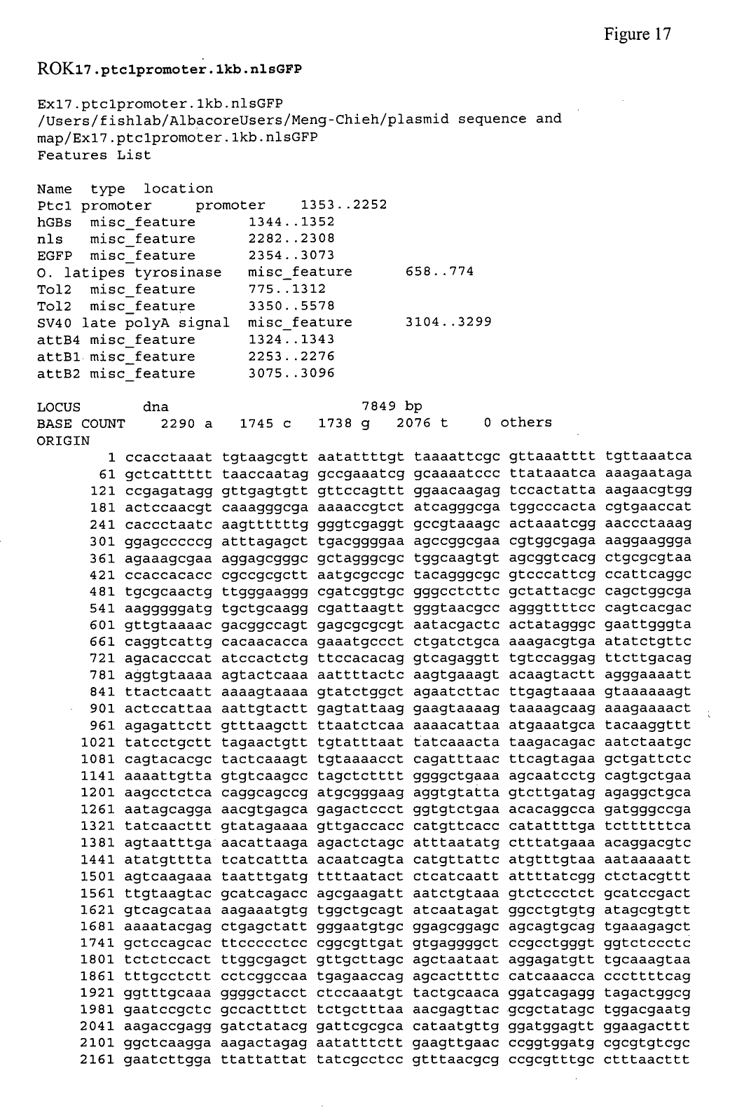

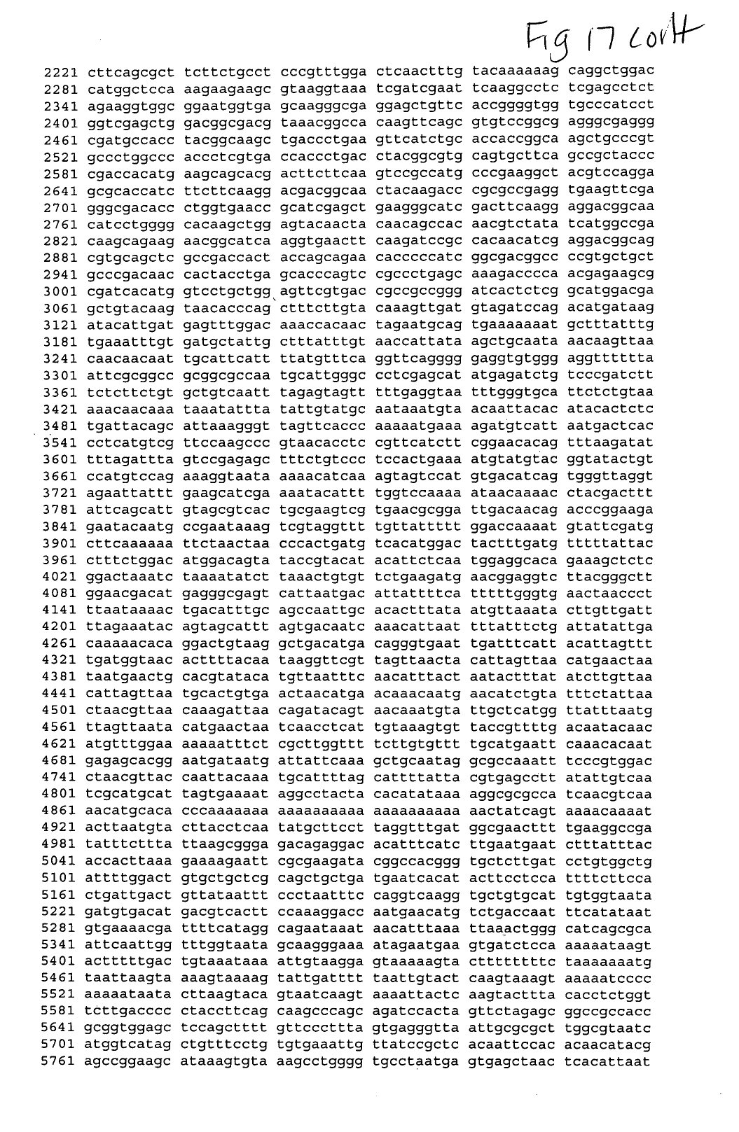

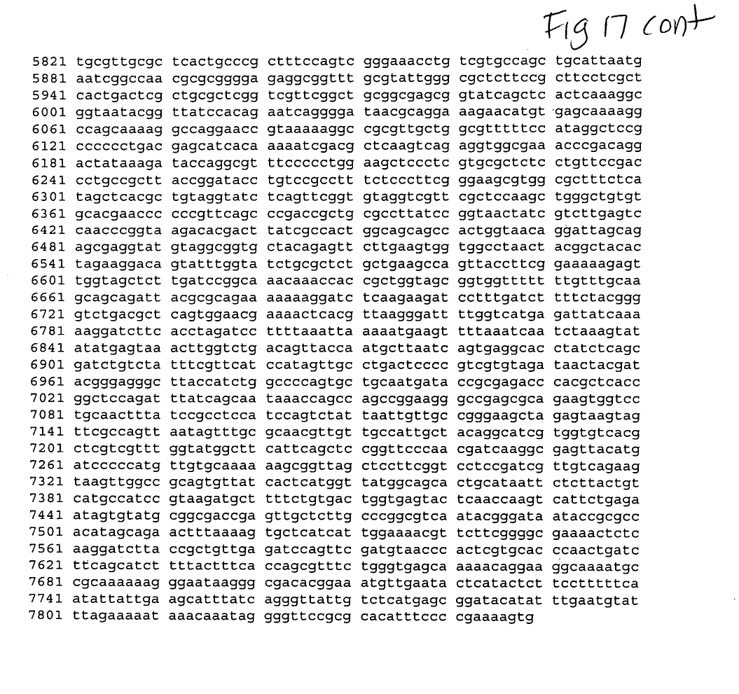

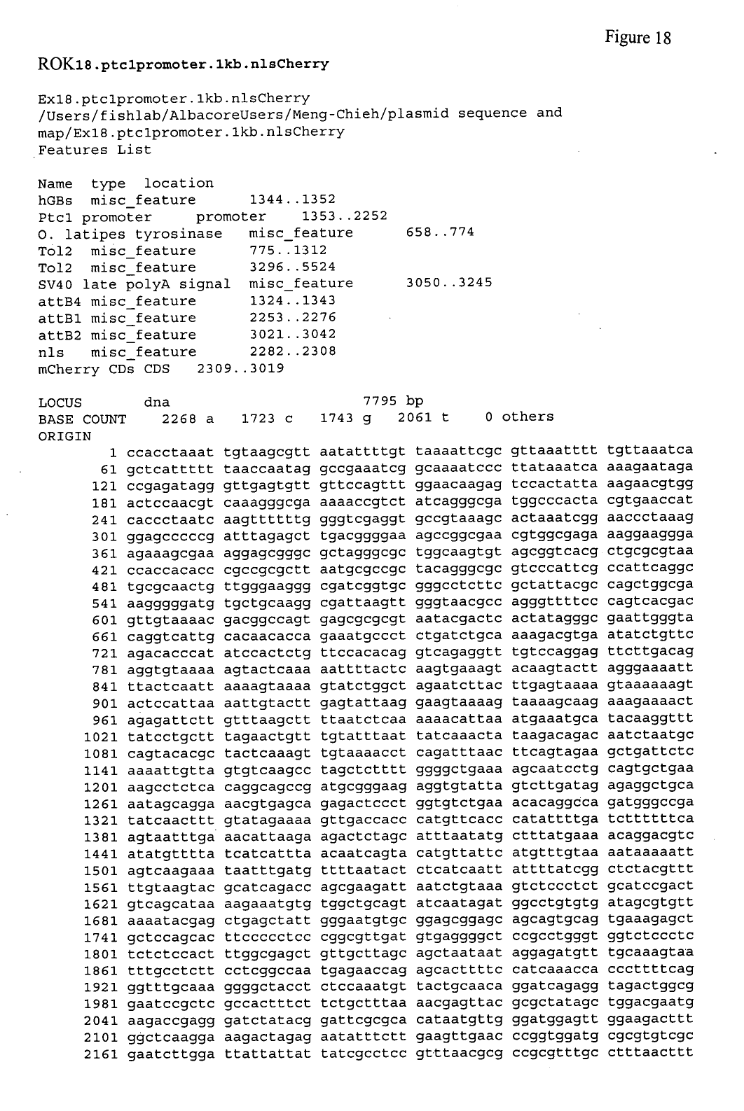

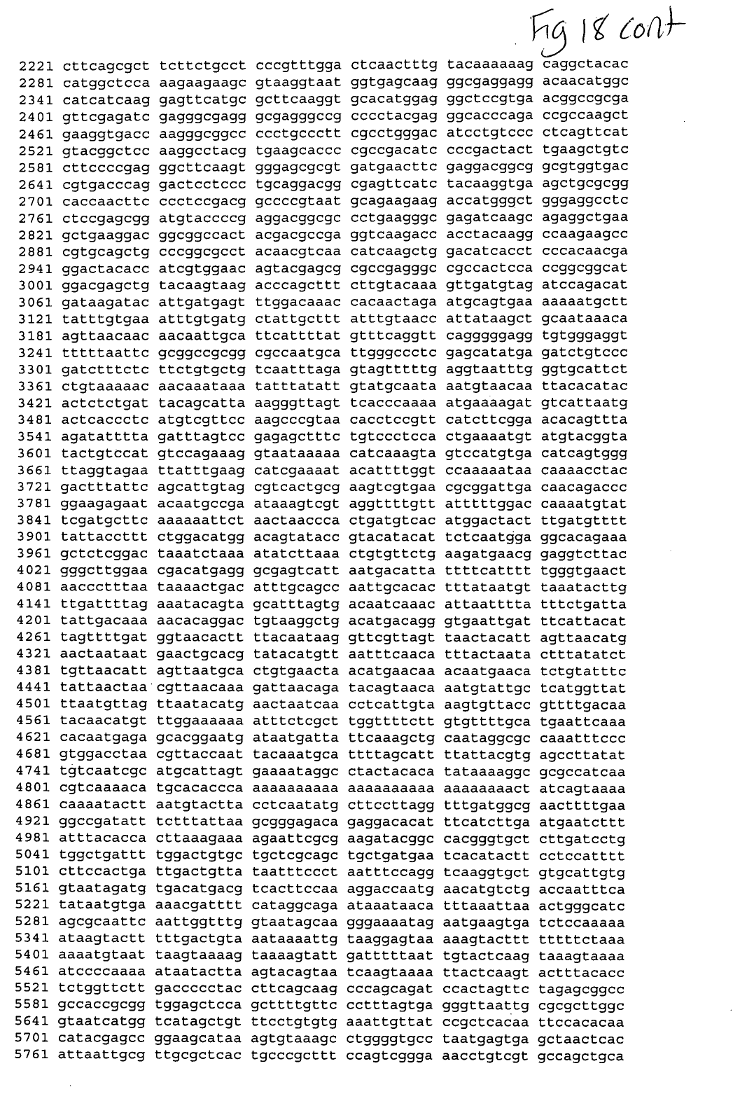

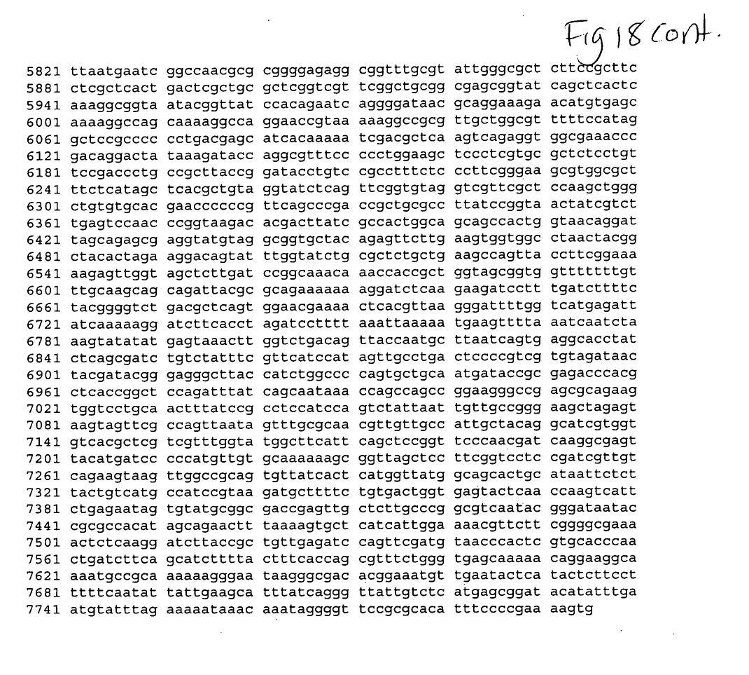

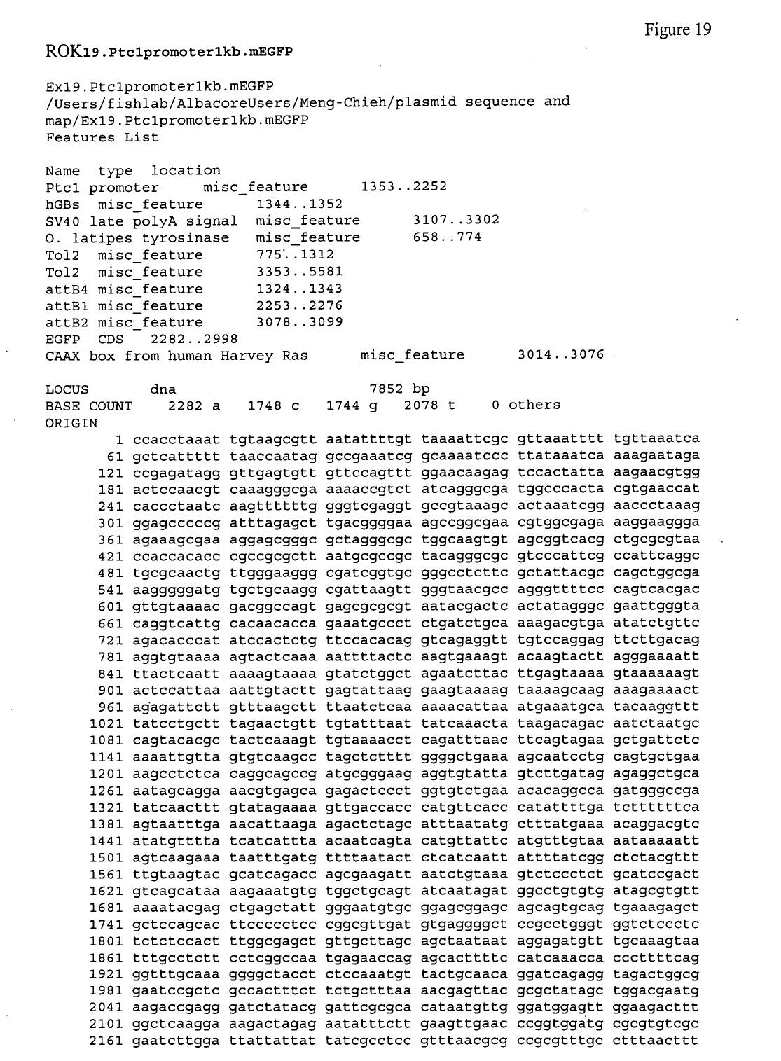

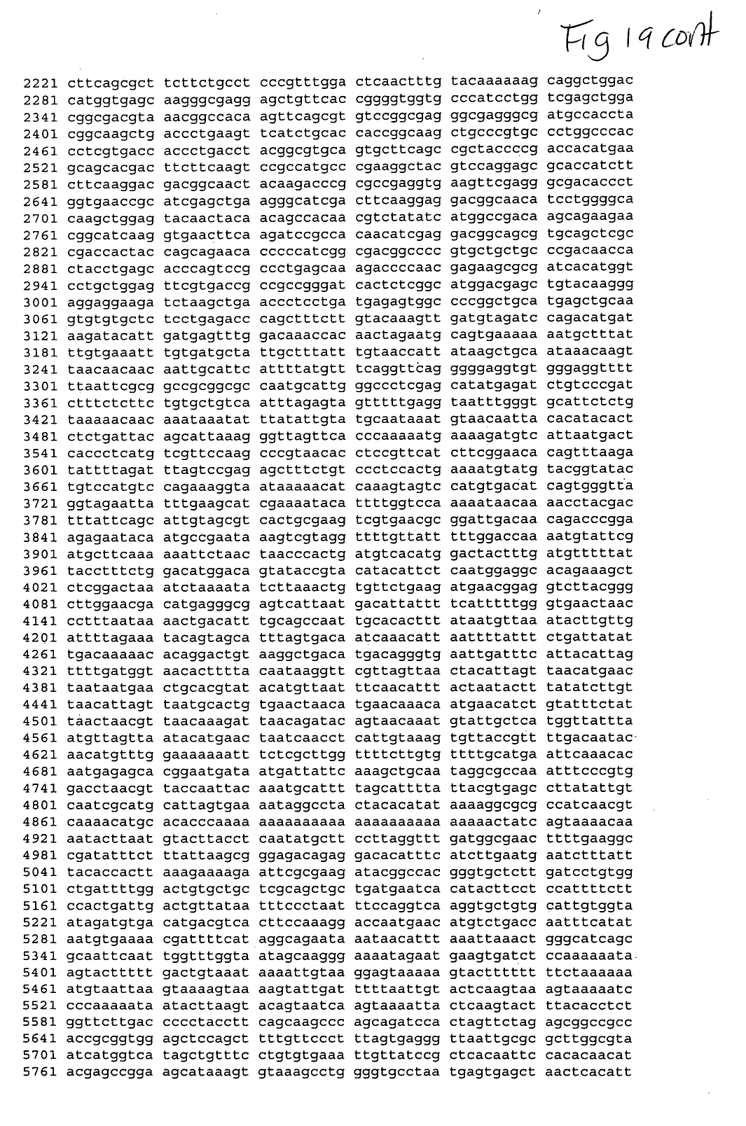

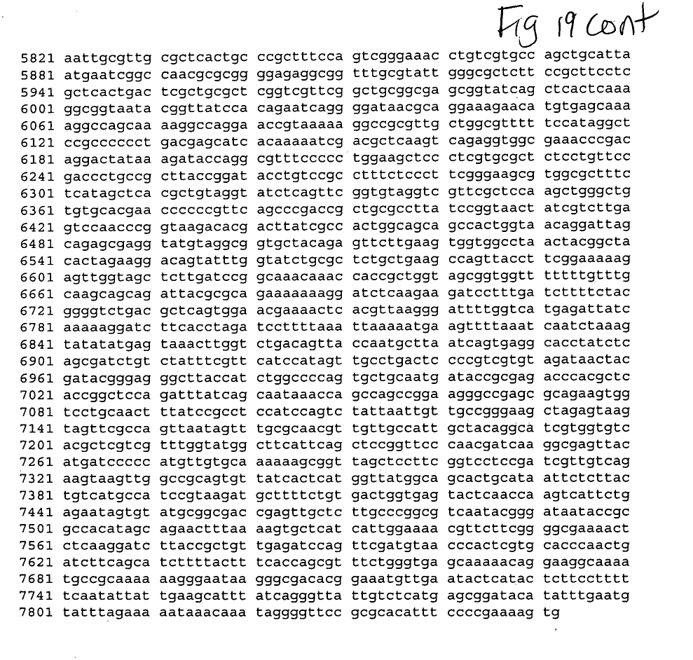

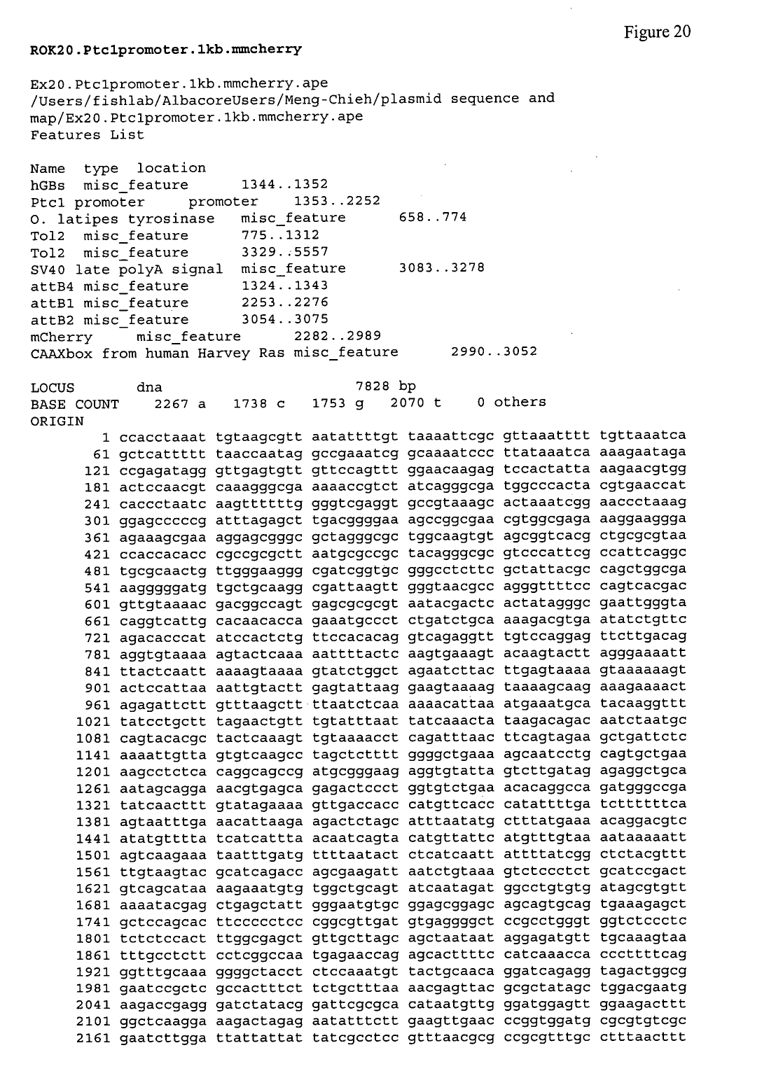

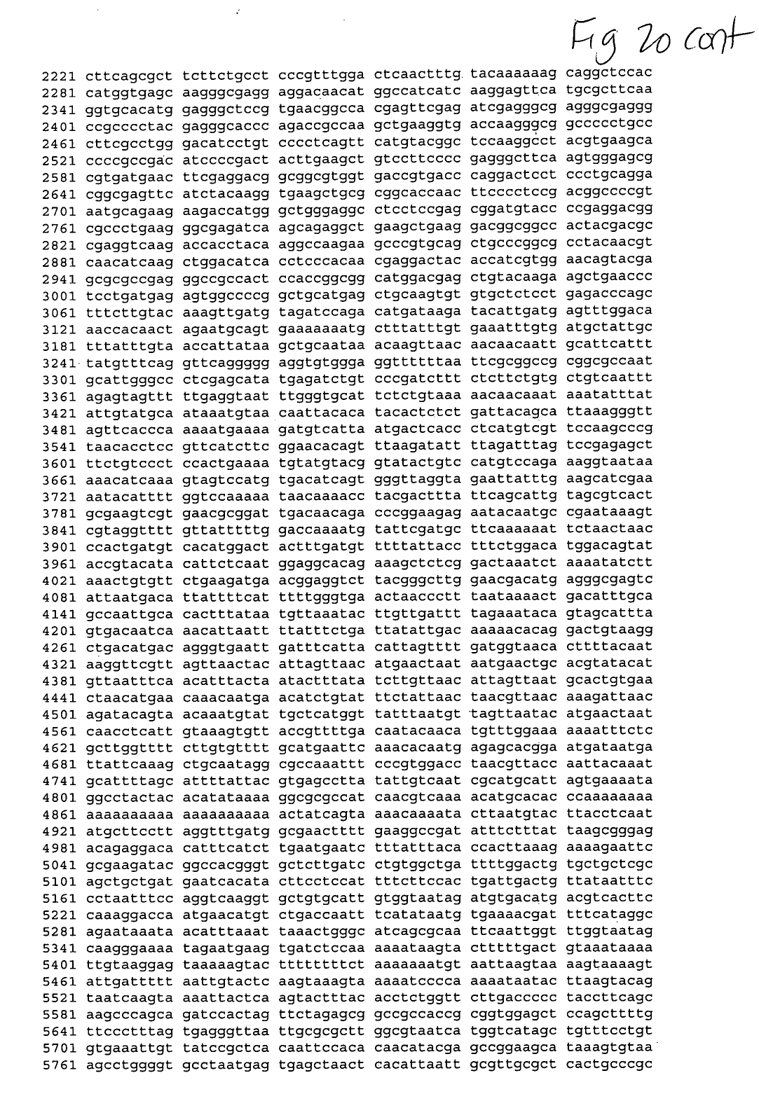

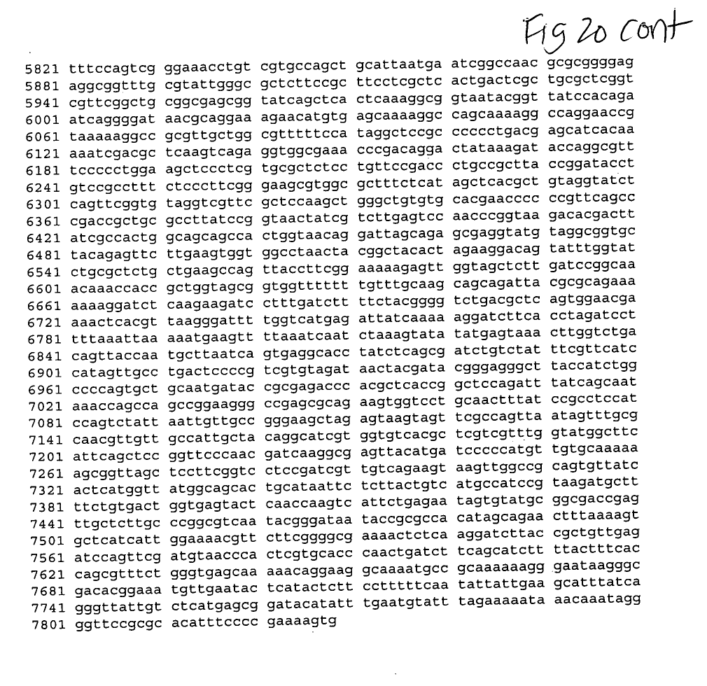

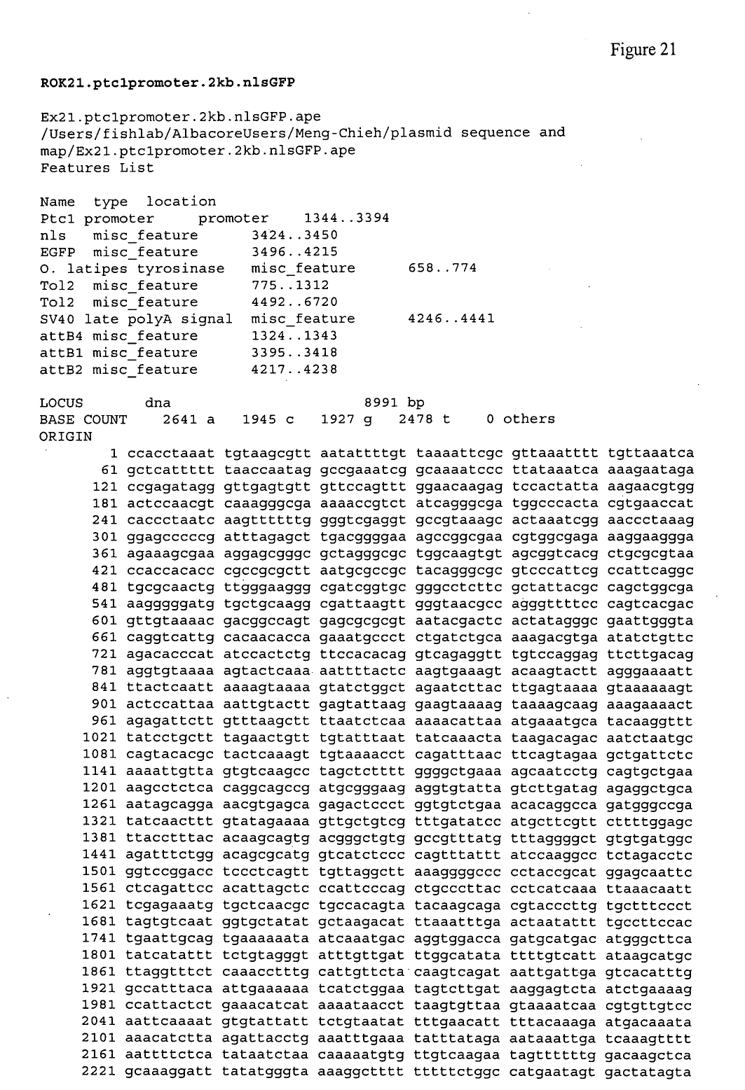

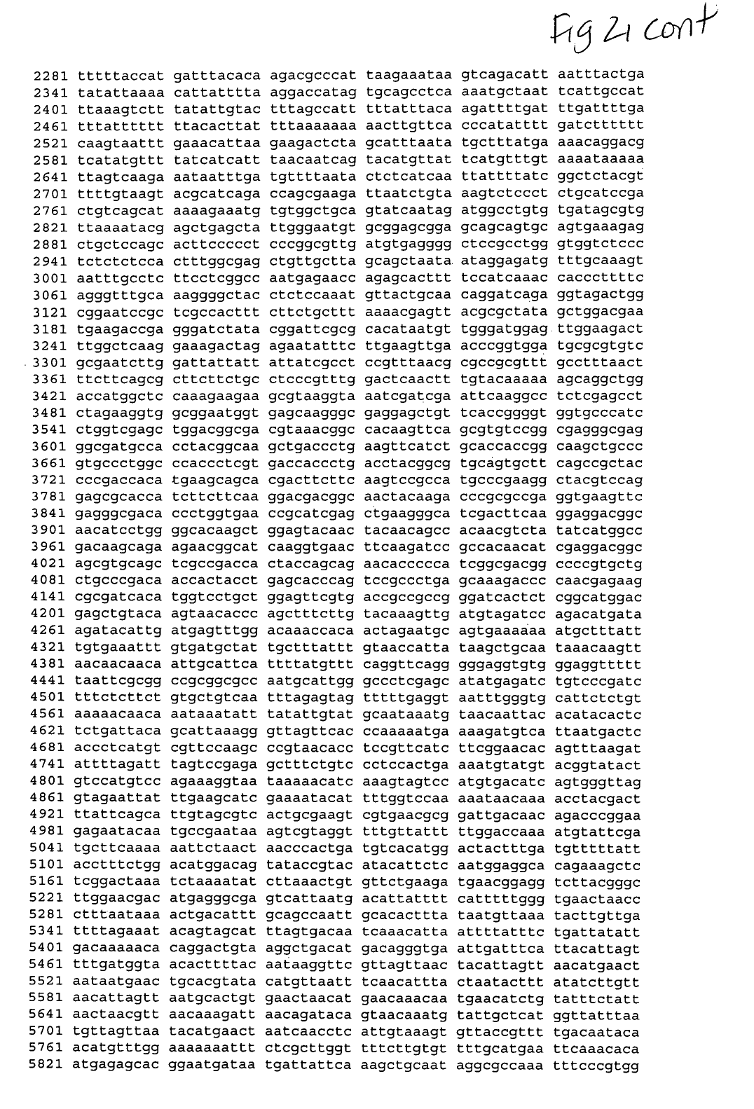

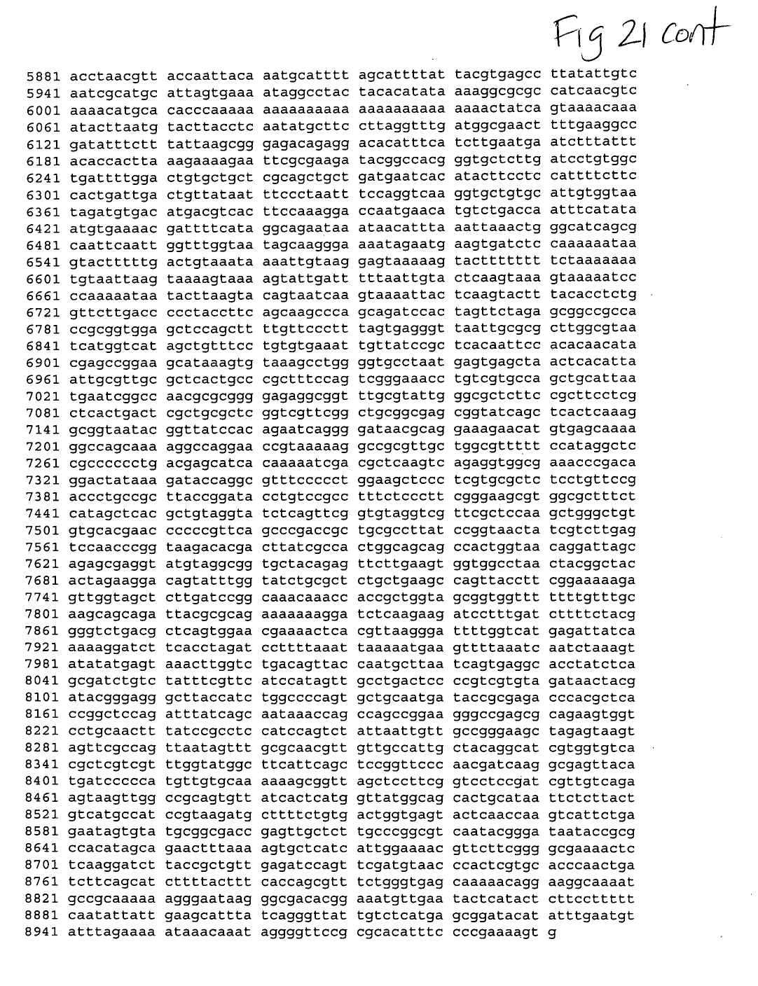

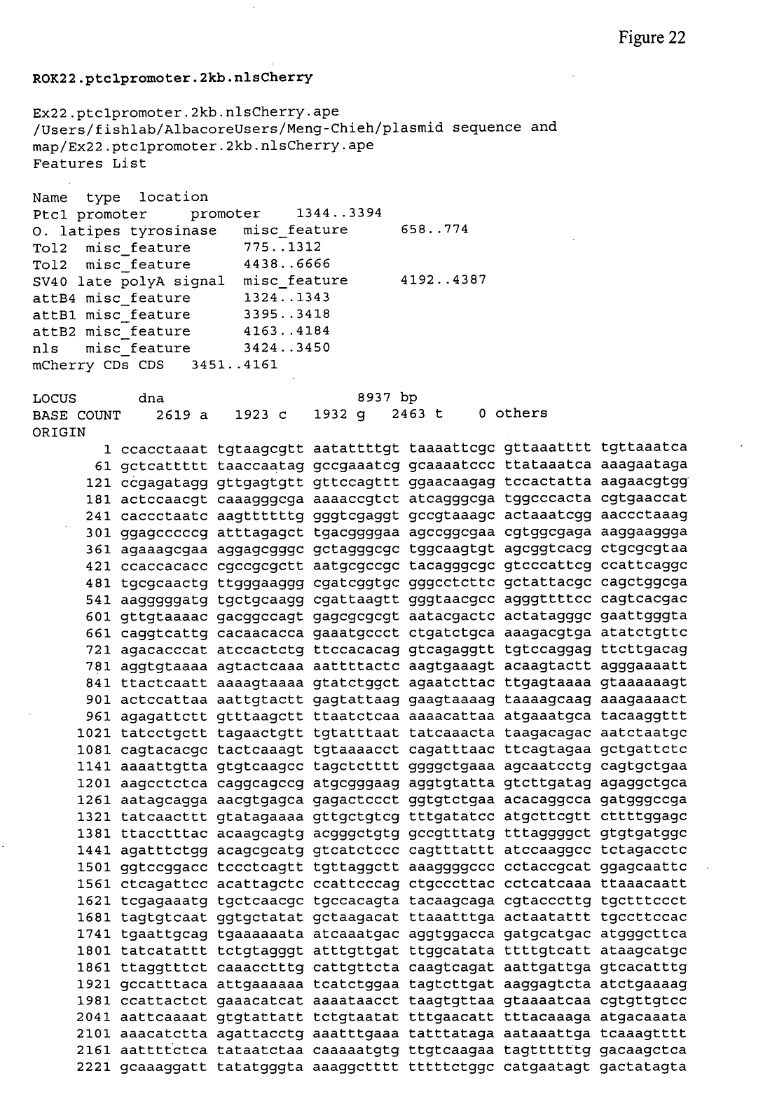

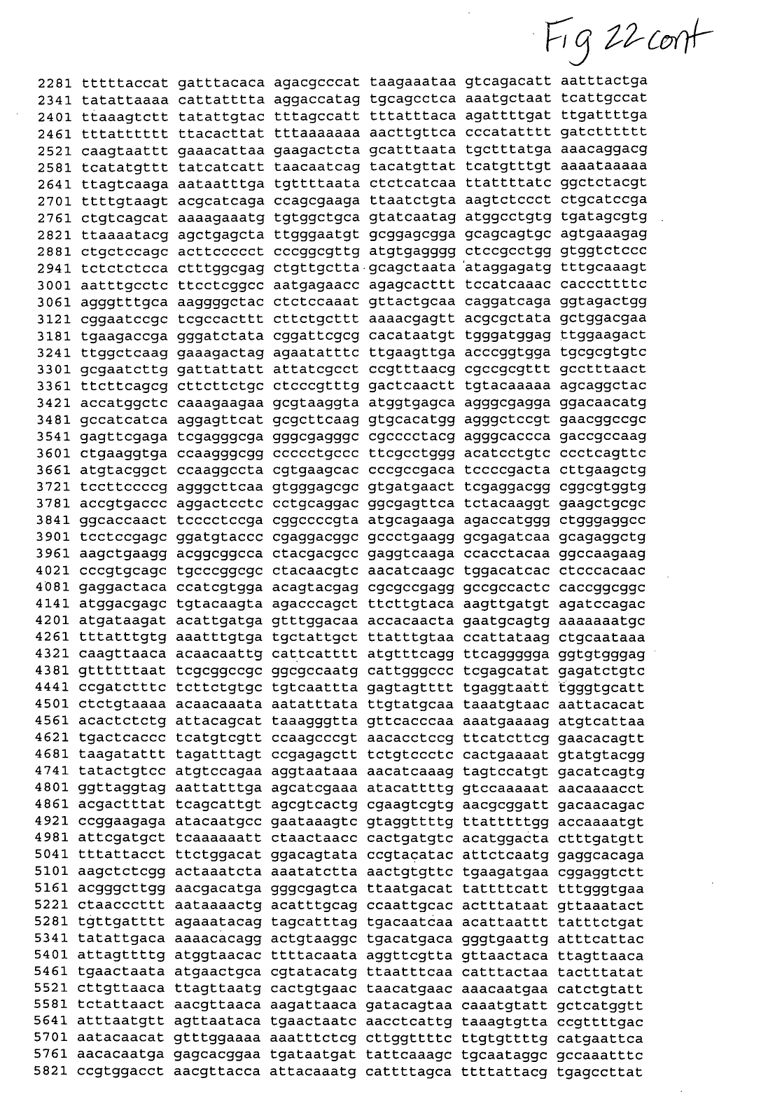

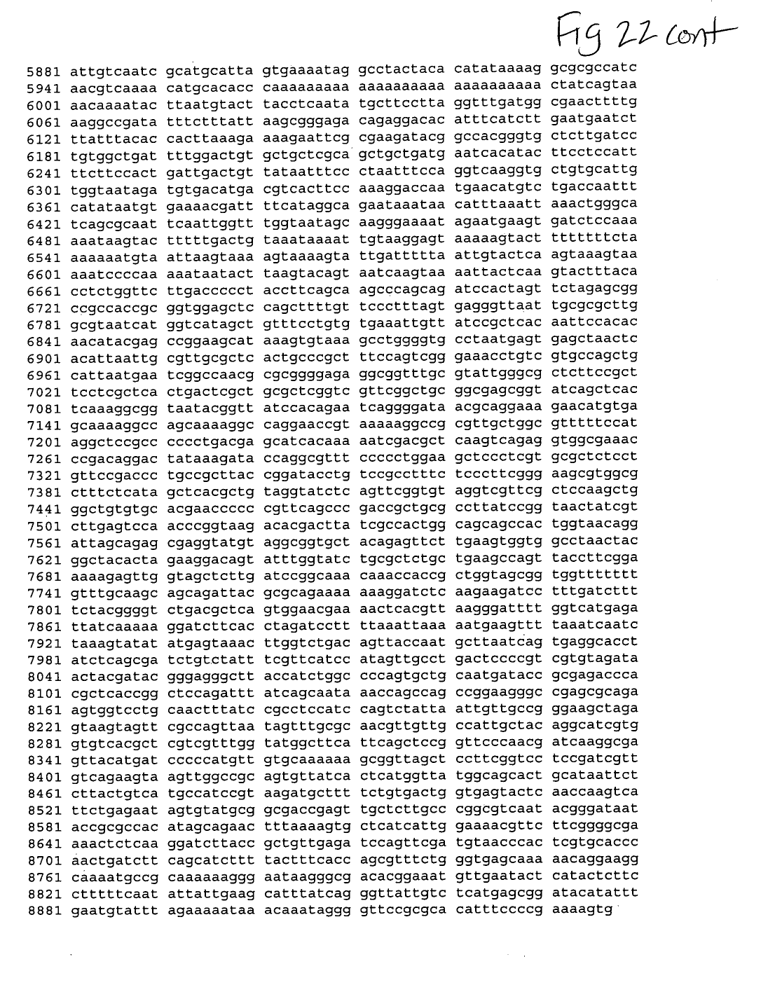

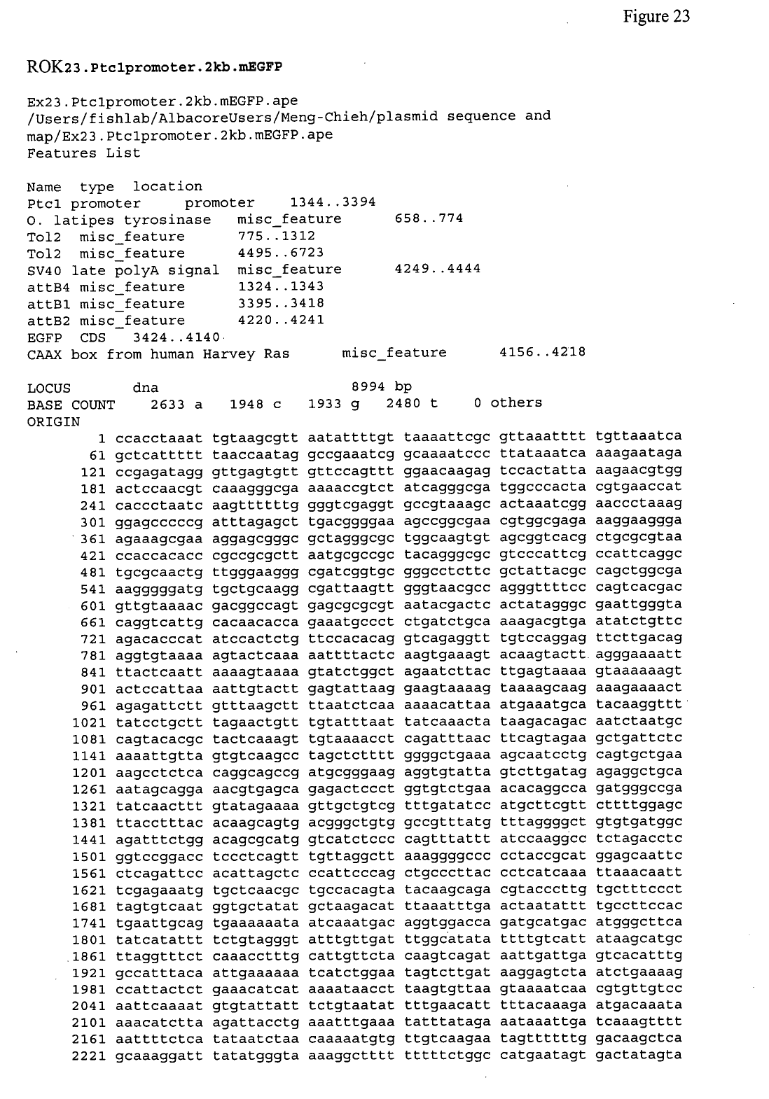

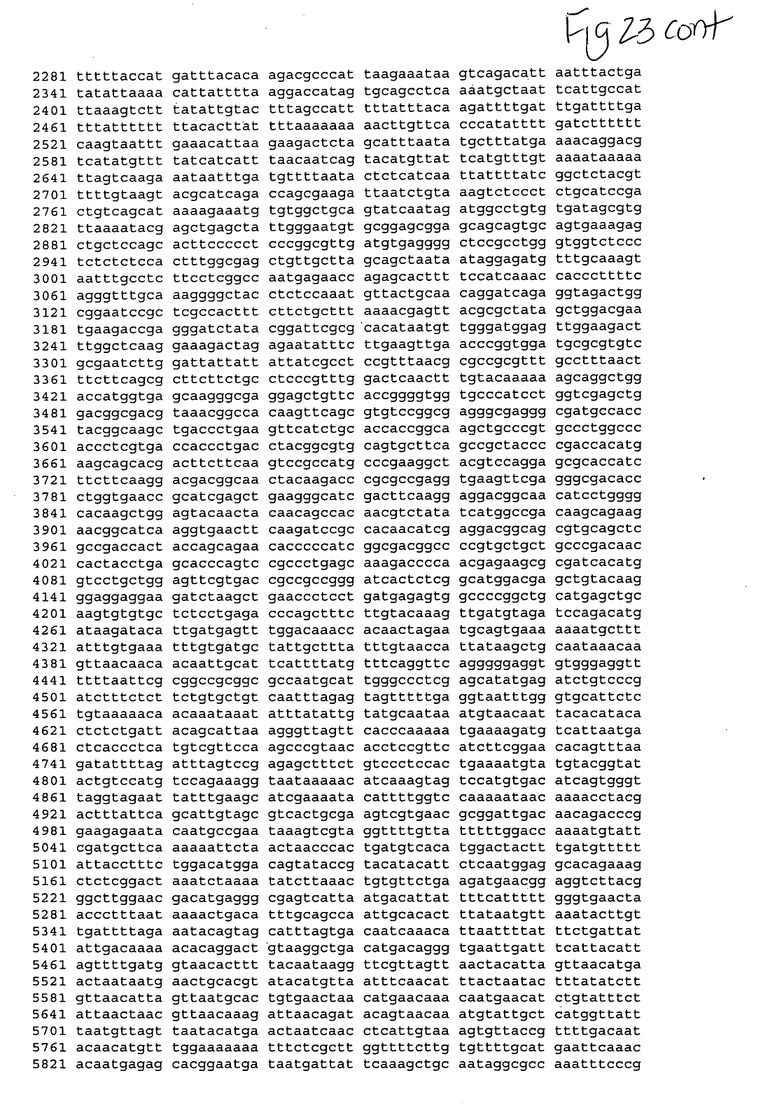

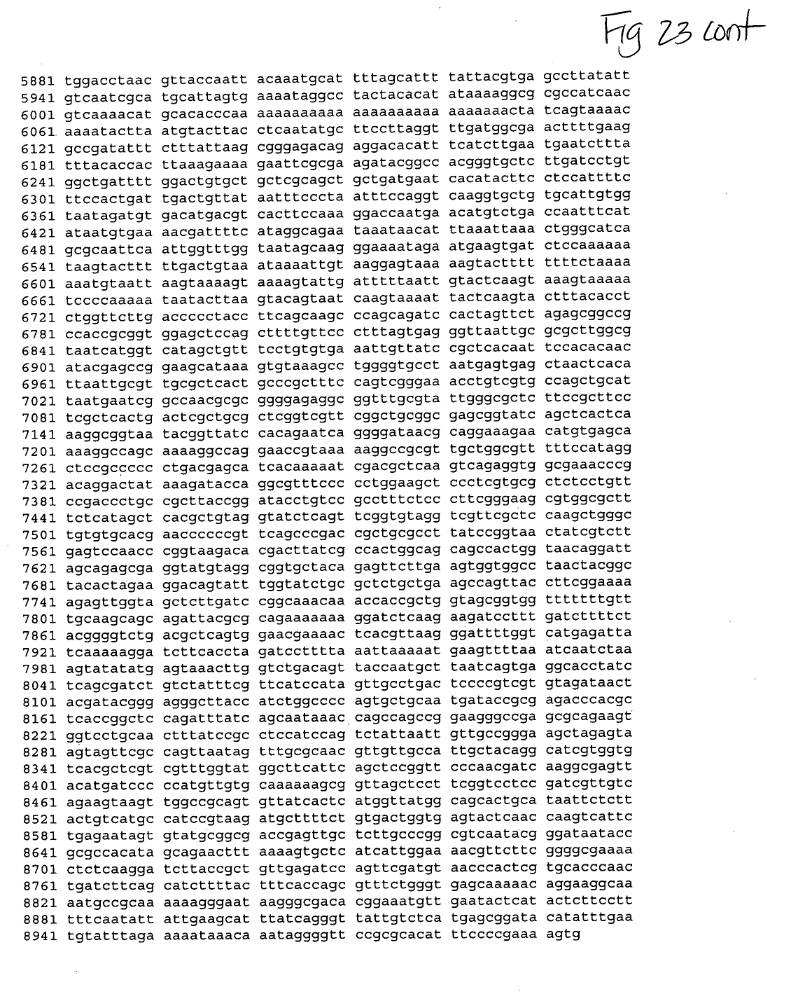

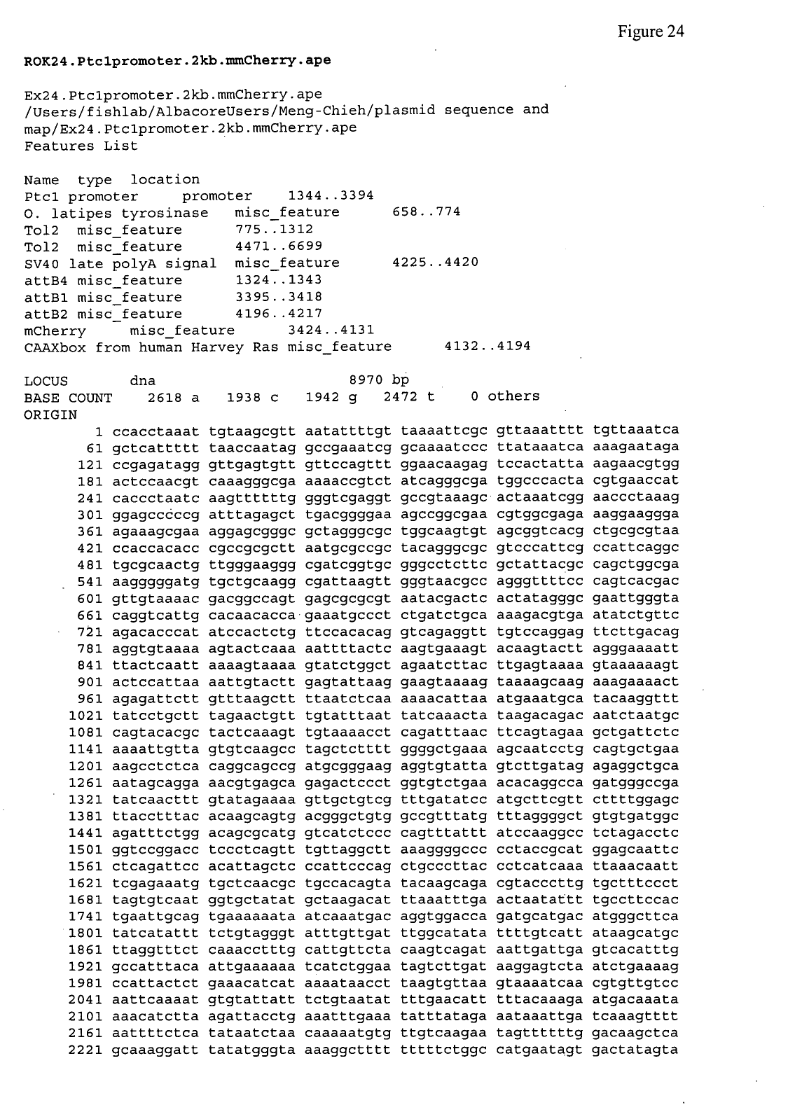

FIGS. 13-24: Ptc1: Patched 1 promoter region; 500 bp: the length of it is around 500 base pairs, and it actually contains 517 base pair nucleotide up stream of 1st CDs; 1 kb: the length of it is around 1000 base pairs, and it actually contains 900 base pair nucleotide up stream of 1st CDs; 2 kb: the length of it is around 2000 base pairs, and it actually contains 2051 base pair nucleotide up stream of 1st CDs; nlsGFP: nucleus enhanced green fluorescent protein; nlsCherry: nucleus monomeric Cherry; mEGFP: membrane enhanced green fluorescent protein; mmCherry: membrane monomeric Cherry.

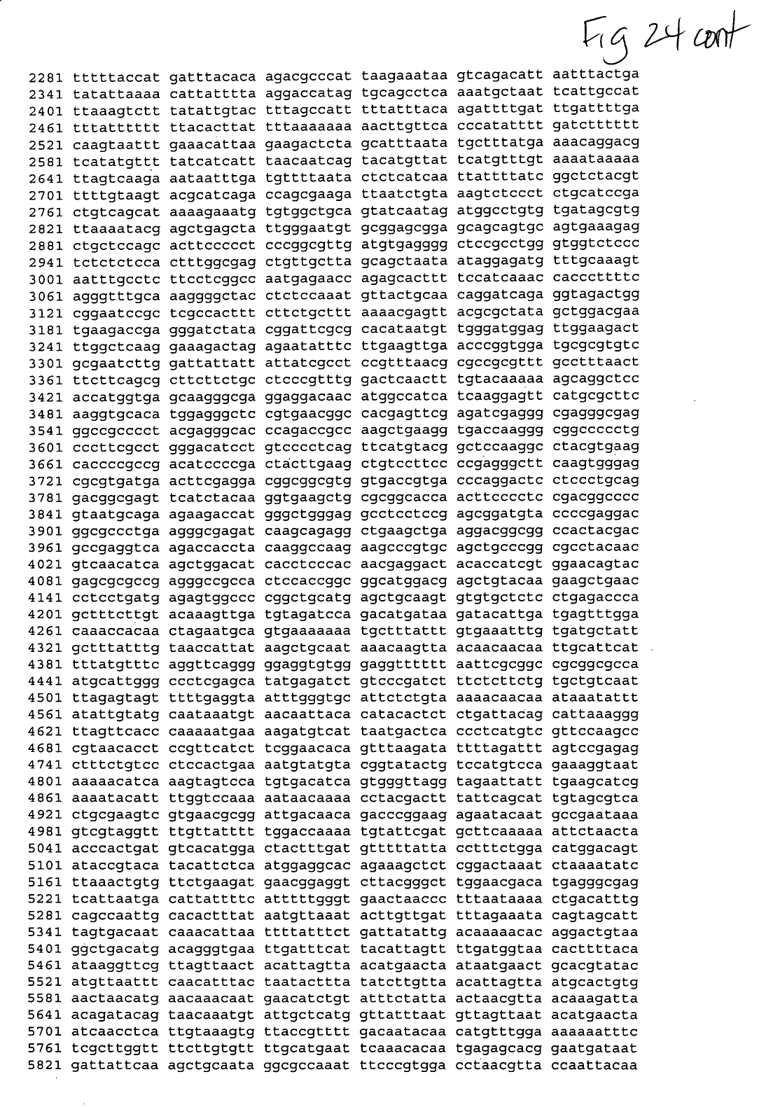

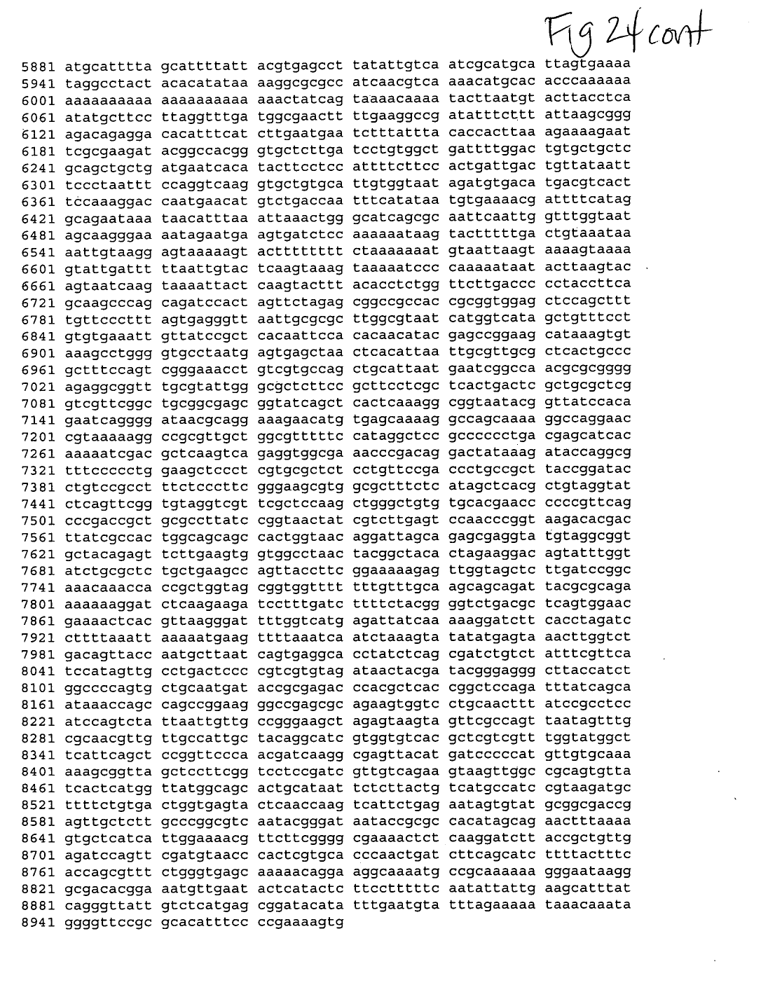

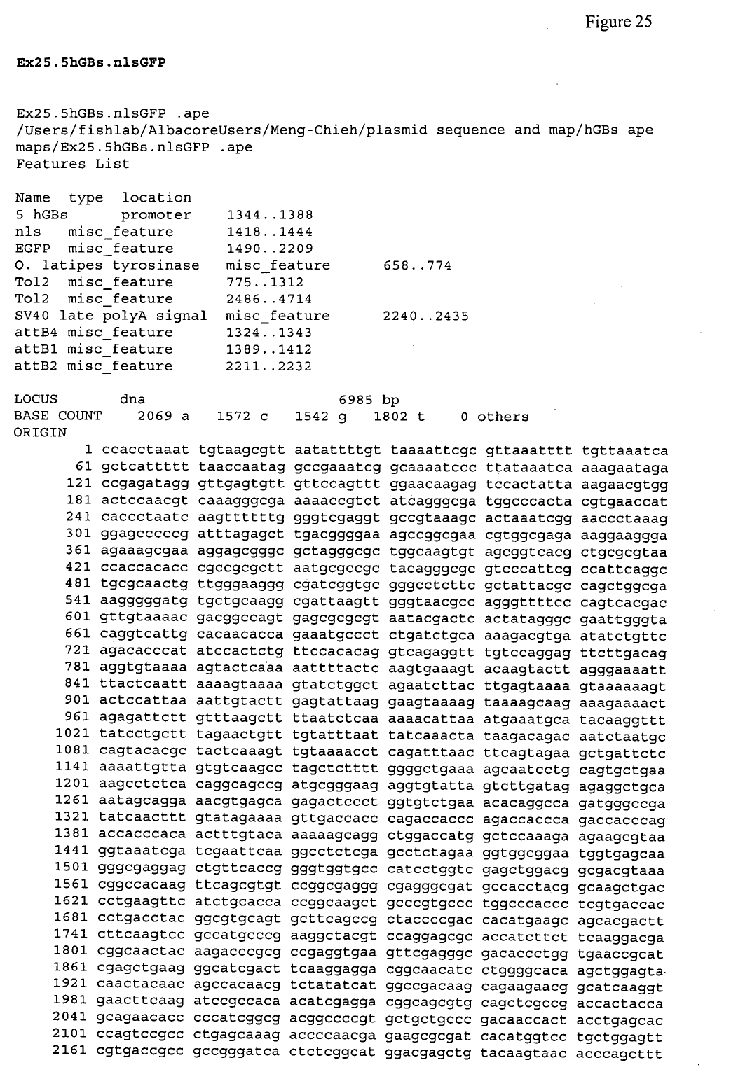

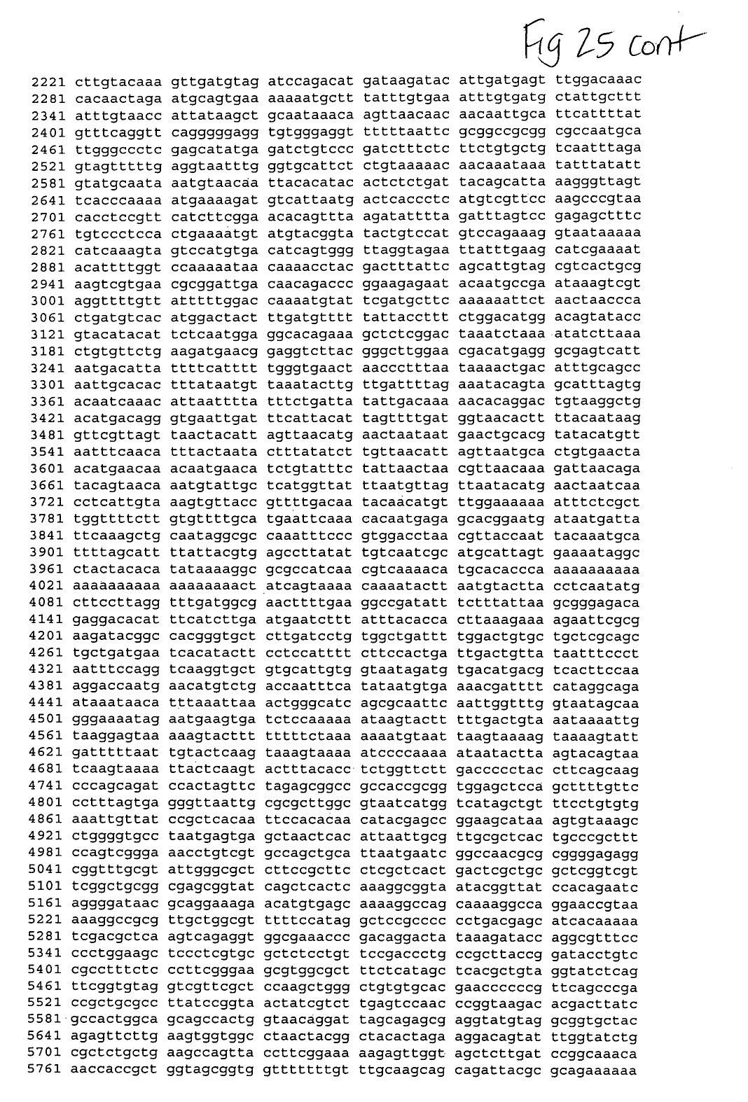

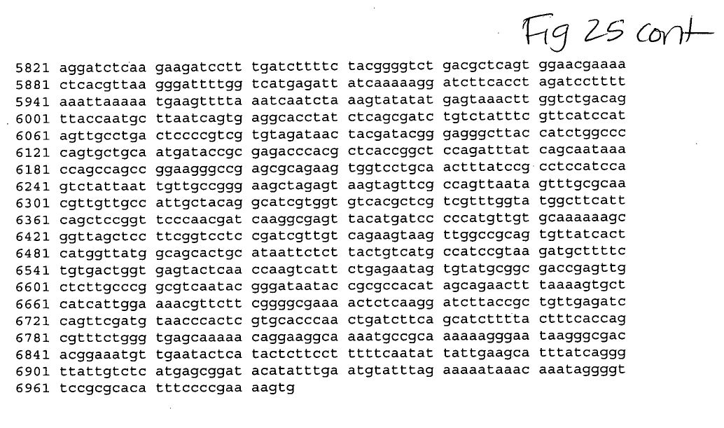

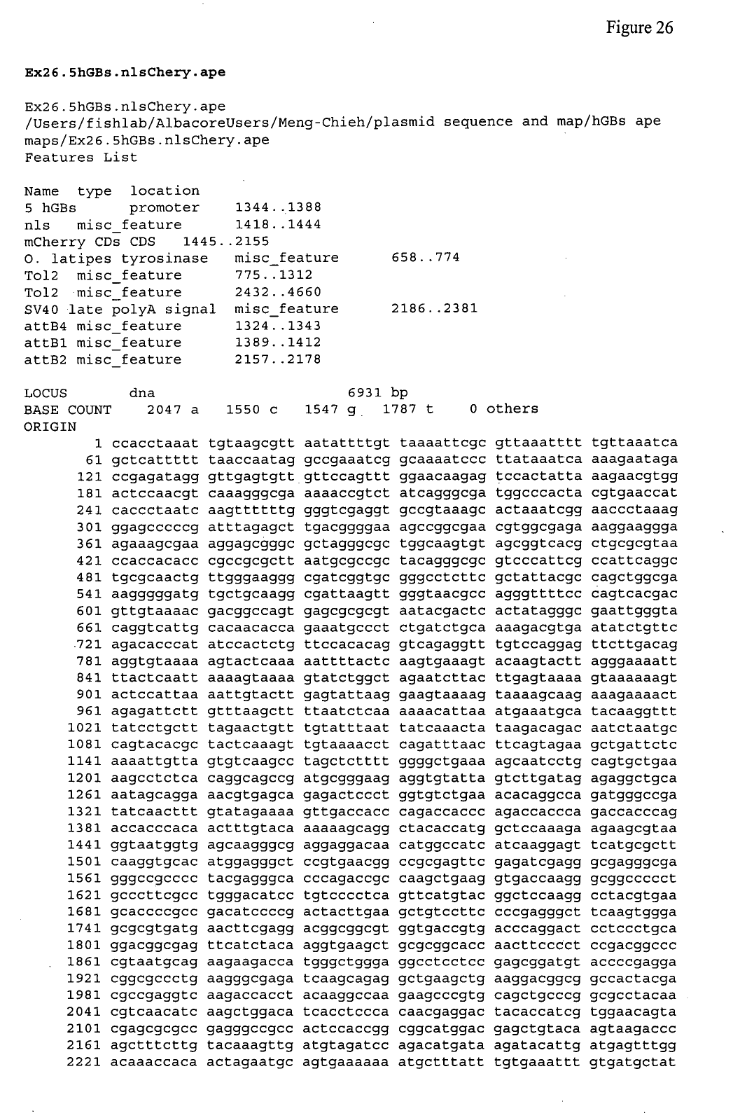

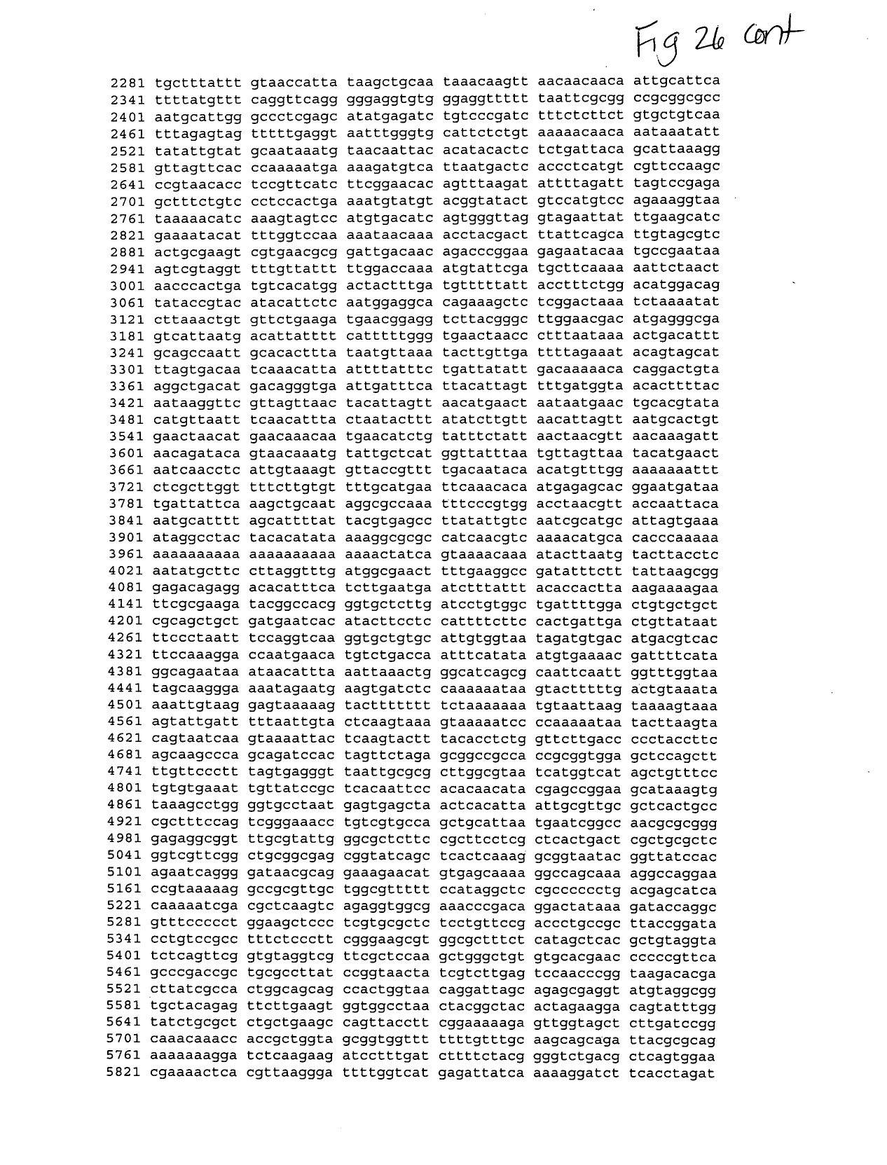

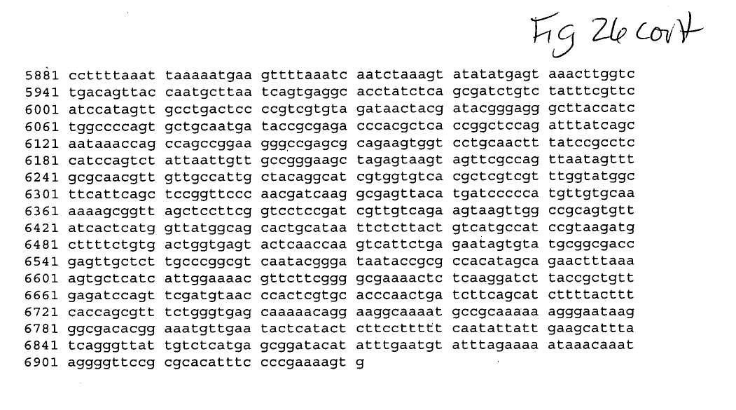

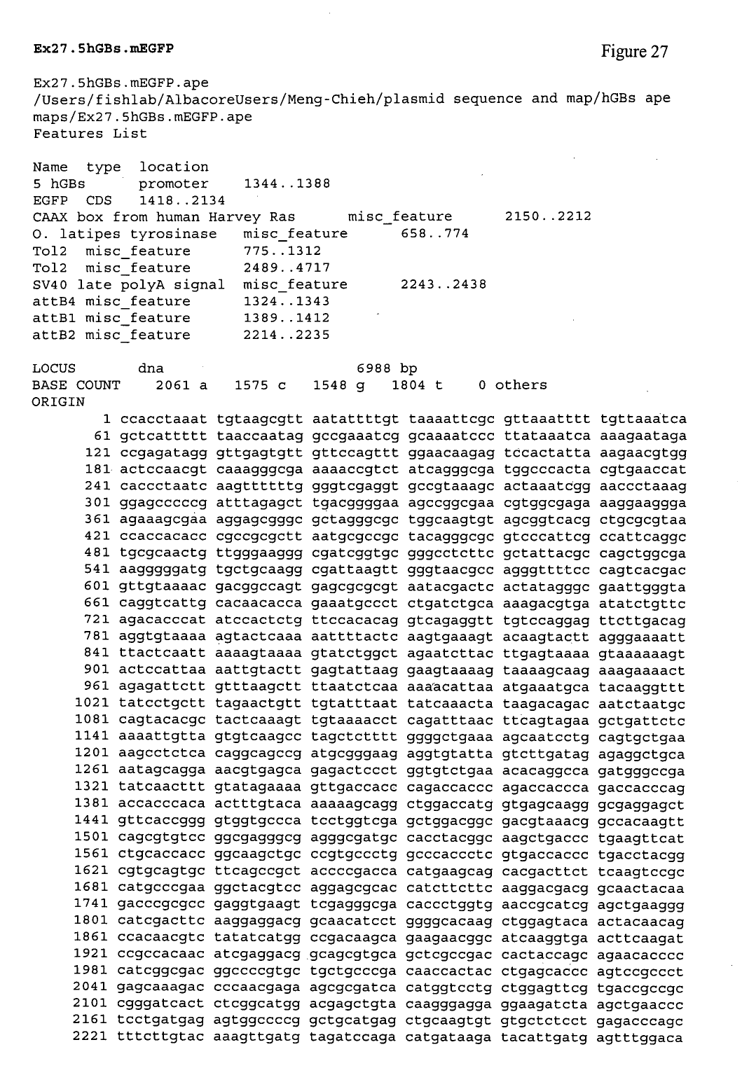

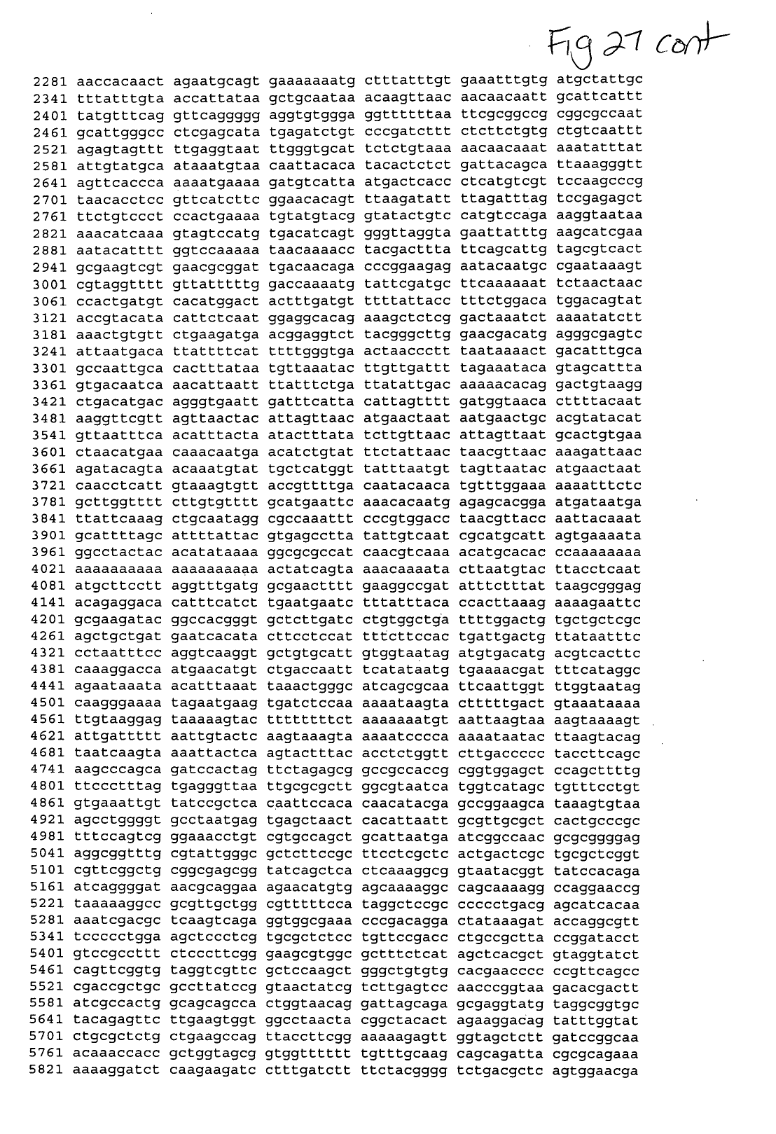

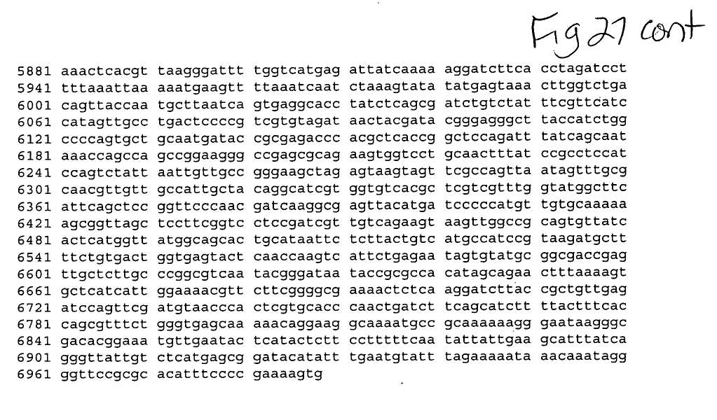

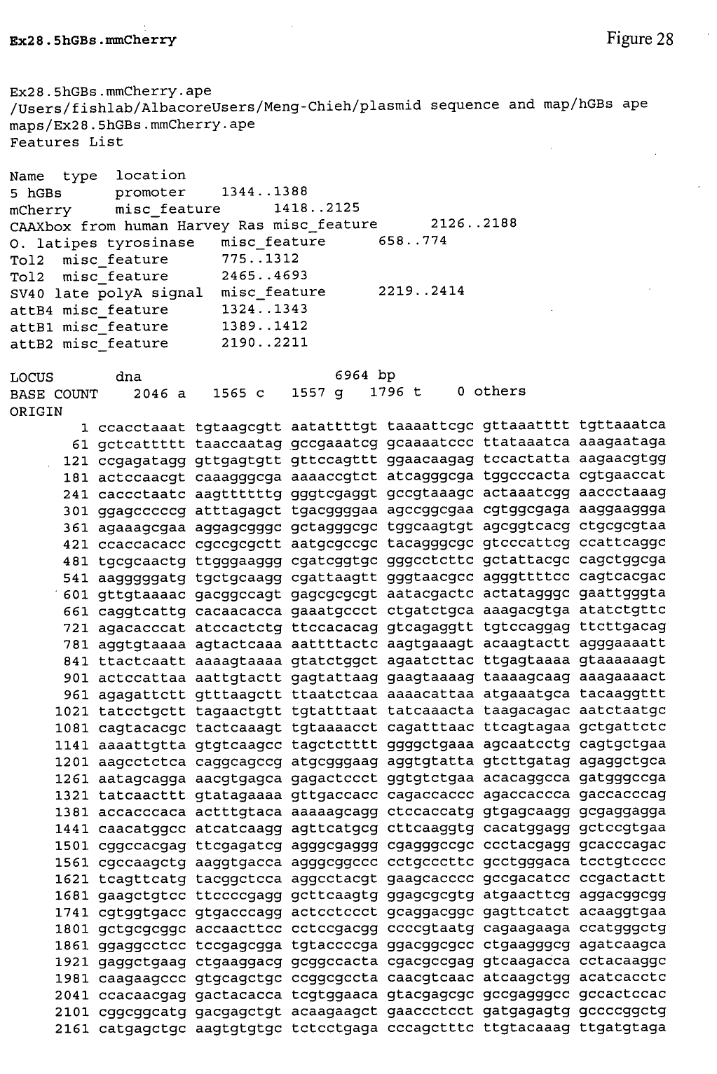

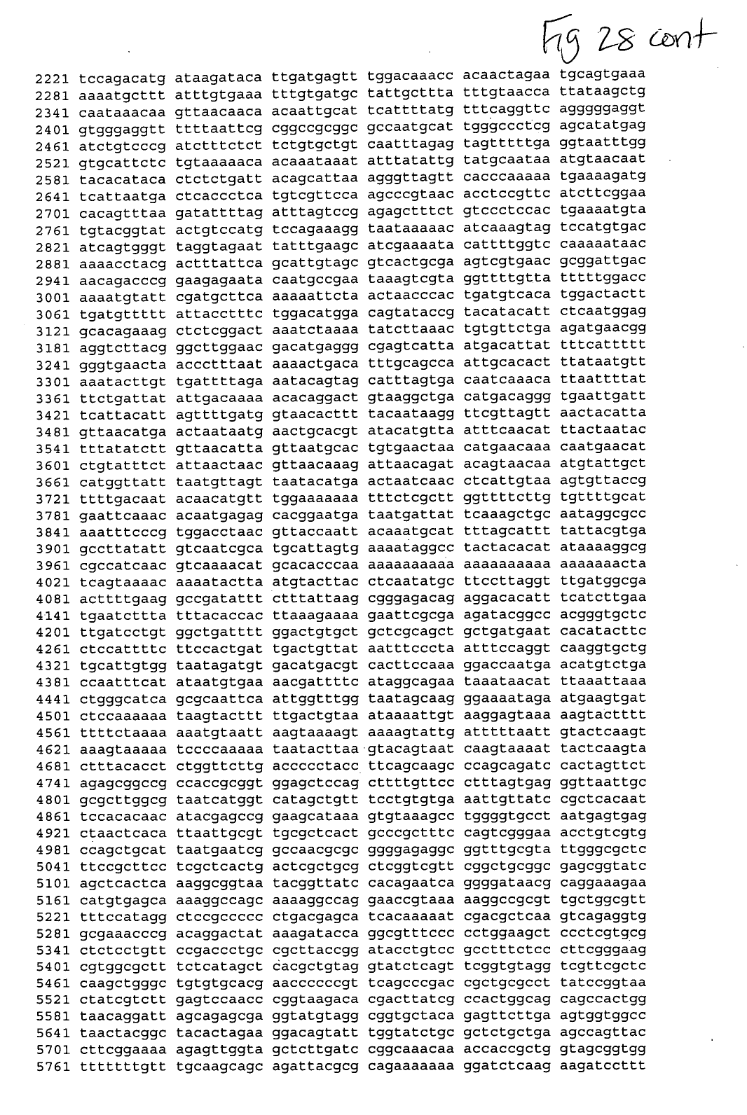

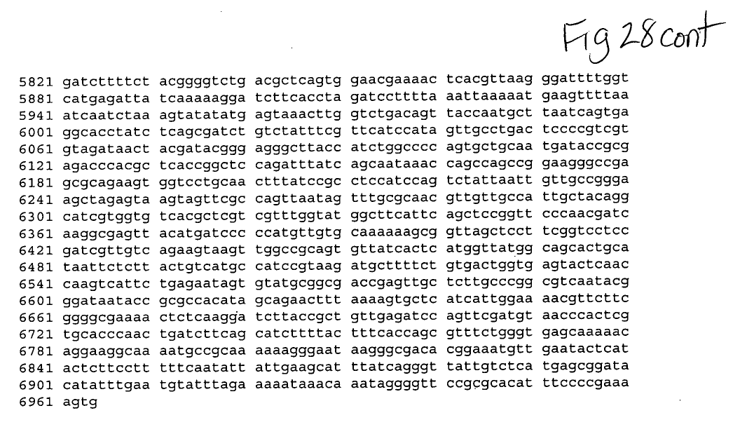

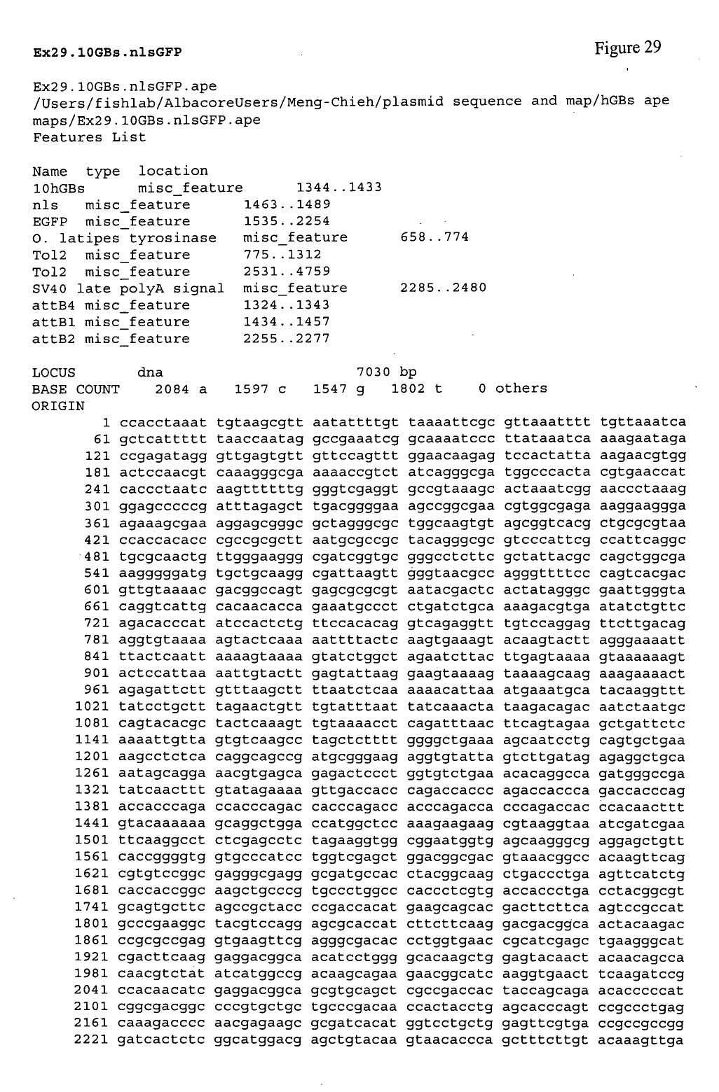

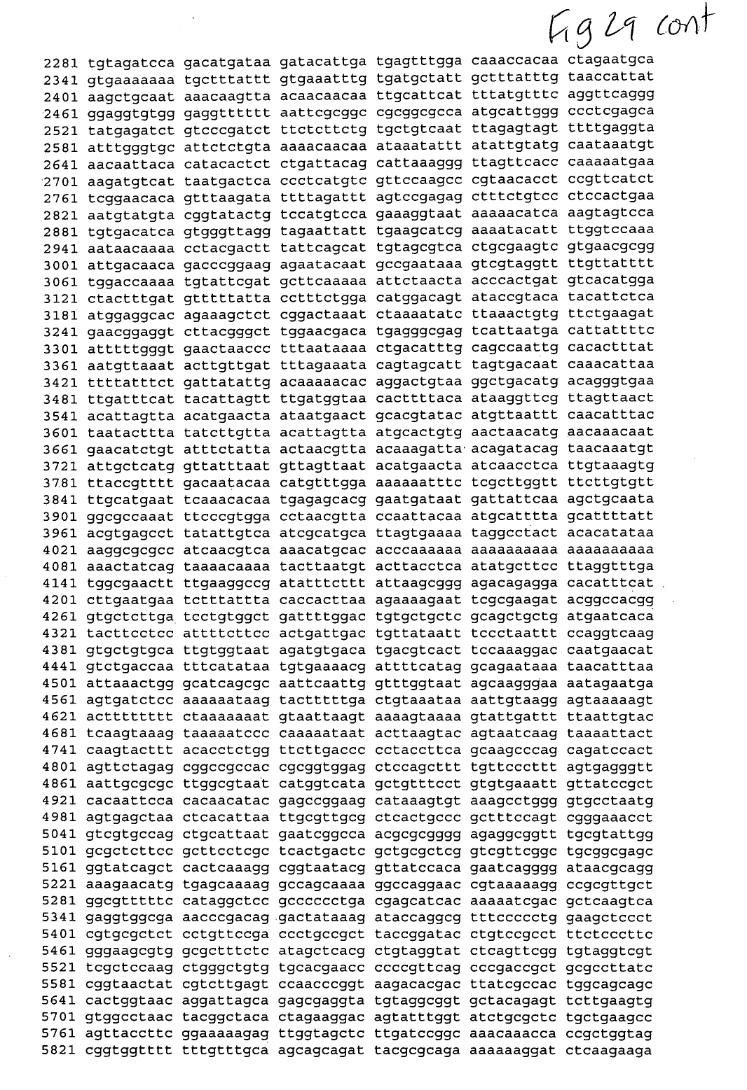

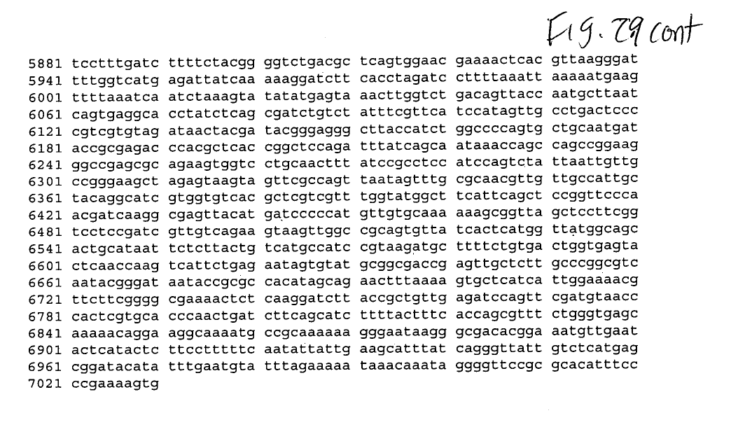

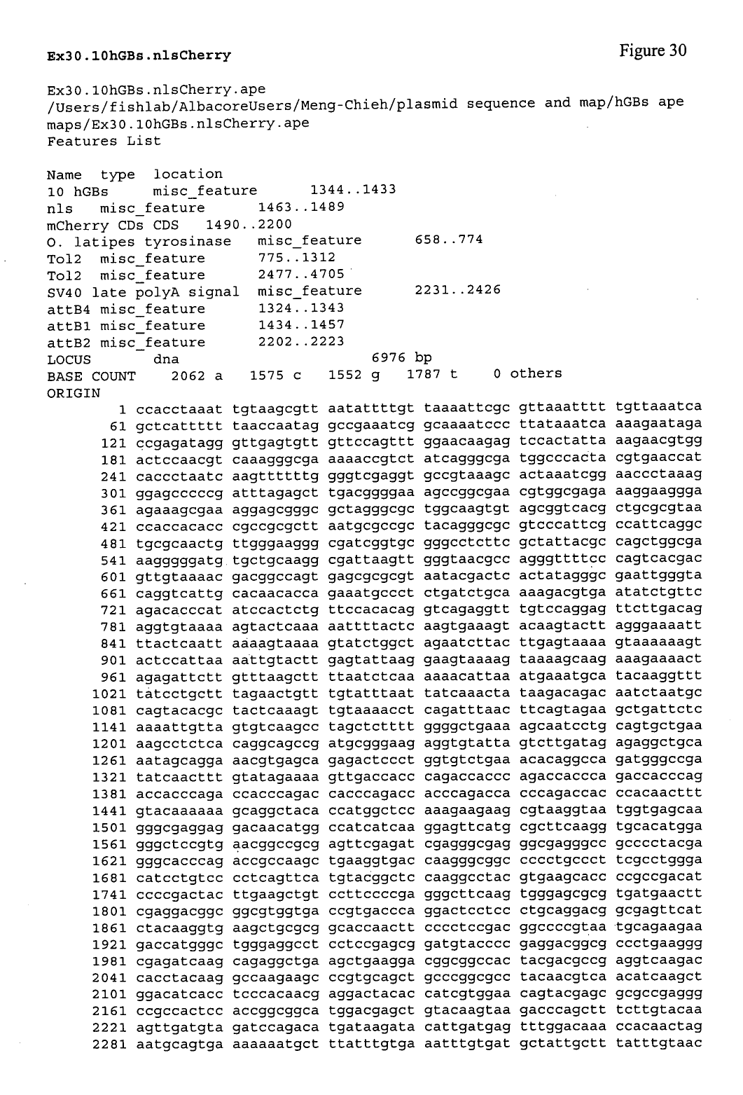

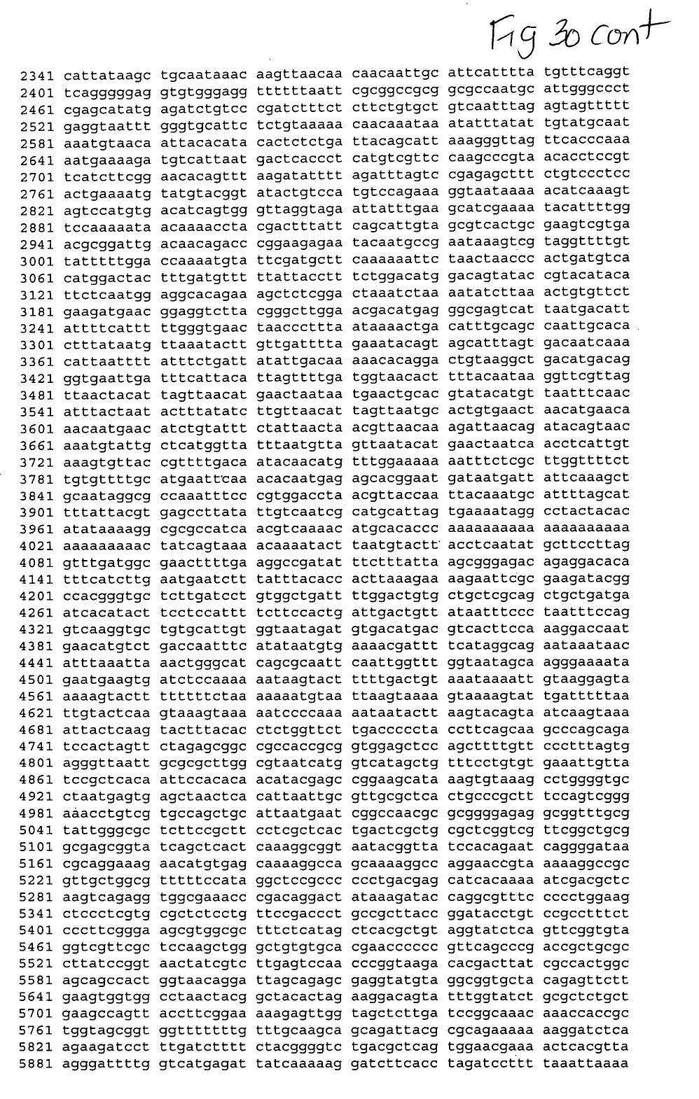

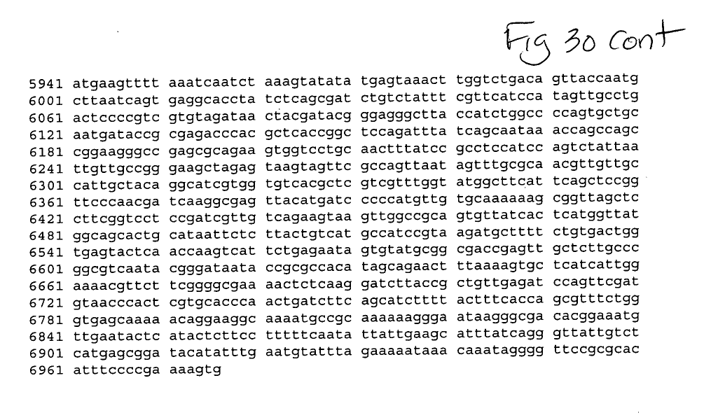

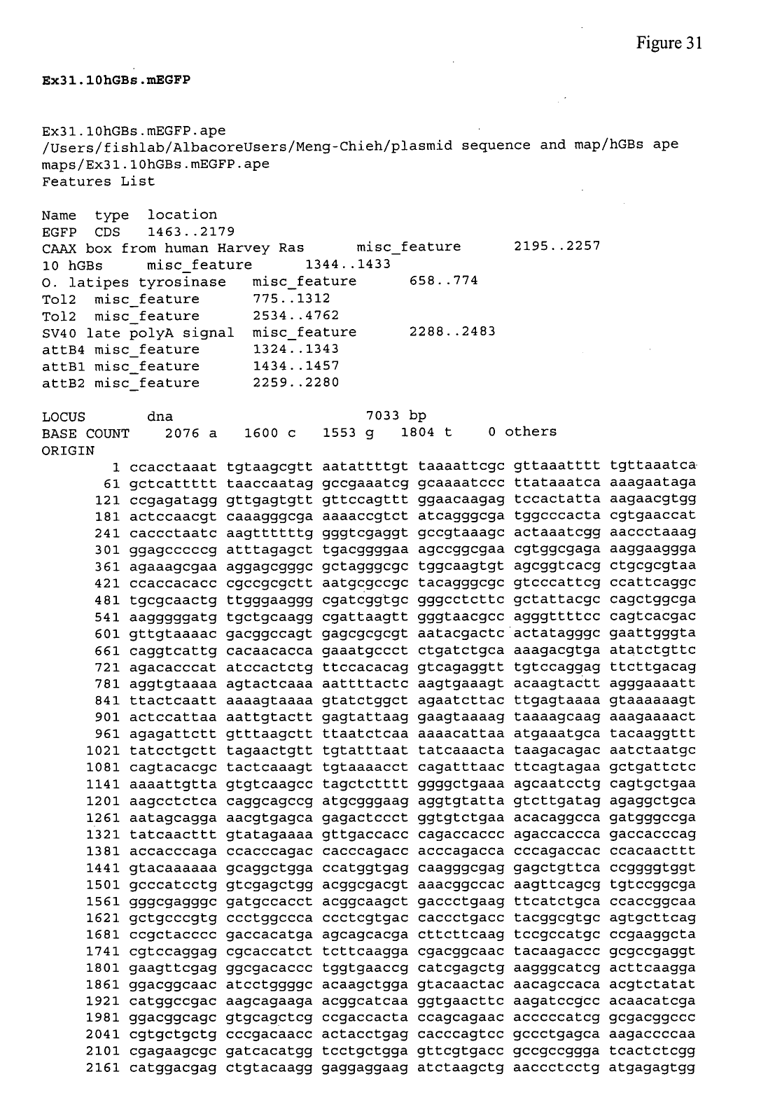

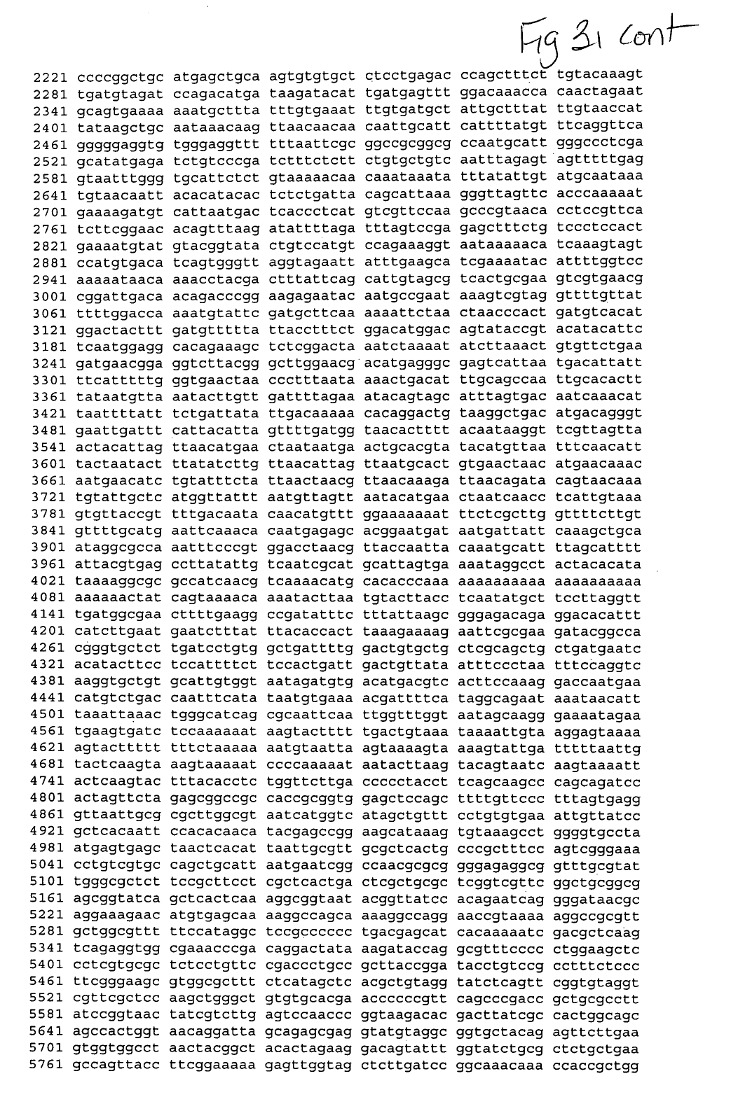

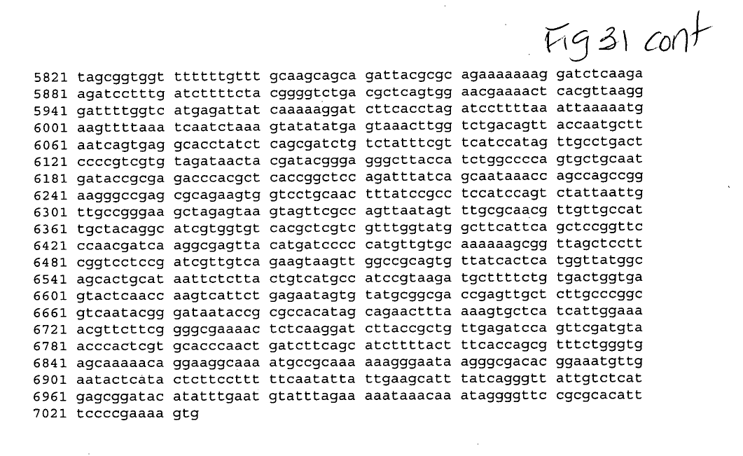

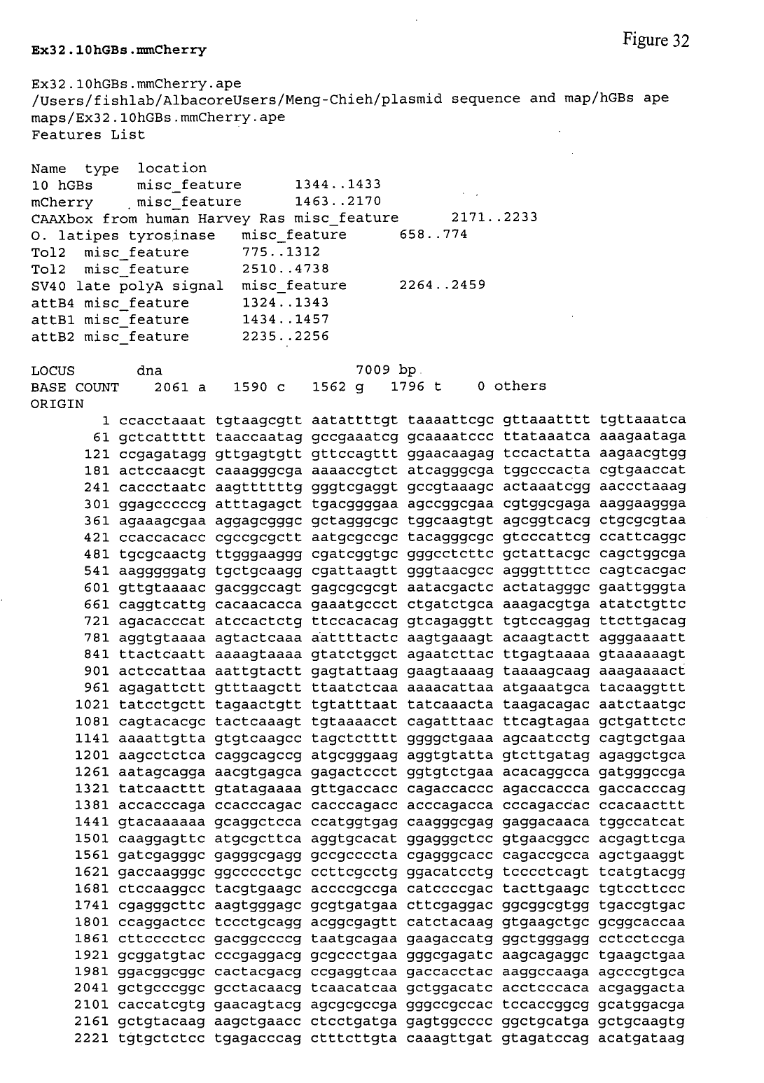

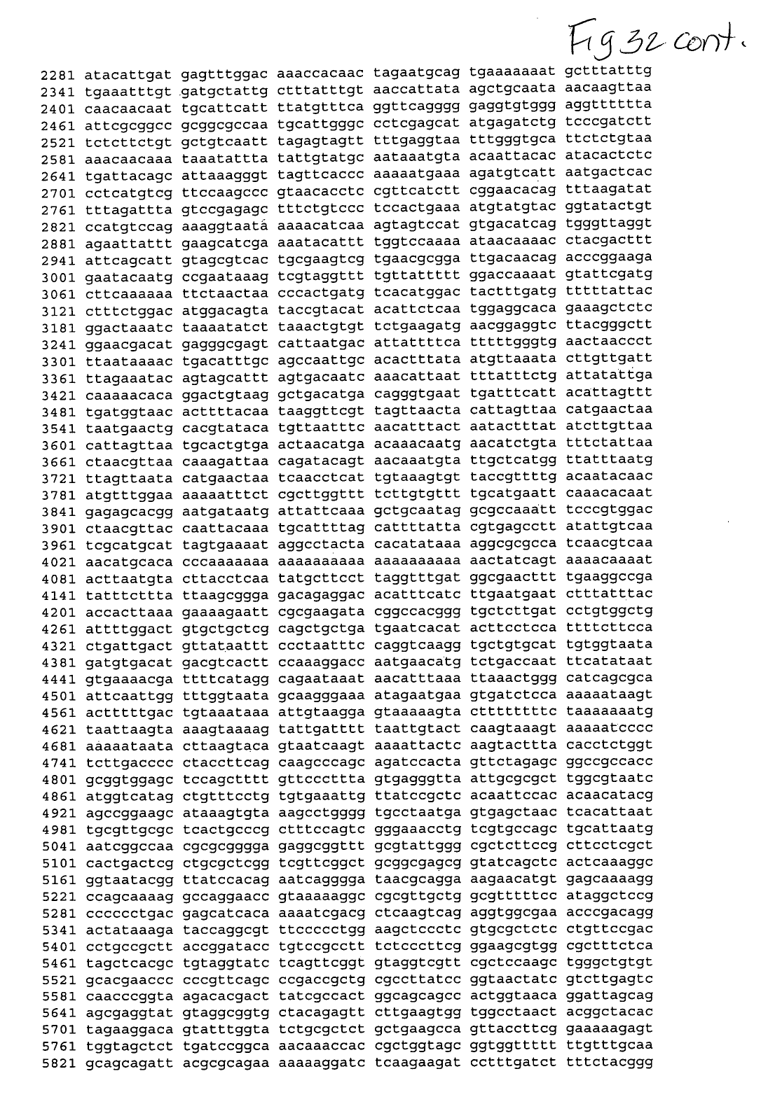

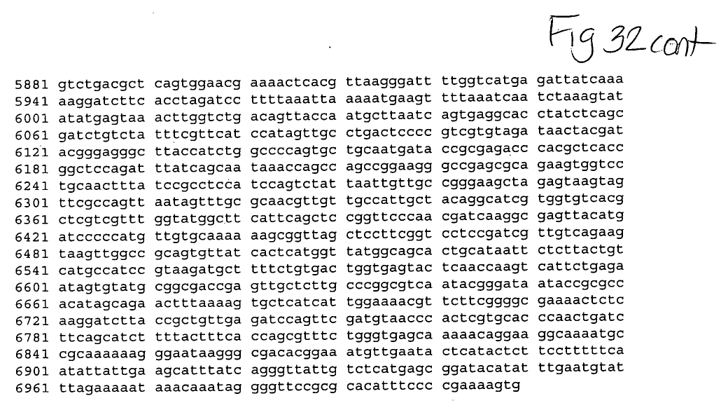

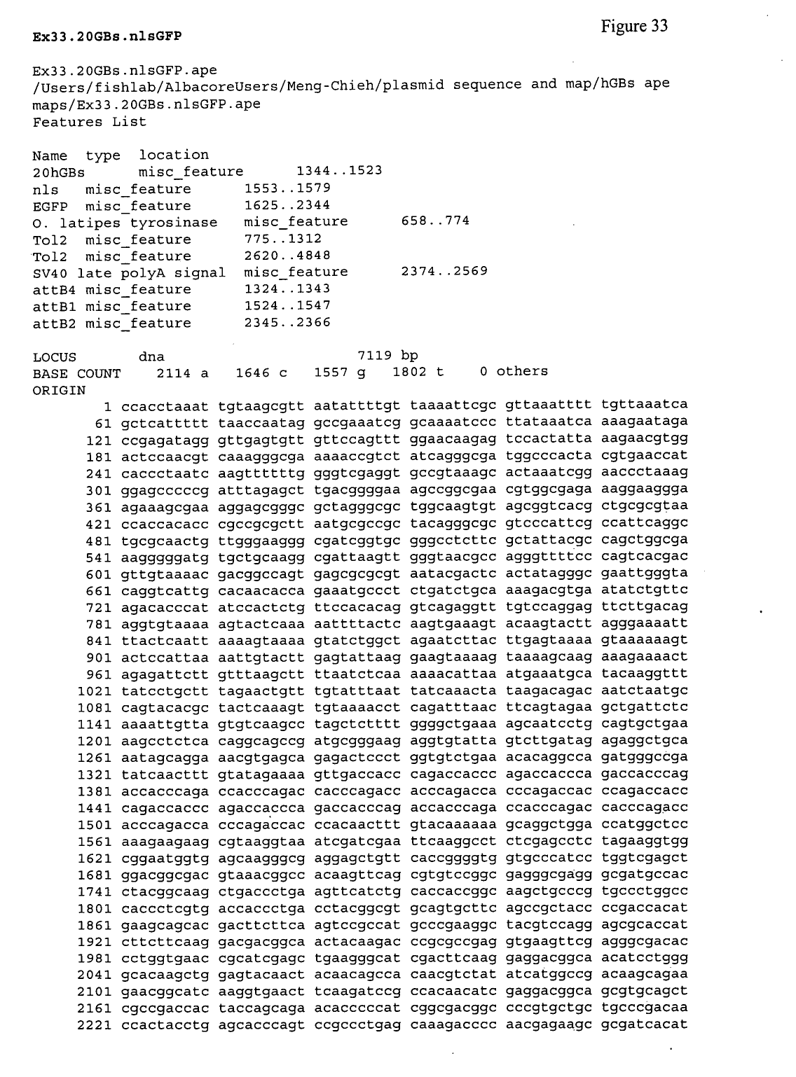

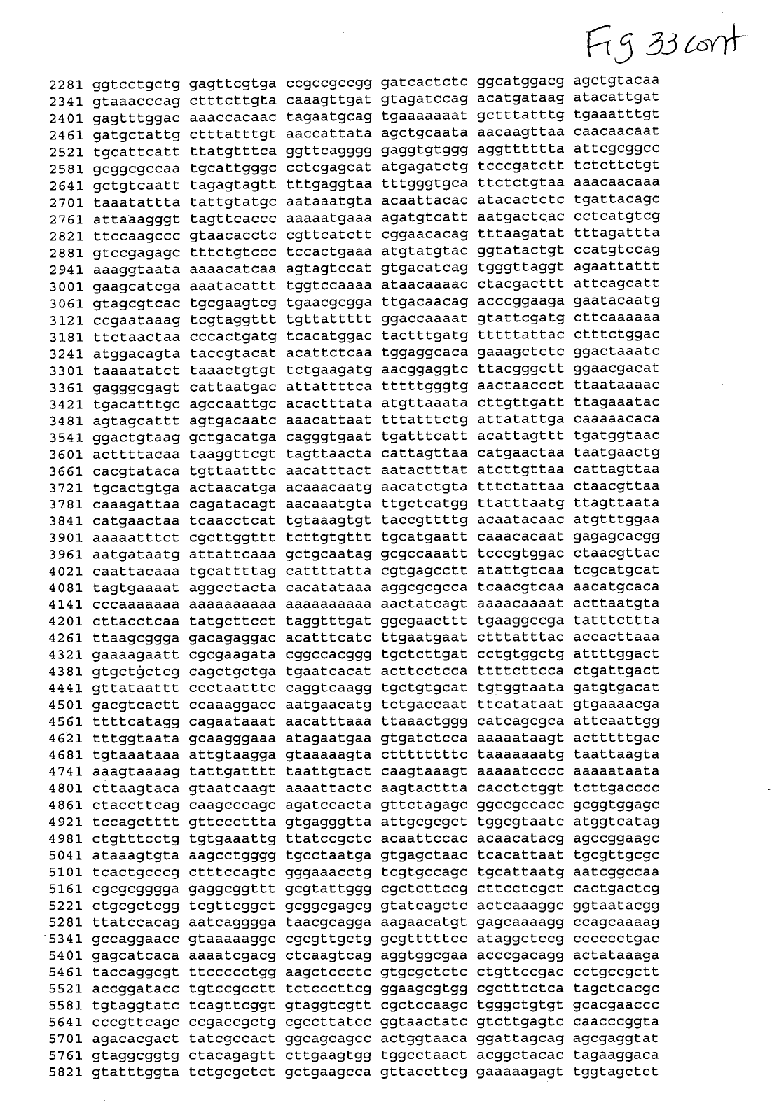

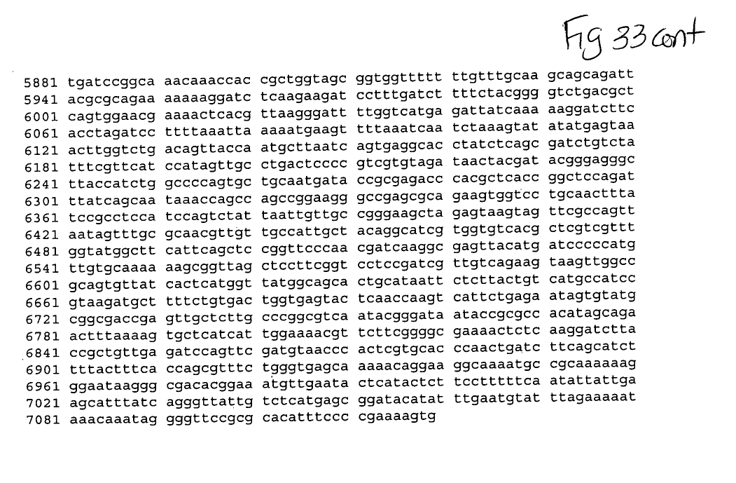

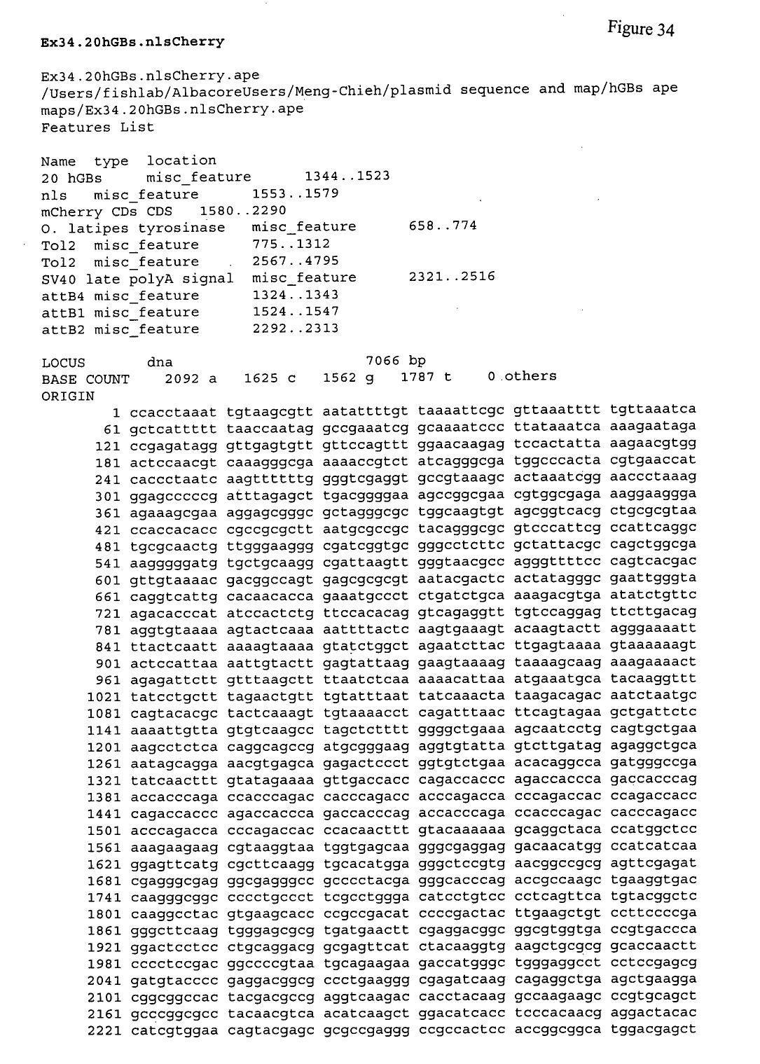

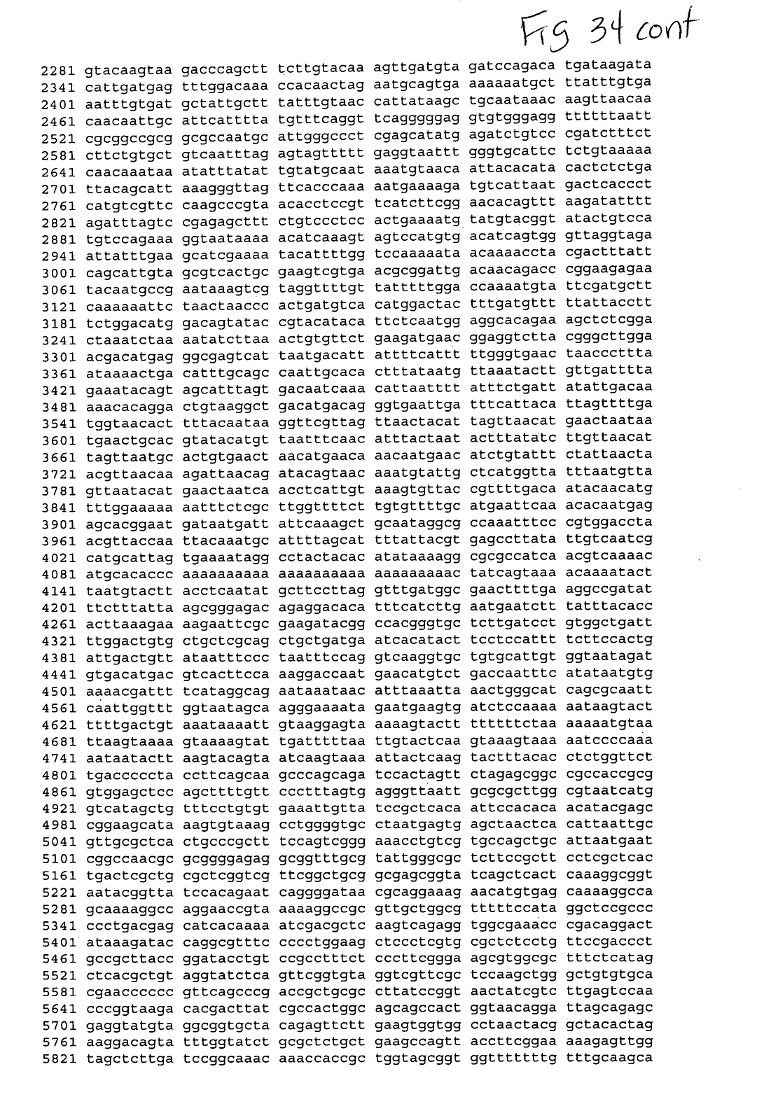

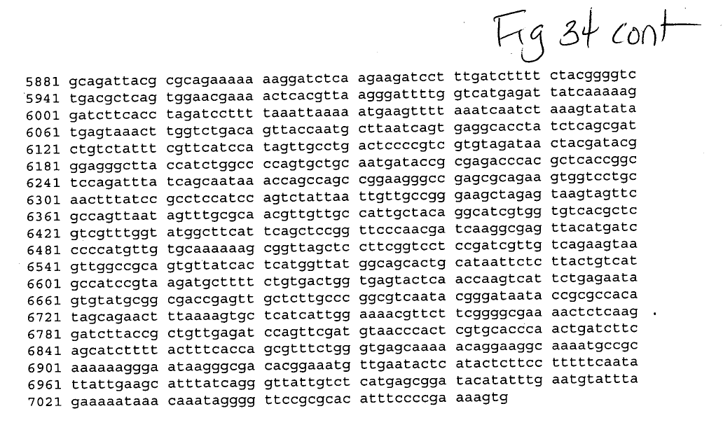

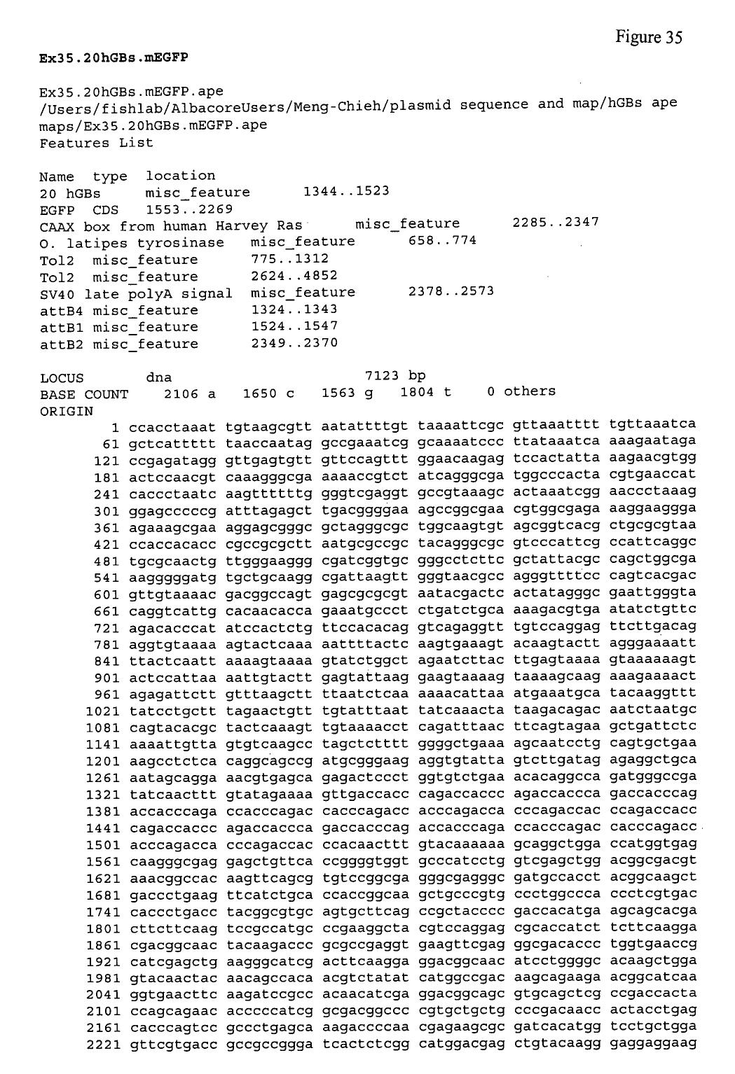

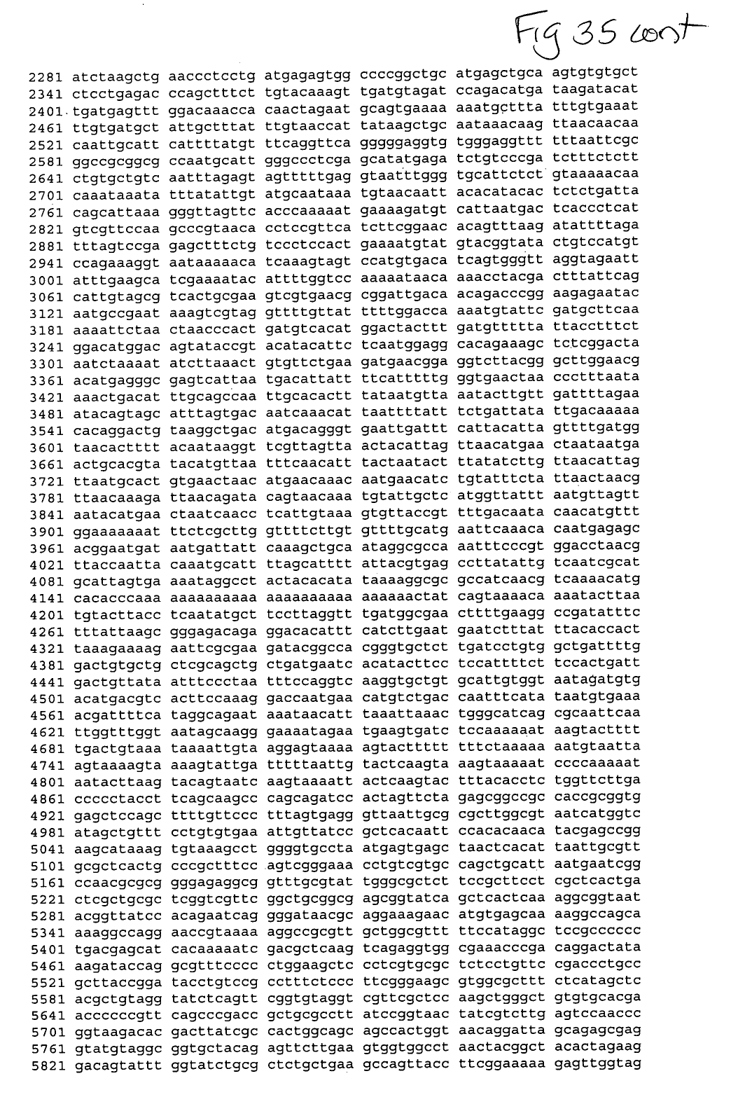

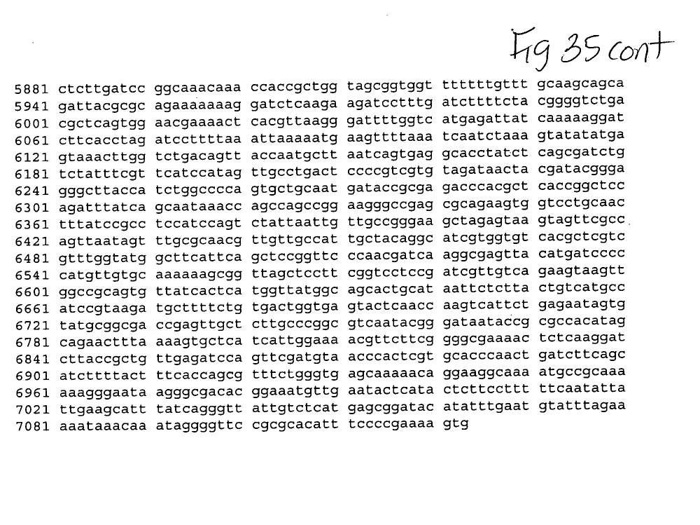

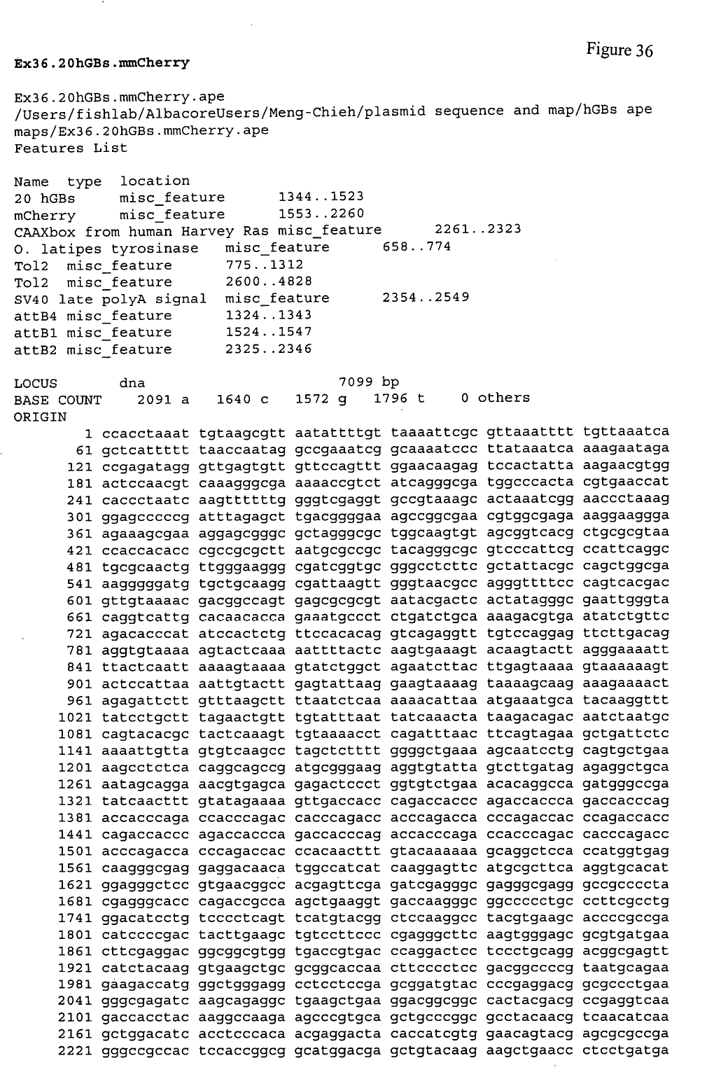

FIGS. 25-36: 5hGBs: five human Gli binding sites; 10hGBs: ten human Gli binding sites; 20hGBs: twenty human Gli binding sites.

These lines would allow high throughput screening for anti-cancer drugs that might block tumors that arise because of Hh mis-regulation. These include basal cell carcinoma (BCC) and certain brain tumors. High throughput screens of small molecules would be made possible by these lines. These lines would also allow a careful analysis of cellular defects that are associated with induced mis-regulation of Hh, and these studies might also lead to novel cancer treatments. These lines would also allow in-vivo, whole organism testing of pharmaceuticals designed to block Hh mediated tumorigenesis. We could monitor effectiveness in blocking Hh signaling, as well as effects on other tissues.

Hh signaling regulates stem cell proliferation in the brain and most likely muscle, as well as other tissues. Thus these lines will provide a model to see how Hh controls neural and myogenic stem cells that in turn might be important for treating neural and muscle degenerative diseases.

Finally, environmental pollutants might affect Hh signaling in the adult, leading to mis-regulation and cancer. These zebrafish transgenic lines could be used to see how different pollutants/compounds affect the ability to cells to respond to Hh activation or inactivation. The Hh reporter lines would allow a direct analysis of Hh activation in vivo after exposure to any compound, while the Hh activator and repressor lines could be used to begin the analysis of how these agents affect Hh signaling and where in the signaling pathway they alter the cellular response to Hh.

Since these lines should be useful for an analysis of Hh signaling from all vertebrate Hh molecules (Sonic Hh, Indian Hh, Desert Hh, echidna Hh, tiggywinkle Hh), they will be useful for the study of every tissue influenced by the Hh family of signaling molecules.

### Example 3

### Local Heatshock Tool

The purpose of a local heatshock tool is to apply a controlled temperature (˜37-42° C.) to a small region of a zebrafish embryo in order to activate genes controlled by the hsp70, or other heat shock, promoters. This device allows both temporal and spatial regulation of hsp70 controlled transgene expression throughout embryogenesis, as well as in larval, and adult stages. The heating probe is mounted on a micromanipulator attached to a dissecting microscope. Zebrafish embryos are mounted in low melt temperature agarose on a Petri dish, and the heating probe positioned using the micromanipulator near the tissue of interest. A digital thermometer mounted in agarose is used to monitor and calibrate the temperature at the probe tip.

The Heat shock device consists of a controller box, sensor and heating wires, and a heating probe. The control box holds a power supply, solid state relay, thermo controller device, and wires. The power supply was specifically chosen to match the resistance of the NiCr heating wires. An electronic current-limiting circuit block was then added to the heater circuit. This protects the power supply and the solid-state relay from damage if the heater leads happen to get shorted together. A tiny indicator was also added on the front panel of the control box that turns on when the Omega Thermo-Controller is calling for heat.

We purchased an Omega Thermo-Controller device (CNi16D) that is capable to monitor and control the temperature in a “heating probe”. It stays fixed in the controller box, and has numbered inputs, outputs, and voltage connections. In order to use the “PID-mode” (auto-mode) of the controller device, we used input #1 and #3 for the J-type T/C, and output #3 and #6 for the igniter wires. The T/Cs and igniter wires do not connect directly to the Omega Thermo-Controller, but through the relay (and wires) that are inside the control box.

We used J-type thermocouples to sense the heat at the probe tip, and igniter wires that provide the heat. The tip of the igniter wires is composed of alpha and beta particles. The alpha particle (located at the very tip) does not produce heat at all and is soldered to the beta particle. The only heating element is the beta particle coiled to the insulated igniter wire. When the beta particle is uncoiled it has a length of approximately 3 cm, which is enough to create a heating probe.

Since we want the “heating tip-probe” (the working beta end) to be physically as small as possible, the NiCr beta particle of the insulated igniter wire was uncoiled, and then bent to make a tiny naked coiled tip. A highly conductive room-temperature epoxy (OB-101-2) was used to attach the J-type thermocouple to the NiCr wire. Since the igniters were sold as a functional assembly for another purpose, we do not have the specs on the nichrome resistance wire that was used. This is why we used a micrometer on a piece of the NiCr itself and looked up the gauge in a reference and information AWG cable description—“American Wire Gauge”. The beta particle has (35AWG). The J-type thermocouple was than positioned to the nearest tip of the NiCr wire and then coated with a very thin layer of epoxy.

Since the control box was built to self-limit the current to a “safe-operating-area” for the driver circuitry components, an additional NiCr “piece” was added. If reducing the resistance in this way—the control box will still supply an amp+ of current. The additional NiCr piece (40 AWG) was then attached (coiled around) to the naked NiCr particle of the igniter wire. The length of the 40 AWG NiCr piece is 1.5 mm and has an oval tip (cut under the scope). A micropipette tip was finally used to attach the finished heating probe to a plastic syringe that serves as a body. The wires of the heating-tip-probe run through the syringe directly to the controller box.

### EQUIVALENTS

It will be apparent to those skilled in the art that the examples and embodiments described herein are by way of illustration and not of limitation, and that other examples may be used without departing from the spirit and scope of the present invention, as set forth in the claims.

1. A nucleic acid comprising a nucleotide sequence encoding (i) a Hedgehog (Hh) regulating protein; (ii) a heterologous peptide; wherein the nucleotide sequence is operably linked to an inducible promoter.

2. The nucleic acid of claim 1, wherein the Hh regulating protein is a Gli or PKA protein or a biologically active portion thereof.

3. The nucleic acid of claim 2, wherein the Hh regulating protein is a dominant repressor of Gli mediated Hh signaling.

4. The nucleic acid of claim 3, wherein the dominant repressor of Gli mediated Hh signaling is a C-terminally truncated Gli2 protein.

5. The nucleic acid of claim 2, wherein the Hh regulating protein is an activator of Hh signaling.

6. The nucleic acid of claim 5, wherein the activator of Hh signaling is an N-terminally truncated form of Gli2 or Gli3.

7. The nucleic acid of claim 5, wherein the activator of Hh signaling is a dominant negative form of protein kinase A (PKA).

8. The nucleic acid of claim 1, wherein the heterologous peptide is an enzyme.

9. (canceled)

10. The nucleic acid of claim 1, wherein the inducible promoter is a heat-shock promoter.

11. (canceled)

12. A vector comprising the nucleic acid of claim 1.

13. A cell comprising the nucleic acid of claim 1.

14. The cell of claim 13, which is a zebrafish cell.

15. An organism comprising the nucleic acid of claim 1.

16. The organism of claim 15, which is a zebrafish.

17. (canceled)

18. A zebrafish or cell thereof comprising a nucleic acid comprising (i) a nucleotide sequence comprising one or more Hh regulatory elements that are regulated by a regulatory protein, and (ii) a nucleotide sequence encoding a reporter protein.

19. The zebrafish or cell thereof of claim 18, wherein the one or more Hh regulatory elements are binding sites for Gli1.

20. The zebrafish or cell thereof of claim 18, wherein the one or more Hh regulatory elements is the DNA regulator region that regulates Patched (Ptc) expression.

21. (canceled)

22. A method for modulating Hh signaling response in a cell, comprising contacting a cell of claim 13 with an agent, or subjecting the cell to a condition, that induces the inducible promoter.

23. (canceled)

24. (canceled)

25. (canceled)

26. A method for identifying an agent that modulates Hh signaling, comprising (i) contacting a cell of claim 13 with a test agent; (ii) inducing the inducible promoter; and (iii) determining the level of Hh signaling; wherein a different level of Hh signaling in a cell contacted with a test agent relative to a cell that was not contacted with a test agent indicates that the test agent is an agent that modulates Hh signaling.

27. (canceled)

28. (canceled)

29. (canceled)

30. (canceled)

31. The method of claim 26 and 27, further comprising contacting the test agent with a cell or organism that is a model for a disease that is associated with an abnormal Hh regulation.

32. The method of claim 31, wherein the disease that is associated with an abnormal Hh regulation is cancer.

33. A method for determining whether an agent modulates Hh signaling in a cell, comprising (i) contacting a cell of claim 13 with an agent; (ii) inducing the inducible promoter; and (iii) determining the level of Hh signaling; wherein a different level of Hh signaling in a cell contacted with the agent relative to a cell that was not contacted with an agent indicates that the agent modulates Hh signaling in a cell.

34. (canceled)

35. (canceled)

36. A method for determining whether an agent modulates Hh signaling in a cell or organism, comprising (i) contacting a cell or organism comprising a nucleic acid comprising (a) a nucleotide sequence comprising one or more Hh regulatory elements that are regulated by the regulatory protein, and (b) a nucleotide sequence encoding a reporter protein, with an agent; and (ii) determining the level of expression of the reporter protein, wherein a different level of expression of the reporter protein in a cell or organism that was contacted with the agent relative to a cell or organism that was not contacted with the agent indicates that the agent modulates Hh signaling in a cell or organism.

37. The method of claim 36, wherein the agent is an environmental sample, and the method is for determining whether the agent is toxic to a cell or organism, wherein an agent is toxic to a cell or organism if the agent modulates Hh signaling.

38. A heating probe comprising:
an uncoiled portion of an igniter wire having a length of about 3 cm and bent to form a heating element;
a J-type thermocouple attached to an end of the igniter wire;
a controller connected to the heating element and thermocouple for limiting the temperature of the heating element based on heat sensed by the thermocouple;
a micropipette tip for attaching the heating element and thermocouple to a syringe, wherein wiring from the heating element and thermocouple pass through the syringe to the controller.
